(19) United States
(12) Patent Application Publication
(10) Pub. No.: US 2015/0118222 A1
(43) Pub. Date: Apr. 30, 2015

(54) TREATMENT USING BRUTON'S TYROSINE KINASE INHIBITORS AND IMMUNOTHERAPY

(71) Applicant: Pharmacyclics, Inc., Sunnyvale, CA (US)

(72) Inventors: Ronald LEVY, Stanford, CA (US); Betty CHANG, Cupertino, CA (US); Patrick NG, Sunnyvale, CA (US); Idit SAGIV-BARFI, Menlo Park, CA (US); Holbrook KOHRT, San Francisco, CA (US)

(21) Appl. No.: 14/523,782

(22) Filed: Oct. 24, 2014

Related U.S. Application Data
(60) Provisional application No. 61/895,988, filed on Oct. 25, 2013.
(60) Provisional application No. 61/899,764, filed on Nov. 4, 2013.
(60) Provisional application No. 61/911,953, filed on Dec. 4, 2013.
(60) Provisional application No. 61/937,392, filed on Feb. 7, 2014.
(60) Provisional application No. 61/968,312, filed on Mar. 20, 2014.
(60) Provisional application No. 62/023,742, filed on Jul. 11, 2014.
(60) Provisional application No. 62/023,705, filed on Jul. 11, 2014.

(51) Int. Cl. A61K 39/395; A61K 45/06; A61K 31/519

(52) U.S. Cl. CPC A61K 39/3955; A61K 31/519; A61K 45/06; A61K 2039/505
USPC 424/130.1

(57) ABSTRACT

Combinations of Bruton's tyrosine kinase (Btk) inhibitors, e.g., 1-((R)-3-(4-amino-3-(4-phenoxyphenyl)-1H-pyrazolo[3,4-d]pyrimidin-1-yl)piperidin-1-yl)prop-2-en-1-one, with immunotherapy are provided. Also provided are methods of treating cancers, and autoimmune disorders by administering combinations of Bruton's tyrosine kinase (Btk) inhibitors, e.g., 1-((R)-3-(4-amino-3-(4-phenoxyphenyl)-1H-pyrazolo[3,4-d]pyrimidin-1-yl)piperidin-1-yl)prop-2-en-1-one, and an immune checkpoint inhibitor.

### CROSS-REFERENCE

This application claims the benefit of priority of U.S. provisional patent application Nos. 61/895,988, filed on Oct. 25, 2013; 61/899,764, filed Nov. 4, 2013; 61/911,953, filed Dec. 4, 2013; 61/937,392, filed Feb. 7, 2014; 61/968,312, filed Mar. 20, 2014; 62/023,742, filed Jul. 11, 2014; and 62/023,705, filed Jul. 11, 2014, all of which are herein incorporated by reference in their entirety.

### BACKGROUND OF THE INVENTION

Bruton's tyrosine kinase (BTK), a member of the Tec family of non-receptor tyrosine kinases, is a key signaling enzyme expressed in all hematopoietic cells types except T lymphocytes and natural killer cells. Btk plays an essential role in the B-cell signaling pathway linking cell surface B-cell receptor (BCR) stimulation to downstream intracellular responses.

1-((R)-3-(4-amino-3-(4-phenoxyphenyl)-1H-pyrazolo[3,4-d]pyrimidin-1-yl)piperidin-1-yl)prop-2-en-1-one is also known by its IUPAC name as 1-{(3R)-3-[4-amino-3-(4-phenoxyphenyl)-1H-pyrazolo[3,4-d]pyrimidin-1-yl]piperidin-1-yl}prop-2-en-1-one or 2-Propen-1-one, 1-[(3R)-3-[4-amino-3-(4-phenoxyphenyl)-1H-pyrazolo[3,4-d]pyrimidin-1-yl]-1-piperidinyl-, and has been given the USAN name, Ibrutinib. The various names given for Ibrutinib are used interchangeably herein.

### SUMMARY OF THE INVENTION

Disclosed herein, in certain embodiments, is a use of a combination that comprises a BTK inhibitor and an immune checkpoint inhibitor for the treatment of a cancer. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1, PD-1, CTLA-4, LAG3, or TIM3. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the cancer is a hematologic cancer. In some embodiments, the hematologic cancer is a leukemia, a lymphoma, a myeloma, a non-Hodgkin's lymphoma, a Hodgkin's lymphoma, or a B-cell malignancy. In some embodiments, the hematologic cancer is a B-cell malignancy. In some embodiments, the B-cell malignancy is follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the B-cell malignancy is diffuse large B-cell lymphoma (DLBCL). In some embodiments, DLBCL is activated B-cell diffuse large B-cell lymphoma (ABC-DLBCL). In some embodiments, the B-cell malignancy is chronic lymphocytic leukemia (CLL), small lymphocytic lymphoma (SLL), B cell prolymphocytic leukemia (B-PLL), non-CLL/SLL lymphoma, mantle cell lymphoma, multiple myeloma, Waldenström's macroglobulinemia, or a combination thereof. In some embodiments, the B-cell malignancy is a relapsed or refractory B-cell malignancy. In some embodiments, the relapsed or refractory B-cell malignancy is diffuse large B-cell lymphoma (DLBCL). In some embodiments, the relapsed or refractory DLBCL is activated B-cell diffuse large B-cell lymphoma (ABC-DLBCL). In some embodiments, the relapsed or refractory B-cell malignancy is chronic lymphocytic leukemia (CLL), small lymphocytic lymphoma (SLL), B cell prolymphocytic leukemia (B-PLL), non-CLL/SLL lymphoma, mantle cell lymphoma, multiple myeloma, Waldenström's macroglobulinemia, or a combination thereof. In some embodiments, the B-cell malignancy is a metastasized B-cell malignancy. In some embodiments, the metastasized B-cell malignancy is diffuse large B-cell lymphoma (DLBCL), chronic lymphocytic leukemia (CLL), small lymphocytic lymphoma (SLL), B cell prolymphocytic leukemia (B-PLL), non-CLL/SLL lymphoma, mantle cell lymphoma, multiple myeloma, Waldenström's macroglobulinemia, or a combination thereof. In some embodiments, the cancer is a sarcoma, or carcinoma. In some embodiments, the cancer is selected from anal cancer; appendix cancer; bile duct cancer (i.e., cholangiocarcinoma); bladder cancer; breast cancer; cervical cancer; colon cancer; cancer of Unknown Primary (CUP); esophageal cancer; eye cancer; fallopian tube cancer; gastroenterological cancer; kidney cancer; liver cancer; lung cancer; medulloblastoma; melanoma; oral cancer; ovarian cancer; pancreatic cancer; parathyroid disease; penile cancer; pituitary tumor; prostate cancer; rectal cancer; skin cancer; stomach cancer; testicular cancer; throat cancer; thyroid cancer; uterine cancer; vaginal cancer; or vulvar cancer. In some embodiments, the cancer is selected from bladder cancer, breast cancer, colon cancer, gastroenterological cancer, kidney cancer, lung cancer, ovarian cancer, pancreatic cancer, prostate cancer, proximal or distal bile duct cancer, and melanoma. In some embodiments, the cancer is a breast cancer. In some embodiments, the breast cancer is ductal carcinoma in situ, lobular carcinoma in situ, invasive or infiltrating ductal carcinoma, invasive or infiltrating lobular carcinoma, inflammatory breast cancer, triple-negative breast cancer, paget disease of the nipple, phyllodes tumor, angiosarcoma or invasive breast carcinoma. In some embodiments, the cancer is a colon cancer. In some embodiments, the colon cancer is adenocarcinoma, gastrointestinal carcinoid tumors, gastrointestinal stromal tumors, primary colorectal lymphoma, leiomyosarcoma, melanoma, squamous cell-carcinoma, mucinous adenocarcinoma, or Signet ring cell adenocarcinoma. In some embodiments, the cancer is a relapsed or refractory cancer. In some embodiments, the relapsed or refractory cancer is selected from bladder cancer, breast cancer, colon cancer, gastroenterological cancer, kidney cancer, lung cancer, ovarian cancer, pancreatic cancer, prostate cancer, proximal or distal bile duct cancer, and melanoma. In some embodiments, the cancer is a metastasized cancer. In some embodiments, the metastasized cancer is selected from bladder cancer, breast cancer, colon cancer, gastroenterological cancer, kidney cancer, lung cancer, ovarian cancer, pancreatic cancer, prostate cancer, proximal or distal bile duct cancer, and melanoma. In some embodiments, the immune checkpoint inhibitor is an antibody. In some embodiments, the immune checkpoint inhibitor is a monoclonal antibody. In some embodiments, the BTK inhibitor is ibrutinib. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered orally. In some embodiments, ibrutinib and the immune checkpoint inhibitor are administered simultaneously, sequentially or intermittently. In some embodiments, the use of a combination comprising a BTK inhibitor and an immune checkpoint inhibitor for the treatment of a cancer further comprises administering an additional anticancer agent. In some embodiments, the additional anticancer agent is selected from among a chemotherapeutic agent or radiation therapy. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

Disclosed herein, in certain embodiments, is a pharmaceutical combination that comprises (a) a BTK inhibitor; and (b) an immune checkpoint inhibitor; and (c) a pharmaceutically-acceptable excipient. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1, PD-1, CTLA-4, LAG3, or TIM3. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the immune checkpoint inhibitor is an antibody. In some embodiments, the immune checkpoint inhibitor is a monoclonal antibody. In some embodiments, the BTK inhibitor is ibrutinib. In some embodiments, the combination is in a combined dosage form. In some embodiments, the combination is in separate dosage forms. In some embodiments, the pharmaceutical combination further comprises an additional anticancer agent.

Disclosed herein, in certain embodiments, is a use of a combination that comprises ibrutinib and an immune checkpoint inhibitor for the treatment of an ibrutinib-resistant cancer. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1, PD-1, CTLA-4, LAG3, or TIM3. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the ibrutinib-resistant cancer is a hematologic cancer. In some embodiments, the hematologic cancer is a leukemia, a lymphoma, a myeloma, a non-Hodgkin's lymphoma, a Hodgkin's lymphoma, or a B-cell malignancy. In some embodiments, the hematologic cancer is a B-cell malignancy. In some embodiments, the B-cell malignancy is follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the B-cell malignancy is diffuse large B-cell lymphoma (DLBCL). In some embodiments, DLBCL is activated B-cell diffuse large B-cell lymphoma (ABC-DLBCL). In some embodiments, the B-cell malignancy is chronic lymphocytic leukemia (CLL), small lymphocytic lymphoma (SLL), B cell prolymphocytic leukemia (B-PLL), non-CLL/SLL lymphoma, mantle cell lymphoma, multiple myeloma, Waldenström's macroglobulinemia, or a combination thereof. In some embodiments, the B-cell malignancy is a relapsed or refractory B-cell malignancy. In some embodiments, the relapsed or refractory B-cell malignancy is diffuse large B-cell lymphoma (DLBCL). In some embodiments, the relapsed or refractory DLBCL is activated B-cell diffuse large B-cell lymphoma (ABC-DLBCL). In some embodiments, the relapsed or refractory B-cell malignancy is chronic lymphocytic leukemia (CLL), small lymphocytic lymphoma (SLL), B cell prolymphocytic leukemia (B-PLL), non-CLL/SLL lymphoma, mantle cell lymphoma, multiple myeloma, Waldenström's macroglobulinemia, or a combination thereof. In some embodiments, the B-cell malignancy is a metastasized B-cell malignancy. In some embodiments, the metastasized B-cell malignancy is diffuse large B-cell lymphoma (DLBCL), chronic lymphocytic leukemia (CLL), small lymphocytic lymphoma (SLL), B cell prolymphocytic leukemia (B-PLL), non-CLL/SLL lymphoma, mantle cell lymphoma, multiple myeloma, Waldenström's macroglobulinemia, or a combination thereof. In some embodiments, the ibrutinib-resistant cancer is a sarcoma, or carcinoma. In some embodiments, the ibrutinib-resistant cancer is selected from anal cancer; appendix cancer; bile duct cancer (i.e., cholangiocarcinoma); bladder cancer; breast cancer; cervical cancer; colon cancer; cancer of Unknown Primary (CUP); esophageal cancer; eye cancer; fallopian tube cancer; gastroenterological cancer; kidney cancer; liver cancer; lung cancer; medulloblastoma; melanoma; oral cancer; ovarian cancer; pancreatic cancer; parathyroid disease; penile cancer; pituitary tumor; prostate cancer; rectal cancer; skin cancer; stomach cancer; testicular cancer; throat cancer; thyroid cancer; uterine cancer; vaginal cancer; or vulvar cancer. In some embodiments, the ibrutinib-resistant cancer is selected from bladder cancer, breast cancer, colon cancer, gastroenterological cancer, kidney cancer, lung cancer, ovarian cancer, pancreatic cancer, prostate cancer, proximal or distal bile duct cancer, and melanoma. In some embodiments, the ibrutinib-resistant cancer is a breast cancer. In some embodiments, the breast cancer is ductal carcinoma in situ, lobular carcinoma in situ, invasive or infiltrating ductal carcinoma, invasive or infiltrating lobular carcinoma, inflammatory breast cancer, triple-negative breast cancer, paget disease of the nipple, phyllodes tumor, angiosarcoma or invasive breast carcinoma. In some embodiments, the ibrutinib-resistant cancer is a colon cancer. In some embodiments, the colon cancer is adenocarcinoma, gastrointestinal carcinoid tumors, gastrointestinal stromal tumors, primary colorectal lymphoma, leiomyosarcoma, melanoma, squamous cell-carcinoma, mucinous adenocarcinoma, or Signet ring cell adenocarcinoma. In some embodiments, the ibrutinib-resistant cancer is a relapsed or refractory cancer. In some embodiments, the relapsed or refractory cancer is selected from bladder cancer, breast cancer, colon cancer, gastroenterological cancer, kidney cancer, lung cancer, ovarian cancer, pancreatic cancer, prostate cancer, proximal or distal bile duct cancer, and melanoma. In some embodiments, the ibrutinib-resistant cancer is a metastasized cancer. In some embodiments, the metastasized cancer is selected from bladder cancer, breast cancer, colon cancer, gastroenterological cancer, kidney cancer, lung cancer, ovarian cancer, pancreatic cancer, prostate cancer, proximal or distal bile duct cancer, and melanoma. In some embodiments, the immune checkpoint inhibitor is an antibody. In some embodiments, the immune checkpoint inhibitor is a monoclonal antibody. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered orally. In some embodiments, ibrutinib and the immune checkpoint inhibitor are administered simultaneously, sequentially or intermittently. In some embodiments, the use of a combination comprising ibrutinib and an immune checkpoint inhibitor further comprises administering an additional anticancer agent. In some embodiments, the additional anticancer agent is selected from among a chemotherapeutic agent or radiation therapy. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

Disclosed herein, in certain embodiments, is a use of a combination that comprises a BTK inhibitor and an immune checkpoint inhibitor for increasing the Th1:Th2 biomarker ratio in a cancer patient, wherein the combination decreases the Th2 response in the cancer patient and increases the Th1 response in the cancer patient. In some embodiments, the cancer is characterized by a biomarker profile in which the Th1 response is suppressed and the Th2 response is enhanced. In some embodiments, the use of a combination comprising a BTK inhibitor and an immune checkpoint inhibitor further comprises measuring the expression of one or more Th1 or Th2 biomarkers in the subject prior to administering the combination comprising ibrutinib and an immune checkpoint inhibitor. In some embodiments, the Th2 biomarker is selected from among IL-10, IL-4, IL-13, or a combination thereof. In some embodiments, the Th1 biomarker is selected from among IFN-γ, IL-2, IL-12, or a combination thereof. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1, PD-1, CTLA-4, LAG3, or TIM3. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the cancer is a hematologic cancer. In some embodiments, the hematologic cancer is a leukemia, a lymphoma, a myeloma, a non-Hodgkin's lymphoma, a Hodgkin's lymphoma, or a B-cell malignancy. In some embodiments, the hematologic cancer is a B-cell malignancy. In some embodiments, the B-cell malignancy is follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the B-cell malignancy is diffuse large B-cell lymphoma (DLBCL). In some embodiments, DLBCL is activated B-cell diffuse large B-cell lymphoma (ABC-DLBCL). In some embodiments, the B-cell malignancy is chronic lymphocytic leukemia (CLL), small lymphocytic lymphoma (SLL), B cell prolymphocytic leukemia (B-PLL), non-CLL/SLL lymphoma, mantle cell lymphoma, multiple myeloma, Waldenström's macroglobulinemia, or a combination thereof. In some embodiments, the B-cell malignancy is a relapsed or refractory B-cell malignancy. In some embodiments, the relapsed or refractory B-cell malignancy is diffuse large B-cell lymphoma (DLBCL). In some embodiments, the relapsed or refractory DLBCL is activated B-cell diffuse large B-cell lymphoma (ABC-DLBCL). In some embodiments, the relapsed or refractory B-cell malignancy is chronic lymphocytic leukemia (CLL), small lymphocytic lymphoma (SLL), B cell prolymphocytic leukemia (B-PLL), non-CLL/SLL lymphoma, mantle cell lymphoma, multiple myeloma, Waldenström's macroglobulinemia, or a combination thereof. In some embodiments, the B-cell malignancy is a metastasized B-cell malignancy. In some embodiments, the metastasized B-cell malignancy is diffuse large B-cell lymphoma (DLBCL), chronic lymphocytic leukemia (CLL), small lymphocytic lymphoma (SLL), B cell prolymphocytic leukemia (B-PLL), non-CLL/SLL lymphoma, mantle cell lymphoma, multiple myeloma, Waldenström's macroglobulinemia, or a combination thereof. In some embodiments, the cancer is a sarcoma or carcinoma. In some embodiments, the cancer is selected from anal cancer; appendix cancer; bile duct cancer (i.e., cholangiocarcinoma); bladder cancer; breast cancer; cervical cancer; colon cancer; cancer of Unknown Primary (CUP); esophageal cancer; eye cancer; fallopian tube cancer; gastroenterological cancer; kidney cancer; liver cancer; lung cancer; medulloblastoma; melanoma; oral cancer; ovarian cancer; pancreatic cancer; parathyroid disease; penile cancer; pituitary tumor; prostate cancer; rectal cancer; skin cancer; stomach cancer; testicular cancer; throat cancer; thyroid cancer; uterine cancer; vaginal cancer; or vulvar cancer. In some embodiments, the cancer is selected from bladder cancer, breast cancer, colon cancer, gastroenterological cancer, kidney cancer, lung cancer, ovarian cancer, pancreatic cancer, prostate cancer, proximal or distal bile duct cancer, and melanoma. In some embodiments, the cancer is a breast cancer. In some embodiments, the breast cancer is ductal carcinoma in situ, lobular carcinoma in situ, invasive or infiltrating ductal carcinoma, invasive or infiltrating lobular carcinoma, inflammatory breast cancer, triple-negative breast cancer, paget disease of the nipple, phyllodes tumor, angiosarcoma or invasive breast carcinoma. In some embodiments, the cancer is a colon cancer. In some embodiments, the colon cancer is adenocarcinoma, gastrointestinal carcinoid tumors, gastrointestinal stromal tumors, primary colorectal lymphoma, leiomyosarcoma, melanoma, squamous cell-carcinoma, mucinous adenocarcinoma, or Signet ring cell adenocarcinoma. In some embodiments, the cancer is a relapsed or refractory cancer. In some embodiments, the relapsed or refractory cancer is selected from bladder cancer, breast cancer, colon cancer, gastroenterological cancer, kidney cancer, lung cancer, ovarian cancer, pancreatic cancer, prostate cancer, proximal or distal bile duct cancer, and melanoma. In some embodiments, the cancer is a metastasized cancer. In some embodiments, the metastasized cancer is selected from bladder cancer, breast cancer, colon cancer, gastroenterological cancer, kidney cancer, lung cancer, ovarian cancer, pancreatic cancer, prostate cancer, proximal or distal bile duct cancer, and melanoma. In some embodiments, the immune checkpoint inhibitor is an antibody. In some embodiments, the immune checkpoint inhibitor is a monoclonal antibody. In some embodiments, the BTK inhibitor is ibrutinib. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered orally. In some embodiments, ibrutinib and the immune checkpoint inhibitor are administered simultaneously, sequentially or intermittently. In some embodiments, the use of a combination comprising a BTK inhibitor and an immune checkpoint inhibitor further comprises administering an additional anticancer agent. In some embodiments, the additional anticancer agent is selected from among a chemotherapeutic agent or radiation therapy. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

Disclosed herein, in certain embodiments, is a use of a combination that comprises a BTK inhibitor and an immune checkpoint inhibitor for treating a breast cancer. In some embodiments, the breast cancer is ductal carcinoma in situ, lobular carcinoma in situ, invasive or infiltrating ductal carcinoma, invasive or infiltrating lobular carcinoma, inflammatory breast cancer, triple-negative breast cancer, paget disease of the nipple, phyllodes tumor, angiosarcoma or invasive breast carcinoma. In some embodiments, the breast cancer is a relapsed or refractory breast cancer. In some embodiments, the breast cancer is a metastasized breast cancer. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1, PD-1, CTLA-4, LAG3, or TIM3. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the immune checkpoint inhibitor is an antibody. In some embodiments, the immune checkpoint inhibitor is a monoclonal antibody. In some embodiments, the BTK inhibitor is ibrutinib. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered orally. In some embodiments, ibrutinib and the immune checkpoint inhibitor are administered simultaneously, sequentially or intermittently. In some embodiments, the use of a combination comprising a BTK inhibitor and an immune checkpoint inhibitor further comprises administering an additional anticancer agent. In some embodiments, the additional anticancer agent is selected from among a chemotherapeutic agent or radiation therapy. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

Disclosed herein, in certain embodiments, is a use of a combination that comprises a BTK inhibitor and an immune checkpoint inhibitor for treating a colon cancer. In some embodiments, the colon cancer is adenocarcinoma, gastrointestinal carcinoid tumors, gastrointestinal stromal tumors, primary colorectal lymphoma, leiomyosarcoma, melanoma, squamous cell-carcinoma, mucinous adenocarcinoma, or Signet ring cell adenocarcinoma. In some embodiments, the colon cancer is a relapsed or refractory colon cancer. In some embodiments, the colon cancer is a metastasized colon cancer. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1, PD-1, CTLA-4, LAG3, or TIM3. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the immune checkpoint inhibitor is an antibody. In some embodiments, the immune checkpoint inhibitor is a monoclonal antibody. In some embodiments, the BTK inhibitor is ibrutinib. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered orally. In some embodiments, ibrutinib and the immune checkpoint inhibitor are administered simultaneously, sequentially or intermittently. In some embodiments, the use of a combination comprising a BTK inhibitor and an immune checkpoint inhibitor further comprises administering an additional anticancer agent. In some embodiments, the additional anticancer agent is selected from among a chemotherapeutic agent or radiation therapy. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

Disclosed herein, in certain embodiments, is a use of a combination that comprises a BTK inhibitor and an immune checkpoint inhibitor for treating a diffuse large B-cell lymphoma (DLBCL). In some embodiments, DLBCL is activated B-cell diffuse large B-cell lymphoma (ABC-DLBCL). In some embodiments, DLBCL is a relapsed or refractory DLBCL. In some embodiments, DLBCL is a metastasized DLBCL. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1, PD-1, CTLA-4, LAG3, or TIM3. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the immune checkpoint inhibitor is an antibody. In some embodiments, the immune checkpoint inhibitor is a monoclonal antibody. In some embodiments, the BTK inhibitor is ibrutinib. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered orally. In some embodiments, ibrutinib and the immune checkpoint inhibitor are administered simultaneously, sequentially or intermittently. In some embodiments, the use of a combination comprising a BTK inhibitor and an immune checkpoint inhibitor further comprises administering an additional anticancer agent. In some embodiments, the additional anticancer agent is selected from among a chemotherapeutic agent or radiation therapy. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

### BRIEF DESCRIPTION OF THE FIGURES

FIG. 1 exemplifies an ibrutinib and anti-PD-L1 antibody administration schedule in a mouse model injected with A20 (ibrutinib resistant) cell line on two sides of the abdomen. Ibrutinib was administered on days 8-15 post injection of A20 cells. Anti-PD-L1 antibody was administered on days 8, 10 and 13 post injection of A20 cells, while anti-CTLA-4 antibody was administered on days 8 and 12 post injection of A20 cells. Blood was drawn on day 16 post injection.

FIG. 2A exemplifies tumor volume from non-treated control mice after injection with A20 cells. FIG. 2B exemplifies mean tumor volume from non-treated control mice after injection with A20 cells.

FIG. 3A-B exemplify tumor volume (A) and mean tumor volume (B) from mice treated with anti-PD-L1 antibody alone after injection with A20 cells.

FIG. 4A-B exemplify tumor volume (A) and mean tumor volume (B) from mice treated with a combination of ibrutinib and anti-PD-L1 antibody after injection with A20 cells.

FIG. 5A-B exemplify tumor volume (A) and mean tumor volume (B) from mice treated with a combination of ibrutinib and anti-CTLA-4 antibody after injection with A20 cells.

FIG. 6A-D exemplify expression of PD-1 and/or PDL-1 in follicular lymphoma (FL) patients treated with ibrutinib. Generally, no effect on PDL-1 expression was observed in lymphoma cells treated with ibrutinib (B). Some FL patients treated with ibrutinib were found to have increased PD-1 levels on their CD8+ T-cells (D) but not on FL B cells (A) or CD4+ T-cells (C). Generally, PD-1 levels of patients treated with ibrutinib were not decreased. The anti-PD-L1 antibody used was the antibody clone MIH1. The anti-PD-1 antibody used was the antibody clone MIH4. Accordingly, because PD-1 or PDL-1 levels in follicular lymphoma patients were not decreased, it is expected that human follicular lymphoma patients would benefit from combining anti-PD1/PDL1 with ibrutinib.

FIG. 7 exemplifies mean tumor volume from mice treated with a combination of ibrutinib and anti-PD1/PDL1 antibody after injection with TMD8 (ABC-DLBCL) cells. The combination of ibrutinib and anti-PD1/PD-L1 therapy was found to have a synergistic effect in reducing tumor volume as compared to treatment with ibrutinib or anti-PD1/PD-L1 antibody alone.

FIG. 8A and FIG. 8B-D exemplify tumor volume from mice treated with a combination of ibrutinib and anti-PD1/PDL1 antibody. FIG. 8A illustrates the tumor volume from mice treated with vehicle+IgG. FIG. 8B illustrates the tumor volume from mice treated with vehicle and anti-PD1+anti-PD-L1. FIG. 8C illustrates the tumor volume from mice treated with ibrutinib (PCI-32765)+IgG. FIG. 8D illustrates the tumor volume from mice treated with ibrutinib (PCI-32765) and anti-PD1+anti-PD-L1.

FIG. 9A-B exemplify the upregulation of PD-L1 levels in cancer patients (CLL, CLL/PLL and CLL/SLL) resistant to ibrutinib alone. The level of PD-L1 was observed to be upregulated in patients resistant to ibrutinib (A; B represents the same data as A but with expanded y-axis).

FIG. 10A-C, FIG. 10D-F, and FIG. 10G-I exemplify the upregulation of PD1 levels in cancer patients (CLL, CLL/PLL and CLL/SLL) resistant to ibrutinib alone. The level of PD1 was observed to be upregulated in patients resistant to ibrutinib.

FIG. 11A-B exemplify treatment of ibrutinib in combination with anti-PD-1/PD-L1 in a mouse tumor model. Panel A exemplifies mean tumor volume from mice after injection with A20 cells. Panel B exemplifies percentage survival rate of mice after injection with A20 cells.

FIG. 12A-C, FIG. 12D-F, and FIG. 12G exemplify tumor volume of mice after injection of A20 cells. Panel A exemplifies tumor volume from non-treated (N/T) control group. Panel B exemplifies tumor volume from IC control group. Panel C exemplifies tumor volume from ibrutinib alone group. Panel D exemplifies tumor volume from anti-PD-1 alone group. Panel E exemplifies tumor volume from anti-PD-L1 alone group. Panel F exemplifies tumor volume from ibrutinib and anti-PD-1 group. FIG. 12G exemplifies tumor volume from ibrutinib and anti-PD-L1 group.

FIG. 13A-B exemplify treatment of ibrutinib in combination with two different concentrations of anti-PD-L1 in a mouse tumor model. Panel A exemplifies mean tumor volume from mice after injection with A20 cells. Panel B exemplifies percentage survival rate of mice after injection with A20 cells.

FIG. 14A-D and FIG. 14E-F exemplify tumor volume of mice after injection of A20 cells. Panel A exemplifies tumor volume from non-treated (N/T) control group. Panel B exemplifies tumor volume from ibrutinib alone group. Panel C exemplifies tumor volume from 100 μg of anti-PD-L1 group. Panel D exemplifies tumor volume from 200 μg of anti-PD-L1 group. Panel E exemplifies tumor volume from ibrutinib and 100 μg of anti-PD-L1 group. Panel F exemplifies tumor volume from ibrutinib and 200 μg of anti-PD-L1 group.

FIG. 15A-X illustrate flow cytometry analysis of CD8+ T cells with ibrutinib or ibrutinib and anti-PD-L1 treatments. Cells were either not treated (A-D) or pretreated with the indicated concentration of ibrutinib (E-H), anti-PD-L1 at 100 μg (1-L) or 200 μg (M-P) or ibrutinib and anti-PD-L1 (Q-T at 100 μg anti-PD-L1; U—X at 200 μg anti-PD-L1) and were either stimulated (or unstimulated) with anti-CD3/anti-CD28 or were irradiated. Percentages are represented in each quadrant.

FIG. 16A-X illustrate flow cytometry analysis of CD4+ T cells with ibrutinib or ibrutinib and anti-PD-L1 treatments. Cells were either not treated (A-D) or pretreated with the indicated concentration of ibrutinib (E-H), anti-PD-L1 at 100 μg (1-L) or 200 μg (M-P) or ibrutinib and anti-PD-L1 (Q-T at 100 μg anti-PD-L1; U—X at 200 μg anti-PD-L1) and were either stimulated (or unstimulated) with anti-CD3/anti-CD28 or were irradiated. Percentages are represented in each quadrant.

FIG. 17A-B exemplify treatment of ibrutinib in combination with anti-PD-L1 in a mouse tumor model. Panel A exemplifies mean tumor volume from mice after injection with 4T1 cells. Panel B exemplifies percentage survival rate of mice after injection with 4T1 cells.

FIG. 18A-B and FIG. 18C-D exemplifies tumor volume of mice after injection of 4T1 cells. Panel A exemplifies tumor volume from non-treated (N/T) control group. Panel B exemplifies tumor volume from ibrutinib alone group. Panel C exemplifies tumor volume from anti-PD-L1 alone group. Panel D exemplifies tumor volume from ibrutinib and anti-PD-L1 group.

FIG. 19A-C and FIG. 19D illustrate the combination of anti-PD-L1 and ibrutinib in A20 mouse lymphoma model. Panel A exemplifies a gel expression of Btk. Panel B illustrates the IC₅₀ of ibrutinib is greater than 10 μM. Panel C illustrates the locations of the A20 tumors in non-treated and ibrutinib alone groups. FIG. 19D illustrates the mean tumor volume from non-treated and ibrutinib alone mice after injection with A20 cells.

FIG. 20A-B illustrate a first set of experiments using the 4T1 breast cancer model. Panel A exemplifies an ibrutinib and anti-PD-L1 antibody administration schedule in a mouse model injected with 4T1-Luc (0.05×10⁶) cells into the right side of the mouse abdomen. Ibrutinib was administered at 6 mg/kg on days 6-20 post injection of 4T1-Luc cells. Anti-PD-L1 (200 μg) was administered on days 6, 8, 11, 13, 15 and 18 post-injection of 4T1-Luc cells. The 4T1 cell line is a model of triple negative breast cancer, and it is not sensitive to ibrutinib. After about 3-4 weeks of injection, the breast cancer metastasizes to the lung. Panel B illustrates the mean tumor volume from non-treated, Ibrutinib alone, anti-PD-L1 alone, and Ibrutinib+anti-PD-L1 mice after injection with 4T1-Luc cells.

FIG. 21A-D exemplify the tumor volume from non-treated, Ibrutinib alone, anti-PD-L1 alone, and Ibrutinib+anti-PD-L1 mice after injection with 4T1-Luc cells.

FIG. 22A-B illustrate a second set of experiments using the 4T1 breast cancer model. Panel A exemplifies an ibrutinib and anti-PD-L1 antibody administration schedule in a mouse model injected with 4T1-Luc (0.01×10⁶) cells into the right side of the mouse abdomen. Ibrutinib was administered at 6 mg/kg on days 6-20 post injection of 4T1-Luc cells. Anti-PD-L1 (200 μg) was administered on days 6, 8, 11, 13, 15 and 18 post-injection of 4T1-Luc cells. The 4T1 cell line is a model of triple negative breast cancer, and it is not sensitive to ibrutinib. After about 3-4 weeks of injection, the breast cancer metastasizes to the lung. Panel B illustrates the mean tumor volume from non-treated, Ibrutinib alone, anti-PD-L1 alone, Ibrutinib+anti-PD-L1, and ibrutinib+anti-PD-L1 (started 3 days later) mice after injection with 4T1-Luc cells.

FIG. 23 exemplifies lung metastasis, bioluminescence imaging, and subcutaneous tumor growth for control (vehicle) group, ibrutinib alone group, anti-PD-L1 group, and ibrutinib+anti-PD-L1 group. The combination of ibrutinib and anti-PD-L1 effectively inhibits primary tumor growth and lung metastasis in a syngeneic 4T1 model.

FIG. 24 exemplifies the number of lung metastasis in non-treated, Ibrutinib alone, anti-PD-L1 alone, Ibrutinib+anti-PD-L1, and ibrutinib+anti-PD-L1 (started 3 days later) mice after injection with 4T1-Luc cells.

FIG. 25A-B illustrate a third set of experiment using the 4T1 breast cancer model. Panel A exemplifies an ibrutinib and anti-PD-L1 antibody administration schedule in a mouse model injected with 4T1-Luc (0.05×10⁶) cells into the right side of the mouse abdomen. Ibrutinib was administered at 6 mg/kg on days 6-20 post injection of 4T1-Luc cells. Anti-PD-L1 (200 μg) was administered on days 6, 8, 11, 13, 15 and 18 post-injection of 4T1-Luc cells. The 4T1 cell line is a model of triple negative breast cancer, and it is not sensitive to ibrutinib. After about 3-4 weeks of injection, the breast cancer metastasizes to the lung. Panel B illustrates the mean tumor volume from non-treated, Ibrutinib alone, anti-PD-L1 alone, and Ibrutinib+anti-PD-L1 mice after injection with 4T1-Luc cells.

FIG. 26A-D exemplify the tumor volume from non-treated, Ibrutinib alone, anti-PD-L1 alone, and Ibrutinib+anti-PD-L1 mice after injection with 4T1-Luc cells.

FIG. 27A-FIG. 27D exemplify bioluminescence imaging from non-treated, Ibrutinib alone, anti-PD-L1 alone, and Ibrutinib+anti-PD-L1 mice after injection with 4T1-Luc cells.

FIG. 28 exemplifies the number of lung metastasis in non-treated, Ibrutinib alone, anti-PD-L1 alone, and Ibrutinib+anti-PD-L1 mice after injection with 4T1-Luc cells.

FIG. 29A-B illustrate a first set of experiment using the CT26 colon cancer model. Panel A exemplifies an ibrutinib and anti-PD-L1 antibody administration schedule in a mouse model injected with CT26 (1×10⁶) cells into the right side of the mouse abdomen. Ibrutinib was administered at 6 mg/kg on days 5-20 post injection of CT26 cells. Anti-PD-L1 (200 μg) was administered on days 5, 7, 10, 12, 14, and 17 post-injection of CT26 cells. The CT26 cell line is not sensitive to ibrutinib. Panel B illustrates the mean tumor volume from non-treated, Ibrutinib alone, anti-PD-L1 alone, and Ibrutinib+anti-PD-L1 mice after injection with CT26 cells.

FIG. 30A-D exemplify the tumor volume from non-treated, Ibrutinib alone, anti-PD-L1 alone, and Ibrutinib+anti-PD-L1 mice after injection with CT26 cells.

FIG. 31A illustrates a second set of experiment using the CT26 colon cancer model. FIG. 31A exemplifies an ibrutinib and anti-PD-L1 antibody administration schedule in a mouse model injected with CT26 (0.5×10⁶) cells into the right side of the mouse abdomen. Ibrutinib was administered at 6 mg/kg on days 5-20 post injection of CT26 cells. Anti-PD-L1 (200 μg) was administered on days 5, 7, 10, 12, 14, and 17 post-injection of CT26 cells. The CT26 cell line is not sensitive to ibrutinib. FIG. 31B exemplifies the tumor volume and tumor location from non-treated, Ibrutinib alone, anti-PD-L1 alone, and Ibrutinib+anti-PD-L1 mice after injection with CT26 cells. FIG. 31C exemplifies the mean tumor volume from non-treated, Ibrutinib alone, anti-PD-L1 alone, and Ibrutinib+anti-PD-L1 mice after injection with CT26 cells. FIG. 31D exemplifies the percent survival from non-treated, Ibrutinib alone, anti-PD-L1 alone, and Ibrutinib+anti-PD-L1 mice after injection with CT26 cells.

FIG. 32A-B exemplify a third set of experiment using the CT26 colon cancer model. FIG. 32A exemplifies an ibrutinib and anti-PD-L1 antibody administration schedule in a mouse model injected with CT26 (0.5×10⁶) cells into the right side of the mouse abdomen. Ibrutinib was administered at 6 mg/kg on days 5-20 post injection of CT26 cells. Anti-PD-L1 (200 μg) and anti-PD-1 (200 μg) were administered on days 5, 7, 10, 12, 14, and 17 post-injection of CT26 cells. The CT26 cell line is not sensitive to ibrutinib. FIG. 32B exemplifies the mean tumor volume from non-treated, anti-PD-1 alone, anti-PD-L1 alone, Ibrutinib+anti-PD-L1, and ibrutinib+anti-PD-1 mice after injection with CT26 cells.

FIG. 33 exemplifies the tumor volume from non-treated, ibrutinib alone, anti-PD-1 alone, anti-PD-L1 alone, Ibrutinib+anti-PD-L1, and ibrutinib+anti-PD-1 mice after injection with CT26 cells.

FIG. 34A-B exemplify a fourth set of experiment using the CT26 colon cancer model. Panel A exemplifies an ibrutinib and anti-PD-L1 antibody administration schedule in a mouse model injected with CT26 (0.5×10⁶) cells into the right side of the mouse abdomen. Ibrutinib was administered at 6 mg/kg on days 5-20 post injection of CT26 cells. Anti-PD-L1 (100 μg or 50 ng) was administered on days 5, 7, 10, 12, 14, and 17 post-injection of CT26 cells. The CT26 cell line is not sensitive to ibrutinib. Panel B exemplifies the mean tumor volume from non-treated, anti-PD-L1 alone at 100 μg, anti-PD-L1 alone at 50 μg, Ibrutinib+anti-PD-L1 (100 μg), and ibrutinib+anti-PD-L1 (50 μg) mice after injection with CT26 cells.

FIG. 35A-C and FIG. 35D-E exemplify the tumor volume from non-treated, anti-PD-L1 alone at 100 μg, anti-PD-L1 alone at 50 μg, Ibrutinib+anti-PD-L1 (100 μg), and ibrutinib+anti-PD-L1 (50 μg) mice after injection with CT26 cells.

FIG. 36A-B exemplify tumor volumes in mice after injection with CT26 cells. Panel A exemplifies the mean tumor volume from non-treated, anti-PD-L1 alone at 100 μg, anti-PD-L1 alone at 50 μg, Ibrutinib+anti-PD-L1 (100 μg), and ibrutinib+anti-PD-L1 (50 μg) mice after injection with CT26 cells. Panel B exemplifies the percent survival from non-treated, anti-PD-L1 alone at 100 μg, anti-PD-L1 alone at 50 μg, Ibrutinib+anti-PD-L1 (100 μg), and ibrutinib+anti-PD-L1 (50 μg) mice after injection with CT26 cells.

FIG. 37A-C and FIG. 37D-E exemplify the tumor volume from non-treated, anti-PD-L1 alone at 100 μg, anti-PD-L1 alone at 50 μg, Ibrutinib+anti-PD-L1 (100 μg), and ibrutinib+anti-PD-L1 (50 μg) mice after injection with CT26 cells.

FIG. 38 illustrates the flow cytometry analysis of CD8+ T cells with ibrutinib. Cells were either non treated or pretreated with ibrutinib and were stimulated (or unstimulated) with anti-CD3/anti-CD28. Percentages are represented in each quadrant.

FIG. 39 illustrates the flow cytometry analysis of CD8+ T cells with anti-PD-L1 alone or ibrutinib+anti-PD-L1. Cells were either pretreated with anti-PD-L1 alone or with ibrutinib+anti-PD-L1 and were stimulated (or unstimulated) with anti-CD3/anti-CD28. Percentages are represented in each quadrant.

FIG. 40A-B illustrate IFN-γ-expressing T_{eff} cells analysis with non-treated, Ibrutinib alone, anti-PD-L1 alone, and Ibrutinib+anti-PD-L1 in CD8 and CD4 T cells.

FIG. 41A-C illustrate the percentage of antigen specific T cells from treatment with non-treated, Ibrutinib alone, anti-PD-L1 alone, and Ibrutinib+anti-PD-L1 in CD8, CD4 and CD4+/CD25+ T cells in spleen, blood, and tumor.

FIG. 42A-B and FIG. 42C exemplify tumor volume from mice injected with 1 million (42A), 5 million (42B), and 10 million (42C), CT26 tumor cells.

FIG. 43A and FIG. 43B-C exemplify tumor volumes from mice treated with IgG alone (A), or in combination with ibrutinib, according to schedule 1 (B), or schedule 2 (C).

FIG. 44A-B and FIG. 44C exemplify tumor volumes from mice treated with anti-PD-L1 antibody alone (A), or in combination with ibrutinib, according to schedule 1 (B), or schedule 2 (C).

FIG. 45A and FIG. 45B-C exemplify tumor volumes from mice treated with anti-CTLA-4 antibody alone (A), or in combination with ibrutinib, according to schedule 1 (B), or schedule 2 (C).

FIG. 46A-B exemplify tumor volumes from mice treated with a combination of anti-PD-L1, and anti-CTLA-4 antibody (A), or a combination of anti-PD-L1, anti-CTLA-4 antibody together with ibrutinib, according to Schedule 2 (B).

FIG. 47A-B exemplify tumor volumes from mice treated with IgG alone (A), or in combination with ibrutinib (B).

FIG. 48A-B exemplify tumor volumes from mice treated with anti-CTLA-4(αCTLA-4) alone (A), or in combination with ibrutinib (B).

FIG. 49 exemplifies the percentage survival of mice treated with either IgG or anti-CTLA-4 (αCTLA-4), alone or in combination with ibrutinib (PCI-32765).

FIG. 50A-B exemplifies tumor volumes from mice injected with A20 tumor cells and treated with IgG alone (A), or in combination with ibrutinib (B).

FIG. 51A-B exemplifies tumor volumes from mice injected with A20 tumor cells and treated with anti-CTLA-4 alone (A), or in combination with ibrutinib (B).

FIG. 52 exemplifies the level of immune checkpoint proteins, in CD44+, Ki67+, and CD4+ cells.

FIG. 53A-B exemplifies tumor volumes from mice injected with J558 tumor cells and treated with IgG alone (A), or in combination with ibrutinib (B).

FIG. 54A-B exemplifies tumor volumes from mice injected with J558 tumor cells and treated with anti-PD-L1 alone (A), or in combination with ibrutinib (B).

FIG. 55 exemplifies the percentage survival of mice injected with J558 tumor cells and treated with either IgG or anti-PD-L1(α-PD-L1), alone or in combination with ibrutinib (PCI-32765).

FIG. 56 illustrates a conceptual schematic of an exemplary computer sever to be used for processing a system and a method described herein.

### DETAILED DESCRIPTION OF THE INVENTION

Small molecule Btk inhibitors, such as Ibrutinib, are useful for reducing the risk of or treating a variety of diseases affected by or affecting many cell types of the hematopoietic lineage including, e.g., autoimmune diseases, heteroimmune conditions or diseases, inflammatory diseases, cancer (e.g., B-cell proliferative disorders), and thromboembolic disorders.

Described herein, in certain embodiments, are methods, combinations, compositions, biomarkers, and kits for treatment of a cancer which comprises administration of a combination of a BTK inhibitor and an immune checkpoint inhibitor. In some embodiments, described herein are methods, combinations, compositions, biomarkers, and kits for treatment of a breast cancer which comprises administration of a combination of a BTK inhibitor and an immune checkpoint inhibitor. In some embodiments, described herein are methods, combinations, compositions, biomarkers, and kits for treatment of a colon cancer which comprises administration of a combination of a BTK inhibitor and an immune checkpoint inhibitor. In some embodiments, described herein are methods, combinations, compositions, biomarkers, and kits for treatment of a diffuse large B-cell lymphoma (DLBCL) which comprises administration of a combination of a BTK inhibitor and an immune checkpoint inhibitor.

Also described herein, in certain embodiments, are methods, combinations, compositions, biomarkers, and kits for treatment of an ibrutinib-resistant cancer which comprises administration of a combination of ibrutinib and an immune checkpoint inhibitor.

In some aspects, described herein are methods for increasing the Th1:Th2 biomarker ratio in a cancer patient, which comprises administration of a combination of a BTK inhibitor and an immune checkpoint inhibitor, wherein the combination decreases the Th2 response in the cancer patient and increases the Th1 response in the cancer patient.

In some aspects, described herein are a pharmaceutical combination which comprises a BTK inhibitor, an immune checkpoint inhibitor, and a pharmaceutically-acceptable excipient. In some embodiments, the pharmaceutical combination further comprises an additional anticancer agent.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

The term “acceptable” or “pharmaceutically acceptable”, with respect to a formulation, composition or ingredient, as used herein, means having no persistent detrimental effect on the general health of the subject being treated or does not abrogate the biological activity or properties of the compound, and is relatively nontoxic.

“Bioavailability” refers to the percentage of Ibrutinib dosed that is delivered into the general circulation of the animal or human being studied. The total exposure (AUC(0-∞)) of a drug when administered intravenously is usually defined as 100% bioavailable (F %). “Oral bioavailability” refers to the extent to which Ibrutinib is absorbed into the general circulation when the pharmaceutical composition is taken orally as compared to intravenous injection.

“Blood plasma concentration” refers to the concentration of Ibrutinib in the plasma component of blood of a subject. It is understood that the plasma concentration of Ibrutinib may vary significantly between subjects, due to variability with respect to metabolism and/or possible interactions with other therapeutic agents. In accordance with one embodiment disclosed herein, the blood or plasma concentration of Ibrutinib may vary from subject to subject. Likewise, values such as maximum plasma concentration (Cmax) or time to reach maximum plasma concentration (Tmax), or total area under the plasma concentration time curve (AUC(0-∞)) may vary from subject to subject. Due to this variability, the amount necessary to constitute “a therapeutically effective amount” of Ibrutinib may vary from subject to subject.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of Ibrutinib, age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

The terms “enhance” or “enhancing” means to increase or prolong either in potency or duration a desired effect. By way of example, “enhancing” the effect of therapeutic agents refers to the ability to increase or prolong, either in potency or duration, the effect of therapeutic agents on during treatment of a disease, disorder or condition. An “enhancing-effective amount,” as used herein, refers to an amount adequate to enhance the effect of a therapeutic agent in the treatment of a disease, disorder or condition. When used in a patient, amounts effective for this use will depend on the severity and course of the disease, disorder or condition, previous therapy, the patient's health status and response to the drugs, and the judgment of the treating physician.

The terms “subject”, “patient” and “individual” are used interchangeably. As used herein, they refer to an animal. By way of example only, a subject may be, but is not limited to, a mammal including, but not limited to, a human. The terms do not require the supervision (whether continuous or intermittent) of a medical professional.

The terms “treat,” “treating” or “treatment”, as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments.

As used herein, the IC50 refers to an amount, concentration or dosage of a particular test compound that achieves a 50% inhibition of a maximal response, such as inhibition of Btk, in an assay that measures such response.

As used herein, EC50 refers to a dosage, concentration or amount of a particular test compound that elicits a dose-dependent response at 50% of maximal expression of a particular response that is induced, provoked or potentiated by the particular test compound.

As used herein, “cancer recurrence”, “cancer relapse”, “relapsed or refractory disease” are used interchangeably herein to refer to a return of cancer following treatment, and includes return of cancer in the primary organ, as well as distant recurrence, where the cancer returns outside of the primary organ.

### Btk Inhibitor Compounds Including Ibrutinib, and Pharmaceutically Acceptable Salts Thereof

The Btk inhibitor compound described herein (i.e. Ibrutinib) is selective for Btk and kinases having a cysteine residue in an amino acid sequence position of the tyrosine kinase that is homologous to the amino acid sequence position of cysteine 481 in Btk. The Btk inhibitor compound can form a covalent bond with Cys 481 of Btk (e.g., via a Michael reaction).

In some embodiments, the Btk inhibitor is a compound of Formula (A) having the structure:

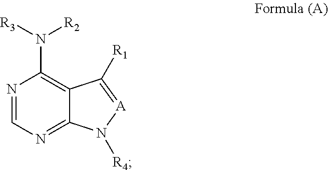

wherein:

A is N;

R₁ is phenyl-O-phenyl or phenyl-S-phenyl;

R₂ and R₃ are independently H;

R₄ is L₃-X-L₄-G, wherein,

L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;

X is optional, and when present is a bond, —O—, —C(═O)—, —S—, —S(═O)—, —S(═O)₂—, —NH—, —NR₉—, —NHC(O)—, —C(O)NH—, —NR₉C(O)—, —C(O)NR₉—, —S(═O)₂NH—, —NHS(═O)₂—, —S(═O)₂NR₉—, —NR₉S(═O)₂—, —OC(O)NH—, —NHC(O)O—, —OC(O)NR₉—, —NR₉C(O)O—, —CH═NO—, —ON═CH—, —NR₁₀C(O)NR₁₀-, heteroaryl-, aryl-, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;

L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;

or L₃, X and L₄ taken together form a nitrogen containing heterocyclic ring;

G is

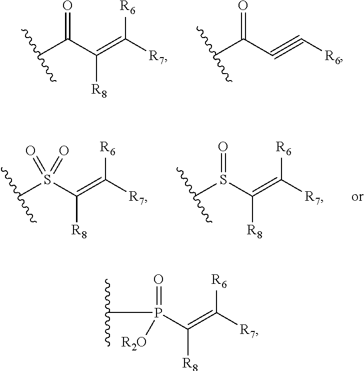

wherein,

R₆, R₇ and R₈ are independently selected from among H, halogen, CN, OH, substituted or unsubstituted alkyl or substituted or unsubstituted heteroalkyl or substituted or unsubstituted cycloalkyl, substituted or unsubstituted heterocycloalkyl, substituted or unsubstituted aryl, substituted or unsubstituted heteroaryl;

each R₉ is independently selected from among H, substituted or unsubstituted lower alkyl, and substituted or unsubstituted lower cycloalkyl;

each R₁₀ is independently H, substituted or unsubstituted lower alkyl, or substituted or unsubstituted lower cycloalkyl; or

two R₁₀ groups can together form a 5-, 6-, 7-, or 8-membered heterocyclic ring; or

R₁₀ and R₁₁ can together form a 5-, 6-, 7-, or 8-membered heterocyclic ring; or each R₁₁ is independently selected from H or substituted or unsubstituted alkyl; or a pharmaceutically acceptable salt thereof. In some embodiments, L₃, X and L₄ taken together form a nitrogen containing heterocyclic ring. In some embodiments, the nitrogen containing heterocyclic ring is a piperidine group. In some embodiments, G is

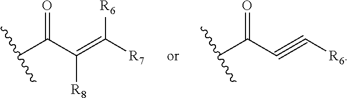

In some embodiments, the compound of Formula (A) is 1-[(3R)-3-[4-amino-3-(4-phenoxyphenyl)pyrazolo[3,4-d]pyrimidin-1-yl]piperidin-1-yl]prop-2-en-1-one.

“Ibrutinib” or “1-((R)-3-(4-amino-3-(4-phenoxyphenyl)-1H-pyrazolo[3,4-d]pyrimidin-1-yl)piperidin-1-yl)prop-2-en-1-one” or “1-{(3R)-3-[4-amino-3-(4-phenoxyphenyl)-1H-pyrazolo[3,4-d]pyrimidin-1-yl]piperidin-1-yl}prop-2-en-1-one” or “2-Propen-1-one, 1-[(3R)-3-[4-amino-3-(4-phenoxyphenyl)-1H-pyrazolo[3,4-d]pyrimidin-1-yl]-1-piperidinyl-” or Ibrutinib or any other suitable name refers to the compound with the following structure:

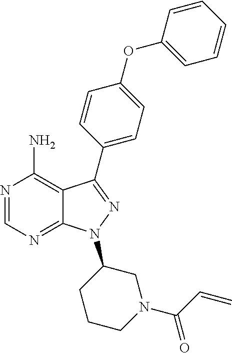 [SMILES: C=CC(=O)N1CCC[C@@H](n2nc(-c3ccc(Oc4ccccc4)cc3)c3c(N)ncnc32)C1]

A wide variety of pharmaceutically acceptable salts is formed from Ibrutinib and includes:
acid addition salts formed by reacting Ibrutinib with an organic acid, which includes aliphatic mono- and dicarboxylic acids, phenyl-substituted alkanoic acids, hydroxyl alkanoic acids, alkanedioic acids, aromatic acids, aliphatic and aromatic sulfonic acids, amino acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like;
acid addition salts formed by reacting Ibrutinib with an inorganic acid, which includes hydrochloric acid, hydrobromic acid, sulfuric acid, nitric acid, phosphoric acid, hydroiodic acid, hydrofluoric acid, phosphorous acid, and the like.

The term “pharmaceutically acceptable salts” in reference to Ibrutinib refers to a salt of Ibrutinib, which does not cause significant irritation to a mammal to which it is administered and does not substantially abrogate the biological activity and properties of the compound.

It should be understood that a reference to a pharmaceutically acceptable salt includes the solvent addition forms (solvates). Solvates contain either stoichiometric or non-stoichiometric amounts of a solvent, and are formed during the process of product formation or isolation with pharmaceutically acceptable solvents such as water, ethanol, methanol, methyl tert-butyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. In one aspect, solvates are formed using, but limited to, Class 3 solvent(s). Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005). Hydrates are formed when the solvent is water, or alcoholates are formed when the solvent is alcohol. In some embodiments, solvates of Ibrutinib, or pharmaceutically acceptable salts thereof, are conveniently prepared or formed during the processes described herein. In some embodiments, solvates of Ibrutinib are anhydrous. In some embodiments, Ibrutinib, or pharmaceutically acceptable salts thereof, exist in unsolvated form. In some embodiments, Ibrutinib, or pharmaceutically acceptable salts thereof, exist in unsolvated form and are anhydrous.

In yet other embodiments, Ibrutinib, or a pharmaceutically acceptable salt thereof, is prepared in various forms, including but not limited to, amorphous phase, crystalline forms, milled forms and nano-particulate forms. In some embodiments, Ibrutinib, or a pharmaceutically acceptable salt thereof, is amorphous. In some embodiments, Ibrutinib, or a pharmaceutically acceptable salt thereof, is amorphous and anhydrous. In some embodiments, Ibrutinib, or a pharmaceutically acceptable salt thereof, is crystalline. In some embodiments, Ibrutinib, or a pharmaceutically acceptable salt thereof, is crystalline and anhydrous.

In some embodiments, Ibrutinib is prepared as outlined in U.S. Pat. No. 7,514,444.

In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13.

In some embodiments, the Btk inhibitor is 4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide (CGI-1746); 7-benzyl-1-(3-(piperidin-1-yl)propyl)-2-(4-(pyridin-4-yl)phenyl)-1H-imidazo[4,5-g]quinoxalin-6(5H)-one (CTA-056); (R)—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide (GDC-0834); 6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one (RN-486); N-[5-[5-(4-acetylpiperazine-1-carbonyl)-4-methoxy-2-methylphenyl]sulfanyl-1,3-thiazol-2-yl]-4-[(3,3-dimethylbutan-2-ylamino)methyl]benzamide (BMS-509744, HY-11092); or N-(5-((5-(4-Acetylpiperazine-1-carbonyl)-4-methoxy-2-methylphenyl)thio)thiazol-2-yl)-4-(((3-methylbutan-2-yl)amino)methyl)benzamide (HY11066); or a pharmaceutically acceptable salt thereof.

In some embodiments, the Btk inhibitor is:

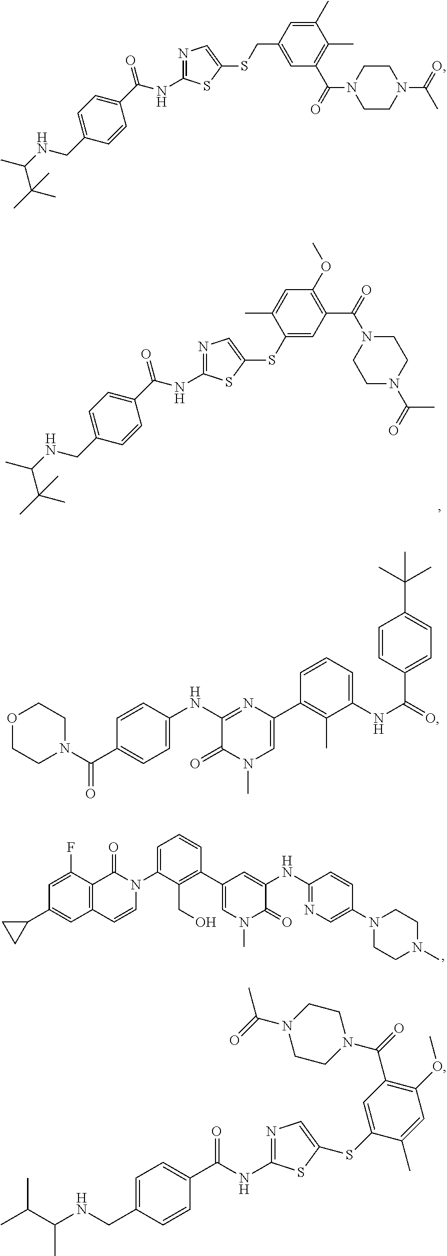

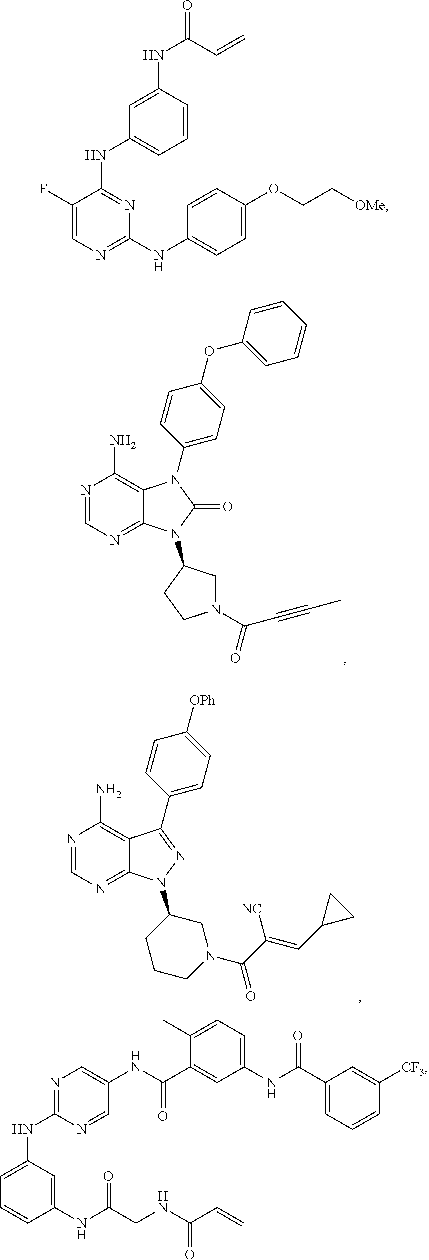

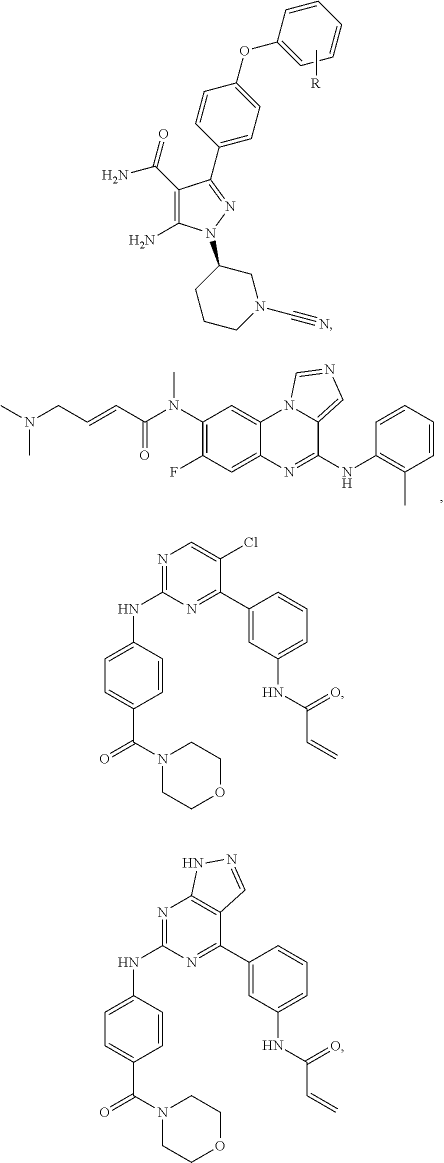

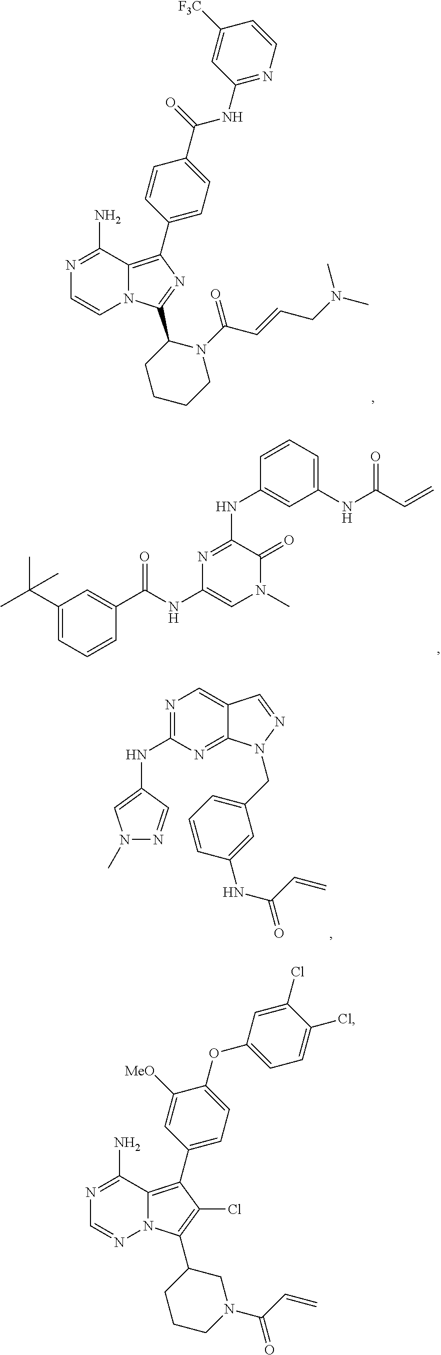

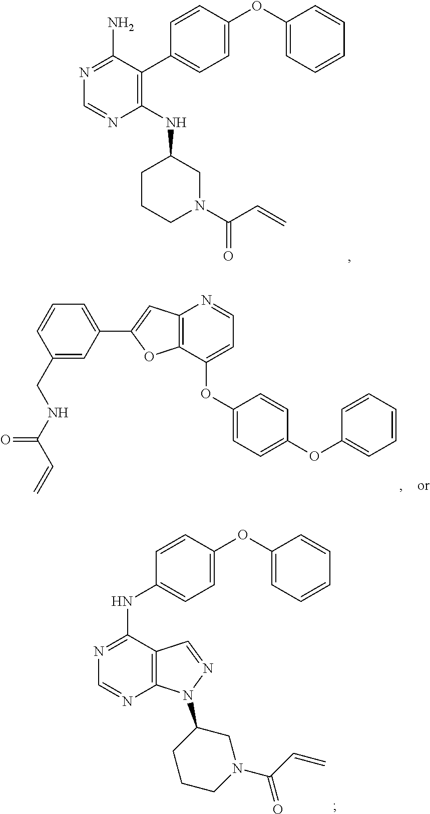 [SMILES: C=CC(=O)N1CCC[C@@H](Nc2ncnc(N)c2-c2ccc(Oc3ccccc3)cc2)C1 ; C=CC(=O)NCc1cccc(-c2cc3nccc(Oc4ccc(Oc5ccccc5)cc4)c3o2)c1 ; C=CC(=O)N1CCC[C@@H](n2ncc3c(Nc4ccc(Oc5ccccc5)cc4)ncnc32)C1]

or a pharmaceutically acceptable salt thereof.

### Additional TEC Family Kinase Inhibitors

BTK is a member of the Tyrosine-protein kinase (TEC) family of kinases. In some embodiments, the TEC family comprises BTK, ITK, TEC, RLK and BMX. In some embodiments, a TEC family kinase inhibitor inhibits the kinase activity of BTK, ITK, TEC, RLK and BMX. In some embodiments, a TEC family kinase inhibitor is a BTK inhibitor, which is disclosed elsewhere herein. In some embodiments, a TEC family kinase inhibitor is an ITK inhibitor. In some embodiments, a TEC family kinase inhibitor is a TEC inhibitor. In some embodiments, a TEC family kinase inhibitor is a RLK inhibitor. In some embodiments, a TEC family kinase inhibitor is a BMK inhibitor.

In some embodiments, the ITK inhibitor covalently binds to Cysteine 442 of ITK. In some embodiments, the Itk inhibitor is an Itk inhibitor compound described in WO2002/0500071, which is incorporated by reference in its entirety. In some embodiments, the Itk inhibitor is an Itk inhibitor compound described in WO2005/070420, which is incorporated by reference in its entirety. In some embodiments, the Itk inhibitor is an Itk inhibitor compound described in WO2005/079791, which is incorporated by reference in its entirety. In some embodiments, the Itk inhibitor is an Itk inhibitor compound described in WO2007/076228, which is incorporated by reference in its entirety. In some embodiments, the Itk inhibitor is an Itk inhibitor compound described in WO2007/058832, which is incorporated by reference in its entirety. In some embodiments, the Itk inhibitor is an Itk inhibitor compound described in WO2004/016610, which is incorporated by reference in its entirety. In some embodiments, the Itk inhibitor is an Itk inhibitor compound described in WO2004/016611, which is incorporated by reference in its entirety. In some embodiments, the Itk inhibitor is an Itk inhibitor compound described in WO2004/016600, which is incorporated by reference in its entirety. In some embodiments, the Itk inhibitor is an Itk inhibitor compound described in WO2004/016615, which is incorporated by reference in its entirety. In some embodiments, the Itk inhibitor is an Itk inhibitor compound described in WO2005/026175, which is incorporated by reference in its entirety. In some embodiments, the Itk inhibitor is an Itk inhibitor compound described in WO2006/065946, which is incorporated by reference in its entirety. In some embodiments, the Itk inhibitor is an Itk inhibitor compound described in WO2007/027594, which is incorporated by reference in its entirety. In some embodiments, the Itk inhibitor is an Itk inhibitor compound described in WO2007/017455, which is incorporated by reference in its entirety. In some embodiments, the Itk inhibitor is an Itk inhibitor compound described in WO2008/025820, which is incorporated by reference in its entirety. In some embodiments, the Itk inhibitor is an Itk inhibitor compound described in WO2008/025821, which is incorporated by reference in its entirety. In some embodiments, the Itk inhibitor is an Itk inhibitor compound described in WO2008/025822, which is incorporated by reference in its entirety. In some embodiments, the Itk inhibitor is an Itk inhibitor compound described in WO2011/017219, which is incorporated by reference in its entirety. In some embodiments, the Itk inhibitor is an Itk inhibitor compound described in WO2011/090760, which is incorporated by reference in its entirety. In some embodiments, the Itk inhibitor is an Itk inhibitor compound described in WO2009/158571, which is incorporated by reference in its entirety. In some embodiments, the Itk inhibitor is an Itk inhibitor compound described in WO2009/051822, which is incorporated by reference in its entirety. In some embodiments, the Itk inhibitor is an Itk inhibitor compound described in U.S. Ser. No. 13/177,657, which is incorporated by reference in its entirety.

In some embodiments, the Itk inhibitor has a structure selected from:

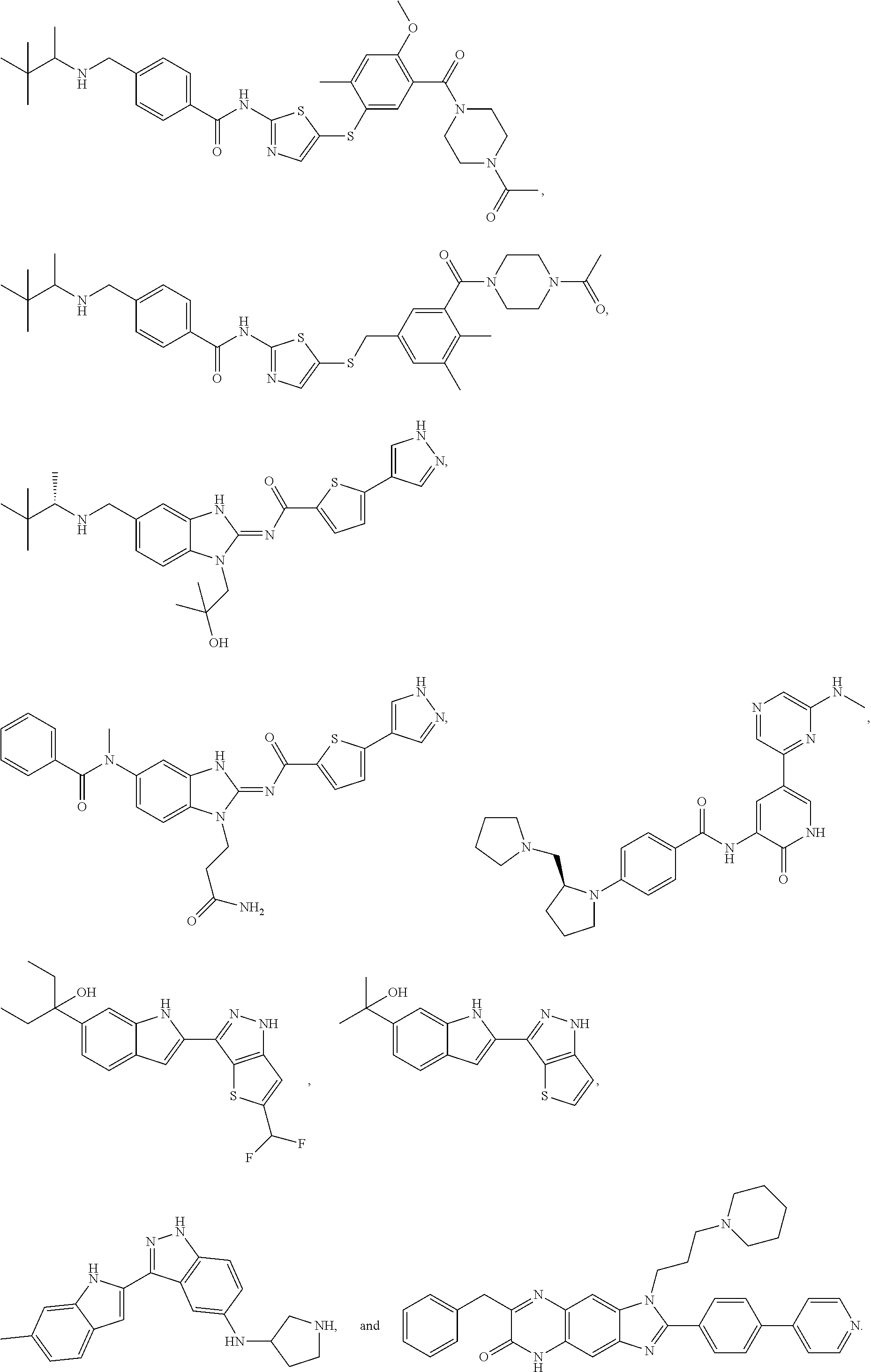

Combination with Immunotherapy

Disclosed herein, in certain embodiments, are pharmaceutical combinations which comprise a TEC inhibitor and an immunotherapeutic agent. In some embodiments, the TEC inhibitor is a BTK, ITK, TEC, RLK, or BMX inhibitor. In some embodiments, the TEC inhibitor is a BTK inhibitor or an ITK inhibitor. In some embodiments, the TEC inhibitor is a BTK inhibitor. In some embodiments, the Btk inhibitor is ibrutinib. In some embodiments, the immunotherapeutic agent is an immune checkpoint inhibitor.

As used herein, the term “immune checkpoints” refers to a group of molecules on the cell surface of CD4 and CD8 T cells. These molecules effectively serve as “brakes” to down-modulate or inhibit an anti-tumor immune response Immune checkpoint molecules include, but are not limited to, Programmed Death-Ligand 1 (PD-L1, also known as B7-H1, CD274), Programmed Death 1 (PD-1), CTLA-4, B7H1, B7H4, OX-40, CD137, CD40, 2B4, IDO1, IDO2, VISTA, CD27, CD28, PD-L2 (B7-DC, CD273), LAG3, CD80, CD86, PDL2, B7H3, HVEM, BTLA, KIR, GAL9, TIM3, A2aR, MARCO (macrophage receptor with collageneous structure), PS (phosphatidylserine), ICOS (inducible T cell costimulator), HAVCR2, CD276, VTCN1, CD70, and CD160.

“Immune checkpoint inhibitors,” as used herein refer to any modulator that inhibits the activity of the immune checkpoint molecule Immune checkpoint inhibitors include small molecule inhibitors, antibodies, antibody-derivatives (including Fab fragments and scFvs), antibody-drug conjugates, antisense oligonucleotides, siRNA, aptamers, peptides and peptide mimetics Inhibitory nucleic acids that decrease the expression and/or activity of immune checkpoint molecules can also be used in the methods disclosed herein. One embodiment is a small inhibitory RNA (siRNA) for interference or inhibition of expression of a target gene. Nucleic acid sequences encoding PD-1, PD-L1 and PD-L2 are disclosed in GENBANK® Accession Nos. NM_{—}005018, AF344424, NP_{—}079515, and NP_{—}054862.

As described elsewhere herein, in some instances a Btk inhibitor (e.g., ibrutinib) and an immune checkpoint inhibitor are co-administration concurrently (e.g., simultaneously, essentially simultaneously or within the same treatment protocol) or sequentially.

In some embodiments, a Btk inhibitor (e.g., ibrutinib) and an immune checkpoint inhibitor are co-administered in separate dosage forms. In some embodiments, Ibrutinib and an immune checkpoint inhibitor are co-administered in combined dosage forms.

In some embodiments, the Btk inhibitor (e.g., ibrutinib), functions to suppress the Th1 response while enhancing the Th2 response. In some embodiments, ibrutinib functions to decrease the number of Th2 polarized T cells in a subject. In some embodiments, ibrutinib functions to increase the number of Th1 polarized T cells in a subject. In some embodiments, ibrutinib functions to increase the number of activated CD8+ cytotoxic T cells in a subject. In some embodiments, ibrutinib functions to increase the ratio of Th1 polarized T cells to Th2 polarized T cells in a subject. In some embodiments, ibrutinib functions to increase IFN-γ expression in a subject.

In some embodiments, the co-administration of a Btk inhibitor (e.g., ibrutinib) and an immune checkpoint inhibitor increases the oral bioavailability of Ibrutinib. In some embodiments, the co-administration of Ibrutinib and an immune checkpoint inhibitor increases the Cmaxof Ibrutinib. In some embodiments, the co-administration of Ibrutinib and an immune checkpoint inhibitor increases the AUC of Ibrutinib.

In some embodiments, co-administration of a Btk inhibitor (e.g., ibrutinib) and an immune checkpoint inhibitor does not significantly affect the Tmax or T1/2 of Ibrutinib as compared to the Tmax and T1/2 of Ibrutinib administered without an immune checkpoint inhibitor.

In some embodiments, the daily dosage of a Btk inhibitor (e.g., ibrutinib) when administered in combination with an immune checkpoint inhibitor is about 10 mg to about 1000 mg. In some embodiments, the daily dosage of Ibrutinib when administered in combination with an immune checkpoint inhibitor is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 250 mg, about 300 mg, about 350 mg, about 400 mg, about 450 mg, about 500 mg, about 550 mg, about 600 mg, about 700 mg or about 800 mg. In some embodiments, the daily dosage of Ibrutinib when administered in combination with an immune checkpoint inhibitor is about 40 mg to about 140 mg. In some embodiments, the daily dosage of Ibrutinib when administered in combination with an immune checkpoint inhibitor is about 40 mg to about 100 mg. In some embodiments, the daily dosage of Ibrutinib when administered in combination with an immune checkpoint inhibitor is about 40 mg to about 70 mg. In some embodiments, the daily dosage of Ibrutinib when administered in combination with an immune checkpoint inhibitor is about 40 mg.

Any suitable daily dose of an immune checkpoint inhibitor is contemplated for use with the compositions, dosage forms, and methods disclosed herein. Daily dose of the immune checkpoint inhibitor depends on multiple factors, the determination of which is within the skills of one of skill in the art. For example, the daily dose of the immune checkpoint inhibitor depends of the strength of the immune checkpoint inhibitor. Weak immune checkpoint inhibitors will require higher daily doses than moderate immune checkpoint inhibitors, and moderate immune checkpoint inhibitors will require higher daily doses than strong immune checkpoint inhibitors.

### Exemplary Immune Checkpoint Inhibitors

In some embodiments, a TEC inhibitor is co-administered with an immune checkpoint inhibitor, wherein the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the TEC inhibitor is a BTK inhibitor or an ITK inhibitor. In some embodiments, the TEC inhibitor is a BTK inhibitor. In some embodiments, the TEC inhibitor is an ITK inhibitor.

In some embodiments, the ITK inhibitor is co-administered with an immune checkpoint inhibitor, wherein the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof.

In some embodiments, the BTK inhibitor is co-administered with an immune checkpoint inhibitor, wherein the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the immune checkpoint inhibitor is an antibody. In some embodiments, the immune checkpoint inhibitor is a monoclonal antibody. In some embodiments, the BTK inhibitor is ibrutinib.

In some embodiments, ibrutinib is co-administered with an immune checkpoint inhibitor, wherein the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the immune checkpoint inhibitor is an antibody. In some embodiments, the immune checkpoint inhibitor is a monoclonal antibody.

Any suitable immune checkpoint inhibitor is contemplated for use with the compositions, dosage forms, and methods disclosed herein. The selection of the immune checkpoint inhibitor depends on multiple factors, and the selection of the immune checkpoint inhibitor is within the skills of one of skill in the art. For example, factors to be considered include the desired reduction in the daily dose of Ibrutinib, any additional drug interactions of the immune checkpoint inhibitor, and the length for which the immune checkpoint inhibitor may be taken. In certain instances, the immune checkpoint inhibitor is an immune checkpoint inhibitor which may be taken long-term, for example chronically. Immune checkpoint inhibitors, as referred to herein, refers to any agent that inhibits the immune checkpoint blockade signal that the immune checkpoint molecule in question regulates. Immune checkpoint inhibitors can include, but are not limited to, immune checkpoint molecule binding proteins, antibodies (or fragments or variants thereof) that bind to immune checkpoint molecules, nucleic acids that downregulate expression of the immune checkpoint molecules, or any other molecules that bind to immune checkpoint molecules (i.e. small organic molecules, peptidomimetics, aptamers, etc.).

In some embodiments, the immune checkpoint inhibitor is an antibody. The antibodies for use in the present invention include, but are not limited to, monoclonal antibodies, synthetic antibodies, polyclonal antibodies, multispecific antibodies (including bi-specific antibodies), human antibodies, humanized antibodies, chimeric antibodies, single-chain Fvs (scFv) (including bi-specific scFvs), single chain antibodies, Fab fragments, F(ab′) fragments, disulfide-linked Fvs (sdFv), and epitope-binding fragments of any of the above. In particular, antibodies for use in the present invention include immunoglobulin molecules and immunologically active portions of immunoglobulin molecules, i.e., molecules that contain a binding site for an immune checkpoint molecule that immunospecifically bind to the immune checkpoint molecule. The immunoglobulin molecules for use in the invention can be of any type {e.g., IgG, IgE, IgM, IgD, IgA and IgY), class {e.g., IgG1, IgG2, IgG3, IgG4, IgA1 and IgA2) or subclass of immunoglobulin molecule. Preferably, the antibodies for use in the invention are IgG, more preferably, IgG1.

An antibody against an immune checkpoint molecule suitable for use with the methods disclosed herein may be from any animal origin including birds and mammals {e.g., human, murine, donkey, sheep, rabbit, goat, guinea pig, camel, horse, shark or chicken). Preferably, the antibodies are human or humanized monoclonal antibodies. As used herein, “human” antibodies include antibodies having the amino acid sequence of a human immunoglobulin and include antibodies isolated from human immunoglobulin libraries or from mice or other animals that express antibodies from human genes.

An antibody against an immune checkpoint molecule suitable for use with the methods disclosed herein may be monospecific, bispecific, trispecific or of greater multispecificity. Multispecific antibodies may immunospecifically bind to different epitopes of a polypeptide or may immunospecifically bind to both a polypeptide as well as a heterologous epitope, such as a heterologous polypeptide or solid support material.

### PD-L1 Inhibitors

In some embodiments, the immune checkpoint inhibitor is an inhibitor of PD-L1. In some embodiments, the immune checkpoint inhibitor is an antibody against PD-L1. In some embodiments, the immune checkpoint inhibitor is a monoclonal antibody against PD-L1. In other or additional embodiments, the immune checkpoint inhibitor is a human or humanized antibody against PD-L1. In one embodiment, the immune checkpoint inhibitor reduces the expression or activity of one or more immune checkpoint proteins, such as PD-L1. In another embodiment, the immune checkpoint inhibitor reduces the interaction between PD-1 and PD-L1. Exemplary immune checkpoint inhibitors include antibodies (e.g., an anti-PD-L1 antibody), RNAi molecules (e.g., anti-PD-L1 RNAi), antisense molecules (e.g., an anti-PD-L1 antisense RNA), dominant negative proteins (e.g., a dominant negative PD-L1 protein), and small molecule inhibitors. Antibodies include monoclonal antibodies, humanized antibodies, deimmunized antibodies, and Ig fusion proteins. An exemplary anti-PD-L1 antibody includes clone EH12. Exemplary antibodies against PD-L1 include: Genentech's MPDL3280A (RG7446); Anti-mouse PD-L1 antibody Clone 10F.9G2 (Cat #BE0101) from BioXcell; anti-PD-L1 monoclonal antibody MDX-1105 (BMS-936559) and BMS-935559 from Bristol-Meyer's Squibb; MSB0010718C; mouse anti-PD-L1 Clone 29E.2A3; and AstraZeneca's MEDI4736. In some embodiments, the anti-PD-L1 antibody is an anti-PD-L1 antibody disclosed in any of the following patent publications (herein incorporated by reference): WO2013079174; CN101104640; WO2010036959; WO2013056716; WO2007005874; WO2010089411; WO2010077634; WO2004004771; WO2006133396; WO201309906; US 20140294898; WO2013181634 or WO2012145493.

In some embodiments, the PD-L1 inhibitor is a nucleic acid inhibitor of PD-L1 expression. In some embodiments, the PD-L1 inhibitor is disclosed in one of the following patent publications (incorporated herein by reference): WO2011127180 or WO2011000841. In some embodiments, the PD-L1 inhibitor is rapamycin.

In some embodiments, a TEC inhibitor is administered in combination with a PD-L1 inhibitor described above and elsewhere for the treatment of a cancer. In some embodiments, the TEC inhibitor is a BTK inhibitor or an ITK inhibitor. In some embodiments, the TEC inhibitor is a BTK inhibitor. In some embodiments, the BTK inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib.

In some embodiments, a BTK inhibitor is administered in combination with a PD-L1 inhibitor for the treatment of a cancer. In some embodiments, the PD-L1 inhibitor is selected from Genentech's MPDL3280A (RG7446); Anti-mouse PD-L1 antibody Clone 10F.9G2 (Cat #BE0101) from BioXcell; anti-PD-L1 monoclonal antibody MDX-1105 (BMS-936559) and BMS-935559 from Bristol-Meyer's Squibb; MSB0010718C; mouse anti-PD-L1 Clone 29E.2A3; AstraZeneca's MEDI4736; EH12; and rapamycin. In some embodiments, a BTK inhibitor is administered in combination with a PD-L1 inhibitor selected from Genentech's MPDL3280A (RG7446); Anti-mouse PD-L1 antibody Clone 10F.9G2 (Cat #BE0101) from BioXcell; anti-PD-L1 monoclonal antibody MDX-1105 (BMS-936559) and BMS-935559 from Bristol-Meyer's Squibb; MSB0010718C; mouse anti-PD-L1 Clone 29E.2A3; AstraZeneca's MEDI4736; EH12; and rapamycin for the treatment of a cancer.

In some embodiments, ibrutinib is administered in combination with a PD-L1 inhibitor for the treatment of a cancer. In some embodiments, the PD-L1 inhibitor is selected from Genentech's MPDL3280A (RG7446); Anti-mouse PD-L1 antibody Clone 10F.9G2 (Cat #BE0101) from BioXcell; anti-PD-L1 monoclonal antibody MDX-1105 (BMS-936559) and BMS-935559 from Bristol-Meyer's Squibb; MSB0010718C; mouse anti-PD-L1 Clone 29E.2A3; AstraZeneca's MEDI4736; EH12; and rapamycin. In some embodiments, ibrutinib is administered in combination with a PD-L1 inhibitor selected from Genentech's MPDL3280A (RG7446); Anti-mouse PD-L1 antibody Clone 10F.9G2 (Cat #BE0101) from BioXcell; anti-PD-L1 monoclonal antibody MDX-1105 (BMS-936559) and BMS-935559 from Bristol-Meyer's Squibb; MSB0010718C; mouse anti-PD-L1 Clone 29E.2A3; AstraZeneca's MEDI4736; EH12; and rapamycin for the treatment of a cancer.

### PD-L2 Inhibitors

In some embodiments, the immune checkpoint inhibitor is an inhibitor of PD-L2. In some embodiments, the immune checkpoint inhibitor is an antibody against PD-L2. In some embodiments, the immune checkpoint inhibitor is a monoclonal antibody against PD-L2. In other or additional embodiments, the immune checkpoint inhibitor is a human or humanized antibody against PD-L2. In some embodiments, the immune checkpoint inhibitor reduces the expression or activity of one or more immune checkpoint proteins, such as PD-L2. In other embodiments, the immune checkpoint inhibitor reduces the interaction between PD-1 and PD-L2. Exemplary immune checkpoint inhibitors include antibodies (e.g., an anti-PD-L2 antibody), RNAi molecules (e.g., an anti-PD-L2 RNAi), antisense molecules (e.g., an anti-PD-L2 antisense RNA), dominant negative proteins (e.g., a dominant negative PD-L2 protein), and small molecule inhibitors. Antibodies include monoclonal antibodies, humanized antibodies, deimmunized antibodies, and Ig fusion proteins.

In some embodiments, the PD-L2 inhibitor is GlaxoSmithKline's AMP-224 (Amplimmune). In some embodiments, the PD-L2 inhibitor is rHIgM12B7.

In some embodiments, a TEC inhibitor is administered in combination with a PD-L2 inhibitor described above and elsewhere for the treatment of a cancer. In some embodiments, the TEC inhibitor is a BTK inhibitor or an ITK inhibitor. In some embodiments, the TEC inhibitor is a BTK inhibitor. In some embodiments, the BTK inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib.

In some embodiments, a BTK inhibitor is administered in combination with a PD-L2 inhibitor for the treatment of cancer. In some embodiments, the PD-L2 inhibitor is selected from GlaxoSmithKline's AMP-224 (Amplimmune) and rHIgM12B7. In some embodiments, a BTK inhibitor is administered in combination with a PD-L2 inhibitor selected from GlaxoSmithKline's AMP-224 (Amplimmune) and rHIgM12B7 for the treatment of a cancer.

In some embodiments, ibrutinib is administered in combination with a PD-L2 inhibitor for the treatment of cancer. In some embodiments, the PD-L2 inhibitor is selected from GlaxoSmithKline's AMP-224 (Amplimmune) and rHIgM12B7. In some embodiments, ibrutinib is administered in combination with a PD-L2 inhibitor selected from GlaxoSmithKline's AMP-224 (Amplimmune) and rHIgM12B7 for the treatment of a cancer.

### PD-1 Inhibitors

In some embodiments, the immune checkpoint inhibitor is an inhibitor of PDL1. In some embodiments, the immune checkpoint inhibitor is an antibody against PD-1. In some embodiments, the immune checkpoint inhibitor is a monoclonal antibody against PD-1. In other or additional embodiments, the immune checkpoint inhibitor is a human or humanized antibody against PD-1. For example, the inhibitors of PD-1 biological activity (or its ligands) disclosed in U.S. Pat. Nos. 7,029,674; 6,808,710; or U.S. Patent Application Nos: 20050250106 and 20050159351 can be used in the methods provided herein. Exemplary antibodies against PD-1 include: Anti-mouse PD-1 antibody Clone J43 (Cat #BE0033-2) from BioXcell; Anti-mouse PD-1 antibody Clone RMP1-14 (Cat #BE0146) from BioXcell; mouse anti-PD-1 antibody Clone EH12; Merck's MK-3475 anti-mouse PD-1 antibody (Keytruda, pembrolizumab, lambrolizumab); and AnaptysBio's anti-PD-1 antibody, known as ANB011; antibody MDX-1 106 (ONO-4538); Bristol-Myers Squibb's human IgG4 monoclonal antibody nivolumab (Opdivo®, BMS-936558, MDX1106); AstraZeneca's AMP-514, and AMP-224; and Pidilizumab (CT-011), CureTech Ltd.

Additional exemplary anti-PD-1 antibodies and methods for their use are described by Goldberg et al, Blood 1 10(1): 186-192 (2007), Thompson et al, Clin. Cancer Res. 13(6): 1757-1761 (2007), and Korman et al, International Application No. PCT/JP2006/309606 (publication no. WO 2006/121168 A1), each of which are expressly incorporated by reference herein. In some embodiments, the anti-PD-1 antibody is an anti-PD-1 antibody disclosed in any of the following patent publications (herein incorporated by reference): WO014557; WO2011110604; WO2008156712; US2012023752; WO2011110621; WO2004072286; WO2004056875; WO20100036959; WO2010029434; WO201213548; WO2002078731; WO2012145493; WO2010089411; WO2001014557; WO2013022091; WO2013019906; WO2003011911; US20140294898; and WO2010001617.

In some embodiments, the PD-1 inhibitor is a PD-1 binding protein as disclosed in WO200914335 (herein incorporated by reference).

In some embodiments, the PD-1 inhibitor is a peptidomimetic inhibitor of PD-1 as disclosed in WO2013132317 (herein incorporated by reference).

In some embodiments, the PD-1 inhibitor is a PD-L1 protein, a PD-L2 protein, or fragments, as well as antibody MDX-1 106 (ONO-4538) tested in clinical studies for the treatment of certain malignancies (Brahmer et al., J Clin Oncol. 2010 28(19): 3167-75, Epub 2010 Jun. 1). Other blocking antibodies may be readily identified and prepared by the skilled person based on the known domain of interaction between PD-1 and PD-L1/PD-L2, as discussed above. For example, a peptide corresponding to the IgV region of PD-1 or PD-L1/PD-L2 (or to a portion of this region) could be used as an antigen to develop blocking antibodies using methods well known in the art.

In some embodiments, a TEC inhibitor is administered in combination with a PD-1 inhibitor described above and elsewhere for the treatment of a cancer. In some embodiments, the TEC inhibitor is a BTK inhibitor or an ITK inhibitor. In some embodiments, the TEC inhibitor is a BTK inhibitor. In some embodiments, the BTK inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib.

In some embodiments, a BTK inhibitor is administered in combination with a PD-1 inhibitor for the treatment of a cancer. In some embodiments, the PD-1 inhibitor is selected from anti-mouse PD-1 antibody Clone J43 (Cat #BE0033-2) from BioXcell; Anti-mouse PD-1 antibody Clone RMP1-14 (Cat #BE0146) from BioXcell; mouse anti-PD-1 antibody Clone EH12; Merck's MK-3475 anti-mouse PD-1 antibody (Keytruda, pembrolizumab, lambrolizumab); and AnaptysBio's anti-PD-1 antibody, known as ANB011; antibody MDX-1 106 (ONO-4538); Bristol-Myers Squibb's human IgG4 monoclonal antibody nivolumab (Opdivo®, BMS-936558, MDX1106); AstraZeneca's AMP-514 and AMP-224; Pidilizumab (CT-011), CureTech Ltd; MDX-1 106 (ONO-4538); PD-L1; and PD-L2. In some embodiments, a BTK inhibitor is administered in combination with a PD-1 inhibitor selected from anti-mouse PD-1 antibody Clone J43 (Cat #BE0033-2) from BioXcell; Anti-mouse PD-1 antibody Clone RMP1-14 (Cat #BE0146) from BioXcell; mouse anti-PD-1 antibody Clone EH12; Merck's MK-3475 anti-mouse PD-1 antibody (Keytruda, pembrolizumab, lambrolizumab); and AnaptysBio's anti-PD-1 antibody, known as ANB011; antibody MDX-1 106 (ONO-4538); Bristol-Myers Squibb's human IgG4 monoclonal antibody nivolumab (Opdivo®, BMS-936558, MDX1106); AstraZeneca's AMP-514 and AMP-224; Pidilizumab (CT-011), CureTech Ltd; MDX-1 106 (ONO-4538); PD-L1; and PD-L2 for the treatment of a cancer.

In some embodiments, ibrutinib is administered in combination with a PD-1 inhibitor for the treatment of a cancer. In some embodiments, the PD-1 inhibitor is selected from anti-mouse PD-1 antibody Clone J43 (Cat #BE0033-2) from BioXcell; Anti-mouse PD-1 antibody Clone RMP1-14 (Cat #BE0146) from BioXcell; mouse anti-PD-1 antibody Clone EH12; Merck's MK-3475 anti-mouse PD-1 antibody (Keytruda, pembrolizumab, lambrolizumab); and AnaptysBio's anti-PD-1 antibody, known as ANB011; antibody MDX-1 106 (ONO-4538); Bristol-Myers Squibb's human IgG4 monoclonal antibody nivolumab (Opdivo®, BMS-936558, MDX1106); AstraZeneca's AMP-514 and AMP-224; Pidilizumab (CT-011), CureTech Ltd; MDX-1 106 (ONO-4538); PD-L1; and PD-L2. In some embodiments, ibrutinib is administered in combination with a PD-1 inhibitor selected from anti-mouse PD-1 antibody Clone J43 (Cat #BE0033-2) from BioXcell; Anti-mouse PD-1 antibody Clone RMP1-14 (Cat #BE0146) from BioXcell; mouse anti-PD-1 antibody Clone EH12; Merck's MK-3475 anti-mouse PD-1 antibody (Keytruda, pembrolizumab, lambrolizumab); and AnaptysBio's anti-PD-1 antibody, known as ANB011; antibody MDX-1 106 (ONO-4538); Bristol-Myers Squibb's human IgG4 monoclonal antibody nivolumab (Opdivo®, BMS-936558, MDX1106); AstraZeneca's AMP-514 and AMP-224; Pidilizumab (CT-011), CureTech Ltd; MDX-1 106 (ONO-4538); PD-L1; and PD-L2 for the treatment of a cancer.

### CTLA-4 Inhibitors

In some embodiments, the immune checkpoint inhibitor is an inhibitor of CTLA-4. In some embodiments, the immune checkpoint inhibitor is an antibody against CTLA-4. In some embodiments, the immune checkpoint inhibitor is a monoclonal antibody against CTLA-4. In other or additional embodiments, the immune checkpoint inhibitor is a human or humanized antibody against CTLA-4. In one embodiment, the anti-CTLA-4 antibody blocks the binding of CTLA-4 to CD80 (B7-1) and/or CD86 (B7-2) expressed on antigen presenting cells. Exemplary antibodies against CTLA-4 include: Bristol Meyers Squibb's anti-CTLA-4 antibody ipilimumab (also known as Yervoy®, MDX-010, BMS-734016 and MDX-101); anti-CTLA4 Antibody, clone 9H10 from Millipore; Pfizer's tremelimumab (CP-675,206, ticilimumab); and anti-CTLA4 antibody clone BNI3 from Abcam.

In some embodiments, the anti-CTLA-4 antibody is an anti-CTLA-4 antibody disclosed in any of the following patent publications (herein incorporated by reference):WO 2001014424; WO 2004035607; US2005/0201994; EP 1212422 B1; WO2003086459; WO2012120125; WO2000037504; WO2009100140; WO200609649; WO2005092380; WO2007123737; WO2006029219; WO20100979597; WO200612168; and WO1997020574. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014; and/or U.S. Pat. Nos. 5,977,318, 6,682,736, 7, 109,003, and 7,132,281, incorporated herein by reference). In some embodiments, the anti-CTLA-4 antibody is an, for example, those disclosed in: WO 98/42752; U.S. Pat. Nos. 6,682,736 and 6,207,156; Hurwitz et al, Proc. Natl. Acad. Sci. USA, 95(17): 10067-10071 (1998); Camacho et al, J. Clin. Oncol., 22(145): Abstract No. 2505 (2004) (antibody CP-675206); Mokyr et al, Cancer Res., 58:5301-5304 (1998) (incorporated herein by reference).

In some embodiments, the CTLA-4 inhibitor is a CTLA-4 ligand as disclosed in WO1996040915.

In some embodiments, the CTLA-4 inhibitor is a nucleic acid inhibitor of CTLA-4 expression. For example, anti-CTLA4 RNAi molecules may take the form of the molecules described by Mello and Fire in PCT Publication Nos. WO 1999/032619 and WO 2001/029058; U.S. Publication Nos. 2003/0051263, 2003/0055020, 2003/0056235, 2004/265839, 2005/0100913, 2006/0024798, 2008/0050342, 2008/0081373, 2008/0248576, and 2008/055443; and/or U.S. Pat. Nos. 6,506,559, 7,282,564, 7,538,095, and 7,560,438 (incorporated herein by reference). In some instances, the anti-CTLA4 RNAi molecules take the form of double stranded RNAi molecules described by Tuschl in European Patent No. EP 1309726 (incorporated herein by reference). In some instances, the anti-CTLA4 RNAi molecules take the form of double stranded RNAi molecules described by Tuschl in U.S. Pat. Nos. 7,056,704 and 7,078,196 (incorporated herein by reference). In some embodiments, the CRLA4 inhibitor is an aptamer described in PCT Publication No. WO2004081021, such as Del 60 or M9-14 del 55.

Additionally, the anti-CTLA4 RNAi molecules of the present invention may take the form be RNA molecules described by Crooke in U.S. Pat. Nos. 5,898,031, 6,107,094, 7,432,249, and 7,432,250, and European Application No. EP 0928290 (incorporated herein by reference).

In some embodiments, a TEC inhibitor is administered in combination with a CTLA-4 inhibitor described above and elsewhere for the treatment of a cancer. In some embodiments, the TEC inhibitor is a BTK inhibitor or an ITK inhibitor. In some embodiments, the TEC inhibitor is a BTK inhibitor. In some embodiments, the BTK inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib.

In some embodiments, a BTK inhibitor is administered in combination with a CTLA-4 inhibitor for the treatment of a cancer. In some embodiments, the CTLA-4 inhibitor is selected from Bristol Meyers Squibb's anti-CTLA-4 antibody ipilimumab (also known as Yervoy®, MDX-010, BMS-734016 and MDX-101); anti-CTLA4 Antibody, clone 9H10 from Millipore; Pfizer's tremelimumab (CP-675,206, ticilimumab); anti-CTLA4 antibody clone BNI3 from Abcam; Del 60; and M9-14 del 55. In some embodiments, a BTK inhibitor is administered in combination with a CTLA-4 inhibitor selected from Bristol Meyers Squibb's anti-CTLA-4 antibody ipilimumab (also known as Yervoy®, MDX-010, BMS-734016 and MDX-101); anti-CTLA4 Antibody, clone 9H10 from Millipore; Pfizer's tremelimumab (CP-675,206, ticilimumab); anti-CTLA4 antibody clone BNI3 from Abcam; Del 60; and M9-14 del 55 for the treatment of a cancer.

In some embodiments, ibrutinib is administered in combination with a CTLA-4 inhibitor for the treatment of a cancer. In some embodiments, the CTLA-4 inhibitor is selected from Bristol Meyers Squibb's anti-CTLA-4 antibody ipilimumab (also known as Yervoy®, MDX-010, BMS-734016 and MDX-101); anti-CTLA4 Antibody, clone 9H10 from Millipore; Pfizer's tremelimumab (CP-675,206, ticilimumab); anti-CTLA4 antibody clone BNI3 from Abcam; Del 60; and M9-14 del 55. In some embodiments, ibrutinib is administered in combination with a CTLA-4 inhibitor selected from Bristol Meyers Squibb's anti-CTLA-4 antibody ipilimumab (also known as Yervoy®, MDX-010, BMS-734016 and MDX-101); anti-CTLA4 Antibody, clone 9H10 from Millipore; Pfizer's tremelimumab (CP-675,206, ticilimumab); anti-CTLA4 antibody clone BNI3 from Abcam; Del 60; and M9-14 del 55 for the treatment of a cancer.

### LAG3 Inhibitors

In some embodiments, the immune checkpoint inhibitor is an inhibitor of LAG3 (CD223). In some embodiments, the immune checkpoint inhibitor is an antibody against LAG3. In some embodiments, the immune checkpoint inhibitor is a monoclonal antibody against LAG3. In other or additional embodiments, the immune checkpoint inhibitor is a human or humanized antibody against LAG3. In additional embodiments, an antibody against LAG3 blocks the interaction of LAG3 with major histocompatibility complex (MHC) class II molecules. Exemplary antibodies against LAG3 include: anti-Lag-3 antibody clone eBioC9B7W (C9B7W) from eBioscience; anti-Lag3 antibody LS-B2237 from LifeSpan Biosciences; IMP321 (ImmuFact) from Immutep; anti-Lag3 antibody BMS-986016; and the LAG-3 chimeric antibody A9H12. In some embodiments, the anti-LAG3 antibody is an anti-LAG3 antibody disclosed in any of the following patent publications (herein incorporated by reference): WO2010019570; WO2008132601; or WO2004078928.

In some embodiments, a TEC inhibitor is administered in combination with a LAG3 inhibitor described above and elsewhere for the treatment of a cancer. In some embodiments, the TEC inhibitor is a BTK inhibitor or an ITK inhibitor. In some embodiments, the TEC inhibitor is a BTK inhibitor. In some embodiments, the BTK inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib.

In some embodiments, a BTK inhibitor is administered in combination with a LAG3 inhibitor for the treatment of a cancer. In some embodiments, the LAG3 inhibitor is selected from anti-Lag-3 antibody clone eBioC9B7W (C9B7W) from eBioscience; anti-Lag3 antibody LS-B2237 from LifeSpan Biosciences; IMP321 (ImmuFact) from Immutep; anti-Lag3 antibody BMS-986016; and the LAG-3 chimeric antibody A9H12. In some embodiments, a BTK inhibitor is administered in combination with a LAG3 inhibitor selected from anti-Lag-3 antibody clone eBioC9B7W (C9B7W) from eBioscience; anti-Lag3 antibody LS-B2237 from LifeSpan Biosciences; IMP321 (ImmuFact) from Immutep; anti-Lag3 antibody BMS-986016; and the LAG-3 chimeric antibody A9H12 for the treatment of a cancer.

In some embodiments, ibrutinib is administered in combination with a LAG3 inhibitor for the treatment of a cancer. In some embodiments, the LAG3 inhibitor is selected from anti-Lag-3 antibody clone eBioC9B7W (C9B7W) from eBioscience; anti-Lag3 antibody LS-B2237 from LifeSpan Biosciences; IMP321 (ImmuFact) from Immutep; anti-Lag3 antibody BMS-986016; and the LAG-3 chimeric antibody A9H12. In some embodiments, ibrutinib is administered in combination with a LAG3 inhibitor selected from anti-Lag-3 antibody clone eBioC9B7W (C9B7W) from eBioscience; anti-Lag3 antibody LS-B2237 from LifeSpan Biosciences; IMP321 (ImmuFact) from Immutep; anti-Lag3 antibody BMS-986016; and the LAG-3 chimeric antibody A9H12 for the treatment of a cancer.

### TIM3 Inhibitors

In some embodiments, the immune checkpoint inhibitor is an antibody against TIM3 (also known as HAVCR2). In some embodiments, the immune checkpoint inhibitor is a monoclonal antibody against TIM3. In other or additional embodiments, the immune checkpoint inhibitor is a human or humanized antibody against TIM3. In additional embodiments, an antibody against TIM3 blocks the interaction of TIM3 with galectin-9 (Gal9). In some embodiments, the anti-TIM3 antibody is an anti-TIM3 antibody disclosed in any of the following patent publications (herein incorporated by reference): WO2013006490; WO201155607; WO2011159877; or WO200117057. In another embodiment, a TIM3 inhibitor is a TIM3 inhibitor disclosed in WO2009052623.

In some embodiments, a TEC inhibitor is administered in combination with a TIM3 inhibitor described above and elsewhere for the treatment of a cancer. In some embodiments, the TEC inhibitor is a BTK inhibitor or an ITK inhibitor. In some embodiments, the TEC inhibitor is a BTK inhibitor. In some embodiments, the BTK inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, a BTK inhibitor is administered in combination with a TIM3 inhibitor for the treatment of a cancer. In some embodiments, ibrutinib is administered in combination with a TIM3 inhibitor for the treatment of a cancer.

### B7-H3 Inhibitors

In some embodiments, the immune checkpoint inhibitor is an antibody against B7-H3. In one embodiment, the immune checkpoint inhibitor is MGA271. In some embodiments, a TEC inhibitor is administered in combination with a B7-H3 inhibitor (e.g. MGA271) for the treatment of a cancer. In some embodiments, the TEC inhibitor is a BTK inhibitor or an ITK inhibitor. In some embodiments, the TEC inhibitor is a BTK inhibitor. In some embodiments, the BTK inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, a BTK inhibitor is administered in combination with a TIM3 inhibitor for the treatment of a cancer. In some embodiments, ibrutinib is administered in combination with a TIM3 inhibitor for the treatment of a cancer. In some embodiments, a BTK inhibitor is administered in combination with a B7-H3 inhibitor (e.g. MGA271) for the treatment of a cancer. In some embodiments, ibrutinib is administered in combination with a B7-H3 inhibitor (e.g. MGA271) for the treatment of a cancer.

### KIR Inhibitors

In some embodiments, the immune checkpoint inhibitor is an antibody against MR. In one embodiment, the immune checkpoint inhibitor is Lirilumab (IPH2101). In some embodiments, an antibody against MR blocks the interaction of KIR with HLA. In some embodiments, a TEC inhibitor is administered in combination with a KIR inhibitor (e.g. Lirilumab) for the treatment of a cancer. In some embodiments, the TEC inhibitor is a BTK inhibitor or an ITK inhibitor. In some embodiments, the TEC inhibitor is a BTK inhibitor. In some embodiments, the BTK inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, a BTK inhibitor is administered in combination with a KIR inhibitor (e.g. Lirilumab) for the treatment of a cancer. In some embodiments, ibrutinib is administered in combination with a KIR inhibitor (e.g. Lirilumab) for the treatment of a cancer.

### CD137 Inhibitors

In some embodiments, the immune checkpoint inhibitor is an antibody against CD137 (also known as 4-1BB or TNFRSF9). In one embodiment, the immune checkpoint inhibitor is urelumab (BMS-663513, Bristol-Myers Squibb), PF-05082566 (anti-4-1BB, PF-2566, Pfizer), or XmAb-5592 (Xencor). In one embodiment, an anti-CD137 antibody is an antibody disclosed in U.S. Published Application No. US 2005/0095244; an antibody disclosed in issued U.S. Pat. No. 7,288,638 (such as 20H4.9-IgG4 [10C7 or BMS-663513] or 20H4.9-IgG1 [BMS-663031]); an antibody disclosed in issued U.S. Pat. No. 6,887,673 [4E9 or BMS-554271]; an antibody disclosed in issued U.S. Pat. No. 7,214,493; an antibody disclosed in issued U.S. Pat. No. 6,303,121; an antibody disclosed in issued U.S. Pat. No. 6,569,997; an antibody disclosed in issued U.S. Pat. No. 6,905,685; an antibody disclosed in issued U.S. Pat. No. 6,355,476; an antibody disclosed in issued U.S. Pat. No. 6,362,325 [1D8 or BMS-469492; 3H3 or BMS-469497; or 3E1]; an antibody disclosed in issued U.S. Pat. No. 6,974,863 (such as 53A2); or an antibody disclosed in issued U.S. Pat. No. 6,210,669 (such as 1D8, 3B8, or 3E1). In a further embodiment, the immune checkpoint inhibitor is one disclosed in WO 2014036412. In another embodiment, an antibody against CD137 blocks the interaction of CD137 with CD137L.

In some embodiments, a TEC inhibitor is administered in combination with a CD137 inhibitor (e.g. urelumab, PF-05082566, XmAb-5592) for the treatment of a cancer. In some embodiments, the TEC inhibitor is a BTK inhibitor or an ITK inhibitor. In some embodiments, the TEC inhibitor is a BTK inhibitor. In some embodiments, the BTK inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, a BTK inhibitor is administered in combination with a CD137 inhibitor (e.g. urelumab, PF-05082566, XmAb-5592) for the treatment of a cancer. In some embodiments, ibrutinib is administered in combination with a CD137 inhibitor (e.g. urelumab, PF-05082566, XmAb-5592) for the treatment of a cancer.

### PS Inhibitors

In some embodiments, the immune checkpoint inhibitor is an antibody against PS. In one embodiment, the immune checkpoint inhibitor is Bavituximab. In some embodiments, a TEC inhibitor is administered in combination with a PS inhibitor (e.g. Bavituximab) for the treatment of a cancer. In some embodiments, the TEC inhibitor is a BTK inhibitor or an ITK inhibitor. In some embodiments, the TEC inhibitor is a BTK inhibitor. In some embodiments, the BTK inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, a BTK inhibitor is administered in combination with a PS inhibitor (e.g. Bavituximab) for the treatment of a cancer. In some embodiments, ibrutinib is administered in combination with a PS inhibitor (e.g. Bavituximab) for the treatment of a cancer.

### CD52 Inhibitors

In some embodiments, the immune checkpoint inhibitor is an antibody against CD52. In one embodiment, the immune checkpoint inhibitor is alemtuzumab. In some embodiments, a TEC inhibitor is administered in combination with a CD52 inhibitor (e.g. alemtuzumab) for the treatment of a cancer. In some embodiments, the TEC inhibitor is a BTK inhibitor or an ITK inhibitor. In some embodiments, the TEC inhibitor is a BTK inhibitor. In some embodiments, the BTK inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, a BTK inhibitor is administered in combination with a CD52 inhibitor (e.g. alemtuzumab) for the treatment of a cancer. In some embodiments, ibrutinib is administered in combination with a CD52 inhibitor (e.g. alemtuzumab) for the treatment of a cancer.

### CD30 Inhibitors

In some embodiments, the immune checkpoint inhibitor is an antibody against CD30. In one embodiment, the immune checkpoint inhibitor is brentuximab vedotin. In another embodiment, an antibody against CD30 blocks the interaction of CD30 with CD30L. In some embodiments, a TEC inhibitor is administered in combination with a CD30 inhibitor (e.g. brentuximab vedotin) for the treatment of a cancer. In some embodiments, the TEC inhibitor is a BTK inhibitor or an ITK inhibitor. In some embodiments, the TEC inhibitor is a BTK inhibitor. In some embodiments, the BTK inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, a BTK inhibitor is administered in combination with a CD30 inhibitor (e.g. brentuximab vedotin) for the treatment of a cancer. In some embodiments, ibrutinib is administered in combination with a CD30 inhibitor (e.g. brentuximab vedotin) for the treatment of a cancer.

### CD33 Inhibitors

In some embodiments, the immune checkpoint inhibitor is an antibody against CD33. In one embodiment, the immune checkpoint inhibitor is gemtuzumab ozogamicin. In some embodiments, a TEC inhibitor is administered in combination with a CD33 inhibitor (e.g. gemtuzumab ozogamicin) for the treatment of a cancer. In some embodiments, the TEC inhibitor is a BTK inhibitor or an ITK inhibitor. In some embodiments, the TEC inhibitor is a BTK inhibitor. In some embodiments, the BTK inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, a BTK inhibitor is administered in combination with a CD33 inhibitor (e.g. gemtuzumab ozogamicin) for the treatment of a cancer. In some embodiments, ibrutinib is administered in combination with a CD33 inhibitor (e.g. gemtuzumab ozogamicin) for the treatment of a cancer.

### CD20 Inhibitors

In some embodiments, the immune checkpoint inhibitor is an antibody against CD20. In one embodiment, the immune checkpoint inhibitor is ibritumomab tiuxetan. In another embodiment, the immune checkpoint inhibitor is ofatumumab. In another embodiment, the immune checkpoint inhibitor is rituximab. In another embodiment, the immune checkpoint inhibitor is tositumomab. In some embodiments, a TEC inhibitor is administered in combination with a CD20 inhibitor (e.g. ibritumomab tiuxetan, ofatumumab, rituximab, tositumomab) for the treatment of a cancer. In some embodiments, the TEC inhibitor is a BTK inhibitor or an ITK inhibitor. In some embodiments, the TEC inhibitor is a BTK inhibitor. In some embodiments, the BTK inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, a BTK inhibitor is administered in combination with a CD20 inhibitor (e.g. ibritumomab tiuxetan, ofatumumab, rituximab, tositumomab) for the treatment of a cancer. In some embodiments, ibrutinib is administered in combination with a CD20 inhibitor (e.g. ibritumomab tiuxetan, ofatumumab, rituximab, tositumomab) for the treatment of a cancer.

### CD27 Inhibitors

In some embodiments, the immune checkpoint inhibitor is an antibody against CD27 (also known as TNFRSF7). In one embodiment, the immune checkpoint inhibitor is CDX-1127 (Celldex Therapeutics). In another embodiment, an antibody against CD27 blocks the interaction of CD27 with CD70. In some embodiments, a TEC inhibitor is administered in combination with a CD27 inhibitor (e.g. CDX-1127) for the treatment of a cancer. In some embodiments, the TEC inhibitor is a BTK inhibitor or an ITK inhibitor. In some embodiments, the TEC inhibitor is a BTK inhibitor. In some embodiments, the BTK inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, a BTK inhibitor is administered in combination with a CD27 inhibitor (e.g. CDX-1127) for the treatment of a cancer. In some embodiments, ibrutinib is administered in combination with an OX40 inhibitor (e.g. CDX-1127) for the treatment of a cancer.

### OX40 Inhibitors

In some embodiments, the immune checkpoint inhibitor is an antibody against OX40 (also known as TNFRSF4 or CD134). In one embodiment, the immune checkpoint inhibitor is anti-OX40 mouse IgG. In another embodiment, an antibody against OX40 blocks the interaction of OX40 with OX40L. In some embodiments, a TEC inhibitor is administered in combination with an OX40 inhibitor (e.g. anti-OX40 mouse IgG) for the treatment of a cancer. In some embodiments, the TEC inhibitor is a BTK inhibitor or an ITK inhibitor. In some embodiments, the TEC inhibitor is a BTK inhibitor. In some embodiments, the BTK inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, a BTK inhibitor is administered in combination with an OX40 inhibitor (e.g. anti-OX40 mouse IgG) for the treatment of a cancer. In some embodiments, ibrutinib is administered in combination with an OX40 inhibitor (e.g. anti-OX40 mouse IgG) for the treatment of a cancer.

### GITR Inhibitors

In some embodiments, the immune checkpoint inhibitor is an antibody against glucocorticoid-induced tumor necrosis factor receptor (GITR). In one embodiment, the immune checkpoint inhibitor is TRX518 (GITR, Inc.). In another embodiment, an antibody against GITR blocks the interaction of GITR with GITRL. In some embodiments, a TEC inhibitor is administered in combination with a GITR inhibitor (e.g. TRX518) for the treatment of a cancer. In some embodiments, the TEC inhibitor is a BTK inhibitor or an ITK inhibitor. In some embodiments, the TEC inhibitor is a BTK inhibitor. In some embodiments, the BTK inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, a BTK inhibitor is administered in combination with a GITR inhibitor (e.g. TRX518) for the treatment of a cancer. In some embodiments, ibrutinib is administered in combination with an OX40 inhibitor (e.g. TRX518) for the treatment of a cancer.

### ICOS Inhibitors

In some embodiments, the immune checkpoint inhibitor is an antibody against inducible T-cell COStimulator (ICOS, also known as CD278). In one embodiment, the immune checkpoint inhibitor is MEDI570 (MedImmune, LLC) or AMG557 (Amgen). In another embodiment, an antibody against ICOS blocks the interaction of ICOS with ICOSL and/or B7-H2. In some embodiments, a TEC inhibitor is administered in combination with an ICOS inhibitor (e.g. MEDI570 or AMG557) for the treatment of a cancer. In some embodiments, the TEC inhibitor is a BTK inhibitor or an ITK inhibitor. In some embodiments, the TEC inhibitor is a BTK inhibitor. In some embodiments, the BTK inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, a BTK inhibitor is administered in combination with an ICOS inhibitor (e.g. MEDI570 or AMG557) for the treatment of a cancer. In some embodiments, ibrutinib is administered in combination with an OX40 inhibitor (e.g. MEDI570 or AMG557) for the treatment of a cancer.

### Additional Immune Checkpoint Inhibitors

In some embodiments, the immune checkpoint inhibitor is an inhibitor against BTLA (CD272), CD160, 2B4, LAIR1, TIGHT, LIGHT, DR3, CD226, CD2, or SLAM. As described elsewhere herein, an immune checkpoint inhibitor can be one or more binding proteins, antibodies (or fragments or variants thereof) that bind to immune checkpoint molecules, nucleic acids that downregulate expression of the immune checkpoint molecules, or any other molecules that bind to immune checkpoint molecules (i.e. small organic molecules, peptidomimetics, aptamers, etc.). In some instances, an inhibitor of BTLA (CD272) is HVEM. In some instances, an inhibitor of CD160 is HVEM. In some cases, an inhibitor of 2B4 is CD48. In some instances, an inhibitor of LAIR1 is collagen. In some instances, an inhibitor of TIGHT is CD112, CD113, or CD155. In some instances, an inhibitor of CD28 is CD80 or CD86. In some instances, an inhibitor of LIGHT is HVEM. In some instances, an inhibitor of DR3 is TL1A. In some instances, an inhibitor of CD226 is CD155 or CD112. In some cases, an inhibitor of CD2 is CD48 or CD58. In some cases, SLAM is self inhibitory and an inhibitor of SLAM is SLAM.

In some embodiments, a TEC inhibitor is administered in combination with an inhibitor against BTLA (CD272), CD160, 2B4, LAIR1, TIGHT, LIGHT, DR3, CD226, CD2, or SLAM for the treatment of a cancer. In some embodiments, the TEC inhibitor is a BTK inhibitor or an ITK inhibitor. In some embodiments, the TEC inhibitor is a BTK inhibitor. In some embodiments, the BTK inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, a BTK inhibitor is administered in combination with an inhibitor against BTLA (CD272), CD160, 2B4, LAIR1, TIGHT, LIGHT, DR3, CD226, CD2, or SLAM for the treatment of a cancer. In some embodiments, ibrutinib is administered in combination with an inhibitor against BTLA (CD272), CD160, 2B4, LAIR1, TIGHT, LIGHT, DR3, CD226, CD2, or SLAM for the treatment of a cancer.

### Methods of Use

Disclosed herein, in certain embodiments, is a method of treating a cancer in an individual in need thereof which comprises administering a combination of a TEC inhibitor and an immune checkpoint inhibitor. In some embodiments, the TEC inhibitor is a BTK, ITK, TEC, RLK, or BMX inhibitor. In some embodiments, the TEC inhibitor is a BTK inhibitor or an ITK inhibitor. In some embodiments, the TEC inhibitor is a BTK inhibitor. In some embodiments, the Btk inhibitor is ibrutinib. In some embodiments, the combination provides a synergistic therapeutic effect compared to administration of ibrutinib or the immune checkpoint inhibitor alone. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the cancer is a solid tumor. In some embodiments, the cancer is a hematologic cancer.

Also disclosed herein, in some embodiments, is a method of treating an ibrutinib-resistant cancer which comprises administering to a subject in need thereof a therapeutically effective amount of a combination comprising: a) ibrutinib; and b) an immune checkpoint inhibitor. In some embodiments, the combination provides a synergistic therapeutic effect compared to administration of ibrutinib or the immune checkpoint inhibitor alone. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the ibrutinib-resistant cancer is a solid tumor. In some embodiments, the ibrutinib-resistant cancer is a hematologic cancer.

### Solid Tumor

Disclosed herein, in certain embodiments, is a method of treating a solid tumor in an individual in need thereof which comprises administering a combination of a TEC inhibitor and an immune checkpoint inhibitor. In some embodiments, the solid tumor is a sarcoma or carcinoma. In some embodiments, the solid tumor is a sarcoma. In some embodiments, the solid tumor is a carcinoma.

In some embodiments, the sarcoma is selected from alveolar rhabdomyosarcoma; alveolar soft part sarcoma; ameloblastoma; angiosarcoma; chondrosarcoma; chordoma; clear cell sarcoma of soft tissue; dedifferentiated liposarcoma; desmoid; desmoplastic small round cell tumor; embryonal rhabdomyosarcoma; epithelioid fibrosarcoma; epithelioid hemangioendothelioma; epithelioid sarcoma; esthesioneuroblastoma; Ewing sarcoma; extrarenal rhabdoid tumor; extraskeletal myxoid chondrosarcoma; extraskeletal osteosarcoma; fibrosarcoma; giant cell tumor; hemangiopericytoma; infantile fibrosarcoma; inflammatory myofibroblastic tumor; Kaposi sarcoma; leiomyosarcoma of bone; liposarcoma; liposarcoma of bone; malignant fibrous histiocytoma (MFH); malignant fibrous histiocytoma (MFH) of bone; malignant mesenchymoma; malignant peripheral nerve sheath tumor; mesenchymal chondrosarcoma; myxofibrosarcoma; myxoid liposarcoma; myxoinflammatory fibroblastic sarcoma; neoplasms with perivascular epitheioid cell differentiation; osteosarcoma; parosteal osteosarcoma; neoplasm with perivascular epitheioid cell differentiation; periosteal osteosarcoma; pleomorphic liposarcoma; pleomorphic rhabdomyosarcoma; PNET/extraskeletal Ewing tumor; rhabdomyosarcoma; round cell liposarcoma; small cell osteosarcoma; solitary fibrous tumor; synovial sarcoma; telangiectatic osteosarcoma.

In some embodiments, the carcinoma is selected from an adenocarcinoma, squamous cell carcinoma, adenosquamous carcinoma, anaplastic carcinoma, large cell carcinoma, or small cell carcinoma. In some embodiments, the carcinoma is selected from anal cancer; appendix cancer; bile duct cancer (i.e., cholangiocarcinoma); bladder cancer; breast cancer; cervical cancer; colon cancer; cancer of Unknown Primary (CUP); esophageal cancer; eye cancer; fallopian tube cancer; gastroenterological cancer; kidney cancer; liver cancer; lung cancer; medulloblastoma; melanoma; oral cancer; ovarian cancer; pancreatic cancer; parathyroid disease; penile cancer; pituitary tumor; prostate cancer; rectal cancer; skin cancer; stomach cancer; testicular cancer; throat cancer; thyroid cancer; uterine cancer; vaginal cancer; or vulvar cancer. In some embodiments, the carcinoma is breast cancer. In some embodiments, the breast cancer is invasive ductal carcinoma, ductal carcinoma in situ, invasive lobular carcinoma, or lobular carcinoma in situ. In some embodiments, the carcinoma is pancreatic cancer. In some embodiments, the pancreatic cancer is adenocarcinoma, or islet cell carcinoma. In some embodiments, the carcinoma is colorectal (colon) cancer. In some embodiments, the colorectal cancer is adenocarcinoma. In some embodiments, the solid tumor is a colon polyp. In some embodiments, the colon polyp is associated with familial adenomatous polyposis. In some embodiments, the carcinoma is bladder cancer. In some embodiments, the bladder cancer is transitional cell bladder cancer, squamous cell bladder cancer, or adenocarcinoma. In some embodiments, the bladder cancer is encompassed by the genitourinary tract cancers. In some embodiments, the genitourinary tract cancers also encompass kidney cancer, prostate cancer, and cancers associated with the reproductive organs. In some embodiments, the carcinoma is lung cancer. In some embodiments, the lung cancer is a non-small cell lung cancer. In some embodiments, the non-small cell lung cancer is adenocarcinoma, squamous-cell lung carcinoma, or large-cell lung carcinoma. In some embodiments, the lung cancer is a small cell lung cancer. In some embodiments, the carcinoma is prostate cancer. In some embodiments, the prostate cancer is adenocarcinoma or small cell carcinoma. In some embodiments, the carcinoma is ovarian cancer. In some embodiments, the ovarian cancer is epithelial ovarian cancer. In some embodiments, the carcinoma is bile duct cancer. In some embodiments, the bile duct cancer is proximal bile duct carcinoma or distal bile duct carcinoma.

In some embodiments, the solid tumor is selected from alveolar soft part sarcoma, bladder cancer, breast cancer, colorectal (colon) cancer, Ewing's bone sarcoma, gastroenterological cancer, head and neck cancer, kidney cancer, leiomyosarcoma, lung cancer, melanoma, osteosarcoma, ovarian cancer, pancreatic cancer, prostate cancer, proximal or distal bile duct cancer, and neuroblastoma. In some embodiments, the solid tumor is prostate cancer. In some embodiments, the solid tumor is breast cancer. In some embodiments, the solid tumor is lung cancer. In some embodiments, the solid tumor is colorectal (colon) cancer. In some embodiments, the solid tumor is gastroenterological cancer. In some embodiments, the solid tumor is melanoma. In some embodiments, the solid tumor is lung cancer. In some embodiments, the solid tumor is kidney cancer. In some embodiments, the solid tumor is head and neck cancer. In some embodiments, the solid tumor is proximal or distal bile duct cancer. In some embodiments, the solid tumor is alveolar soft part sarcoma. In some embodiments, the solid tumor is Ewing's bone sarcoma. In some embodiments, the solid tumor is bladder cancer. In some embodiments, the solid tumor is ovarian cancer. In some embodiments, the solid tumor is leiomyosarcoma. In some embodiments, the solid tumor is osteosarcoma. In some embodiments, the solid tumor is neuroblastoma.

In some embodiments, the breast cancer is ductal carcinoma in situ (intraductal carcinoma), lobular carcinoma in situ, invasive (or infiltrating) ductal carcinoma, invasive (or infiltrating) lobular carcinoma, inflammatory breast cancer, triple-negative breast cancer, paget disease of the nipple, phyllodes tumor, angiosarcoma or invasive breast carcinoma. In some embodiments, the invasive breast carcinoma is further categorized into subtypes. In some embodiments, the subtypes include adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, micropapillary carcinoma or mixed carcinoma.

In some embodiments, the breast cancer is classified according to stages or how far the tumor cells have spread within the breast tissues and to other portions of the body. In some embodiments, there are five stages of breast cancer, Stage 0-IV. In some embodiments, Stage 0 breast cancer refers to non-invasive breast cancers or that there are no evidence of cancer cells or abnormal non-cancerous cells breaking out of the origin site. In some embodiments, Stage I breast cancer refers to invasive breast cancer in which the cancer cells have invaded into surrounding tissues. In some embodiments, Stage I is subclassified into Stage IA and IB, in which Stage IA describes tumor measures up to 2 cm with no spread of cancer cells. Stage IB describes absence of tumor in breast but have small lumps of cancer cells between 0.2 mm to 2 mm within the lymph nodes. In some embodiments, Stage II breast cancer is further subdivided into Stage IIA and IIB. In some embodiments, Stage IIA describes tumor between 2 cm to 5 cm in breast only, or absence of tumor in breast but with cancer between 2 mm to 2 cm in axillary lymph nodes. In some embodiments, Stage IIB describes tumor larger than 5 cm in breast only, or tumor between 2 cm to 5 cm in breast with presence of small tumors from 0.2 mm to 2 mm in axillary lymph nodes. In some embodiments, Stage III breast cancer is further subdivided into Stage IIIA, IIIB, and IIIC. In some embodiments, Stage IIIA describes absence of tumor or tumor greater than 5 cm in breast with small tumors in 4-9 axillary lymph nodes or small tumors 0.2 mm-2 mm in size in axillary lymph nodes. In some embodiments, Stage IIIB describes tumor spreading into the chest wall or skin of the breast causing swelling or ulcer and with presence of tumor in up to 9 axillary lymph nodes. In some embodiments, inflammatory breast cancer is also considered as Stage IIIB In some embodiments, Stage IIIC describes absence of tumor or tumor spreading into the chest wall or to the skin of the breast, with tumor present in 10 or more axillary lymph nodes. In some embodiments, Stage IV breast cancer refers to invasive breast cancer that has metastasized into the lymph nodes and other portions of the body.

In some embodiments, the colon cancer is a colorectal cancer. As used herein and throughout, colon cancer is used interchangeably with colorectal cancer. In some embodiments, colorectal (colon) cancer refers to rectal cancer. In some embodiments, the colon cancer is adenocarcinoma, gastrointestinal carcinoid tumors, gastrointestinal stromal tumors, primary colorectal lymphoma, leiomyosarcoma, melanoma, or squamous cell-carcinoma. In some embodiments, adenocarcinoma is a mucinous adenocarcinoma or a Signet ring cell adenocarcinoma.

In some embodiments, the colon cancer is classified according to stages or how far they have spread through the walls of the colon and rectum. In some embodiments, there are five stages of colon cancer, Stage 0-IV. In some embodiments, Stage 0 colon cancer refers to the very early stage of cancer. In some embodiments, Stage I colon cancer refers to when the cancer has spread beyond the innermost lining of the colon to the second and third layers and also involves the inside wall of the colon. In some embodiments, Stage II colon cancer refers to when the tumor has extended through the muscular wall but has not yet spread into the lymph nodes. In some embodiments, Stage III colon cancer refers to when the tumor has metastasized the colon into one or more lymph nodes. In some embodiments, Stage IV colon cancer refers to when the tumor has metastasized to other parts of the body. In some embodiments, there are two stages of rectal cancer, classified as Stage 0 and Stage I. In some embodiments, Stage 0 rectal cancer refers to when the tumor is located only on the inner lining of the rectum. In some embodiments, Stage I refers to when the tumor has advanced through the inner lining of the rectum but not yet reach past the muscular wall.

In some embodiments, described herein is a method of treating a solid tumor in an individual in need thereof which comprises administering a combination of a TEC inhibitor and an immune checkpoint inhibitor. In some embodiments, the TEC inhibitor is a BTK, ITK, TEC, RLK, or BMX inhibitor. In some embodiments, the TEC inhibitor is a BTK inhibitor or an ITK inhibitor. In some embodiments, the TEC inhibitor is a BTK inhibitor. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the solid tumor is selected from alveolar soft part sarcoma, bladder cancer, breast cancer, colorectal (colon) cancer, Ewing's bone sarcoma, gastroenterological cancer, head and neck cancer, kidney cancer, leiomyosarcoma, lung cancer, melanoma, osteosarcoma, ovarian cancer, pancreatic cancer, prostate cancer, proximal or distal bile duct cancer, and neuroblastoma. In some embodiments, the solid tumor is prostate cancer. In some embodiments, the solid tumor is breast cancer. In some embodiments, the solid tumor is lung cancer. In some embodiments, the solid tumor is colorectal (colon) cancer. In some embodiments, the solid tumor is gastroenterological cancer. In some embodiments, the solid tumor is melanoma. In some embodiments, the solid tumor is lung cancer. In some embodiments, the solid tumor is kidney cancer. In some embodiments, the solid tumor is head and neck cancer. In some embodiments, the solid tumor is proximal or distal bile duct cancer. In some embodiments, the solid tumor is alveolar soft part sarcoma. In some embodiments, the solid tumor is Ewing's bone sarcoma. In some embodiments, the solid tumor is bladder cancer. In some embodiments, the solid tumor is ovarian cancer. In some embodiments, the solid tumor is leiomyosarcoma. In some embodiments, the solid tumor is osteosarcoma. In some embodiments, the solid tumor is neuroblastoma.

In some embodiments, described herein is a method of treating a solid tumor in an individual in need thereof which comprises administering a combination of an ITK inhibitor and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the solid tumor is selected from alveolar soft part sarcoma, bladder cancer, breast cancer, colorectal (colon) cancer, Ewing's bone sarcoma, gastroenterological cancer, genitourinary tract cancer, head and neck cancer, kidney cancer, leiomyosarcoma, lung cancer, melanoma, osteosarcoma, ovarian cancer, pancreatic cancer, prostate cancer, proximal or distal bile duct cancer, and neuroblastoma. In some embodiments, the solid tumor is prostate cancer. In some embodiments, the solid tumor is breast cancer. In some embodiments, the solid tumor is lung cancer. In some embodiments, the solid tumor is colorectal (colon) cancer. In some embodiments, the solid tumor is gastroenterological cancer. In some embodiments, the solid tumor is melanoma. In some embodiments, the solid tumor is lung cancer. In some embodiments, the solid tumor is kidney cancer. In some embodiments, the solid tumor is head and neck cancer. In some embodiments, the solid tumor is proximal or distal bile duct cancer. In some embodiments, the solid tumor is alveolar soft part sarcoma. In some embodiments, the solid tumor is Ewing's bone sarcoma. In some embodiments, the solid tumor is bladder cancer. In some embodiments, the solid tumor is ovarian cancer. In some embodiments, the solid tumor is leiomyosarcoma. In some embodiments, the solid tumor is osteosarcoma. In some embodiments, the solid tumor is neuroblastoma.

In some embodiments, described herein is a method of treating a solid tumor in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the solid tumor is selected from alveolar soft part sarcoma, bladder cancer, breast cancer, colorectal (colon) cancer, Ewing's bone sarcoma, gastroenterological cancer, head and neck cancer, kidney cancer, leiomyosarcoma, lung cancer, melanoma, osteosarcoma, ovarian cancer, pancreatic cancer, prostate cancer, proximal or distal bile duct cancer, and neuroblastoma. In some embodiments, the solid tumor is prostate cancer. In some embodiments, the solid tumor is breast cancer. In some embodiments, the solid tumor is lung cancer. In some embodiments, the solid tumor is colorectal (colon) cancer. In some embodiments, the solid tumor is gastroenterological cancer. In some embodiments, the solid tumor is melanoma. In some embodiments, the solid tumor is lung cancer. In some embodiments, the solid tumor is kidney cancer. In some embodiments, the solid tumor is head and neck cancer. In some embodiments, the solid tumor is proximal or distal bile duct cancer. In some embodiments, the solid tumor is alveolar soft part sarcoma. In some embodiments, the solid tumor is Ewing's bone sarcoma. In some embodiments, the solid tumor is bladder cancer. In some embodiments, the solid tumor is ovarian cancer. In some embodiments, the solid tumor is leiomyosarcoma. In some embodiments, the solid tumor is osteosarcoma. In some embodiments, the solid tumor is neuroblastoma.

In some embodiments, described herein is a method of treating a solid tumor in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the solid tumor is selected from alveolar soft part sarcoma, bladder cancer, breast cancer, colorectal (colon) cancer, Ewing's bone sarcoma, gastroenterological cancer, head and neck cancer, kidney cancer, leiomyosarcoma, lung cancer, melanoma, osteosarcoma, ovarian cancer, pancreatic cancer, prostate cancer, proximal or distal bile duct cancer, and neuroblastoma. In some embodiments, the solid tumor is prostate cancer. In some embodiments, the solid tumor is breast cancer. In some embodiments, the solid tumor is lung cancer. In some embodiments, the solid tumor is colorectal (colon) cancer. In some embodiments, the solid tumor is gastroenterological cancer. In some embodiments, the solid tumor is melanoma. In some embodiments, the solid tumor is lung cancer. In some embodiments, the solid tumor is kidney cancer. In some embodiments, the solid tumor is head and neck cancer. In some embodiments, the solid tumor is proximal or distal bile duct cancer. In some embodiments, the solid tumor is alveolar soft part sarcoma. In some embodiments, the solid tumor is Ewing's bone sarcoma. In some embodiments, the solid tumor is bladder cancer. In some embodiments, the solid tumor is ovarian cancer. In some embodiments, the solid tumor is leiomyosarcoma. In some embodiments, the solid tumor is osteosarcoma. In some embodiments, the solid tumor is neuroblastoma.

In some embodiments, described herein is a method of treating an ibrutinib-resistant solid tumor in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the ibrutinib-resistant solid tumor is selected from alveolar soft part sarcoma, bladder cancer, breast cancer, colorectal (colon) cancer, Ewing's bone sarcoma, gastroenterological cancer, genitourinary tract cancer, head and neck cancer, kidney cancer, leiomyosarcoma, lung cancer, melanoma, osteosarcoma, ovarian cancer, pancreatic cancer, prostate cancer, proximal or distal bile duct cancer, and neuroblastoma. In some embodiments, the ibrutinib-resistant solid tumor is prostate cancer. In some embodiments, the ibrutinib-resistant solid tumor is breast cancer. In some embodiments, the ibrutinib-resistant solid tumor is lung cancer. In some embodiments, the ibrutinib-resistant solid tumor is colorectal (colon) cancer. In some embodiments, the ibrutinib-resistant solid tumor is gastroenterological cancer. In some embodiments, the ibrutinib-resistant solid tumor is melanoma. In some embodiments, the ibrutinib-resistant solid tumor is lung cancer. In some embodiments, the ibrutinib-resistant solid tumor is kidney cancer. In some embodiments, the ibrutinib-resistant solid tumor is head and neck cancer. In some embodiments, the ibrutinib-resistant solid tumor is proximal or distal bile duct cancer. In some embodiments, the ibrutinib-resistant solid tumor is alveolar soft part sarcoma. In some embodiments, the ibrutinib-resistant solid tumor is Ewing's bone sarcoma. In some embodiments, the ibrutinib-resistant solid tumor is bladder cancer. In some embodiments, the ibrutinib-resistant solid tumor is ovarian cancer. In some embodiments, the ibrutinib-resistant solid tumor is leiomyosarcoma. In some embodiments, the ibrutinib-resistant solid tumor is osteosarcoma. In some embodiments, the ibrutinib-resistant solid tumor is neuroblastoma.

In some embodiments, described herein is a method of treating a breast cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a breast cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a colon cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a colon cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a lung cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a lung cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a prostate cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a prostate cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a pancreatic cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a pancreatic cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating an ovarian cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating an ovarian cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a bladder cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a bladder cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a proximal or distal bile duct cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a proximal or distal bile duct cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a melanoma cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a melanoma cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, a cancer is a treatment-naive cancer. In some instances, a treatment-naive cancer is a cancer that has not been treated by a therapy, such as for example by a TEC inhibitor, an immune checkpoint inhibitor, and/or by an additional therapeutic agent disclosed elsewhere herein. In some embodiments, a treatment-naive cancer is a solid tumor. In some embodiments, a treatment-naive solid tumor is a solid tumor such as bladder, breast, colon, pancreatic, lung, prostate, ovarian, proximal or distal bile duct cancer, or melanoma. In some embodiments, described herein is a method of treating a treatment-naive solid tumor in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

### Relapsed or Refractory Solid Tumor

In some embodiments, the solid tumor is a relapsed or refractory solid tumor. In some embodiments, the relapsed or refractory solid tumor is a sarcoma or carcinoma. In some embodiments, the relapsed or refractory solid tumor is a sarcoma. In some embodiments, the relapsed or refractory solid tumor is a carcinoma. In some embodiments, the sarcoma is selected from alveolar rhabdomyosarcoma; alveolar soft part sarcoma; ameloblastoma; angiosarcoma; chondrosarcoma; chordoma; clear cell sarcoma of soft tissue; dedifferentiated liposarcoma; desmoid; desmoplastic small round cell tumor; embryonal rhabdomyosarcoma; epithelioid fibrosarcoma; epithelioid hemangioendothelioma; epithelioid sarcoma; esthesioneuroblastoma; Ewing sarcoma; extrarenal rhabdoid tumor; extraskeletal myxoid chondrosarcoma; extraskeletal osteosarcoma; fibrosarcoma; giant cell tumor; hemangiopericytoma; infantile fibrosarcoma; inflammatory myofibroblastic tumor; Kaposi sarcoma; leiomyosarcoma of bone; liposarcoma; liposarcoma of bone; malignant fibrous histiocytoma (MFH); malignant fibrous histiocytoma (MFH) of bone; malignant mesenchymoma; malignant peripheral nerve sheath tumor; mesenchymal chondrosarcoma; myxofibrosarcoma; myxoid liposarcoma; myxoinflammatory fibroblastic sarcoma; neoplasms with perivascular epitheioid cell differentiation; osteosarcoma; parosteal osteosarcoma; neoplasm with perivascular epitheioid cell differentiation; periosteal osteosarcoma; pleomorphic liposarcoma; pleomorphic rhabdomyosarcoma; PNET/extraskeletal Ewing tumor; rhabdomyosarcoma; round cell liposarcoma; small cell osteosarcoma; solitary fibrous tumor; synovial sarcoma; telangiectatic osteosarcoma. In some embodiments, the carcinoma is selected from an adenocarcinoma, squamous cell carcinoma, adenosquamous carcinoma, anaplastic carcinoma, large cell carcinoma, or small cell carcinoma. In some embodiments, the carcinoma is selected from anal cancer; appendix cancer; bile duct cancer (i.e., cholangiocarcinoma); bladder cancer; breast cancer; cervical cancer; colon cancer; cancer of Unknown Primary (CUP); esophageal cancer; eye cancer; fallopian tube cancer; gastroenterological cancer; kidney cancer; liver cancer; lung cancer; medulloblastoma; melanoma; oral cancer; ovarian cancer; pancreatic cancer; parathyroid disease; penile cancer; pituitary tumor; prostate cancer; rectal cancer; skin cancer; stomach cancer; testicular cancer; throat cancer; thyroid cancer; uterine cancer; vaginal cancer; or vulvar cancer. In some embodiments, the carcinoma is breast cancer. In some embodiments, the breast cancer is invasive ductal carcinoma, ductal carcinoma in situ, invasive lobular carcinoma, or lobular carcinoma in situ. In some embodiments, the carcinoma is pancreatic cancer. In some embodiments, the pancreatic cancer is adenocarcinoma, or islet cell carcinoma. In some embodiments, the carcinoma is colorectal (colon) cancer. In some embodiments, the colorectal cancer is adenocarcinoma. In some embodiments, the solid tumor is a colon polyp. In some embodiments, the colon polyp is associated with familial adenomatous polyposis. In some embodiments, the carcinoma is bladder cancer. In some embodiments, the bladder cancer is transitional cell bladder cancer, squamous cell bladder cancer, or adenocarcinoma. In some embodiments, the carcinoma is lung cancer. In some embodiments, the lung cancer is a non-small cell lung cancer. In some embodiments, the non-small cell lung cancer is adenocarcinoma, squamous-cell lung carcinoma, or large-cell lung carcinoma. In some embodiments, the lung cancer is a small cell lung cancer. In some embodiments, the carcinoma is prostate cancer. In some embodiments, the prostate cancer is adenocarcinoma or small cell carcinoma. In some embodiments, the carcinoma is ovarian cancer. In some embodiments, the ovarian cancer is epithelial ovarian cancer. In some embodiments, the carcinoma is bile duct cancer. In some embodiments, the bile duct cancer is proximal bile duct carcinoma or distal bile duct carcinoma.

In some embodiments, the relapsed or refractory solid tumor is selected from alveolar soft part sarcoma, bladder cancer, breast cancer, colorectal (colon) cancer, Ewing's bone sarcoma, gastroenterological cancer, head and neck cancer, kidney cancer, leiomyosarcoma, lung cancer, melanoma, osteosarcoma, ovarian cancer, pancreatic cancer, prostate cancer, proximal or distal bile duct cancer, and neuroblastoma. In some embodiments, the relapsed or refractory solid tumor is prostate cancer. In some embodiments, the relapsed or refractory solid tumor is breast cancer. In some embodiments, the relapsed or refractory solid tumor is lung cancer. In some embodiments, the relapsed or refractory solid tumor is colorectal (colon) cancer. In some embodiments, the relapsed or refractory solid tumor is gastroenterological cancer. In some embodiments, the relapsed or refractory solid tumor is melanoma. In some embodiments, the relapsed or refractory solid tumor is lung cancer. In some embodiments, the relapsed or refractory solid tumor is kidney cancer. In some embodiments, the relapsed or refractory solid tumor is head and neck cancer. In some embodiments, the relapsed or refractory solid tumor is proximal or distal bile duct cancer. In some embodiments, the relapsed or refractory solid tumor is alveolar soft part sarcoma. In some embodiments, the relapsed or refractory solid tumor is Ewing's bone sarcoma. In some embodiments, the relapsed or refractory solid tumor is bladder cancer. In some embodiments, the relapsed or refractory solid tumor is ovarian cancer. In some embodiments, the relapsed or refractory solid tumor is leiomyosarcoma. In some embodiments, the relapsed or refractory solid tumor is osteosarcoma. In some embodiments, the relapsed or refractory solid tumor is neuroblastoma.

In some embodiments, the relapsed or refractory solid tumor is a relapsed or refractory breast cancer. In some embodiments, the relapsed or refractory breast cancer is ductal carcinoma in situ (intraductal carcinoma), lobular carcinoma in situ, invasive (or infiltrating) ductal carcinoma, invasive (or infiltrating) lobular carcinoma, inflammatory breast cancer, triple-negative breast cancer, paget disease of the nipple, phyllodes tumor, angiosarcoma or invasive breast carcinoma. In some embodiments, the invasive breast carcinoma is further categorized into subtypes. In some embodiments, the subtypes include adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, micropapillary carcinoma or mixed carcinoma.

In some embodiments, the relapsed or refractory solid tumor is a relapsed or refractory colon cancer. In some embodiments, the relapsed or refractory colon cancer is adenocarcinoma, gastrointestinal carcinoid tumors, gastrointestinal stromal tumors, primary colorectal lymphoma, leiomyosarcoma, melanoma, squamous cell-carcinoma, mucinous adenocarcinoma, or Signet ring cell adenocarcinoma.

In some embodiments, described herein is a method of treating a relapsed or refractory solid tumor in an individual in need thereof which comprises administering a combination of a TEC inhibitor and an immune checkpoint inhibitor. In some embodiments, the individual has relapsed or has developed a refractory solid tumor to an existing therapy. In some embodiments, the TEC inhibitor is a BTK, ITK, TEC, RLK, or BMX inhibitor. In some embodiments, the TEC inhibitor is a BTK inhibitor or an ITK inhibitor. In some embodiments, the TEC inhibitor is a BTK inhibitor. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the relapsed or refractory solid tumor is selected from alveolar soft part sarcoma, bladder cancer, breast cancer, colorectal (colon) cancer, Ewing's bone sarcoma, gastroenterological cancer, head and neck cancer, kidney cancer, leiomyosarcoma, lung cancer, melanoma, osteosarcoma, ovarian cancer, pancreatic cancer, prostate cancer, proximal or distal bile duct cancer, and neuroblastoma. In some embodiments, the relapsed or refractory solid tumor is prostate cancer. In some embodiments, the relapsed or refractory solid tumor is breast cancer. In some embodiments, the relapsed or refractory solid tumor is lung cancer. In some embodiments, the relapsed or refractory solid tumor is colorectal (colon) cancer. In some embodiments, the relapsed or refractory solid tumor is gastroenterological cancer. In some embodiments, the relapsed or refractory solid tumor is melanoma. In some embodiments, the relapsed or refractory solid tumor is lung cancer. In some embodiments, the relapsed or refractory solid tumor is kidney cancer. In some embodiments, the relapsed or refractory solid tumor is head and neck cancer. In some embodiments, the relapsed or refractory solid tumor is proximal or distal bile duct cancer. In some embodiments, the relapsed or refractory solid tumor is alveolar soft part sarcoma. In some embodiments, the relapsed or refractory solid tumor is Ewing's bone sarcoma. In some embodiments, the relapsed or refractory solid tumor is bladder cancer. In some embodiments, the relapsed or refractory solid tumor is ovarian cancer. In some embodiments, the relapsed or refractory solid tumor is leiomyosarcoma. In some embodiments, the relapsed or refractory solid tumor is osteosarcoma. In some embodiments, the relapsed or refractory solid tumor is neuroblastoma.

In some embodiments, described herein is a method of treating a relapsed or refractory solid tumor in an individual in need thereof which comprises administering a combination of an ITK inhibitor and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the relapsed or refractory solid tumor is selected from alveolar soft part sarcoma, bladder cancer, breast cancer, colorectal (colon) cancer, Ewing's bone sarcoma, gastroenterological cancer, head and neck cancer, kidney cancer, leiomyosarcoma, lung cancer, melanoma, osteosarcoma, ovarian cancer, pancreatic cancer, prostate cancer, proximal or distal bile duct cancer, and neuroblastoma. In some embodiments, the relapsed or refractory solid tumor is prostate cancer. In some embodiments, the relapsed or refractory solid tumor is breast cancer. In some embodiments, the relapsed or refractory solid tumor is lung cancer. In some embodiments, the relapsed or refractory solid tumor is colorectal (colon) cancer. In some embodiments, the relapsed or refractory solid tumor is gastroenterological cancer. In some embodiments, the relapsed or refractory solid tumor is melanoma. In some embodiments, the relapsed or refractory solid tumor is lung cancer. In some embodiments, the relapsed or refractory solid tumor is kidney cancer. In some embodiments, the relapsed or refractory solid tumor is head and neck cancer. In some embodiments, the relapsed or refractory solid tumor is proximal or distal bile duct cancer. In some embodiments, the relapsed or refractory solid tumor is alveolar soft part sarcoma. In some embodiments, the relapsed or refractory solid tumor is Ewing's bone sarcoma. In some embodiments, the relapsed or refractory solid tumor is bladder cancer. In some embodiments, the relapsed or refractory solid tumor is ovarian cancer. In some embodiments, the relapsed or refractory solid tumor is leiomyosarcoma. In some embodiments, the relapsed or refractory solid tumor is osteosarcoma. In some embodiments, the relapsed or refractory solid tumor is neuroblastoma.

In some embodiments, described herein is a method of treating a relapsed or refractory solid tumor in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the relapsed or refractory solid tumor is selected from alveolar soft part sarcoma, bladder cancer, breast cancer, colorectal (colon) cancer, Ewing's bone sarcoma, gastroenterological cancer, head and neck cancer, kidney cancer, leiomyosarcoma, lung cancer, melanoma, osteosarcoma, ovarian cancer, pancreatic cancer, prostate cancer, proximal or distal bile duct cancer, and neuroblastoma. In some embodiments, the relapsed or refractory solid tumor is prostate cancer. In some embodiments, the relapsed or refractory solid tumor is breast cancer. In some embodiments, the relapsed or refractory solid tumor is lung cancer. In some embodiments, the relapsed or refractory solid tumor is colorectal (colon) cancer. In some embodiments, the relapsed or refractory solid tumor is gastroenterological cancer. In some embodiments, the relapsed or refractory solid tumor is melanoma. In some embodiments, the relapsed or refractory solid tumor is lung cancer. In some embodiments, the relapsed or refractory solid tumor is kidney cancer. In some embodiments, the relapsed or refractory solid tumor is head and neck cancer. In some embodiments, the relapsed or refractory solid tumor is proximal or distal bile duct cancer. In some embodiments, the relapsed or refractory solid tumor is alveolar soft part sarcoma. In some embodiments, the relapsed or refractory solid tumor is Ewing's bone sarcoma. In some embodiments, the relapsed or refractory solid tumor is bladder cancer. In some embodiments, the relapsed or refractory solid tumor is ovarian cancer. In some embodiments, the relapsed or refractory solid tumor is leiomyosarcoma. In some embodiments, the relapsed or refractory solid tumor is osteosarcoma. In some embodiments, the relapsed or refractory solid tumor is neuroblastoma.

In some embodiments, described herein is a method of treating a relapsed or refractory solid tumor in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the relapsed or refractory solid tumor is selected from alveolar soft part sarcoma, bladder cancer, breast cancer, colorectal (colon) cancer, Ewing's bone sarcoma, gastroenterological cancer, head and neck cancer, kidney cancer, leiomyosarcoma, lung cancer, melanoma, osteosarcoma, ovarian cancer, pancreatic cancer, prostate cancer, proximal or distal bile duct cancer, and neuroblastoma. In some embodiments, the relapsed or refractory solid tumor is prostate cancer. In some embodiments, the relapsed or refractory solid tumor is breast cancer. In some embodiments, the relapsed or refractory solid tumor is lung cancer. In some embodiments, the relapsed or refractory solid tumor is colorectal (colon) cancer. In some embodiments, the relapsed or refractory solid tumor is gastroenterological cancer. In some embodiments, the relapsed or refractory solid tumor is melanoma. In some embodiments, the relapsed or refractory solid tumor is lung cancer. In some embodiments, the relapsed or refractory solid tumor is kidney cancer. In some embodiments, the relapsed or refractory solid tumor is head and neck cancer. In some embodiments, the relapsed or refractory solid tumor is proximal or distal bile duct cancer. In some embodiments, the relapsed or refractory solid tumor is alveolar soft part sarcoma. In some embodiments, the relapsed or refractory solid tumor is Ewing's bone sarcoma. In some embodiments, the relapsed or refractory solid tumor is bladder cancer. In some embodiments, the relapsed or refractory solid tumor is ovarian cancer. In some embodiments, the relapsed or refractory solid tumor is leiomyosarcoma. In some embodiments, the relapsed or refractory solid tumor is osteosarcoma. In some embodiments, the relapsed or refractory solid tumor is neuroblastoma.

In some embodiments, a relapsed or refractory solid tumor is a relapsed or refractory ibrutinib-resistant solid tumor. In some embodiments, described herein is a method of treating a relapsed or refractory ibrutinib-resistant solid tumor in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the relapsed or refractory ibrutinib-resistant solid tumor is selected from alveolar soft part sarcoma, bladder cancer, breast cancer, colorectal (colon) cancer, Ewing's bone sarcoma, gastroenterological cancer, head and neck cancer, kidney cancer, leiomyosarcoma, lung cancer, melanoma, osteosarcoma, ovarian cancer, pancreatic cancer, prostate cancer, proximal or distal bile duct cancer, and neuroblastoma. In some embodiments, the relapsed or refractory ibrutinib-resistant solid tumor is prostate cancer. In some embodiments, the relapsed or refractory ibrutinib-resistant solid tumor is breast cancer. In some embodiments, the relapsed or refractory ibrutinib-resistant solid tumor is lung cancer. In some embodiments, the relapsed or refractory ibrutinib-resistant solid tumor is colorectal (colon) cancer. In some embodiments, the relapsed or refractory ibrutinib-resistant solid tumor is gastroenterological cancer. In some embodiments, the relapsed or refractory ibrutinib-resistant solid tumor is melanoma. In some embodiments, the relapsed or refractory ibrutinib-resistant solid tumor is lung cancer. In some embodiments, the relapsed or refractory ibrutinib-resistant solid tumor is kidney cancer. In some embodiments, the relapsed or refractory ibrutinib-resistant solid tumor is head and neck cancer. In some embodiments, the relapsed or refractory ibrutinib-resistant solid tumor is proximal or distal bile duct cancer. In some embodiments, the relapsed or refractory ibrutinib-resistant solid tumor is alveolar soft part sarcoma. In some embodiments, the relapsed or refractory ibrutinib-resistant solid tumor is Ewing's bone sarcoma. In some embodiments, the relapsed or refractory ibrutinib-resistant solid tumor is bladder cancer. In some embodiments, the relapsed or refractory ibrutinib-resistant solid tumor is ovarian cancer. In some embodiments, the relapsed or refractory ibrutinib-resistant solid tumor is leiomyosarcoma. In some embodiments, the relapsed or refractory ibrutinib-resistant solid tumor is osteosarcoma. In some embodiments, the relapsed or refractory ibrutinib-resistant solid tumor is neuroblastoma.

In some embodiments, described herein is a method of treating a relapsed or refractory breast cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory breast cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory colon cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory colon cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory lung cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory lung cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory prostate cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory prostate cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory pancreatic cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory pancreatic cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory ovarian cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory ovarian cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory bladder cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory bladder cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory proximal or distal bile duct cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory proximal or distal bile duct cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory melanoma in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory melanoma in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

### Metastasized Solid Tumor

In some embodiments, the solid tumor is a metastasized solid tumor. In some embodiments, the metastasized solid tumor is a sarcoma or carcinoma. In some embodiments, the metastasized solid tumor is a sarcoma. In some embodiments, the metastasized solid tumor is a carcinoma. In some embodiments, the sarcoma is selected from alveolar rhabdomyosarcoma; alveolar soft part sarcoma; ameloblastoma; angiosarcoma; chondrosarcoma; chordoma; clear cell sarcoma of soft tissue; dedifferentiated liposarcoma; desmoid; desmoplastic small round cell tumor; embryonal rhabdomyosarcoma; epithelioid fibrosarcoma; epithelioid hemangioendothelioma; epithelioid sarcoma; esthesioneuroblastoma; Ewing sarcoma; extrarenal rhabdoid tumor; extraskeletal myxoid chondrosarcoma; extraskeletal osteosarcoma; fibrosarcoma; giant cell tumor; hemangiopericytoma; infantile fibrosarcoma; inflammatory myofibroblastic tumor; Kaposi sarcoma; leiomyosarcoma of bone; liposarcoma; liposarcoma of bone; malignant fibrous histiocytoma (MFH); malignant fibrous histiocytoma (MFH) of bone; malignant mesenchymoma; malignant peripheral nerve sheath tumor; mesenchymal chondrosarcoma; myxofibrosarcoma; myxoid liposarcoma; myxoinflammatory fibroblastic sarcoma; neoplasms with perivascular epitheioid cell differentiation; osteosarcoma; parosteal osteosarcoma; neoplasm with perivascular epitheioid cell differentiation; periosteal osteosarcoma; pleomorphic liposarcoma; pleomorphic rhabdomyosarcoma; PNET/extraskeletal Ewing tumor; rhabdomyosarcoma; round cell liposarcoma; small cell osteosarcoma; solitary fibrous tumor; synovial sarcoma; telangiectatic osteosarcoma. In some embodiments, the carcinoma is selected from an adenocarcinoma, squamous cell carcinoma, adenosquamous carcinoma, anaplastic carcinoma, large cell carcinoma, or small cell carcinoma. In some embodiments, the carcinoma is selected from anal cancer; appendix cancer; bile duct cancer (i.e., cholangiocarcinoma); bladder cancer; breast cancer; cervical cancer; colon cancer; cancer of Unknown Primary (CUP); esophageal cancer; eye cancer; fallopian tube cancer; gastroenterological cancer; kidney cancer; liver cancer; lung cancer; medulloblastoma; melanoma; oral cancer; ovarian cancer; pancreatic cancer; parathyroid disease; penile cancer; pituitary tumor; prostate cancer; rectal cancer; skin cancer; stomach cancer; testicular cancer; throat cancer; thyroid cancer; uterine cancer; vaginal cancer; or vulvar cancer. In some embodiments, the carcinoma is breast cancer. In some embodiments, the breast cancer is invasive ductal carcinoma, ductal carcinoma in situ, invasive lobular carcinoma, or lobular carcinoma in situ. In some embodiments, the carcinoma is pancreatic cancer. In some embodiments, the pancreatic cancer is adenocarcinoma, or islet cell carcinoma. In some embodiments, the carcinoma is colorectal (colon) cancer. In some embodiments, the colorectal cancer is adenocarcinoma. In some embodiments, the solid tumor is a colon polyp. In some embodiments, the colon polyp is associated with familial adenomatous polyposis. In some embodiments, the carcinoma is bladder cancer. In some embodiments, the bladder cancer is transitional cell bladder cancer, squamous cell bladder cancer, or adenocarcinoma. In some embodiments, the carcinoma is lung cancer. In some embodiments, the lung cancer is a non-small cell lung cancer. In some embodiments, the non-small cell lung cancer is adenocarcinoma, squamous-cell lung carcinoma, or large-cell lung carcinoma. In some embodiments, the lung cancer is a small cell lung cancer. In some embodiments, the carcinoma is prostate cancer. In some embodiments, the prostate cancer is adenocarcinoma or small cell carcinoma. In some embodiments, the carcinoma is ovarian cancer. In some embodiments, the ovarian cancer is epithelial ovarian cancer. In some embodiments, the carcinoma is bile duct cancer. In some embodiments, the bile duct cancer is proximal bile duct carcinoma or distal bile duct carcinoma.

In some embodiments, the metastasized solid tumor is selected from breast cancer, lung cancer, ovarian cancer, prostate cancer, genitourinary tract cancers, osteosarcoma, leiomyosarcoma, malignant fibrous histiocytoma, alveolar soft part sarcoma, Ewing's bone sarcomas, melanoma, head and neck cancer, kidney cancer, colorectal cancer, pancreatic cancer, and neuroblastoma. In some embodiments, the metastasized solid tumor is breast cancer. In some embodiments, the metastasized solid tumor is lung cancer. In some embodiments, the metastasized solid tumor is ovarian cancer. In some embodiments, the metastasized solid tumor is prostate cancer. In some embodiments, the metastasized solid tumor is genitourinary tract cancer. In some embodiments, the metastasized solid tumor is osteosarcoma. In some embodiments, the metastasized solid tumor is leiomyosarcoma. In some embodiments, the metastasized solid tumor is malignant fibrous histiocytoma. In some embodiments, the metastasized solid tumor is alveolar soft part sarcoma. In some embodiments, the metastasized solid tumor is Ewing's bone sarcomas. In some embodiments, the metastasized solid tumor is melanoma. In some embodiments, the metastasized solid tumor is head and neck cancer. In some embodiments, the metastasized solid tumor is kidney cancer. In some embodiments, the metastasized solid tumor is colorectal cancer. In some embodiments, the metastasized solid tumor is pancreatic cancer. In some embodiments, the metastasized solid tumor is neuroblastoma.

In some embodiments, described herein is a method of treating a metastasized solid tumor in an individual in need thereof which comprises administering a combination of a TEC inhibitor and an immune checkpoint inhibitor. In some embodiments, the TEC inhibitor is a BTK, ITK, TEC, RLK, or BMX inhibitor. In some embodiments, the TEC inhibitor is a BTK inhibitor or an ITK inhibitor. In some embodiments, the TEC inhibitor is a BTK inhibitor. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the metastasized solid tumor is selected from breast cancer, lung cancer, ovarian cancer, prostate cancer, genitourinary tract cancers, osteosarcoma, leiomyosarcoma, malignant fibrous histiocytoma, alveolar soft part sarcoma, Ewing's bone sarcomas, melanoma, head and neck cancer, kidney cancer, colorectal cancer, pancreatic cancer, and neuroblastoma.

In some embodiments, described herein is a method of treating a metastasized solid tumor in an individual in need thereof which comprises administering a combination of an ITK inhibitor and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the metastasized solid tumor is selected from breast cancer, lung cancer, ovarian cancer, prostate cancer, genitourinary tract cancers, osteosarcoma, leiomyosarcoma, malignant fibrous histiocytoma, alveolar soft part sarcoma, Ewing's bone sarcomas, melanoma, head and neck cancer, kidney cancer, colorectal cancer, pancreatic cancer, and neuroblastoma.

In some embodiments, described herein is a method of treating a metastasized solid tumor in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the metastasized solid tumor is selected from breast cancer, lung cancer, ovarian cancer, prostate cancer, genitourinary tract cancers, osteosarcoma, leiomyosarcoma, malignant fibrous histiocytoma, alveolar soft part sarcoma, Ewing's bone sarcomas, melanoma, head and neck cancer, kidney cancer, colorectal cancer, pancreatic cancer, and neuroblastoma.

In some embodiments, described herein is a method of treating a metastasized solid tumor in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the metastasized solid tumor is selected from breast cancer, lung cancer, ovarian cancer, prostate cancer, genitourinary tract cancers, osteosarcoma, leiomyosarcoma, malignant fibrous histiocytoma, alveolar soft part sarcoma, Ewing's bone sarcomas, melanoma, head and neck cancer, kidney cancer, colorectal cancer, pancreatic cancer, and neuroblastoma.

In some embodiments, the metastasized solid tumor is an ibrutinib-resistant solid tumor. In some embodiments, described herein is a method of treating a metastasized ibrutinib-resistant solid tumor in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the metastasized ibrutinib-resistant solid tumor is selected from breast cancer, lung cancer, ovarian cancer, prostate cancer, genitourinary tract cancers, osteosarcoma, leiomyosarcoma, malignant fibrous histiocytoma, alveolar soft part sarcoma, Ewing's bone sarcomas, melanoma, head and neck cancer, kidney cancer, colorectal cancer, pancreatic cancer, and neuroblastoma.

In some embodiments, described herein is a method of treating a metastasized breast cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized breast cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized colon cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized colon cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized lung cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized lung cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized prostate cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized prostate cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized pancreatic cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized pancreatic cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized ovarian cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized ovarian cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized bladder cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized bladder cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized proximal or distal bile duct cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized proximal or distal bile duct cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized melanoma in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized melanoma in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

### Hematologic Cancer

Disclosed herein, in certain embodiments, is a method of treating a hematologic cancer in an individual in need thereof which comprises administering a combination of a TEC inhibitor and an immune checkpoint inhibitor. In some embodiments, the hematologic cancer is a leukemia, a lymphoma, a myeloma, a non-Hodgkin's lymphoma, a Hodgkin's lymphoma, a T-cell malignancy, or a B-cell malignancy.

In some embodiments, the hematologic cancer is a T-cell malignancy. In some embodiments, the T-cell malignancy is peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some embodiments, the hematologic cancer is a B-cell proliferative disorder. In some embodiments, the cancer is chronic lymphocytic leukemia (CLL), small lymphocytic lymphoma (SLL), high risk CLL, or a non-CLL/SLL lymphoma. In some embodiments, the cancer is follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, DLBCL is further divided into subtypes: activated B-cell diffuse large B-cell lymphoma (ABC-DLBCL), germinal center diffuse large B-cell lymphoma (GCB DLBCL), and Double-Hit (DH) DLBCL. In some embodiments, ABC-DLBCL is characterized by a CD79B mutation. In some embodiments, ABC-DLBCL is characterized by a CD79A mutation. In some embodiments, the ABC-DLBCL is characterized by a mutation in MyD88, A20, or a combination thereof. In some embodiments, the cancer is acute or chronic myelogenous (or myeloid) leukemia, myelodysplastic syndrome, or acute lymphoblastic leukemia.

In some embodiments, the cancer is diffuse large B-cell lymphoma (DLBCL). In some embodiments, the cancer is activated B-cell diffuse large B-cell lymphoma (ABC-DLBCL). In some embodiments, the cancer is follicular lymphoma (FL). In some embodiments, the cancer is multiple myeloma. In some embodiments, the cancer is chronic lymphocytic leukemia (CLL). In some embodiments, the cancer is small lymphocytic lymphoma (SLL). In some embodiments, the cancer is non-CLL/SLL lymphoma. In some embodiments, the cancer is high risk CLL or high risk SLL.

In some embodiments, described herein is a method of treating a hematologic cancer in an individual in need thereof which comprises administering a combination of a TEC inhibitor and an immune checkpoint inhibitor. In some embodiments, the TEC inhibitor is a BTK, ITK, TEC, RLK, or BMX inhibitor. In some embodiments, the TEC inhibitor is a BTK inhibitor or an ITK inhibitor. In some embodiments, the TEC inhibitor is a BTK inhibitor. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the hematologic cancer is a leukemia, a lymphoma, a myeloma, a non-Hodgkin's lymphoma, a Hodgkin's lymphoma, a T-cell malignancy, or a B-cell malignancy. In some embodiments, the hematologic cancer is a B-cell malignancy. In some embodiments, the B-cell malignancy is chronic lymphocytic leukemia (CLL), small lymphocytic lymphoma (SLL), high risk CLL, non-CLL/SLL lymphoma, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the hematologic cancer is CLL. In some embodiments, the hematologic cancer is SLL. In some embodiments, the hematologic cancer is DLBCL. In some embodiments, the hematologic cancer is mantle cell lymphoma. In some embodiments, the hematologic cancer is FL. In some embodiments, the hematologic cancer is Waldenstrom's macroglobulinemia. In some embodiments, the hematologic cancer is multiple myeloma. In some embodiments, the hematologic cancer is Burkitt's lymphoma.

In some embodiments, described herein is a method of treating a hematologic cancer in an individual in need thereof which comprises administering a combination of an ITK inhibitor and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the hematologic cancer is a leukemia, a lymphoma, a myeloma, a non-Hodgkin's lymphoma, a Hodgkin's lymphoma, a T-cell malignancy, or a B-cell malignancy. In some embodiments, the hematologic cancer is a B-cell malignancy. In some embodiments, the B-cell malignancy is chronic lymphocytic leukemia (CLL), small lymphocytic lymphoma (SLL), high risk CLL, non-CLL/SLL lymphoma, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the hematologic cancer is CLL. In some embodiments, the hematologic cancer is SLL. In some embodiments, the hematologic cancer is DLBCL. In some embodiments, the hematologic cancer is mantle cell lymphoma. In some embodiments, the hematologic cancer is FL. In some embodiments, the hematologic cancer is Waldenstrom's macroglobulinemia. In some embodiments, the hematologic cancer is multiple myeloma. In some embodiments, the hematologic cancer is Burkitt's lymphoma.

In some embodiments, described herein is a method of treating a hematologic cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the hematologic cancer is a leukemia, a lymphoma, a myeloma, a non-Hodgkin's lymphoma, a Hodgkin's lymphoma, a T-cell malignancy, or a B-cell malignancy. In some embodiments, the hematologic cancer is a B-cell malignancy. In some embodiments, the B-cell malignancy is chronic lymphocytic leukemia (CLL), small lymphocytic lymphoma (SLL), high risk CLL, non-CLL/SLL lymphoma, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the hematologic cancer is CLL. In some embodiments, the hematologic cancer is SLL. In some embodiments, the hematologic cancer is DLBCL. In some embodiments, the hematologic cancer is mantle cell lymphoma. In some embodiments, the hematologic cancer is FL. In some embodiments, the hematologic cancer is Waldenstrom's macroglobulinemia. In some embodiments, the hematologic cancer is multiple myeloma. In some embodiments, the hematologic cancer is Burkitt's lymphoma.

In some embodiments, described herein is a method of treating a hematologic cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the hematologic cancer is a leukemia, a lymphoma, a myeloma, a non-Hodgkin's lymphoma, a Hodgkin's lymphoma, a T-cell malignancy, or a B-cell malignancy. In some embodiments, the hematologic cancer is a B-cell malignancy. In some embodiments, the B-cell malignancy is chronic lymphocytic leukemia (CLL), small lymphocytic lymphoma (SLL), high risk CLL, non-CLL/SLL lymphoma, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the hematologic cancer is CLL. In some embodiments, the hematologic cancer is SLL. In some embodiments, the hematologic cancer is DLBCL. In some embodiments, the hematologic cancer is mantle cell lymphoma. In some embodiments, the hematologic cancer is FL. In some embodiments, the hematologic cancer is Waldenstrom's macroglobulinemia. In some embodiments, the hematologic cancer is multiple myeloma. In some embodiments, the hematologic cancer is Burkitt's lymphoma.

In some embodiments, the hematologic cancer is an ibrutinib-resistant hematologic cancer. In some embodiments, described herein is a method of treating an ibrutinib-resistant hematologic cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the ibrutinib-resistant hematologic cancer is a leukemia, a lymphoma, a myeloma, a non-Hodgkin's lymphoma, a Hodgkin's lymphoma, a T-cell malignancy, or a B-cell malignancy. In some embodiments, the ibrutinib-resistant hematologic cancer is a B-cell malignancy. In some embodiments, the B-cell malignancy is chronic lymphocytic leukemia (CLL), small lymphocytic lymphoma (SLL), high risk CLL, non-CLL/SLL lymphoma, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the ibrutinib-resistant hematologic cancer is CLL. In some embodiments, the ibrutinib-resistant hematologic cancer is SLL. In some embodiments, the ibrutinib-resistant hematologic cancer is DLBCL. In some embodiments, the ibrutinib-resistant hematologic cancer is mantle cell lymphoma. In some embodiments, the ibrutinib-resistant hematologic cancer is FL. In some embodiments, the ibrutinib-resistant hematologic cancer is Waldenstrom's macroglobulinemia. In some embodiments, the ibrutinib-resistant hematologic cancer is multiple myeloma. In some embodiments, the ibrutinib-resistant hematologic cancer is Burkitt's lymphoma.

In some embodiments, described herein is a method of treating CLL in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating CLL in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating SLL in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating SLL in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating mantle cell lymphoma in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating mantle cell lymphoma in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating DLBCL in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the DLBCL is ABC-DLBCL, GCB-DLBCL, or DH-DLBCL.

In some embodiments, described herein is a method of treating DLBCL in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the DLBCL is ABC-DLBCL, GCB-DLBCL, or DH-DLBCL.

In some embodiments, described herein is a method of treating Waldenström's macroglobulinemia in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating Waldenström's macroglobulinemia in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, a cancer is a treatment-naive cancer. In some instances, a treatment-naive cancer is a cancer that has not been treated by a therapy, such as for example by a TEC inhibitor, an immune checkpoint inhibitor, and/or by an additional therapeutic agent disclosed elsewhere herein. In some embodiments, a treatment-naive cancer is a hematologic cancer. In some embodiments, described herein is a method of treating a treatment-naive hematologic cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the treatment-naive hematologic cancer is a leukemia, a lymphoma, a myeloma, a non-Hodgkin's lymphoma, a Hodgkin's lymphoma, a T-cell malignancy, or a B-cell malignancy. In some embodiments, the ibrutinib-resistant hematologic cancer is a B-cell malignancy. In some embodiments, the B-cell malignancy is chronic lymphocytic leukemia (CLL), small lymphocytic lymphoma (SLL), high risk CLL, non-CLL/SLL lymphoma, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the treatment-naive hematologic cancer is CLL. In some embodiments, the treatment-naive hematologic cancer is SLL. In some embodiments, the treatment-naive hematologic cancer is DLBCL. In some embodiments, the treatment-naive hematologic cancer is mantle cell lymphoma. In some embodiments, the treatment-naive hematologic cancer is FL. In some embodiments, the treatment-naive hematologic cancer is Waldenstrom's macroglobulinemia. In some embodiments, the treatment-naive hematologic cancer is multiple myeloma. In some embodiments, the treatment-naive hematologic cancer is Burkitt's lymphoma.

### Relapsed or Refractory Hematologic Cancer

In some embodiments, the hematologic cancer is a relapsed or refractory hematologic cancer. In some embodiments, the relapsed or refractory hematologic cancer is a leukemia, a lymphoma, a myeloma, a non-Hodgkin's lymphoma, a Hodgkin's lymphoma, T-cell malignancy, or a B-cell malignancy.

In some embodiments, the relapsed or refractory hematologic cancer is a T-cell malignancy. In some embodiments, the relapsed or refractory T-cell malignancy is peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some embodiments, the relapsed or refractory hematologic cancer is a B-cell proliferative disorder. In some embodiments, the relapsed or refractory cancer is chronic lymphocytic leukemia (CLL), small lymphocytic lymphoma (SLL), high risk CLL, or a non-CLL/SLL lymphoma. In some embodiments, the cancer is follicular lymphoma, diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the relapsed or refractory DLBCL is further divided into subtypes: activated B-cell diffuse large B-cell lymphoma (ABC-DLBCL), germinal center diffuse large B-cell lymphoma (GCB DLBCL), and Double-Hit (DH) DLBCL. In some embodiments, ABC-DLBCL is characterized by a CD79B mutation. In some embodiments, ABC-DLBCL is characterized by a CD79A mutation. In some embodiments, the ABC-DLBCL is characterized by a mutation in MyD88, A20, or a combination thereof. In some embodiments, the cancer is acute or chronic myelogenous (or myeloid) leukemia, myelodysplastic syndrome, or acute lymphoblastic leukemia.

In some embodiments, the cancer is relapsed or refractory diffuse large B-cell lymphoma (DLBCL). In some embodiments, the cancer is relapsed or refractory activated B-cell diffuse large B-cell lymphoma (ABC-DLBCL). In some embodiments, the cancer is relapsed or refractory follicular lymphoma (FL). In some embodiments, the cancer is relapsed or refractory multiple myeloma. In some embodiments, the cancer is relapsed or refractory chronic lymphocytic leukemia (CLL). In some embodiments, the cancer is relapsed or refractory small lymphocytic lymphoma (SLL). In some embodiments, the cancer is relapsed or refractory non-CLL/SLL lymphoma. In some embodiments, the cancer is relapsed or refractory high risk CLL or high risk SLL.

In some embodiments, described herein is a method of treating a relapsed or refractory hematologic cancer in an individual in need thereof which comprises administering a combination of a TEC inhibitor and an immune checkpoint inhibitor. In some embodiments, the individual has relapsed or has developed a refractory hematologic cancer to an existing therapy. In some embodiments, the TEC inhibitor is a BTK, ITK, TEC, RLK, or BMX inhibitor. In some embodiments, the TEC inhibitor is a BTK inhibitor or an ITK inhibitor. In some embodiments, the TEC inhibitor is a BTK inhibitor. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the relapsed or refractory hematologic cancer is a leukemia, a lymphoma, a myeloma, a non-Hodgkin's lymphoma, a Hodgkin's lymphoma, a T-cell malignancy, or a B-cell malignancy. In some embodiments, the relapsed or refractory hematologic cancer is a relapsed or refractory B-cell malignancy. In some embodiments, the relapsed or refractory B-cell malignancy is chronic lymphocytic leukemia (CLL), small lymphocytic lymphoma (SLL), high risk CLL, non-CLL/SLL lymphoma, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the relapsed or refractory hematologic cancer is relapsed or refractory CLL. In some embodiments, the relapsed or refractory hematologic cancer is relapsed or refractory SLL. In some embodiments, the relapsed or refractory hematologic cancer is relapsed or refractory DLBCL. In some embodiments, the relapsed or refractory hematologic cancer is relapsed or refractory mantle cell lymphoma. In some embodiments, the relapsed or refractory hematologic cancer is relapsed or refractory FL. In some embodiments, the relapsed or refractory hematologic cancer is relapsed or refractory Waldenstrom's macroglobulinemia. In some embodiments, the relapsed or refractory hematologic cancer is relapsed or refractory multiple myeloma. In some embodiments, the relapsed or refractory hematologic cancer is relapsed or refractory Burkitt's lymphoma.

In some embodiments, described herein is a method of treating a relapsed or refractory hematologic cancer in an individual in need thereof which comprises administering a combination of an ITK inhibitor and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the relapsed or refractory hematologic cancer is a leukemia, a lymphoma, a myeloma, a non-Hodgkin's lymphoma, a Hodgkin's lymphoma, a T-cell malignancy, or a B-cell malignancy. In some embodiments, the relapsed or refractory hematologic cancer is a relapsed or refractory B-cell malignancy. In some embodiments, the relapsed or refractory B-cell malignancy is chronic lymphocytic leukemia (CLL), small lymphocytic lymphoma (SLL), high risk CLL, non-CLL/SLL lymphoma, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the relapsed or refractory hematologic cancer is relapsed or refractory CLL. In some embodiments, the relapsed or refractory hematologic cancer is relapsed or refractory SLL. In some embodiments, the relapsed or refractory hematologic cancer is relapsed or refractory DLBCL. In some embodiments, the relapsed or refractory hematologic cancer is relapsed or refractory mantle cell lymphoma. In some embodiments, the relapsed or refractory hematologic cancer is relapsed or refractory FL. In some embodiments, the relapsed or refractory hematologic cancer is relapsed or refractory Waldenstrom's macroglobulinemia. In some embodiments, the relapsed or refractory hematologic cancer is relapsed or refractory multiple myeloma. In some embodiments, the relapsed or refractory hematologic cancer is relapsed or refractory Burkitt's lymphoma.

In some embodiments, described herein is a method of treating a relapsed or refractory hematologic cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the relapsed or refractory hematologic cancer is a leukemia, a lymphoma, a myeloma, a non-Hodgkin's lymphoma, a Hodgkin's lymphoma, a T-cell malignancy, or a B-cell malignancy. In some embodiments, the relapsed or refractory hematologic cancer is a relapsed or refractory B-cell malignancy. In some embodiments, the relapsed or refractory B-cell malignancy is chronic lymphocytic leukemia (CLL), small lymphocytic lymphoma (SLL), high risk CLL, non-CLL/SLL lymphoma, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the relapsed or refractory hematologic cancer is relapsed or refractory CLL. In some embodiments, the relapsed or refractory hematologic cancer is relapsed or refractory SLL. In some embodiments, the relapsed or refractory hematologic cancer is relapsed or refractory DLBCL. In some embodiments, the relapsed or refractory hematologic cancer is relapsed or refractory mantle cell lymphoma. In some embodiments, the relapsed or refractory hematologic cancer is relapsed or refractory FL. In some embodiments, the relapsed or refractory hematologic cancer is relapsed or refractory Waldenstrom's macroglobulinemia. In some embodiments, the relapsed or refractory hematologic cancer is relapsed or refractory multiple myeloma. In some embodiments, the relapsed or refractory hematologic cancer is relapsed or refractory Burkitt's lymphoma.

In some embodiments, described herein is a method of treating a relapsed or refractory hematologic cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the relapsed or refractory hematologic cancer is a leukemia, a lymphoma, a myeloma, a non-Hodgkin's lymphoma, a Hodgkin's lymphoma, a T-cell malignancy, or a B-cell malignancy. In some embodiments, the relapsed or refractory hematologic cancer is a relapsed or refractory B-cell malignancy. In some embodiments, the relapsed or refractory B-cell malignancy is chronic lymphocytic leukemia (CLL), small lymphocytic lymphoma (SLL), high risk CLL, non-CLL/SLL lymphoma, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the relapsed or refractory hematologic cancer is relapsed or refractory CLL. In some embodiments, the relapsed or refractory hematologic cancer is relapsed or refractory SLL. In some embodiments, the relapsed or refractory hematologic cancer is relapsed or refractory DLBCL. In some embodiments, the relapsed or refractory hematologic cancer is relapsed or refractory mantle cell lymphoma. In some embodiments, the relapsed or refractory hematologic cancer is relapsed or refractory FL. In some embodiments, the relapsed or refractory hematologic cancer is relapsed or refractory Waldenstrom's macroglobulinemia. In some embodiments, the relapsed or refractory hematologic cancer is relapsed or refractory multiple myeloma. In some embodiments, the relapsed or refractory hematologic cancer is relapsed or refractory Burkitt's lymphoma.

In some embodiments, the relapsed or refractory hematologic cancer is a relapsed or refractory ibrutinib-resistant hematologic cancer. In some embodiments, described herein is a method of treating a relapsed or refractory ibrutinib-resistant hematologic cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the relapsed or refractory ibrutinib-resistant hematologic cancer is a leukemia, a lymphoma, a myeloma, a non-Hodgkin's lymphoma, a Hodgkin's lymphoma, a T-cell malignancy, or a B-cell malignancy. In some embodiments, the ibrutinib-resistant relapsed or refractory hematologic cancer is a relapsed or refractory B-cell malignancy. In some embodiments, the relapsed or refractory B-cell malignancy is chronic lymphocytic leukemia (CLL), small lymphocytic lymphoma (SLL), high risk CLL, non-CLL/SLL lymphoma, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the relapsed or refractory ibrutinib-resistant hematologic cancer is relapsed or refractory CLL. In some embodiments, the relapsed or refractory ibrutinib-resistant hematologic cancer is relapsed or refractory SLL. In some embodiments, the relapsed or refractory ibrutinib-resistant hematologic cancer is relapsed or refractory DLBCL. In some embodiments, the relapsed or refractory ibrutinib-resistant hematologic cancer is relapsed or refractory mantle cell lymphoma. In some embodiments, the relapsed or refractory ibrutinib-resistant hematologic cancer is relapsed or refractory FL. In some embodiments, the relapsed or refractory ibrutinib-resistant hematologic cancer is relapsed or refractory Waldenstrom's macroglobulinemia. In some embodiments, the relapsed or refractory ibrutinib-resistant hematologic cancer is relapsed or refractory multiple myeloma. In some embodiments, the relapsed or refractory ibrutinib-resistant hematologic cancer is relapsed or refractory Burkitt's lymphoma.

In some embodiments, described herein is a method of treating a relapsed or refractory CLL in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory CLL in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory SLL in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory SLL in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory mantle cell lymphoma in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory mantle cell lymphoma in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory DLBCL in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the DLBCL is ABC-DLBCL, GCB-DLBCL, or DH-DLBCL.

In some embodiments, described herein is a method of treating a relapsed or refractory DLBCL in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the DLBCL is ABC-DLBCL, GCB-DLBCL, or DH-DLBCL.

In some embodiments, described herein is a method of treating a relapsed or refractory Waldenström's macroglobulinemia in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a relapsed or refractory Waldenström's macroglobulinemia in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

### Metastasized Hematologic Cancer

In some embodiments, the hematologic cancer is a metastasized hematologic cancer. In some embodiments, the metastasized hematologic cancer is a leukemia, a lymphoma, a myeloma, a non-Hodgkin's lymphoma, a Hodgkin's lymphoma, a T-cell malignancy, or a B-cell malignancy.

In some embodiments, the metastasized hematologic cancer is a T-cell malignancy. In some embodiments, the T-cell malignancy is peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some embodiments, the metastasized hematologic cancer is a B-cell proliferative disorder. In some embodiments, the metastasized hematologic cancer is chronic lymphocytic leukemia (CLL), small lymphocytic lymphoma (SLL), high risk CLL, or a non-CLL/SLL lymphoma. In some embodiments, the metastasized hematologic cancer is follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, DLBCL is further divided into subtypes: activated B-cell diffuse large B-cell lymphoma (ABC-DLBCL), germinal center diffuse large B-cell lymphoma (GCB DLBCL), and Double-Hit (DH) DLBCL. In some embodiments, ABC-DLBCL is characterized by a CD79B mutation. In some embodiments, ABC-DLBCL is characterized by a CD79A mutation. In some embodiments, the ABC-DLBCL is characterized by a mutation in MyD88, A20, or a combination thereof. In some embodiments, the cancer is acute or chronic myelogenous (or myeloid) leukemia, myelodysplastic syndrome, or acute lymphoblastic leukemia.

In some embodiments, the metastasized hematologic cancer is diffuse large B-cell lymphoma (DLBCL). In some embodiments, the metastasized hematologic cancer is activated B-cell diffuse large B-cell lymphoma (ABC-DLBCL). In some embodiments, the metastasized hematologic cancer is follicular lymphoma (FL). In some embodiments, the metastasized hematologic cancer is multiple myeloma. In some embodiments, the metastasized hematologic cancer is chronic lymphocytic leukemia (CLL). In some embodiments, the metastasized hematologic cancer is small lymphocytic lymphoma (SLL). In some embodiments, the metastasized hematologic cancer is non-CLL/SLL lymphoma. In some embodiments, the metastasized hematologic cancer is high risk CLL or high risk SLL.

In some embodiments, described herein is a method of treating a metastasized hematologic cancer in an individual in need thereof which comprises administering a combination of a TEC inhibitor and an immune checkpoint inhibitor. In some embodiments, the TEC inhibitor is a BTK, ITK, TEC, RLK, or BMX inhibitor. In some embodiments, the TEC inhibitor is a BTK inhibitor or an ITK inhibitor. In some embodiments, the TEC inhibitor is a BTK inhibitor. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the metastasized hematologic cancer is a leukemia, a lymphoma, a myeloma, a non-Hodgkin's lymphoma, a Hodgkin's lymphoma, a T-cell malignancy, or a B-cell malignancy. In some embodiments, the metastasized hematologic cancer is a metastasized B-cell malignancy. In some embodiments, the metastasized B-cell malignancy is chronic lymphocytic leukemia (CLL), small lymphocytic lymphoma (SLL), high risk CLL, non-CLL/SLL lymphoma, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the metastasized hematologic cancer is metastasized CLL. In some embodiments, the metastasized hematologic cancer is metastasized SLL. In some embodiments, the metastasized hematologic cancer is metastasized DLBCL. In some embodiments, the metastasized hematologic cancer is metastasized mantle cell lymphoma. In some embodiments, the metastasized hematologic cancer is metastasized FL. In some embodiments, the metastasized hematologic cancer is metastasized Waldenstrom's macroglobulinemia. In some embodiments, the metastasized hematologic cancer is metastasized multiple myeloma. In some embodiments, the metastasized hematologic cancer is metastasized Burkitt's lymphoma.

In some embodiments, described herein is a method of treating a metastasized hematologic cancer in an individual in need thereof which comprises administering a combination of an ITK inhibitor and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the metastasized hematologic cancer is a leukemia, a lymphoma, a myeloma, a non-Hodgkin's lymphoma, a Hodgkin's lymphoma, a T-cell malignancy, or a B-cell malignancy. In some embodiments, the metastasized hematologic cancer is a metastasized B-cell malignancy. In some embodiments, the metastasized B-cell malignancy is chronic lymphocytic leukemia (CLL), small lymphocytic lymphoma (SLL), high risk CLL, non-CLL/SLL lymphoma, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the metastasized hematologic cancer is metastasized CLL. In some embodiments, the metastasized hematologic cancer is metastasized SLL. In some embodiments, the metastasized hematologic cancer is metastasized DLBCL. In some embodiments, the metastasized hematologic cancer is metastasized mantle cell lymphoma. In some embodiments, the metastasized hematologic cancer is metastasized FL. In some embodiments, the metastasized hematologic cancer is metastasized Waldenstrom's macroglobulinemia. In some embodiments, the metastasized hematologic cancer is metastasized multiple myeloma. In some embodiments, the metastasized hematologic cancer is metastasized Burkitt's lymphoma.

In some embodiments, described herein is a method of treating a metastasized hematologic cancer in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the metastasized hematologic cancer is a leukemia, a lymphoma, a myeloma, a non-Hodgkin's lymphoma, a Hodgkin's lymphoma, a T-cell malignancy, or a B-cell malignancy. In some embodiments, the metastasized hematologic cancer is a metastasized B-cell malignancy. In some embodiments, the metastasized B-cell malignancy is chronic lymphocytic leukemia (CLL), small lymphocytic lymphoma (SLL), high risk CLL, non-CLL/SLL lymphoma, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the metastasized hematologic cancer is metastasized CLL. In some embodiments, the metastasized hematologic cancer is metastasized SLL. In some embodiments, the metastasized hematologic cancer is metastasized DLBCL. In some embodiments, the metastasized hematologic cancer is metastasized mantle cell lymphoma. In some embodiments, the metastasized hematologic cancer is metastasized FL. In some embodiments, the metastasized hematologic cancer is metastasized Waldenstrom's macroglobulinemia. In some embodiments, the metastasized hematologic cancer is metastasized multiple myeloma. In some embodiments, the metastasized hematologic cancer is metastasized Burkitt's lymphoma.

In some embodiments, described herein is a method of treating a metastasized hematologic cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the metastasized hematologic cancer is a leukemia, a lymphoma, a myeloma, a non-Hodgkin's lymphoma, a Hodgkin's lymphoma, a T-cell malignancy, or a B-cell malignancy. In some embodiments, the metastasized hematologic cancer is a metastasized B-cell malignancy. In some embodiments, the metastasized B-cell malignancy is chronic lymphocytic leukemia (CLL), small lymphocytic lymphoma (SLL), high risk CLL, non-CLL/SLL lymphoma, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the metastasized hematologic cancer is metastasized CLL. In some embodiments, the metastasized hematologic cancer is metastasized SLL. In some embodiments, the metastasized hematologic cancer is metastasized DLBCL. In some embodiments, the metastasized hematologic cancer is metastasized mantle cell lymphoma. In some embodiments, the metastasized hematologic cancer is metastasized FL. In some embodiments, the metastasized hematologic cancer is metastasized Waldenstrom's macroglobulinemia. In some embodiments, the metastasized hematologic cancer is metastasized multiple myeloma. In some embodiments, the metastasized hematologic cancer is metastasized Burkitt's lymphoma.

In some embodiments, a metastasized hematologic cancer is an ibrutinib-resistant hematologic cancer. In some embodiments, described herein is a method of treating a metastasized ibrutinib-resistant hematologic cancer in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the metastasized ibrutinib-resistant hematologic cancer is a leukemia, a lymphoma, a myeloma, a non-Hodgkin's lymphoma, a Hodgkin's lymphoma, a T-cell malignancy, or a B-cell malignancy. In some embodiments, the metastasized ibrutinib-resistant hematologic cancer is a metastasized B-cell malignancy. In some embodiments, the metastasized B-cell malignancy is chronic lymphocytic leukemia (CLL), small lymphocytic lymphoma (SLL), high risk CLL, non-CLL/SLL lymphoma, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the metastasized ibrutinib-resistant hematologic cancer is metastasized CLL. In some embodiments, the metastasized ibrutinib-resistant hematologic cancer is metastasized SLL. In some embodiments, the metastasized ibrutinib-resistant hematologic cancer is metastasized DLBCL. In some embodiments, the metastasized ibrutinib-resistant hematologic cancer is metastasized mantle cell lymphoma. In some embodiments, the metastasized ibrutinib-resistant hematologic cancer is metastasized FL. In some embodiments, the metastasized ibrutinib-resistant hematologic cancer is metastasized Waldenstrom's macroglobulinemia. In some embodiments, the metastasized ibrutinib-resistant hematologic cancer is metastasized multiple myeloma. In some embodiments, the metastasized ibrutinib-resistant hematologic cancer is metastasized Burkitt's lymphoma.

In some embodiments, described herein is a method of treating a metastasized CLL in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutic s/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized CLL in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized SLL in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized SLL in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized mantle cell lymphoma in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized mantle cell lymphoma in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized DLBCL in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the DLBCL is ABC-DLBCL, GCB-DLBCL, or DH-DLBCL.

In some embodiments, described herein is a method of treating a metastasized DLBCL in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the DLBCL is ABC-DLBCL, GCB-DLBCL, or DH-DLBCL.

In some embodiments, described herein is a method of treating a metastasized Waldenström's macroglobulinemia in an individual in need thereof which comprises administering a combination of a BTK inhibitor and an immune checkpoint inhibitor. In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, described herein is a method of treating a metastasized Waldenström's macroglobulinemia in an individual in need thereof which comprises administering a combination of ibrutinib and an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

### Additional Therapeutic Agents

In some embodiments, a TEC inhibitor and an immune checkpoint inhibitor are administered in combination with an additional therapeutic agent for the treatment of cancer. In some embodiments, the additional therapeutic agent is an anticancer agent for the treatment of a solid tumor. In some embodiments, the additional therapeutic agent is an anticancer agent for the treatment of a hematologic cancer. In some embodiments, the additional anticancer agent is an anticancer agent for the treatment of a B-cell malignancy, such as CLL, SLL, DLBCL, mantle cell lymphoma, or Waldenström's macroglobulinemia. In some embodiments, the additional anticancer agent is an anticancer agent for the treatment of a solid tumor such as bladder, breast, colon, pancreatic, lung, prostate, ovarian, proximal or distal bile duct cancer, or melanoma. Non-limiting examples of anticancer agent include chemotherapeutic agents, biologic agents, radiation therapy, thermal therapy, or surgery. In some embodiments, the TEC inhibitor is a BTK, ITK, TEC, RLK, or BMX inhibitor. In some embodiments, the TEC inhibitor is a BTK inhibitor or an ITK inhibitor. In some embodiments, the TEC inhibitor is a BTK inhibitor. In some embodiments, the BTK inhibitor is ibrutinib. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof.

In some embodiments, a TEC inhibitor (e.g. ITK inhibitor or BTK inhibitor such as ibrutinib) and an immune checkpoint inhibitor are administered in combination with an anticancer agent such as for example irinotecan, cisplatin, carboplatin, methotrexate, etoposide, bleomycin, vinblastine, actinomycin (dactinomycin), cyclophosphamide, ifosfamide, gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, or PD184352, Taxol™, also referred to as “paclitaxel”, which is a well-known anti-cancer drug which acts by enhancing and stabilizing microtubule formation, analogs of Taxol™, such as Taxotere™, or a combination thereof.

In some embodiments, a TEC inhibitor (e.g. ITK inhibitor or BTK inhibitor such as ibrutinib) and an immune checkpoint inhibitor are administered in combination with an anticancer agent such as for example inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; mTOR inhibitors; and antibodies (e.g., rituxan).

In some embodiments, a TEC inhibitor (e.g. ITK inhibitor or BTK inhibitor such as ibrutinib) and an immune checkpoint inhibitor are administered in combination with an anticancer agent such as for example Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin II (including recombinant interleukin II, or r1L2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

In some embodiments, a TEC inhibitor (e.g. ITK inhibitor or BTK inhibitor such as ibrutinib) and an immune checkpoint inhibitor are administered in combination with an anticancer agent such as for example 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

In some embodiments, a TEC inhibitor (e.g. ITK inhibitor or BTK inhibitor such as ibrutinib) and an immune checkpoint inhibitor are administered in combination with an anticancer agent such as for example alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

In some embodiments, a TEC inhibitor (e.g. ITK inhibitor or BTK inhibitor such as ibrutinib) and an immune checkpoint inhibitor are administered in combination with an anticancer agent such as for example vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha, IL-2, IL-21).

In some embodiments, a TEC inhibitor (e.g. ITK inhibitor or BTK inhibitor such as ibrutinib) and an immune checkpoint inhibitor are administered in combination with an anticancer agent such as for example nitrogen mustards (e.g., mechloroethamine, cyclophosphamide, chlorambucil, meiphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, ete.). Examples of antimetabolites include, but are not limited to folic acid analog (e.g., methotrexate), or pyrimidine analogs (e.g., fluorouracil, floxouridine, Cytarabine), purine analogs (e.g., mercaptopurine, thioguanine, pentostatin.

In some embodiments, a TEC inhibitor (e.g. ITK inhibitor or BTK inhibitor such as ibrutinib) and an immune checkpoint inhibitor are administered in combination with an anticancer agent such as for example adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents for use in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

In some embodiments, a TEC inhibitor (e.g. ITK inhibitor or BTK inhibitor such as ibrutinib) and an immune checkpoint inhibitor are administered in combination with an anticancer agent such as for example thrombolytic agents (e.g., alteplase anistreplase, streptokinase, urokinase, or tissue plasminogen activator), heparin, tinzaparin, warfarin, dabigatran (e.g., dabigatran etexilate), factor Xa inhibitors (e.g., fondaparinux, draparinux, rivaroxaban, DX-9065a, otamixaban, LY517717, or YM150), factor VIIa inhibitors, ticlopidine, clopidogrel, CS-747 (prasugrel, LY640315), ximelagatran, or BIBR 1048.

In some embodiments, a TEC inhibitor (e.g. ITK inhibitor or BTK inhibitor such as ibrutinib) and an immune checkpoint inhibitor are administered in combination with an anticancer agent such as for example ABVD (adriamycin, bleomycin, vinblastine and dacarbazine), ChlvPP (chlorambucil, vinblastine, procarbazine and prednisolone), Stanford V (mustine, doxorubicin, vinblastine, vincristine, bleomycin, etoposide and steroids), BEACOPP (bleomycin, etoposide, doxorubicin, cyclophosphamide, vincristine, procarbazine and prednisolone), BEAM (carmustine (BiCNU) etoposide, cytarabine (Ara-C, cytosine arabinoside), and melphalan), CHOP (cyclophosphamide, doxorubicin, vincristine, and prednisone), R-CHOP (rituximab, doxorubicin, cyclophosphamide, vincristine, and prednisone), EPOCH (etoposide, vincristine, doxorubicin, cyclophosphamide, and prednisone), CVP (cyclophosphamide, vincristine, and prednisone), ICE (ifosfamide-carboplatin-etoposide), R-ACVBP (rituximab, doxorubicin, cyclophosphamide, vindesine, bleomycin, and prednisone), DHAP (dexamethasone, high-dose cytarabine, (Ara C), cisplatin), R-DHAP (rituximab, dexamethasone, high-dose cytarabine, (Ara C), cisplatin), ESHAP (etoposide (VP-16), methyl-prednisolone, and high-dose cytarabine (Ara-C), cisplatin), CDE (cyclophosphamide, doxorubicin and etoposide), Velcade® (bortezomib) plus Doxil® (liposomal doxorubicin), Revlimid® (lenalidomide) plus dexamethasone, and bortezomib plus dexamethasone.

In some embodiments, a TEC inhibitor (e.g. ITK inhibitor or BTK inhibitor such as ibrutinib) and an immune checkpoint inhibitor are administered in combination with an anticancer agent such as for example a cancer vaccine. In some instances, a cancer vaccine is a peptide-based vaccine, a nucleic acid based vaccine, a cell-based vaccine, a virus-based or viral fragment based vaccine, an antibody or antibody fragment based vaccine, or an antigen presenting cell (APC) based vaccine (e.g. dendritic cell based vaccine). Exemplary cancer vaccines include Gardasil®, Cervarix®, sipuleucel-T (Provenge®), NeuVax™, HER-2 ICD peptide-based vaccine, HER-2/neu peptide baccine, AdHER2/neu dendritic cell vaccine, HER-2 pulsed DC1 vaccine, Ad-sig-hMUC-1/ecdCD40L fusion protein vaccine, MVX-ONCO-1, hTERT/survivin/CMV multipeptide vaccine, E39, J65, P10s-PADRE, rV-CEA-Tricom, GVAX®, Lucanix®, HER2VRP, AVX901, ONT-10, ISA101, ADXS11-001, VGX-3100, INO-9012, GSK1437173A, BPX-501, AGS-003, IDC-G305, HyperAcute®-Renal (HAR) immunotherapy, Prevenar13, MAGER-3.A1, NA17.A2, DCVax-Direct, latent membrane protein-2 (LMP2)-loaded dendritic cell vaccine (NCT02115126), HS410-101 (NCT02010203, Heat Biologics), EAU RF 2010-01 (NCT01435356, GSK), 140036 (NCT02015104, Rutgers Cancer Institute of New Jersey), 130016 (NCT01730118, National Cancer Institute), MVX-201101 (NCT02193503, Maxivax SA), ITL-007-ATCR-MBC (NCT01741038, Immunovative Therapies, Limited), CDR0000644921 (NCT00923143, Abramson cancer center of the University of Pennsylvania), SuMo-Sec-01 (NCT00108875, Julius Maximilians Universitaet Hospital), or MCC-15651 (NCT01176474, Medarex, Inc, BMS).

In some embodiments, a TEC inhibitor (e.g. BTK inhibitor, ITK inhibitor) and an immune checkpoint inhibitor are administered in combination with an additional anticancer agent or therapy for the treatment of cancer. In some embodiments, the TEC inhibitor is a BTK inhibitor. In some embodiments, a BTK inhibitor and an immune checkpoint inhibitor are administered in combination with an additional anticancer agent or therapy for the treatment of cancer. In some embodiments, the additional therapy for the treatment of cancer is selected from among administration of a chemotherapeutic agent, a biologic agent, radiation therapy, bone marrow transplant or surgery. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof. In some embodiments, the BTK inhibitor is ibrutinib.

In some embodiments, a TEC inhibitor (e.g. BTK inhibitor or ITK inhibitor) is administered in combination with one or more immune checkpoint inhibitors. In some embodiments, a BTK inhibitor (e.g. ibrutinib) is administered in combination with one or more immune checkpoint inhibitors. In some embodiments, a BTK inhibitor (e.g. ibrutinib) is administered in combination with at least two immune checkpoint inhibitors. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, or VTCN1.

In some embodiments, a TEC inhibitor (e.g. BTK inhibitor or ITK inhibitor) and an immune checkpoint inhibitor are administered in combination with an additional therapeutic agent for the treatment of a breast cancer. In some embodiments, a Btk inhibitor (e.g. ibrutinib) and an immune checkpoint inhibitor are administered in combination with an additional therapeutic agent for the treatment of a breast cancer. Exemplary therapeutic agents for the treatment of breast cancer include, but are not limited to, ado-trastuzumab emtansine (Kadcyla), anastrozole (Arimidex), capecitabine (Xeloda), cyclophosphamide (Clafen, Cytoxan, Neosar), docetaxel (Taxotere), doxorubicin hydrochloride (Adriamycin PFS, Adriamycin RDF), epirubicin hydrochloride (Ellence), everolimus, exemestane (Aromasin), fluorouracil (Efudex, Fluoroplex), fulvestrant (Faslodex), gemcitabine hydrochloride (Gemzar), goserelin acetate (Zoladex), ixabepilone (Ixempra), lapatinib ditosylate (Tykerb), letrozole (Femara), megestrol acetate (Megace), methotrexate (Abitrexate, Folex PFS, Folex, Methotrexate LPF, Mexate-AQ, Mexate), paclitaxel (Taxol), paclitaxel albumin-stabilized nanoparticle formulation (Abraxane), pamidronate disodium (Aredia), pertuzumab (Perjeta), tamoxifen citrate (Nolvadex), toremifene (Fareston), trastuzumab (Herceptin), AC (doxorubicin hydrochloride and cyclophosphamide), AC-T (doxorubicin hydrochloride, cyclophosphamide and paclitaxel), CAF (cyclophosphamide, doxorubicin hydrochloride and fluorouracil), CMF (cyclophosphamide, methotrexate and fluorouracil), FEC (fluorouracil, epirubicin hydrochloride and cyclophosphamide) and TAC (docetaxel, doxorubicin hydrochloride and cyclophosphamide).

In some embodiments, ibrutinib and an immune checkpoint inhibitor are administered in combination with ado-trastuzumab emtansine (Kadcyla), anastrozole (Arimidex), capecitabine (Xeloda), cyclophosphamide (Clafen, Cytoxan, Neosar), docetaxel (Taxotere), doxorubicin hydrochloride (Adriamycin PFS, Adriamycin RDF), epirubicin hydrochloride (Ellence), everolimus, exemestane (Aromasin), fluorouracil (Efudex, Fluoroplex), fulvestrant (Faslodex), gemcitabine hydrochloride (Gemzar), goserelin acetate (Zoladex), ixabepilone (Ixempra), lapatinib ditosylate (Tykerb), letrozole (Femara), megestrol acetate (Megace), methotrexate (Abitrexate, Folex PFS, Folex, Methotrexate LPF, Mexate-AQ, Mexate), paclitaxel (Taxol), paclitaxel albumin-stabilized nanoparticle formulation (Abraxane), pamidronate disodium (Aredia), pertuzumab (Perjeta), tamoxifen citrate (Nolvadex), toremifene (Fareston), trastuzumab (Herceptin), AC (doxorubicin hydrochloride and cyclophosphamide), AC-T (doxorubicin hydrochloride, cyclophosphamide and paclitaxel), CAF (cyclophosphamide, doxorubicin hydrochloride and fluorouracil), CMF (cyclophosphamide, methotrexate and fluorouracil), FEC (fluorouracil, epirubicin hydrochloride and cyclophosphamide) or TAC (docetaxel, doxorubicin hydrochloride and cyclophosphamide) for the treatment of a breast cancer. In some embodiments, ibrutinib and an immune checkpoint inhibitor are administered sequentially, simultaneously, or intermittently with the additional therapeutic agent for the treatment of a breast cancer.

In some embodiments, a TEC inhibitor (e.g. BTK inhibitor or ITK inhibitor) and an immune checkpoint inhibitor are administered in combination with an additional therapeutic agent for the treatment of a colon cancer. In some embodiments, a Btk inhibitor (e.g. ibrutinib) and an immune checkpoint inhibitor are administered in combination with an additional therapeutic agent for the treatment of a colon cancer. Exemplary therapeutic agents for the treatment of colon cancer include, but are not limited to, capecitabine (e.g. Xeloda), cetuximab (e.g. Erbitux), bevacizumab (e.g. Avastin), fluorouracil (e.g. Adrucil, Efudex, Fluoroplex), irinotecan hydrochloride (e.g. Camptosar), leucovorin calcium (e.g. Wellcovorin), oxaliplatin (e.g. Eloxatin), panitumumab (e.g. Vectibix), regorafenib (e.g. Stivarga), ziv-aflibercept (e.g. Zaltrap), CAPDX (capecitabine and oxaliplatin), FOLFIRI (leucovorin calcium, fluorouracil, and irinotecan hydrochloride), FOLFIRI-BEVACIZUMAB, FOLFIRI-CETUXIMAB, FOLFOX (leucovorin calcium, fluorouracil, and oxaliplatin), or XELOX (capecitabine and oxaliplatin).

In some embodiments, ibrutinib and an immune checkpoint inhibitor are administered in combination with capecitabine (e.g. Xeloda), cetuximab (e.g. Erbitux), bevacizumab (e.g. Avastin), fluorouracil (e.g. Adrucil, Efudex, Fluoroplex), irinotecan hydrochloride (e.g. Camptosar), leucovorin calcium (e.g. Wellcovorin), oxaliplatin (e.g. Eloxatin), panitumumab (e.g. Vectibix), regorafenib (e.g. Stivarga), ziv-aflibercept (e.g. Zaltrap), CAPDX (capecitabine and oxaliplatin), FOLFIRI (leucovorin calcium, fluorouracil, and irinotecan hydrochloride), FOLFIRI-BEVACIZUMAB, FOLFIRI-CETUXIMAB, FOLFOX (leucovorin calcium, fluorouracil, and oxaliplatin), or XELOX (capecitabine and oxaliplatin) for the treatment of a colon cancer. In some embodiments, ibrutinib and an immune checkpoint inhibitor are administered sequentially, simultaneously, or intermittently with the additional therapeutic agent for the treatment of a colon cancer.

In some embodiments, a TEC inhibitor (e.g. BTK inhibitor or ITK inhibitor) and an immune checkpoint inhibitor are administered in combination with an additional therapeutic agent for the treatment of a bladder cancer. In some embodiments, a Btk inhibitor (e.g. ibrutinib) and an immune checkpoint inhibitor are administered in combination with an additional therapeutic agent for the treatment of a bladder cancer. Exemplary therapeutic agents for the treatment of bladder cancer include, but are not limited to, doxorubicin hydrochloride (Adriamycin PFS/RDF), cisplatin, mitomycin, fluorouracil, gemcitabine, methotrexate, vinblastine, carboplatin, paclitaxel, docetaxel, thiotepa (Thioplex, Tepadina), immunotherapeutic agents (e.g. Bacille Calmette-Guerin, interferon alfa-2b), and radiation therapeutic agents.

In some embodiments, ibrutinib and an immune checkpoint inhibitor are administered in combination with doxorubicin hydrochloride (Adriamycin PFS/RDF), cisplatin, mitomycin, fluorouracil, gemcitabine, methotrexate, vinblastine, carboplatin, paclitaxel, docetaxel, thiotepa (Thioplex, Tepadina), immunotherapeutic agents (e.g. Bacille Calmette-Guerin, interferon alfa-2b), and radiation therapeutic agents. In some embodiments, ibrutinib and an immune checkpoint inhibitor are administered sequentially, simultaneously, or intermittently with the additional therapeutic agent for the treatment of a bladder cancer.

In some embodiments, a TEC inhibitor (e.g. BTK inhibitor or ITK inhibitor) and an immune checkpoint inhibitor are administered in combination with an additional therapeutic agent for the treatment of a colon cancer. In some embodiments, a Btk inhibitor (e.g. ibrutinib) and an immune checkpoint inhibitor are administered in combination with an additional therapeutic agent for the treatment of a colon cancer. Exemplary therapeutic agents for the treatment of colon cancer include, but are not limited to, fluorouracil (Adrucil), bevacizumab (Avastin), irinotecan hydrochloride (Camptosar), capecitabine, cetuximab, Efudex, oxaliplatin (Eloxatin), Erbutix, Fluoroplex, leucovorin calcium (Wellcovorin), panitumamab (Vectibix), regorafenib (Stivarga), ziv-aflibercept, CAPDX, FOLFIRI, FOLFOX, and XELOX.

In some embodiments, ibrutinib and an immune checkpoint inhibitor are administered in combination with fluorouracil (Adrucil), bevacizumab (Avastin), irinotecan hydrochloride

(Camptosar), capecitabine, cetuximab, Efudex, oxaliplatin (Eloxatin), Erbutix, Fluoroplex, leucovorin calcium (Wellcovorin), panitumamab (Vectibix), regorafenib (Stivarga), ziv-aflibercept, CAPDX, FOLFIRI, FOLFOX, and XELOX. In some embodiments, ibrutinib and an immune checkpoint inhibitor are administered sequentially, simultaneously, or intermittently with the additional therapeutic agent for the treatment of a colon cancer.

In some embodiments, a TEC inhibitor (e.g. BTK inhibitor or ITK inhibitor) and an immune checkpoint inhibitor are administered in combination with an additional therapeutic agent for the treatment of a lung cancer. In some embodiments, a Btk inhibitor (e.g. ibrutinib) and an immune checkpoint inhibitor are administered in combination with an additional therapeutic agent for the treatment of a lung cancer. Exemplary therapeutic agents for the treatment of lung cancer include, but are not limited to, Adriamycin IV, Rheumatrex, Mustargen, methotrexate (Abitrexate), Abraxane, afatinib dimaleate (Gilotrif), pemetrexed disodium (Alimta), bevacixumab, carboplatin, cisplatin, crizotinib, erlotinib hydrochloride, Etopophos (etoposide phosphate), Folex, Folex PFS, gefitinib (Iressa), gemcitabine hydrochloride (Gemzar), topotecan hydrochloride (Hycamtin), Methotrexate LPF, Mexate, Mexate-AQ, paclitaxel, Paraplat, Paraplatin, Platinol, Platinol-AQ, Tarceva, Taxol, Xalkori, Toposar, VePesid and MPDL3280A.

In some embodiments, ibrutinib and an immune checkpoint inhibitor are administered in combination with Adriamycin IV, Rheumatrex, Mustargen, methotrexate (Abitrexate), Abraxane, afatinib dimaleate (Gilotrif), pemetrexed disodium (Alimta), bevacixumab, carboplatin, cisplatin, crizotinib, erlotinib hydrochloride, Etopophos (etoposide phosphate), Folex, Folex PFS, gefitinib (Iressa), gemcitabine hydrochloride (Gemzar), topotecan hydrochloride (Hycamtin), Methotrexate LPF, Mexate, Mexate-AQ, paclitaxel, Paraplat, Paraplatin, Platinol, Platinol-AQ, Tarceva, Taxol, Xalkori, Toposar, VePesid and MPDL3280A. In some embodiments, ibrutinib and an immune checkpoint inhibitor are administered sequentially, simultaneously, or intermittently with the additional therapeutic agent for the treatment of a lung cancer.

In some embodiments, a TEC inhibitor (e.g. BTK inhibitor or ITK inhibitor) and an immune checkpoint inhibitor are administered in combination with an additional therapeutic agent for the treatment of an ovarian cancer. In some embodiments, a Btk inhibitor (e.g. ibrutinib) and an immune checkpoint inhibitor are administered in combination with an additional therapeutic agent for the treatment of an ovarian cancer. Exemplary therapeutic agents for the treatment of ovarian cancer include, but are not limited to, doxorubicin hydrochloride (Adriamycin PFS/RDF), carboplatin, cyclophosphamide (Clafen), cisplatin, Cytoxan, Dox-SL, DOXIL, doxorubicin hydrochloride liposome (Evacet), gemcitabine hydrochloride (Gemzar), topotecan hydrochloride (Hycamtin), Neosar, Paclitaxel, Paraplat, Paraplatin, Platinol, Platinol-AQ, Taxol and BEP.

In some embodiments, ibrutinib and an immune checkpoint inhibitor are administered in combination with doxorubicin hydrochloride (Adriamycin PFS/RDF), carboplatin, cyclophosphamide (Clafen), cisplatin, Cytoxan, Dox-SL, DOXIL, doxorubicin hydrochloride liposome (Evacet), gemcitabine hydrochloride (Gemzar), topotecan hydrochloride (Hycamtin), Neosar, Paclitaxel, Paraplat, Paraplatin, Platinol, Platinol-AQ, Taxol and BEP. In some embodiments, ibrutinib and an immune checkpoint inhibitor are administered sequentially, simultaneously, or intermittently with the additional therapeutic agent for the treatment of an ovarian cancer.

In some embodiments, a TEC inhibitor (e.g. BTK inhibitor or ITK inhibitor) and an immune checkpoint inhibitor are administered in combination with an additional therapeutic agent for the treatment of a pancreatic cancer. In some embodiments, a Btk inhibitor (e.g. ibrutinib) and an immune checkpoint inhibitor are administered in combination with an additional therapeutic agent for the treatment of a pancreatic cancer. Exemplary therapeutic agents for the treatment of pancreatic cancer include, but are not limited to, Adriamycin PFS IV, Adrucil, Efudex, erlotinib hydrochloride, Fluoroplex, fluorouracil, gemcitabine hydrochloride (Gemzar), mitomycin C, Tarceva, Oxaliplatin paclitaxel-protein bound IV, anc capecitabine.

In some embodiments, ibrutinib and an immune checkpoint inhibitor are administered in combination with Adriamycin PFS IV, Adrucil, Efudex, erlotinib hydrochloride, Fluoroplex, fluorouracil, gemcitabine hydrochloride (Gemzar), mitomycin C, Tarceva, Oxaliplatin paclitaxel-protein bound IV, anc capecitabine. In some embodiments, ibrutinib and an immune checkpoint inhibitor are administered sequentially, simultaneously, or intermittently with the additional therapeutic agent for the treatment of a pancreatic cancer.

In some embodiments, a TEC inhibitor (e.g. BTK inhibitor or ITK inhibitor) and an immune checkpoint inhibitor are administered in combination with an additional therapeutic agent for the treatment of a prostate cancer. In some embodiments, a Btk inhibitor (e.g. ibrutinib) and an immune checkpoint inhibitor are administered in combination with an additional therapeutic agent for the treatment of a prostate cancer. Exemplary therapeutic agents for the treatment of prostate cancer include, but are not limited to, abiraterone acetate, cabazitaxel, degarelix, docetaxel, enzalutamide, leuprolide acetate, prednisone, denosumab, sipuleucel-T, abraxane and gemzar, and radium 223 dichloride.

In some embodiments, ibrutinib and an immune checkpoint inhibitor are administered in combination with abiraterone acetate, cabazitaxel, degarelix, docetaxel, enzalutamide, leuprolide acetate, prednisone, denosumab, sipuleucel-T, abraxane and gemzar, and radium 223 dichloride. In some embodiments, ibrutinib and an immune checkpoint inhibitor are administered sequentially, simultaneously, or intermittently with the additional therapeutic agent for the treatment of a prostate cancer.

In some embodiments, a TEC inhibitor (e.g. BTK inhibitor or ITK inhibitor) and an immune checkpoint inhibitor are administered in combination with an additional therapeutic agent for the treatment of a proximal or distal bile duct cancer. In some embodiments, a Btk inhibitor (e.g. ibrutinib) and an immune checkpoint inhibitor are administered in combination with an additional therapeutic agent for the treatment of a proximal or distal bile duct cancer. Exemplary therapeutic agents for the treatment of proximal or distal bile duct cancer include, but are not limited to, cisplatin, gemcitabine, fluorouracil, and doxorubicin.

In some embodiments, ibrutinib and an immune checkpoint inhibitor are administered in combination with cisplatin, gemcitabine, fluorouracil, and doxorubicin. In some embodiments, ibrutinib and an immune checkpoint inhibitor are administered sequentially, simultaneously, or intermittently with the additional therapeutic agent for the treatment of a proximal or distal bile duct cancer.

In some embodiments, a TEC inhibitor (e.g. BTK inhibitor or ITK inhibitor) and an immune checkpoint inhibitor are administered in combination with an additional therapeutic agent for the treatment of CLL. In some embodiments, a Btk inhibitor (e.g. ibrutinib) and an immune checkpoint inhibitor are administered in combination with an additional therapeutic agent for the treatment of CLL. Exemplary therapeutic agents for the treatment of CLL include, but are not limited to, alemtuzumab (e.g. Campath), bendamustine hydrochloride (e.g. Treanda), chlorambucil (e.g. Ambochlorin, Amboclorin, Leukeran, Linfolizin), cyclophosphamide (e.g. Clafen, Cytoxan, Neosar), fludarabine phosphate (e.g. Fludara), idelalisib (e.g. Zydelig), mechlorethamine hydrochloride (e.g. Mustargen), obinutuzumab (e.g. Gazyva), ofatumumab (e.g. Arzerra), prednisone, rituximab (e.g. Rituxan), chlorambucil-prednisone, R-CHOP, PCR (pentostatin, cyclophosphamide, rituximab), FR (fludarabine, rituximab), FCR (fludarabine, cyclophosphamide, ritusimab), BR (bendamustine, rituximab), and CVP (cyclophosphamide, vincristine sulfate, prednisone).

In some embodiments, ibrutinib and an immune checkpoint inhibitor are administered in combination with alemtuzumab (e.g. Campath), bendamustine hydrochloride (e.g. Treanda), chlorambucil (e.g. Ambochlorin, Amboclorin, Leukeran, Linfolizin), cyclophosphamide (e.g. Clafen, Cytoxan, Neosar), fludarabine phosphate (e.g. Fludara), idelalisib (e.g. Zydelig), mechlorethamine hydrochloride (e.g. Mustargen), obinutuzumab (e.g. Gazyva), ofatumumab (e.g. Arzerra), prednisone, rituximab (e.g. Rituxan), chlorambucil-prednisone, R-CHOP, PCR (pentostatin, cyclophosphamide, rituximab), FR (fludarabine, rituximab), FCR (fludarabine, cyclophosphamide, ritusimab), BR (bendamustine, rituximab), and CVP (cyclophosphamide, vincristine sulfate, prednisone). In some embodiments, ibrutinib and an immune checkpoint inhibitor are administered sequentially, simultaneously, or intermittently with the additional therapeutic agent for the treatment of CLL.

In some embodiments, a TEC inhibitor (e.g. BTK inhibitor or ITK inhibitor) and an immune checkpoint inhibitor are administered in combination with an additional therapeutic agent for the treatment of SLL. In some embodiments, a Btk inhibitor (e.g. ibrutinib) and an immune checkpoint inhibitor are administered in combination with an additional therapeutic agent for the treatment of SLL. Exemplary therapeutic agents for the treatment of SLL include, but are not limited to, alemtuzumab (e.g. Campath), bendamustine hydrochloride (e.g. Treanda), chlorambucil (e.g. Ambochlorin, Amboclorin, Leukeran, Linfolizin), cyclophosphamide (e.g. Clafen, Cytoxan, Neosar), fludarabine phosphate (e.g. Fludara), idelalisib (e.g. Zydelig), mechlorethamine hydrochloride (e.g. Mustargen), obinutuzumab (e.g. Gazyva), ofatumumab (e.g. Arzerra), prednisone, rituximab (e.g. Rituxan), chlorambucil-prednisone, R-CHOP, PCR (pentostatin, cyclophosphamide, rituximab), FR (fludarabine, rituximab), FCR (fludarabine, cyclophosphamide, ritusimab), BR (bendamustine, rituximab), and CVP (cyclophosphamide, vincristine sulfate, prednisone).

In some embodiments, ibrutinib and an immune checkpoint inhibitor are administered in combination with alemtuzumab (e.g. Campath), bendamustine hydrochloride (e.g. Treanda), chlorambucil (e.g. Ambochlorin, Amboclorin, Leukeran, Linfolizin), cyclophosphamide (e.g. Clafen, Cytoxan, Neosar), fludarabine phosphate (e.g. Fludara), idelalisib (e.g. Zydelig), mechlorethamine hydrochloride (e.g. Mustargen), obinutuzumab (e.g. Gazyva), ofatumumab (e.g. Arzerra), prednisone, rituximab (e.g. Rituxan), chlorambucil-prednisone, R-CHOP, PCR (pentostatin, cyclophosphamide, rituximab), FR (fludarabine, rituximab), FCR (fludarabine, cyclophosphamide, ritusimab), BR (bendamustine, rituximab), and CVP (cyclophosphamide, vincristine sulfate, prednisone). In some embodiments, ibrutinib and an immune checkpoint inhibitor are administered sequentially, simultaneously, or intermittently with the additional therapeutic agent for the treatment of SLL.

In some embodiments, a TEC inhibitor (e.g. BTK inhibitor or ITK inhibitor) and an immune checkpoint inhibitor are administered in combination with an additional therapeutic agent for the treatment of DLBCL. In some embodiments, a Btk inhibitor (e.g. ibrutinib) and an immune checkpoint inhibitor are administered in combination with an additional therapeutic agent for the treatment of DLBCL. Exemplary therapeutic agents for the treatment of DLBCL include, but are not limited to, R-CHOP, rituximab, EPOCH, lenalidomide, cisplatin, cytarabine, dexamethasone, ICE (ifosfamide, carboplatin, etoposide), GDP (gemcitabine, dexamethasone, cisplatin), GEM-P (gemcitabine, methylprednisolone, cisplatin), R+GEMOX (rituximab, gemcitabine, oxaliplatin), ESHAP (etoposide, methylprednisolone, cisplatin, cytarabine), DHAP (dexamethasone, cytarabine, cisplatin), R-DHAP, R-DHAP-VIM-DHAP, DHAP-VIM-DHAP, GV (gemcitabine, vinorelbine), GVP (gemcitabine, vinorelbine, prednisone), ViGePP (vinorelbine, gemcitabine, procarbazine, prednisone), IEV (ifosfamide, etoposide, epirubicin), MINE (ifosfamide, etoposide, mitoxantrone), IVAD (ifosfamide, etoposide, cytarabine, dexamethasone), and Mini-BEAM (busulfan, etoposide, cytarabine, melphalan).

In some embodiments, ibrutinib and an immune checkpoint inhibitor are administered in combination with R-CHOP, rituximab, EPOCH, lenalidomide, cisplatin, cytarabine, dexamethasone, ICE (ifosfamide, carboplatin, etoposide), GDP (gemcitabine, dexamethasone, cisplatin), GEM-P (gemcitabine, methylprednisolone, cisplatin), R+GEMOX (rituximab, gemcitabine, oxaliplatin), ESHAP (etoposide, methylprednisolone, cisplatin, cytarabine), DHAP (dexamethasone, cytarabine, cisplatin), R-DHAP, R-DHAP-VIM-DHAP, DHAP-VIM-DHAP, GV (gemcitabine, vinorelbine), GVP (gemcitabine, vinorelbine, prednisone), ViGePP (vinorelbine, gemcitabine, procarbazine, prednisone), IEV (ifosfamide, etoposide, epirubicin), MINE (ifosfamide, etoposide, mitoxantrone), IVAD (ifosfamide, etoposide, cytarabine, dexamethasone), and Mini-BEAM (busulfan, etoposide, cytarabine, melphalan). In some embodiments, ibrutinib and an immune checkpoint inhibitor are administered sequentially, simultaneously, or intermittently with the additional therapeutic agent for the treatment of DLBCL.

In some embodiments, a TEC inhibitor (e.g. BTK inhibitor or ITK inhibitor) and an immune checkpoint inhibitor are administered in combination with an additional therapeutic agent for the treatment of mantle cell lymphoma. In some embodiments, a Btk inhibitor (e.g. ibrutinib) and an immune checkpoint inhibitor are administered in combination with an additional therapeutic agent for the treatment of mantle cell lymphoma. Exemplary therapeutic agents for the treatment of mantle cell lymphoma include, but are not limited to, CHOP, R—CHOP, CVP (cyclophosphamide, vincristin, prednisolone), fludarabine, cyclophosphamide, chlorambucil, dexamethasone, methylprednisolone, lenalidomide, idelalisib (GS-1101), vorinostat (Zolinza), ofatumumab (Arzerra), everolimus (Afinitor), panobinostat, and temsirolimus (Torisel).

In some embodiments, ibrutinib and an immune checkpoint inhibitor are administered in combination with CHOP, R-CHOP, CVP (cyclophosphamide, vincristin, prednisolone), fludarabine, cyclophosphamide, chlorambucil, dexamethasone, methylprednisolone, lenalidomide, idelalisib (GS-1101), vorinostat (Zolinza), ofatumumab (Arzerra), everolimus (Afinitor), panobinostat, and temsirolimus (Torisel). In some embodiments, ibrutinib and an immune checkpoint inhibitor are administered sequentially, simultaneously, or intermittently with the additional therapeutic agent for the treatment of mantle cell lymphoma.

In some embodiments, a TEC inhibitor (e.g. BTK inhibitor or ITK inhibitor) and an immune checkpoint inhibitor are administered in combination with an additional therapeutic agent for the treatment of Waldenström's macroglobulinemia. In some embodiments, a Btk inhibitor (e.g. ibrutinib) and an immune checkpoint inhibitor are administered in combination with an additional therapeutic agent for the treatment of Waldenström's macroglobulinemia. Exemplary therapeutic agents for the treatment of Waldenström's macroglobulinemia include, but are not limited to, chlorambucil, cyclophosphamide, fludarabine, cladribine, rituximab, prednisone, melphalan, 2-chlorodeoxyadenosine, interferon alfa, and interferon gamma.

In some embodiments, ibrutinib and an immune checkpoint inhibitor are administered in combination with chlorambucil, cyclophosphamide, fludarabine, cladribine, rituximab, prednisone, melphalan, 2-chlorodeoxyadenosine, interferon alfa, and interferon gamma. In some embodiments, ibrutinib and an immune checkpoint inhibitor are administered sequentially, simultaneously, or intermittently with the additional therapeutic agent for the treatment of Waldenström's macroglobulinemia.

### Treatment of a Pathogenic Infection

Pathogenic infections (e.g. viral infections) can contribute to about 15-20% of human cancers. For example, pathogens (e.g. virus) can encode proteins that can modulate host cellular signaling pathways that control proliferation, differentiation, cell death, genomic integrity, and/or the immune system. In some instances, a pathogen inserts its viral genes into a host cell to enhance already existing oncogenic genes in the genome. In some instances, a pathogen exerts chronic nonspecific inflammations in the host which leads to development of cancer.

In some embodiments, disclosed herein is a method of treating an infection in an individual in need thereof which comprises administering a combination of a TEC inhibitor (e.g. BTK inhibitor or ITK inhibitor) and an immune checkpoint inhibitor. In some embodiments, disclosed herein is a method of treating an infection in an individual in need thereof which comprises administering a combination of a Btk inhibitor (e.g. ibrutinib) and an immune checkpoint inhibitor. In some embodiments, the infection is a chronic infection. In some embodiments, the infections include, but are not limited to, infections caused by a virus, bacterium, parasite, protozoan, or fungus. In some embodiments, the pathogen is a cancer-associated pathogen. In some embodiments, the cancer-associated pathogen is any pathogen that can either directly or indirectly cause or induce cancer, or pathogens that are opportunistic. In some instances, the cancer-associated pathogen is a cancer-inducing pathogen. In some instances, “indirectly” refers to the byproduct of a pathogen, such as for example an inflammation caused by the pathogen, or such as toxins produced by the pathogen, that can lead to cancer.

In some embodiments, the infection is caused by a virus. In some instances, the virus is a DNA virus or an RNA virus. In some instances, the DNA virus is a single-stranded (ss) DNA virus, a double-stranded (ds) DNA virus, or a DNA virus that contains both ss and ds DNA regions. In some cases, an RNA virus is a single-stranded (ss) RNA virus or a double-stranded (ds) RNA virus. In some cases, a ssRNA virus is further classified into a positive-sense RNA virus or a negative-sense RNA virus.

Exemplary dsDNA viruses include families from: Myoviridae, Podoviridae, Siphoviridae, Alloherpesviridae, Herpesviridae, Malacoherpesviridae, Lipothrixviridae, Rudiviridae, Adenoviridae, Ampullaviridae, Ascoviridae, Asfaviridae, Baculoviridae, Bicaudaviridae, Clavaviridae, Corticoviridae, Fuselloviridae, Globuloviridae, Guttaviridae, Hytrosaviridae, Iridoviridae, Marseilleviridae, Mimiviridae, Nimaviridae, Pandoraviridae, Papillomaviridae, Phycodnaviridae, Plasmaviridae, Polydnaviruses, Polyomaviridae, Poxyiridae, Sphaerolipoviridae, and Tectiviridae.

Exemplary ssDNA viruses include families from: Anelloviridae, Bacillariodnaviridae, Bidnaviridae, Circoviridae, Geminiviridae, Inoviridae, Microviridae, Nanoviridae, Parvoviridae, and Spiraviridae.

An exemplary DNA virus that contains both ss and ds DNA regions is from the group of pleolipoviruses. In some cases, the pleolipoviruses include Haloarcula hispanica pleomorphic virus 1, Halogeometricum pleomorphic virus 1, Halorubrum pleomorphic virus 1, Halorubrum pleomorphic virus 2, Halorubrum pleomorphic virus 3, and Halorubrum pleomorphic virus 6.

Exemplary dsRNA viruses include families from: Birnaviridae, Chrysoviridae, Cystoviridae, Endornaviridae, Hypoviridae, Megavirnaviridae, Partitiviridae, Picobirnaviridae, Reoviridae, Rotavirus and Totiviridae.

Exemplary positive-sense ssRNA viruses include families from: Alphaflexiviridae, Alphatetraviridae, Alvernaviridae, Arteriviridae, Astroviridae, Barnaviridae, Betaflexiviridae, Bromoviridae, Caliciviridae, Carmotetraviridae, Closteroviridae, Coronaviridae, Dicistroviridae, Flaviviridae, Gammaflexiviridae, Iflaviridae, Leviviridae, Luteoviridae, Marnaviridae, Mesoniviridae, Narnaviridae, Nodaviridae, Permutotetraviridae, Picornaviridae, Potyviridae, Roniviridae, Secoviridae, Togaviridae, Tombusviridae, Tymoviridae, and Virgaviridae.

Exemplary negative-sense ssRNA viruses include families from: Bornaviridae, Filoviridae, Paramyxoviridae, Rhabdoviridae, Nyamiviridae, Arenaviridae, Bunyaviridae, Ophioviridae, and Orthomyxoviridae.

Exemplary virus includes, but is not limited to: Abelson leukemia virus, Abelson murine leukemia virus, Abelson's virus, Acute laryngotracheobronchitis virus, Adelaide River virus, Adeno associated virus group, Adenovirus, African horse sickness virus, African swine fever virus, AIDS virus, Aleutian mink disease parvovirus, Alpharetrovirus, Alphavirus, ALV related virus, Amapari virus, Aphthovirus, Aquareovirus, Arbovirus, Arbovirus C, arbovirus group A, arbovirus group B, Arenavirus group, Argentine hemorrhagic fever virus, Argentine hemorrhagic fever virus, Arterivirus, Astrovirus, Ateline herpesvirus group, Aujezky's disease virus, Aura virus, Ausduk disease virus, Australian bat lyssavirus, Aviadenovirus, avian erythroblastosis virus, avian infectious bronchitis virus, avian leukemia virus, avian leukosis virus, avian lymphomatosis virus, avian myeloblastosis virus, avian paramyxovirus, avian pneumoencephalitis virus, avian reticuloendotheliosis virus, avian sarcoma virus, avian type C retrovirus group, Avihepadnavirus, Avipoxvirus, B virus, B19 virus, Babanki virus, baboon herpesvirus, baculovirus, Barmah Forest virus, Bebaru virus, Berrimah virus, Betaretrovirus, Birnavirus, Bittner virus, BK virus, Black Creek Canal virus, bluetongue virus, Bolivian hemorrhagic fever virus, Boma disease virus, border disease of sheep virus, borna virus, bovine alphaherpesvirus 1, bovine alphaherpesvirus 2, bovine coronavirus, bovine ephemeral fever virus, bovine immunodeficiency virus, bovine leukemia virus, bovine leukosis virus, bovine mammillitis virus, bovine papillomavirus, bovine papular stomatitis virus, bovine parvovirus, bovine syncytial virus, bovine type C oncovirus, bovine viral diarrhea virus, Buggy Creek virus, bullet shaped virus group, Bunyamwera virus supergroup, Bunyavirus, Burkitt's lymphoma virus, Bwamba Fever, CA virus, Calicivirus, California encephalitis virus, camelpox virus, canarypox virus, canid herpesvirus, canine coronavirus, canine distemper virus, canine herpesvirus, canine minute virus, canine parvovirus, Cano Delgadito virus, caprine arthritis virus, caprine encephalitis virus, Caprine Herpes Virus, Capripox virus, Cardiovirus, caviid herpesvirus 1, Cercopithecid herpesvirus 1, cercopithecine herpesvirus 1, Cercopithecine herpesvirus 2, Chandipura virus, Changuinola virus, channel catfish virus, Charleville virus, chickenpox virus, Chikungunya virus, chimpanzee herpesvirus, chub reovirus, chum salmon virus, Cocal virus, Coho salmon reovirus, coital exanthema virus, Colorado tick fever virus, Coltivirus, Columbia SK virus, common cold virus, contagious eethyma virus, contagious pustular dermatitis virus, Coronavirus, Corriparta virus, coryza virus, cowpox virus, coxsackie virus, CPV (cytoplasmic polyhedrosis virus), cricket paralysis virus, Crimean-Congo hemorrhagic fever virus, croup associated virus, Cryptovirus, Cypovirus, Cytomegalovirus, cytomegalovirus group, cytoplasmic polyhedrosis virus, deer papillomavirus, deltaretrovirus, dengue virus, Densovirus, Dependovirus, Dhori virus, diploma virus, Drosophila C virus, duck hepatitis B virus, duck hepatitis virus 1, duck hepatitis virus 2, duovirus, Duvenhage virus, Deformed wing virus DWV, eastern equine encephalitis virus, eastern equine encephalomyelitis virus, EB virus, Ebola virus, Ebola-like virus, echo virus, echovirus, echovirus 10, echovirus 28, echovirus 9, ectromelia virus, EEE virus, EIA virus, EIA virus, encephalitis virus, encephalomyocarditis group virus, encephalomyocarditis virus, Enterovirus, enzyme elevating virus, enzyme elevating virus (LDH), epidemic hemorrhagic fever virus, epizootic hemorrhagic disease virus, Epstein-Barr virus, equid alphaherpesvirus 1, equid alphaherpesvirus 4, equid herpesvirus 2, equine abortion virus, equine arteritis virus, equine encephalosis virus, equine infectious anemia virus, equine morbillivirus, equine rhinopneumonitis virus, equine rhinovirus, Eubenangu virus, European elk papillomavirus, European swine fever virus, Everglades virus, Eyach virus, felid herpesvirus 1, feline calicivirus, feline fibrosarcoma virus, feline herpesvirus, feline immunodeficiency virus, feline infectious peritonitis virus, feline leukemia/sarcoma virus, feline leukemia virus, feline panleukopenia virus, feline parvovirus, feline sarcoma virus, feline syncytial virus, Filovirus, Flanders virus, Flavivirus, foot and mouth disease virus, Fort Morgan virus, Four Corners hantavirus, fowl adenovirus 1, fowlpox virus, Friend virus, Gammaretrovirus, GB hepatitis virus, GB virus, German measles virus, Getah virus, gibbon ape leukemia virus, glandular fever virus, goatpox virus, golden shinner virus, Gonometa virus, goose parvovirus, granulosis virus, Gross' virus, ground squirrel hepatitis B virus, group A arbovirus, Guanarito virus, guinea pig cytomegalovirus, guinea pig type C virus, Hantaan virus, Hantavirus, hard clam reovirus, hare fibroma virus, HCMV (human cytomegalovirus), hemadsorption virus 2, hemagglutinating virus of Japan, hemorrhagic fever virus, hendra virus, Henipaviruses, Hepadnavirus, hepatitis A virus, hepatitis B virus group, hepatitis C virus, hepatitis D virus, hepatitis delta virus, hepatitis E virus, hepatitis F virus, hepatitis G virus, hepatitis nonA nonB virus, hepatitis virus, hepatitis virus (nonhuman), hepatoencephalomyelitis reovirus 3, Hepatovirus, heron hepatitis B virus, herpes B virus, herpes simplex virus, herpes simplex virus 1, herpes simplex virus 2, herpesvirus, herpesvirus 7, Herpesvirus ateles, Herpesvirus hominis, Herpesvirus infection, Herpesvirus saimiri, Herpesvirus suis, Herpesvirus varicellae, Highlands J virus, Hirame rhabdovirus, hog cholera virus, human adenovirus 2, human alphaherpesvirus 1, human alphaherpesvirus 2, human alphaherpesvirus 3, human B lymphotropic virus, human betaherpesvirus 5, human coronavirus, human cytomegalovirus group, human foamy virus, human gammaherpesvirus 4, human gammaherpesvirus 6, human hepatitis A virus, human herpesvirus 1 group, human herpesvirus 2 group, human herpesvirus 3 group, human herpesvirus 4 group, human herpesvirus 6, human herpesvirus 8, human immunodeficiency virus, human immunodeficiency virus 1, human immunodeficiency virus 2, human papillomavirus, human T cell leukemia virus, human T cell leukemia virus I, human T cell leukemia virus II, human T cell leukemia virus III, human T cell lymphoma virus I, human T cell lymphoma virus II, human T cell lymphotropic virus type 1, human T cell lymphotropic virus type 2, human T lymphotropic virus I, human T lymphotropic virus II, human T lymphotropic virus III, Ichnovirus, infantile gastroenteritis virus, infectious bovine rhinotracheitis virus, infectious haematopoietic necrosis virus, infectious pancreatic necrosis virus, influenza virus A, influenza virus B, influenza virus C, influenza virus D, influenza virus pr8, insect iridescent virus, insect virus, iridovirus, Japanese B virus, Japanese encephalitis virus, JC virus, Junin virus, Kaposi's sarcoma-associated herpesvirus, Kemerovo virus, Kilham's rat virus, Klamath virus, Kolongo virus, Korean hemorrhagic fever virus, kumba virus, Kysanur forest disease virus, Kyzylagach virus, La Crosse virus, lactic dehydrogenase elevating virus, lactic dehydrogenase virus, Lagos bat virus, Langur virus, lapine parvovirus, Lassa fever virus, Lassa virus, latent rat virus, LCM virus, Leaky virus, Lentivirus, Leporipoxvirus, leukemia virus, leukovirus, lumpy skin disease virus, lymphadenopathy associated virus, Lymphocryptovirus, lymphocytic choriomeningitis virus, lymphoproliferative virus group, Machupo virus, mad itch virus, mammalian type B oncovirus group, mammalian type B retroviruses, mammalian type C retrovirus group, mammalian type D retroviruses, mammary tumor virus, Mapuera virus, Marburg virus, Marburg-like virus, Mason Pfizer monkey virus, Mastadenovirus, Mayaro virus, ME virus, measles virus, Menangle virus, Mengo virus, Mengovirus, Middelburg virus, milkers nodule virus, mink enteritis virus, minute virus of mice, MLV related virus, MM virus, Mokola virus, Molluscipoxvirus, Molluscum contagiosum virus, monkey B virus, monkeypox virus, Mononegavirales, Morbillivirus, Mount Elgon bat virus, mouse cytomegalovirus, mouse encephalomyelitis virus, mouse hepatitis virus, mouse K virus, mouse leukemia virus, mouse mammary tumor virus, mouse minute virus, mouse pneumonia virus, mouse poliomyelitis virus, mouse polyomavirus, mouse sarcoma virus, mousepox virus, Mozambique virus, Mucambo virus, mucosal disease virus, mumps virus, murid betaherpesvirus 1, murid cytomegalovirus 2, murine cytomegalovirus group, murine encephalomyelitis virus, murine hepatitis virus, murine leukemia virus, murine nodule inducing virus, murine polyomavirus, murine sarcoma virus, Muromegalovirus, Murray Valley encephalitis virus, myxoma virus, Myxovirus, Myxovirus multiforme, Myxovirus parotitidis, Nairobi sheep disease virus, Nairovirus, Nanirnavirus, Nariva virus, Ndumo virus, Neethling virus, Nelson Bay virus, neurotropic virus, New World Arenavirus, newborn pneumonitis virus, Newcastle disease virus, Nipah virus, noncytopathogenic virus, Norwalk virus, nuclear polyhedrosis virus (NPV), nipple neck virus, O'nyong'nyong virus, Ockelbo virus, oncogenic virus, oncogenic viruslike particle, oncornavirus, Orbivirus, Orf virus, Oropouche virus, Orthohepadnavirus, Orthomyxovirus, Orthopoxvirus, Orthoreovirus, Orungo, ovine papillomavirus, ovine catarrhal fever virus, owl monkey herpesvirus, Palyam virus, Papillomavirus, Papillomavirus sylvilagi, Papovavirus, parainfluenza virus, parainfluenza virus type 1, parainfluenza virus type 2, parainfluenza virus type 3, parainfluenza virus type 4, Paramyxovirus, Parapoxvirus, paravaccinia virus, Parvovirus, Parvovirus B19, parvovirus group, Pestivirus, Phlebovirus, phocine distemper virus, Picodnavirus, Picornavirus, pig cytomegalovirus-pigeonpox virus, Piry virus, Pixuna virus, pneumonia virus of mice, Pneumovirus, poliomyelitis virus, poliovirus, Polydnavirus, polyhedral virus, polyoma virus, Polyomavirus, Polyomavirus bovis, Polyomavirus cercopitheci, Polyomavirus hominis 2, Polyomavirus maccacae 1, Polyomavirus muris 1, Polyomavirus muris 2, Polyomavirus papionis 1, Polyomavirus papionis 2, Polyomavirus sylvilagi, Pongine herpesvirus 1, porcine epidemic diarrhea virus, porcine hemagglutinating encephalomyelitis virus, porcine parvovirus, porcine transmissible gastroenteritis virus, porcine type C virus, pox virus, poxvirus, poxvirus variolae, Prospect Hill virus, Provirus, pseudocowpox virus, pseudorabies virus, psittacinepox virus, quailpox virus, rabbit fibroma virus, rabbit kidney vaculolating virus, rabbit papillomavirus, rabies virus, raccoon parvovirus, raccoonpox virus, Ranikhet virus, rat cytomegalovirus, rat parvovirus, rat virus, Rauscher's virus, recombinant vaccinia virus, recombinant virus, reovirus, reovirus 1, reovirus 2, reovirus 3, reptilian type C virus, respiratory infection virus, respiratory syncytial virus, respiratory virus, reticuloendotheliosis virus, Rhabdovirus, Rhabdovirus carnia, Rhadinovirus, Rhinovirus, Rhizidiovirus, Rift Valley fever virus, Riley's virus, rinderpest virus, RNA tumor virus, Ross River virus, Rotavirus, rougeole virus, Rous sarcoma virus, rubella virus, rubeola virus, Rubivirus, Russian autumn encephalitis virus, SA 11 simian virus, SA2 virus, Sabia virus, Sagiyama virus, Saimirine herpesvirus 1, salivary gland virus, sandfly fever virus group, Sandjimba virus, SARS virus, SDAV (sialodacryoadenitis virus), sealpox virus, Semliki Forest Virus, Seoul virus, sheeppox virus, Shope fibroma virus, Shope papilloma virus, simian foamy virus, simian hepatitis A virus, simian human immunodeficiency virus, simian immunodeficiency virus, simian parainfluenza virus, simian T cell lymphotrophic virus, simian virus, simian virus 40, Simplexvirus, Sin Nombre virus, Sindbis virus, smallpox virus, South American hemorrhagic fever viruses, sparrowpox virus, Spumavirus, squirrel fibroma virus, squirrel monkey retrovirus, SSV 1 virus group, STLV (simian T lymphotropic virus) type I, STLV (simian T lymphotropic virus) type II, STLV (simian T lymphotropic virus) type III, stomatitis papulosa virus, submaxillary virus, suid alphaherpesvirus 1, suid herpesvirus 2, Suipoxvirus, swamp fever virus, swinepox virus, Swiss mouse leukemia virus, TAC virus, Tacaribe complex virus, Tacaribe virus, Tanapox virus, Taterapox virus, Tench reovirus, Theiler's encephalomyelitis virus, Theiler's virus, Thogoto virus, Thottapalayam virus, Tick borne encephalitis virus, Tioman virus, Togavirus, Torovirus, tumor virus, Tupaia virus, turkey rhinotracheitis virus, turkeypox virus, type C retroviruses, type D oncovirus, type D retrovirus group, ulcerative disease rhabdovirus, Una virus, Uukuniemi virus group, vaccinia virus, vacuolating virus, varicella zoster virus, Varicellovirus, Varicola virus, variola major virus, variola virus, Vasin Gishu disease virus, VEE virus, Venezuelan equine encephalitis virus, Venezuelan equine encephalomyelitis virus, Venezuelan hemorrhagic fever virus, vesicular stomatitis virus, Vesiculovirus, Vilyuisk virus, viper retrovirus, viral haemorrhagic septicemia virus, Visna Maedi virus, Visna virus, volepox virus, VSV (vesicular stomatitis virus), Wallal virus, Warrego virus, wart virus, WEE virus, West Nile virus, western equine encephalitis virus, western equine encephalomyelitis virus, Whataroa virus, Winter Vomiting Virus, woodchuck hepatitis B virus, woolly monkey sarcoma virus, wound tumor virus, WRSV virus, Yaba monkey tumor virus, Yaba virus, Yatapoxvirus, yellow fever virus, and the Yug Bogdanovac virus.

In some instances, a virus is a cancer-associated virus. In some instances, cancer-associated viruses include, but are not limited to, human T-cell leukemia virus (HTLV-1), hepatitis C virus (HCV), hepatitis B virus (HBV), human papillomavirus (HPV), Epstein-Barr Virus (EBV), Kaposi's sarcoma-associated herpesvirus (KSHV)/Human Herpes Virus 8 (HHV8), human immunodeficiency virus (HIV), and influenza.

In some instances, a cancer-associated pathogen is a bacterium, a fungus, a parasite, or a protozoan. Examples of bacteria include: Helicobacter pyloris, Borelia burgdorferi, Legionella pneumophilia, Mycobacteria spp. (e.g., M. tuberculosis, M. avium, M. intracellulare, M. kansasii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic spp.), Streptococcus pneumoniae, pathogenic Campylobacter sp., Enterococcus sp., Haemophilus influenzae, Bacillus anthracia, Corynebacterium diphtheriae, Corynebacterium sp., Erysipelothrix rhusiopathiae, Clostridium perfringens, Clostridium tetani, Chlamydia trachomatis, Enterobacter aerogenes, Klebsiella pneumoniae, Pasturella multocida, Bacteroides sp., Fusobacterium nucleatum, Streptobacillus moniliformis, Treponema pallidum, Treponema pertenue, Leptospira, and Actinomyces israelli.

Examples of fungi include: Cryptococcus neoformans, Histoplasma capsulatum, Coccidioides immitis, Blastomyces dermatitidis, Chlamydia trachomatis, Candida albicans. Other infectious organisms (i.e., protists) include: Plasmodium falciparum and Toxoplasma gondii.

Examples of parasites include Schistosoma haematobium (squamous cell carcinoma of the bladder), Schistosoma japonicum, and liver flukes, Opisthorchis viverrini and Clonorchis sinensis.

A example of protozoan includes plasmodium (also known as malaria parasite).

In some embodiments, a BTK inhibitor (e.g. ibrutinib) and an immune checkpoint inhibitor are administered in combination with an additional therapeutic agent for the treatment of a pathogenic infection. In some embodiments, the additional therapeutic agent is a therapeutic agent for the treatment of a viral infection, a bacterial infection, a fungus infection, a parasitic infection, or a protozoan infection. In some embodiments, the therapeutic agent for treatment of a viral infection is an antiviral agent. In some embodiments, the therapeutic agent for treatment of a bacterial infection is an antibacterial agent. In some embodiments, the therapeutic agent for treatment of a fungus infection is an antifungal agent. In some embodiments, the therapeutic agent for treatment of a parasitic infection is an antiparasitic agent. In some embodiments, the therapeutic agent for treatment of a protozoan infection is an antiprotozoal agent. In some embodiments, the pathogen is a cancer-associated pathogen.

In some embodiments, a BTK inhibitor (e.g. ibrutinib) and an immune checkpoint inhibitor are administered in combination with an antiviral agent for the treatment of a viral infection. Exemplary antiviral agents include, but are not limited to, immunostimulants such as interferon (e.g., alpha interferons, beta interferons, gamma interferons, pegylated alpha interferons, pegylated beta interferons, pegylated gamma interferons and mixtures of any two or more thereof), granulocyte macrophage colony-stimulating factor, echinacin, isoprinosine, adjuvants, biodegradable microspheres (e.g., polylactic galactide) and liposomes (into which the compound is incorporated), and thymus factors; immunosuppressants such as cyclosporin, azatioprin, methotrexate, cyclophsphamide, FK 506, Cortisol, betametasone, cortisone, desametasone, flunisolide, prednisolone, methylprednisolone, prednisone, triamcinolone, alclometasone, amcinonide desonide, desoxymetasone, prednisone, cyclosporine, mycophenolate mofetil, and tacrolimus; nucleoside and nucleotide antiviral agents such as abacavir, acyclovir (ACV), adefovir, zidovudine (ZDV), ribavirin, lamivudine, adefovir and entecavir, tenofovir, emtricitabine, telbuvidine, clevudine, valtorcitabine, cidofovir, and derivatives thereof; protease inhibitors such as saquinavir, ritonavir, indinavir, nelfinavir, amprenavir, atazanavir, boceprevir, and HCV NS3 protease inhibitors; inosine 5′-monophosphate dehydrogenase (IMPDH) inhibitors such as merimepodib (VX-497); viral entry inhibitors; viral maturation inhibitors; viral uncoating inhibitors such as amantadine, rimantadine, pleconaril, and derivatives thereof; integrase inhibitors; viral enzyme inhibitors; antisense antiviral molecules; ribozyme antiviral agents such as RNase P ribozyme; nanoviricides, antisense antiviral molecules include, but are not limited to, oligonucleotides designed to recognize and inactivate viral genes and antibodies.

In some embodiments, a viral infection is caused by a hepatitis virus, such as a hepatitis C virus, or a hepatitis B virus; human immunodeficiency virus (HIV), or an influenza virus such as influenza A virus, or influenza B virus. In some embodiments, a BTK inhibitor (e.g. ibrutinib) and an immune checkpoint inhibitor are administered in combinatin with an antiviral agent for the treatment of a viral infection caused by such as for example, a hepatitis virus, HIV, or an influenza virus. In some embodiments, a BTK inhibitor (e.g. ibrutinib) and an immune checkpoint inhibitor are administered in combinatin with an antiviral agent for the treatment of a hepatitis infection, such as an infection caused by hepatitis C virus (HCV) or hepatitis B virus (HBV). In some embodiments, a BTK inhibitor (e.g. ibrutinib) and an immune checkpoint inhibitor are administered in combinatin with an antiviral agent for the treatment of HIV infection. In some embodiments, a BTK inhibitor (e.g. ibrutinib) and an immune checkpoint inhibitor are administered in combinatin with an antiviral agent for the treatment of influenza virus infection.

In some embodiments, a BTK inhibitor (e.g. ibrutinib) and an immune checkpoint inhibitor are administered in combinatin with an antiviral agent for the treatment of HCV infection. Exemplary antiviral agents for the treatment of HCV infection include, but are not limited to, interferon or interferon derivatives such as Interferon alfa-2a, Interferon alfa-2b, Peginterferon alfa-2a, Peginterferon alfa-2b, recombinant interferon alfa-2a, Sumiferon (a purified blend of natural alpha interferons), ALFERON® (a mixture of natural alpha interferons), consensus alpha interferon, pegylated interferon lambda; nucleoside analogs such as ribavirin or its derivatives, D-ribavirin, L-ribavirin, or taribavirin; nucleoside and nucleotide NSSB polymerase inhibitors such as sofosbuvir; NSSA inhibitors such as daclatasvir, ledipasvir, ABT-267, ACH-3102, GS-5816, GS-5885, IDX719, MK-8742 or PPI-668; non-nucleoside NSSB polymerase inhibitors such as deleobuvir, ABT-072, ABT-333, BMS-791325, VX-222, or tegobuvir; protease inhibitors such as boceprevir, danoprevir, faldaprevir, incivek, telaprevir, simeprevir, victrelis, ACH-1625, ACH-2684, ABT-450/r or VX-950; polymerase inhibitors such as deleobuvir, sofosbuvir or VX-135; NS3/4A protease inhibitors such as asunaprevir, danoprevir, MK-5172 or VX-950; ALN-VSP; PV-10; HDAC inhibitor such as abexinostat, resminostat, vorinostat, belinostat and panobinostat; thiazolides such as alinia (nitazoxanide); A3AR agonist such as CF102; GI-5005 (Tarmogen); MBL-HCV1; microRNA such as miravirsen; oral interferon; cyclophilin inhibitor such as SCY-635; TG4040; doxorubicin, livatag; immunomodulatory agents, such as Cc-, β-, and γ-interferons or thymosin, pegylated derivatized interferon-α compounds, and thymosin; other anti-viral agents, such as ribavirin, amantadine, and telbivudine; other inhibitors of hepatitis C proteases (NS2-NS3 inhibitors and NS3-NS4A inhibitors); inhibitors of other targets in the HCV life cycle, including helicase, polymerase, and metalloprotease inhibitors; inhibitors of internal ribosome entry; broad-spectrum viral inhibitors, such as IMPDH inhibitors (e.g., compounds described in U.S. Pat. Nos. 5,807,876, 6,498,178, 6,344,465, and 6,054,472; and PCT publications WO 97/40028, WO 98/40381, and WO 00/56331; and mycophenolic acid and derivatives thereof, and including, but not limited to, VX-497, VX-148, and VX-944); cytochrome P-450 inhibitor such as ritonavir (WO 94/14436), ketoconazole, troleandomycin, 4-methylpyrazole, cyclosporin, clomethiazole, cimetidine, itraconazole, fluconazole, miconazole, fluvoxamine, fluoxetine, nefazodone, sertraline, indinavir, nelfinavir, amprenavir, fosamprenavir, saquinavir, lopinavir, delavirdine, erythromycin, VX-944, and VX-497; kinase inhibitors such as methyl 2-cyano-3,12-dioxoolean-1,9-dien-28-oate (for the inhibition of CHUK); cetuximab (for the inhibition of EGFR), AEE 788, panitumumab, BMS-599626, ARRY-334543, XL647, canertinib, gefitinib, HKI-272, PD 153035, lapatinib, vandetanib, and erlotinib (for the inhibition of EGFR); BMS-387032 and fiavopiridol (for the inhibition of CDK2, CDK3, CDK4, and CDK8); XL647 (for the inhibition of EPHB4); dasatinib and AZM-475271 (for the inhibition of SRC); imatinib (for the inhibition of BCR); dasatinib (for the inhibition of EPHA2); and AZD-1152 (for the inhibition of AURKB). Other examples of known kinase inhibitors include, but are not limited to, sorafenib (for the inhibition of BRAF); BMS-599626 (for the inhibition of ERBB4); PD-0332991 and flavopiridol (for the inhibition of CDK4).

In some embodiments, a BTK inhibitor (e.g. ibrutinib) and an immune checkpoint inhibitor are administered in combinatin with an antiviral agent for the treatment of HBV infection. Exemplary antiviral agents for the treatment of HBV infection include, but are not limited to, interferons or interferon derivatives such as interferon alfa-2b and peginterferon alfa-2a; nucleoside analogues such as lamivudine (Epivir-HBV), adfovir dipivoxil (Hepsera), entecavir (Baraclude), telbivudine (Tyzeka/Sebivo), tenofovir (Viread), L-FMAU (Clevudine), LB80380 (Besifovir) and AGX-1009; non-nucleoside antivirals such as BAM 205 (NOV-205), Myrcludex B, HAP compound Bay 41-4109, REP 9AC, nitazoxanide (Alinia), dd-RNAi compound, ARC-520, NVR-1221 and IHVR-25; non-interferon immune enhancers such as thymosin alpha-1 (zadaxin), interleukin-7 (CYT107), DV-601, HBV core antigen vaccine, GS-9620 and GI13000; post-exposure and/or post-liver transplant treatment such as hyperHEP S/D, Nabi-HB and Hepa Gam B; and alternative natural agents such as milk thistle.

In some embodiments, a BTK inhibitor (e.g. ibrutinib) and an immune checkpoint inhibitor are administered in combinatin with an antiviral agent for the treatment of HIV infection. Exemplary antiviral agents for the treatment of HIV infection include, but are not limited to, multi-class combination drugs such as atripla (efavirenz+tenofovir+emtricitabine); complera (eviplera, rilpivirine+tenofovir+emtricitabine); stribild (elvitegravir+cobicistat+tenofovir+emtricitabine); “572-Trii” (dolutegravir+abacavir+lamivudine or DTG+ABC+3TC); nucleoside/nucleotide reverse transcriptase inhibitors (NRTIs) include combivir (zidovudine+lamivudine, AZT+3TC); emtriva (emtricitabine, FTC); epivir (lamivudine, 3TC); epzicom (Livexa, abacavir+lamivudine, ABC+3TC); retrovir (zidovudine, AZT, ZDV); trizivir (abacavir+zidovudine+lamivudine, ABC+AZT+3TC); truvada (tenofovir DF+emtricitabine, TDF+FTC); videx and videx EC (didanosine, ddI); viread (tenofovir disoproxil fumarate, TDF); zerit (stavudine, d4T); ziagen (abacavir, ABC); amadoxovir (AMDX, DAPD); tenofovir alafenamide fumarate (TAF); non-nucleoside reverse transcriptase inhibitors (NNRTIs) include edurant (rilpivirine, RPV, TMC-278); intelence (etravirine, ETR, TMC-125); rescriptor (delavirdine, DLV); sustiva (Stocrin, efavirenz, EFV); viramune and viramune XR (nevirapine, NVP), lersivirine (UK-453061); immune-based therapies include aralen (chloroquine phosphate), dermaVir, interleukin-7, lexgenleucel-T (VRX-496), plaquenil (hydroxychloroquine), proleukin (aldesleukin, IL-2), SB-782-T and Vacc-4×; protease inhibitors such as aptivus (tipranavir, TPV), crixivan (indinavir, IDV), invirase (saquinavir, SQV), kaletra (Aluvia, lopinavir/ritonavir, LPV/r), lexiva (Telzir, fosamprenavir, FPV), norvir (ritonavir, RTV), prezista (darunavir, DRV), reyataz (atazanavir, ATV) and viracept (nelfinavir, NFV); entry inhibitors (including fusion inhibitors) such as fuzeon (enfuvirtide, ENF, T-20), selzentry (Celsentri, maraviroc, UK-427, 857), cenicriviroc (TBR-652, TAK-652), ibalizumab (TNX-355) and PRO140; integrase inhibitors such as isentress (raltegravir, MK-0518), tivicay (dolutegravir, S/GSK-572) and elvitegravir (GS-9137); pharmacokinetic enhancers such as norvir (ritonavir, RTV), cobicistat (GS-9350) and SPI-452; HIV vaccines such as peptide vaccine, recombinant subunit protein vaccine, live vector vaccine, DNA vaccine, viruls-like particle vaccine (pseudovirion vaccine), vaccine combinations, rgp120 (AIDSVAX) (VAX003 and VAX004), ALVAC HIV (vCP1521)/AIDSVAX B/E (gp120) (RV144), Adenovirus type 5 (Ad5)/gag/pol/nef (HVTN 502/Merck 023), Ad5 gag/pol/nef (HVTB 503) and DNA-Ad5 gag/pol/nef/nev (HVTN505); combination therapy to elicit an immune response such as pegylated interferon alfa, hydroxyurea, mycophenolate mofetil (MPA) and its ester derivative mycophenolate mofetil (MMF); ribavirin, IL-2, IL-12, polymer polyethyleneimine (PEI), or a combination thereof; HIV-related opportunistic infection treatments such as Co-trimoxazole; and alternative life-style combination therapy such as acupuncture and exercise.

In some embodiments, a BTK inhibitor (e.g. ibrutinib) and an immune checkpoint inhibitor are administered in combinatin with an antiviral agent for the treatment of influenza virus infection. Exemplary antiviral agents for the treatment of influenza virus infection include, but are not limited to, antiviral drugs such as neuraminidase inhibitors (e.g. oseltamivir, peramivir and zanamivir) and admantanes (e.g. amantadine and rimantadine); seasonal flu vaccines (antigens representing three (trivalent) or four (quadrivalent) influenza virus strains) such as Flumist Quadrivalent (MedImmune, Gaithersburg, Md.), Fluarix Quadrivalent (Glaxo Smith Kline, Research Triangle Park, N.C.), Fluzone Quadrivalent (Sanofi Pasteur, Swiftwater, Pa.), Flulaval Quadrivalent, (ID Biomedical Corportation of Quebec/GlaxoSmith Kline, Research Triangle Park, N.C.), Flucelvax (Novartis Vaccines and Diagnostics, Cambridge, Mass.), and FluBlok (Protein Sciences, Meriden, Conn.); and combination drugs for the treatment of influenza including one or more immunomodulators such as immune suppressors or enhancers and anti-inflammatory agents.

In some embodiments, the anti-inflammatory agent can be non-steroidal, steroidal, or a combination thereof. Representative examples of non-steroidal anti-inflammatory agents include, but are not limited to, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, nifiumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof. In some embodiments, a BTK inhibitor (e.g. ibrutinib) and an immune checkpoint inhibitor are administered in combinatin with an anti-inflammatory agent for the treatment of influenza virus infection.

In some embodiments, a BTK inhibitor (e.g. ibrutinib) and an immune checkpoint inhibitor are administered in combinatin with an antiviral agent for the treatment of a human papillomavirus (HPV) infection. Exemplary antiviral agents for the treatment of HPV infection include, but are not limited to, podofilox or imiquimod.

In some embodiments, a BTK inhibitor (e.g. ibrutinib) and an immune checkpoint inhibitor are administered in combinatin with an antiviral agent for the treatment of an Epstein-Bar virus (EBV) infection. Exemplary antiviral agents for the treament of EBV infection include, but are not limited to, acyclovir, ganciclovir, and foscarnet.

In some embodiments, a BTK inhibitor (e.g. ibrutinib) and an immune checkpoint inhibitor are administered in combinatin with an antiviral agent for the treatment of a human T-cell leukemia virus (HTLV-1) infection. Exemplary antiviral agents for the treatment of HTLV-1 include, but are not limited to, mogamulizumab, interferon alpha, zidovudine, valproic acid, arsenic trioxide, and chemotherapeutic agents such as CHOP, R-CHOP, and the like.

In some embodiments, a BTK inhibitor (e.g. ibrutinib) and an immune checkpoint inhibitor are administered in combinatin with an antiviral agent for the treatment of Kaposi's sarcoma-associated herpesvirus (KSHV)/human herpes virus 8 (HHV8) infection. Exemplary antiviral agents for the treatment of KSHV/HHV8 include, but are not limited to, ganciclovir, valganciclovir, cidofovir, and foscarnet.

In some embodiments, a BTK inhibitor (e.g. ibrutinib) and an immune checkpoint inhibitor are administered in combinatin with an antibacterial agent for the treatment of a bacterial infection. Exemplary antibacterial agents include, but are not limited to, aminoglycosides such as amikacin, arbekacin, bekanamycin, dibekacin, framycetin, gentamicin, kanamycin, neomycin, netilmicin, paromomycin, ribostamycin, rhodostreptomycin, spectinomycin, hygromycin B, paromomycin sulfate, sisomicin, isepamicin, verdamicin, astromicin, streptomycin, tobramycin, and apramycin; ansamycins such as geldanamycin, herbimycin, rifaximin or streptomycin; carbapenem (beta-lactam) such as Imipenem, meropenem, ertapenem, doripenem, panipenem/betamipron, biapenem, razupenem, tebipenem, lenapenem or tomopenem; cephalosporin such as Cefacetrile (cephacetrile), Cefadroxil (cefadroxyl; Duricef), Cephalexin (cefalexin; Keflex), Cefaloglycin (cephaloglycin), Cefalonium (cephalonium), Cefaloridine (cephaloradine), Cefalotin (cephalothin; Keflin), Cefapirin (cephapirin; Cefadryl), Cefatrizine, Cefazaflur, Cefazedone, Cefazolin (cephazolin; Ancef, Kefzol), Cefradine (cephradine; Velosef), Cefroxadine, Ceftezole Cefaclor (Ceclor, Distaclor, Keflor, Raniclor), Cefonicid (Monocid), Cefprozil (cefproxil; Cefzil), Cefuroxime (Zefu, Zinnat, Zinacef, Ceftin, Biofuroksym, Xorimax), Cefoperazone (Cefobid), Ceftazidime (Meezat, Fortum, Fortaz), Ceftobiprole, Ceftaroline; glycopeptide antibiotics such as vancomycin, teicoplanin, telavancin, bleomycin, ramoplanin, and decaplanin; lincosamides such as clindamycin or lincomycin; lipopeptide such as daptomycin; macrolides such as azithromycin, clarithromycin, dirithromycin, erythromycin, roxithromycin, telithromycin, josamycin, kitasamycin, midecamycin, oleandomycin, solithromycin, spiramycin, troleandomycin, or tylosin; ketolides such as telithromycin, cethromycin, solithromycin, spiramycin, ansamycin, oleandomycin, or carbomycin; monobactam such as aztreonam; nitrofurans such as furazolidone, furylfuramide, nitrofurantoin, nitrofurazone, nifuratel, nifurquinazol, nifurtoinol, nifuroxazide or ranbezolid; oxazolidinones such as linezolid, posizolid, torezolid, radezolid, cycloserine, rivaroxaban or oxazolidinone and derivatives of; penicillins such as all natural penicillins (e.g. penicillins that are naturally produced by P. chrysogenum—e.g., penicillin G), biosynthetic penicillin (e.g. penicillins that are produced by P. chrysogenum through directed biosynthesis when a side chain acid is added to the medium—e.g., penicillin V), semi-synthetic penicillin (penicillin that are made by chemical means from natural or biosynthetic penicillin—e.g., ampicillin), synthetic penicillin (e.g. penicillin that are made wholly synthetically), adipyl-6-APA, amoxicillin, ampicillin, butyryl-6-APA, decanoyl-6-APA, heptanoyl-6-APA, hexanoyl-6-APA, nonanoyl-6-APA, octanoyl-6-APA, penicillin F, penicillin G, penicillin V, penicillin mX, penicillin X, 2-thiopheynlacetyl-6-APA, or valeryl-6-APA, azlocillin, flucloxacillin, amoxicillin/clavulanate, ampicillin/sulbactam, piperacillin/tazobactam, ticarcillin/clavulanate; polypeptides such as bacitracin, colistin or polymyxin B; quinolones such as cinoxacin, nalidixic acid, oxolinic acid, piromidic acid, pipemidic acid, rosoxacin, ciprofloxacin, enoxacin, fleroxacin, iomefloxacin, nadifloxacin, norfloxacin, ofloxacin, pefloxacin, rufloxacin, balofloxacin, grepafloxacin, levofloxacin, pazufloxacin, sparfloxacin, temafloxacin, tosufloxacin, clinafloxacin, gatifloxacin, gemifloxacin, moxifloxacin, sitafloxacin, trovafloxacin, prulifloxacin, delafloxacin, JNJ-Q2 or nemonoxacin; sulfonamides such as mafenide, sulfacetamide, sulfadiazine, silver sulfadiazine, sulfadimethoxine, sulfamethizole, sulfamethoxazole, sulfasalazine, sulfisoxazole, TMP-SMX, or sulfonamidochrysoidine; tetracycline such as naturally occurring tetracycline, chlortetracycline, oxytetracycline, demeclocycline, doxycycline, lymecycline, meclocycline, methacycline, minocycline or rolitetracycline; anti-mycobacteria agents such as clofazimine, dapsone, capreomycin, cycloserine, ethambutol, ethionamide, isoniazid, pyrazinamide, rifampin (rifampicin), rifabutin, rifapentine or streptomycin.

In some embodiments, a BTK inhibitor (e.g. ibrutinib) and an immune checkpoint inhibitor are administered in combinatin with an antifungal agent for the treatment of a fungal infection. Exemplary antifungal agents include, but are not limited to, polyene antifungals such as amphotericin B, candicidin, filipin, hamycin, natamycin, nystatin or rimocidin; imidazoles such as bifonazole, butoconazole, clotrimazole, econazole, fenticonazole, isoconazole, ketoconazole, miconazole, omoconazole, oxiconazole, sertaconazole, sulconazole or tioconazole; triazoles such as albaconazole, fluconazole, isavuconazole, itraconazole, posaconazole, ravuconazole, terconazole or voriconazole; thiazoles such as abafungin; allylamines such as amorolfin, butenafine, naftifine or terbinafine; echinocandins include anidulafungin, caspofungin or micafungin; antifungal macrolides such as polyene antimycotics (e.g., amphotericin B, nystatin benzoic acid); ciclopirox; flucytosine; griseofulvin; haloprogin; polygodial; tolnaftate; undecylenic acid; or crystal violet; and natural alternatives such as oregano, allicin, citronella oil, cocnut oil, iodine, lemon myrtle, neem seed oil, olife leaf, orange oil, palmarosa oil, patchouli, selenium, tea tree oil, zinc, horopito, turnip, chives, radish and garlic.

In some embodiments, a BTK inhibitor (e.g. ibrutinib) and an immune checkpoint inhibitor are administered in combinatin with an antiparasitic agent for the treatment of a parasitic infection. Exemplary antiparasitic agents include, but are not limited to, antimony-containing compounds, such as meglumine antimoniate and sodium stibogluconate, amphotericin B, ketoconazole, itraconazole, fluconazole, miltefosine, paromomycin, and pentamidine.

In some embodiments, a BTK inhibitor (e.g. ibrutinib) and an immune checkpoint inhibitor are administered in combinatin with an antiprotozoal agent for the treatment of a protozoan infection. Exemplary antiprotozoal agents include, but are not limited to, Acetarsol, Azanidazole, Chloroquine, Metronidazole, Nifuratel, Nimorazole, Omidazole, Propenidazole, Secnidazole, Sineflngin, Tenonitrozole, Temidazole, Timidazole, and pharmaceutically acceptable salts or esters thereof.

### Modulation of Th1/Th2 Profile

Adaptive immunity is modulated by a complex network of T and B cells and T helper (Th) cells are the regulators of this network. The Th cells can differentiate into Th1 cells which promote cellular immunity or Th2 cells which promote humoral immunity. In certain instances, cancer cells promote a Th2 response which allows survival and evasion of these cancer cells from the host immune system. Described herein, in certain embodiments, are methods of treating a cancer in a subject in need thereof by increasing the Th1:Th2 biomarker ratio in the subject, comprising administering to the subject a therapeutically effective amount of a combination comprising a TEC inhibitor and an immune checkpoint inhibitor, wherein the combination decreases the Th2 response in the subject and increases the Th1 response in the subject. In some embodiments, the TEC inhibitor is a BTK inhibitor or an ITK inhibitor. In some embodiments, the TEC inhibitor is a BTK inhibitor. In some embodiments, the BTK inhibitor is ibrutinib. In some embodiments, the Btk inhibitor (e.g., ibrutinib) functions to suppress the Th1 response while enhancing the Th2 response. In some embodiments, the BTK inhibitor (e.g. ibrutinib) functions to decrease the number of Th2 polarized T cells in a subject. In some embodiments, the BTK inhibitor (e.g. ibrutinib) functions to increase the number of Th1 polarized T cells in a subject. In some embodiments, the BTK inhibitor (e.g. ibrutinib) functions to increase the number of activated CD8+ cytotoxic T cells in a subject. In some embodiments, the BTK inhibitor (e.g. ibrutinib) functions to increase the ratio of Th1 polarized T cells to Th2 polarized T cells in a subject. In some embodiments, the BTK inhibitor (e.g. ibrutinib) functions to increase IFN-γ expression in a subject. In some embodiments, the cancer is a solid tumor. In some embodiments, the solid tumor is selected from alveolar soft part sarcoma, bladder cancer, breast cancer, colorectal (colon) cancer, Ewing's bone sarcoma, gastroenterological cancer, head and neck cancer, kidney cancer, leiomyosarcoma, lung cancer, melanoma, osteosarcoma, ovarian cancer, pancreatic cancer, prostate cancer, proximal or distal bile duct cancer, and neuroblastoma. In some embodiments, the cancer is a hematologic cancer. In some embodiments, the hematologic cancer is a B-cell malignancy. In some embodiments, the B-cell malignancy is chronic lymphocytic leukemia (CLL), small lymphocytic lymphoma (SLL), high risk CLL, non-CLL/SLL lymphoma, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, the Btk inhibitor (e.g., ibrutinib) in combination with an immune checkpoint inhibitor function to suppress the Th1 response while enhancing the Th2 response. In some embodiments, the BTK inhibitor (e.g. ibrutinib) in combination with an immune checkpoint inhibitor function to decrease the number of Th2 polarized T cells in a subject. In some embodiments, the BTK inhibitor (e.g. ibrutinib) in combination with an immune checkpoint inhibitor function to increase the number of Th1 polarized T cells in a subject. In some embodiments, the BTK inhibitor (e.g. ibrutinib) in combination with an immune checkpoint inhibitor function to increase the number of activated CD8+ cytotoxic T cells in a subject. In some embodiments, the BTK inhibitor (e.g. ibrutinib) in combination with an immune checkpoint inhibitor function to increase the ratio of Th1 polarized T cells to Th2 polarized T cells in a subject. In some embodiments, the BTK inhibitor (e.g. ibrutinib) in combination with an immune checkpoint inhibitor functions to increase IFN-γ expression in a subject.

In some embodiments, a Btk inhibitor (e.g., ibrutinib) increases a Th1 immune response against the cancer compared to no treatment with the Btk inhibitor (e.g., ibrutinib). In some embodiments, a Btk inhibitor (e.g., ibrutinib) decreases a Th2 immune response against the cancer compared to no treatment with the Btk inhibitor (e.g., ibrutinib). In some embodiments, a Btk inhibitor (e.g., ibrutinib) alters the ratio of Th1-Th2 immune response against the cancer compared to no treatment with the Btk inhibitor (e.g., ibrutinib). In some embodiments, a Btk inhibitor (e.g., ibrutinib) increases the ratio of Th1-Th2 immune response against the cancer compared to no treatment with the Btk inhibitor (e.g., ibrutinib). In some embodiments, a Btk inhibitor (e.g., ibrutinib) increases the population of Th1 cells by about 1%, 2%, 3%, 4%, 5%, 10%, 20%, 30%, 40%, 50%, 60%, 70%, 80%, 90% or greater compared to no treatment with the Btk inhibitor (e.g., ibrutinib). In some embodiments, a Btk inhibitor (e.g., ibrutinib) decreases the population of Th2 cells by about 1%, 2%, 3%, 4%, 5%, 10%, 20%, 30%, 40%, 50%, 60%, 70%, 80%, 90% or greater compared to no treatment with the Btk inhibitor (e.g., ibrutinib). In some embodiments, a Btk inhibitor (e.g., ibrutinib) increases the expression of one or more Th1 related markers. In some embodiments, a Btk inhibitor (e.g., ibrutinib) increases the expression of one or more Th1 related markers by about 1%, 2%, 3%, 4%, 5%, 10%, 20%, 30%, 40%, 50%, 60%, 70%, 80%, 90% or greater compared to no treatment with the Btk inhibitor (e.g., ibrutinib). In some embodiments, the one or more Th1 related marker includes CCR1, CD4, CD26, CD94, CD119, CD183, CD195, CD212, GM-CSF, Granzyme B, IFN-α, IFN-γ, IL-2, IL-12, IL-15, IL-18R, IL-23, IL-27, IL-27R, Lymphotoxin, perforin, t-bet, Tim-3, TNF-α, TRANCE, sCD40L, or any combination thereof. In some embodiments, the one or more Th1 related markers includes IFN-γ, IL-2, IL-12 or any combination thereof. In some embodiments, a Btk inhibitor (e.g., ibrutinib) decreases the expression of Th2 related markers. In some embodiments, a Btk inhibitor (e.g., ibrutinib) decreases the expression of Th2 related markers by about 1%, 2%, 3%, 4%, 5%, 10%, 20%, 30%, 40%, 50%, 60%, 70%, 80%, 90% or greater compared to no treatment with the Btk inhibitor (e.g., ibrutinib). In some embodiments, the one or more Th2 related markers includes CCR3, CCR4, CCR7, CCR8, CD4, CD30, CD81, CD184, CD278, c-maf, CRTH2, Gata-3, GM-CSF, IFNγR, IgD, IL-1R, IL-4, IL-5, IL-6, IL-9, IL-10, IL-13, IL-15, ST2L/T1, Tim-1, or any combination thereof. In some embodiments, the one or more Th1 related markers includes IL-4, IL-10, IL-13, or any combination thereof.

In some embodiments, the combination of a BTK inhibitor and an immune checkpoint inhibitor increases a Th1 immune response against the cancer compared to no treatment with this combination. In some embodiments, the combination of a BTK inhibitor and an immune checkpoint inhibitor decreases a Th2 immune response against the cancer compared to no treatment with this combination. In some embodiments, the combination of a BTK inhibitor and an immune checkpoint inhibitor alters the ratio of Th1-Th2 immune response against the cancer compared to no treatment with this combination. In some embodiments, the combination of a BTK inhibitor and an immune checkpoint inhibitor increases the ratio of Th1-Th2 immune response against the cancer compared to no treatment with this combination. In some embodiments, the combination of a BTK inhibitor and an immune checkpoint inhibitor increases the population of Th1 cells by about 1%, 2%, 3%, 4%, 5%, 10%, 20%, 30%, 40%, 50%, 60%, 70%, 80%, 90% or greater compared to no treatment with this combination. In some embodiments, the combination of a BTK inhibitor and an immune checkpoint inhibitor decreases the population of Th2 cells by about 1%, 2%, 3%, 4%, 5%, 10%, 20%, 30%, 40%, 50%, 60%, 70%, 80%, 90% or greater compared to no treatment with this combination. In some embodiments, the combination of a BTK inhibitor and an immune checkpoint inhibitor increases the expression of one or more Th1 related markers. In some embodiments, the combination of a BTK inhibitor and an immune checkpoint inhibitor increases the expression of one or more Th1 related markers by about 1%, 2%, 3%, 4%, 5%, 10%, 20%, 30%, 40%, 50%, 60%, 70%, 80%, 90% or greater compared to no treatment with this combination. In some embodiments, the one or more Th1 related marker includes CCR1, CD4, CD26, CD94, CD119, CD183, CD195, CD212, GM-CSF, Granzyme B, IFN-α, IFN-γ, IL-2, IL-12, IL-15, IL-18R, IL-23, IL-27, IL-27R, Lymphotoxin, perforin, t-bet, Tim-3, TNF-α, TRANCE, sCD40L, or any combination thereof. In some embodiments, the one or more Th1 related markers includes IFN-γ, IL-2, IL-12 or any combination thereof. In some embodiments, the combination of a BTK inhibitor and an immune checkpoint inhibitor decreases the expression of Th2 related markers. In some embodiments, the combination of a BTK inhibitor and an immune checkpoint inhibitor decreases the expression of Th2 related markers by about 1%, 2%, 3%, 4%, 5%, 10%, 20%, 30%, 40%, 50%, 60%, 70%, 80%, 90% or greater compared to no treatment with this combination. In some embodiments, the one or more Th2 related markers includes CCR3, CCR4, CCR7, CCR8, CD4, CD30, CD81, CD184, CD278, c-maf, CRTH2, Gata-3, GM-CSF, IFNγR, IgD, IL-1R, IL-4, IL-5, IL-6, IL-9, IL-10, IL-13, IL-15, ST2L/T1, Tim-1, or any combination thereof. In some embodiments, the one or more Th1 related markers includes IL-4, IL-10, IL-13, or any combination thereof.

In some embodiments, the combination of ibrutinib and an immune checkpoint inhibitor increases a Th1 immune response against the cancer compared to no treatment with this combination. In some embodiments, the combination of ibrutinib and an immune checkpoint inhibitor decreases a Th2 immune response against the cancer compared to no treatment with this combination. In some embodiments, the combination of ibrutinib and an immune checkpoint inhibitor alters the ratio of Th1-Th2 immune response against the cancer compared to no treatment with this combination. In some embodiments, the combination of ibrutinib and an immune checkpoint inhibitor increases the ratio of Th1-Th2 immune response against the cancer compared to no treatment with this combination. In some embodiments, the combination of ibrutinib and an immune checkpoint inhibitor increases the population of Th1 cells by about 1%, 2%, 3%, 4%, 5%, 10%, 20%, 30%, 40%, 50%, 60%, 70%, 80%, 90% or greater compared to no treatment with this combination. In some embodiments, the combination of ibrutinib and an immune checkpoint inhibitor decreases the population of Th2 cells by about 1%, 2%, 3%, 4%, 5%, 10%, 20%, 30%, 40%, 50%, 60%, 70%, 80%, 90% or greater compared to no treatment with this combination. In some embodiments, the combination of ibrutinib and an immune checkpoint inhibitor increases the expression of one or more Th1 related markers. In some embodiments, the combination of ibrutinib and an immune checkpoint inhibitor increases the expression of one or more Th1 related markers by about 1%, 2%, 3%, 4%, 5%, 10%, 20%, 30%, 40%, 50%, 60%, 70%, 80%, 90% or greater compared to no treatment with this combination. In some embodiments, the one or more Th1 related marker includes CCR1, CD4, CD26, CD94, CD119, CD183, CD195, CD212, GM-CSF, Granzyme B, IFN-α, IFN-γ, IL-2, IL-12, IL-15, IL-18R, IL-23, IL-27, IL-27R, Lymphotoxin, perforin, t-bet, Tim-3, TNF-α, TRANCE, sCD40L, or any combination thereof. In some embodiments, the one or more Th1 related markers includes IFN-γ, IL-2, IL-12 or any combination thereof. In some embodiments, the combination of ibrutinib and an immune checkpoint inhibitor decreases the expression of Th2 related markers. In some embodiments, the combination of ibrutinib and an immune checkpoint inhibitor decreases the expression of Th2 related markers by about 1%, 2%, 3%, 4%, 5%, 10%, 20%, 30%, 40%, 50%, 60%, 70%, 80%, 90% or greater compared to no treatment with this combination. In some embodiments, the one or more Th2 related markers includes CCR3, CCR4, CCR7, CCR8, CD4, CD30, CD81, CD184, CD278, c-maf, CRTH2, Gata-3, GM-CSF, IFNγR, IgD, IL-1R, IL-4, IL-5, IL-6, IL-9, IL-10, IL-13, IL-15, ST2L/T1, Tim-1, or any combination thereof. In some embodiments, the one or more Th1 related markers includes IL-4, IL-10, IL-13, or any combination thereof.

### Biomarker Profiles

Disclosed herein, in certain embodiments, are methods of patient selection and stratification, therapeutic regimen selection, and/or optimization of a therapeutic regimen based on a biomarker profile. In some embodiments, the biomarker profile indicates the expression of a biomarker, the expression level of a biomarker, mutations in a biomarker, or the presence of a biomarker. In some embodiments, the biomarker profile is compared to a control biomarker profile. In some embodiments, the therapeutic regimen is a combination of a TEC inhibitor and an immune check point inhibitor. In some embodiments, the biomarker profile is analyzed prior, during, and/or post administration of a combination of a TEC inhibitor and an immune checkpoint inhibitor. In some embodiments, the TEC inhibitor is a BTK inhibitor or an ITK inhibitor. In some embodiments, the TEC inhibitor is a BTK inhibitor. In some embodiments, the biomarker profile is analyzed prior, during, and/or post administration of a combination of a BTK inhibitor and an immune checkpoint inhibitor. In some embodiments, the BTK inhibitor is ibrutinib. In some embodiments, the biomarker profile is analyzed prior, during, and/or post administration of a combination of ibrutinib and an immune checkpoint inhibitor. In some embodiments, the biomarker profile is analyzed prior, during, and/or post administration of a combination of a BTK inhibitor (e.g. ibrutinib), an immune checkpoint inhibitor, and an additional therapeutic agent.

In some embodiments, a biomarker is any cytogenetic, cell surface molecular or protein or RNA expression marker. In some embodiments, a biomarker includes Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, and VTCN1.

In some instances, the expression level of a biomarker selected from Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, and VTCN1 is compared to a control. In some embodiments, the expression level of a biomarker selected from Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, and VTCN1 is decreased by 0.5-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 50-fold, 75-fold, 100-fold, 200-fold, 500-fold, 1000-fold, or less compared to the control. In some embodiments, the expression level of a biomarker selected from Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, and VTCN1 is increased by 0.5-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 50-fold, 75-fold, 100-fold, 200-fold, 500-fold, 1000-fold, or more compared to the control. In some embodiments, the control is the expression level of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, and VTCN1 in an individual who does not have a cancer, or the expression level of an individual prior to treatment with a combination of a TEC inhibitor and an immune checkpoint inhibitor.

In some aspects, an elevated expression level of a biomarker selected from: Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, and VTCN1 is associated with a poor prognosis. In some embodiments, an elevated expression level of a biomarker selected from: Programmed Death-Ligand 1 (PD-L1, also known as B7-H1, CD274), Programmed Death 1 (PD-1), CTLA-4, PD-L2, LAG3, and TIM3 is associated with a poor prognosis.

In some embodiments, an elevated expression level of a biomarker selected from: Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, and VTCN1 is associated with decreased survival, tumor size increase, tumor aggressiveness, recurrence, metastasis, and/or decreased tumor-infiltrating lymphocytes. In some embodiments, an elevated expression level of a biomarker selected from: Programmed Death-Ligand 1 (PD-L1, also known as B7-H1, CD274), Programmed Death 1 (PD-1), CTLA-4, PD-L2, LAG3, and TIM3 is associated with decreased survival, tumor size increase, tumor aggressiveness, recurrence, metastasis, and/or decreased tumor-infiltrating lymphocytes.

In some instances, the expression level of a biomarker selected from: Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, and VTCN1 is used for patient selection, stratification, or monitoring for development of resistance for a combination therapy that comprises a TEC inhibitor (e.g. BTK inhibitor such as ibrutinib, ITK inhibitor) and an immune checkpoint inhibitor.

In some cases, the expression level of a biomarker selected from: Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, and VTCN1 is used for a therapeutic regimen selection or optimization that comprises a combination of a TEC inhibitor (e.g. BTK inhibitor such as ibrutinib, ITK inhibitor) and an immune checkpoint inhibitor.

In some embodiments, a biomarker is selected from biomarkers that are expressed by or correlate with a solid tumor, such as for example bladder cancer, colon cancer, breast cancer, lung cancer, ovarian cancer, prostate cancer, pancreatic cancer, and proximal or distal bile duct carcinoma. In some embodiments, biomarkers for bladder cancer include BTA Stat, BTA Track, NMP 22, Bladder Chek, immunocyt, UroVysion, cytokeratins 8, 18 and 19, telomerase TRAP, hTert and hTR, BLCA-4, survivn, hyaluronic acid/hyaluronidase, DD23 monoclonal antibody, fibronectin and HCG. In some embodiments, biomarkers for colon cancer include CEA, CA 19-9, CYFRA 21-1, ferritin, osteopontin, p53, seprase and EGFR. In some embodiments, biomarkers for lung cancer include ERCC-1, NSE, ProGRP, SCC, beta-tubulin, RRM1, EGFR, VEGF, CYFRA-21-1, CEA, CRP, LDH, CAl25, CgA, NCAM and TPA. In some embodiments, biomarkers for ovarian cancer include CAl25, Her-2/neu, Akt-2, inhibin, HLA-G, TATI, CASA, TPA, CEA, LPA, PAI-1, IL-6, kallikreins 5, 6, 7, 8, 9, 10, 11, 13, 14, 15, hCG_{βcf}, prostasin, osteopontin, HE4, mitogen-activated protein kinase, IGFBP-2, RSF-1 and NAC-1. In some embodiments, biomarkers for pancreatic cancer include CA19-9, CEA, TIMP-1, CA50, CA242, MUC1, MUCSAC, Claudin 18 and annexin A8. In some embodiments, biomarkers for prostate cancer include PSA, human kallikrein 2, IGF-1, IGFBP-3, PCA3, AMACR, GSTPi, CDKN1B, Ki-67, PTEN, and PSCA. In some embodiments, biomarkers for proximal or distal bile duct carcinoma include CAl25, CA19-9, CEA, CgA, MUC1, MUCSAC, PML, p53, DPC4, Ki67, matrix metalloproteinases, alpha-fetoprotein, N-cadherin, VEGF-C, claudins, thrombospondin-1, cytokeratins and CYFRA 21-1. In some embodiments, biomarkers for breast cancer include HER-1, -2, -3, -4; EGFR; HER-2/neu; Foxp3⁺; ATAD2; DERL1; ESR1; CCND1; MYC; E2F1; NEK2A; CRYAB; HSPB2; FOXM1; DNMT3B; and MAT1A.

In some embodiments, the expression level of a biomarker associated with a solid tumor (e.g. bladder cancer, colon cancer, breast cancer, lung cancer, ovarian cancer, prostate cancer, pancreatic cancer, and proximal or distal bile duct carcinoma) is compared to a control. In some embodiments, the expression level of a biomarker associated with a solid tumor (e.g. bladder cancer, colon cancer, breast cancer, lung cancer, ovarian cancer, prostate cancer, pancreatic cancer, and proximal or distal bile duct carcinoma) is increased by 0.5-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 50-fold, 75-fold, 100-fold, 200-fold, 500-fold, 1000-fold, or more compared to the control. In some embodiments, the expression level of a biomarker associated with a solid tumor (e.g. bladder cancer, colon cancer, breast cancer, lung cancer, ovarian cancer, prostate cancer, pancreatic cancer, and proximal or distal bile duct carcinoma) is decreased by 0.5-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 50-fold, 75-fold, 100-fold, 200-fold, 500-fold, 1000-fold, or less compared to the control. In some embodiments, the control is the expression level of a biomarker associated with a solid tumor (e.g. bladder cancer, colon cancer, breast cancer, lung cancer, ovarian cancer, prostate cancer, pancreatic cancer, and proximal or distal bile duct carcinoma) in an individual who does not have a cancer, or the expression level of an individual prior to treatment with a combination of a TEC inhibitor and an immune checkpoint inhibitor.

In some instances, the expression level of a biomarker associated with a solid tumor (e.g. bladder cancer, colon cancer, breast cancer, lung cancer, ovarian cancer, prostate cancer, pancreatic cancer, and proximal or distal bile duct carcinoma) is used for patient selection, stratification, or monitoring for development of resistance for a combination therapy that comprises a TEC inhibitor (e.g. BTK inhibitor such as ibrutinib, ITK inhibitor) and an immune checkpoint inhibitor.

In some instances, the expression level of a biomarker associated with a solid tumor (e.g. bladder cancer, colon cancer, breast cancer, lung cancer, ovarian cancer, prostate cancer, pancreatic cancer, and proximal or distal bile duct carcinoma) is used for a therapeutic regimen selection or optimization that comprises a combination of a TEC inhibitor (e.g. BTK inhibitor such as ibrutinib, ITK inhibitor) and an immune checkpoint inhibitor.

In some embodiments, a biomarker is selected from biomarkers that are expressed by or correlate with a hematologic cancer, such as for example CLL, DLBCL, mantle cell lymphoma, and Waldenström's macroglobulinemia. In some embodiments, biomarkers for CLL include del(17p13.1), del(11q22.3), del(11q23), unmutated IgVH together with ZAP-70+ and/or CD38+, trisomy 12, del(13q14), complex karyotype, TP53, NOTCH1, SF3B1, BIRC3, LPL, and CLLU1. In some embodiments, biomarkers for DLBCL include BCL6, GCET1, MUM1, CD10, FOXP1, miR-21, miR-23A, miR-27A, miR-19A, miR-195, miR-LET7G, miR-127, miR-222, miR-221, t(14:18), trisomy 3, del(8p23.1), del(8p23.1-21.2), del(8p), t(6;14)(p25;q32), TP53, TP21, BCL2, BCL6, MYC, Ki-67, and CD43. In some embodiments, biomarkers for mantle cell lymphoma include t(11;14)(q13;q32), MYC, CDKN2A, TNFRSF10B, CCDN1, Ki-67, and SOX11. In some embodiments, biomarker for Waldenström's macroglobulinemia include CD19, CD20, CD22, CD38, CD79a, CD5, CD138, monoclonal surface Ig, MYD88, CXCR4, TP53, ATM, IgH, del(6q), and trisomy 18.

In some embodiments, the biomarker profile of a hematologic cancer is the presence or absence of a biomarker, such as a cytogenetic mutation. In some embodiment, the biomarker profile of a hematologic cancer is the expression level of a biomarker. In some embodiments, the expression level of a biomarker associated with a hematologic cancer (e.g. CLL, DLBCL, mantle cell lymphoma, or Waldenström's macroglobulinemia) is compared to a control. In some embodiments, the expression level of a biomarker associated with a hematologic cancer (e.g. CLL, DLBCL, mantle cell lymphoma, or Waldenström's macroglobulinemia) is increased by 0.5-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 50-fold, 75-fold, 100-fold, 200-fold, 500-fold, 1000-fold, or more compared to the control. In some embodiments, the expression level of a biomarker associated with a hematologic cancer (e.g. CLL, DLBCL, mantle cell lymphoma, or Waldenström's macroglobulinemia) is decreased by 0.5-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 50-fold, 75-fold, 100-fold, 200-fold, 500-fold, 1000-fold, or less compared to the control. In some embodiments, the control is the expression level of a biomarker associated with a hematologic cancer (e.g. CLL, DLBCL, mantle cell lymphoma, or Waldenström's macroglobulinemia) in an individual who does not have a cancer, or the expression level of an individual prior to treatment with a combination of a TEC inhibitor and an immune checkpoint inhibitor.

In some instances, the presence or absence or the expression level of a biomarker associated with a hematologic cancer (e.g. CLL, DLBCL, mantle cell lymphoma, or Waldenström's macroglobulinemia) is used for patient selection, stratification, or monitoring for development of resistance for a combination therapy that comprises a TEC inhibitor (e.g. BTK inhibitor such as ibrutinib, ITK inhibitor) and an immune checkpoint inhibitor.

In some cases, the presence or absence or the expression level of a biomarker associated with a hematologic cancer (e.g. CLL, DLBCL, mantle cell lymphoma, or Waldenström's macroglobulinemia) is used for a therapeutic regimen selection or optimization that comprises a combination of a TEC inhibitor (e.g. BTK inhibitor such as ibrutinib, ITK inhibitor) and an immune checkpoint inhibitor.

In some embodiments, a biomarker is tumor-infiltrating lymphocytes (TILs). In some embodiments, the expression level of immune checkpoint proteins (e.g. PD-1) by tumor-infiltrating lymphocytes is compared with the expression level of control tumor-infiltrating lymphocytes. In some embodiments, the expression level of immune checkpoint proteins (e.g. PD-1) by tumor-infiltrating lymphocytes is increased by 0.5-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 50-fold, 75-fold, 100-fold, 200-fold, 500-fold, 1000-fold, or more compared to the control. In some embodiments, the expression level of immune checkpoint proteins (e.g. PD-1) by tumor-infiltrating lymphocytes is decreased by 0.5-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 50-fold, 75-fold, 100-fold, 200-fold, 500-fold, 1000-fold, or less compared to the control. In some embodiments, the control is obtained from an individual who does not have a cancer or from an individual prior to treatment with a combination of a TEC inhibitor and an immune checkpoint inhibitor. In some embodiments, an elevated expression level of an immune checkpoint protein (e.g. PD-1) by tumor-infiltrating lymphocytes is associated with impaired effector function such as cytokine production and cytotoxic efficacy against tumor cells, and/or poor prognosis.

In some embodiments, a biomarker is absolute lymphocyte count (ALC). In some embodiments, the ALC level is greater than 100, 500, 1000, 1500, 2000, 2500, 3000, 3500, 4000, 4500, 5000 cells/μL, or higher. In some embodiments, the ALC level is less than 100, 500, 1000, 1500, 2000, 2500, 3000, 3500, 4000, 4500, 5000 cells/μL, or lower. In some embodiments, ALC levels higher than about 1000 cells/μL is associated with improved overall survival.

In some embodiments, the biomarker includes a mutation or modification in BTK. In some embodiments, the modification is a mutation at amino acid position 481 in BTK. In some embodiments, the mutation is C481S in BTK. In some embodiments, the therapeutic regimen of a BTK inhibitor (e.g. ibrutinib) and an immune checkpoint inhibitor is modified based on the presence or absence of C481S mutation in BTK. In some embodiments, the therapeutic regimen of a BTK inhibitor (e.g. ibrutinib), an immune checkpoint inhibitor, and an additional therapeutic agent is modified based on the presence or absence of C481S mutation in BTK. In some embodiments, the presence of C481S mutation in a cancer confers resistance of the cancer to a BTK inhibitor (e.g. ibrutinib). In some embodiments, a cancer that has the C481S mutation is characterized as an ibrutinib-resistant cancer.

In some instances, the presence or absence, or expression levels of biomarkers such as TILs, ALC, and C481S of BTK are used for patient selection, stratification, or monitoring for development of resistance for a combination therapy that comprises a TEC inhibitor (e.g. BTK inhibitor such as ibrutinib, ITK inhibitor) and an immune checkpoint inhibitor. In some instances, biomarkers such as TILs, ALC, and C481S of BTK are used for a therapeutic regimen selection or optimization that comprises a combination of a TEC inhibitor (e.g. BTK inhibitor such as ibrutinib, ITK inhibitor) and an immune checkpoint inhibitor.

Biomarker Profile Associated with Th1/Th2

In some embodiments, administration of a combination of a TEC inhibitor (e.g. BTK inhibitor or ITK inhibitor) and an immune checkpoint inhibitor decreases the biomarker profile of one population of cells. In some embodiments, administration of a combination of a BTK inhibitor (e.g. ibrutinib) and an immune checkpoint inhibitor decreases the biomarker profile of one population of cells. In some embodiments, administration of a combination of ibrutinib and an immune checkpoint inhibitor decreases the biomarker profile of one population of cells. In some embodiments, the population of cells is Th2 polarized T cells. In some embodiments, administration of a combination of ibrutinib and an immune checkpoint inhibitor decreases the biomarker profile of Th2 polarized T cell population. In some embodiments, administration of a combination of ibrutinib and an immune checkpoint inhibitor decreases the biomarker profile of Th2 polarized T cell population in a subject.

In some embodiments, administration of a combination of a TEC inhibitor (e.g. BTK inhibitor or ITK inhibitor) and an immune checkpoint inhibitor increases the biomarker profile of a second population of cells. In some embodiments, administration of a combination of a BTK inhibitor (e.g. ibrutinib) and an immune checkpoint inhibitor increases the biomarker profile of a second population of cells. In some embodiments, administration of a combination of ibrutinib and an immune checkpoint inhibitor increases the biomarker profile of a second population of cells. In some embodiments, the second population of cells is Th1 polarized T cells. In some embodiments, administration of a combination of ibrutinib and an immune checkpoint inhibitor increases the biomarker profile of Th1 polarized T cells populations. In some embodiments, administration of a combination of ibrutinib and an immune checkpoint inhibitor increases the biomarker profile of Th1 polarized T cells populations in a subject.

In some embodiments, administration of a combination of a BTK inhibitor (e.g. ibrutinib) and an immune checkpoint inhibitor increases the ratio of Th1 polarized T cells to Th2 polarized T cells in the subject. In some embodiments, administration of a combination of a BTK inhibitor (e.g. ibrutinib) and an immune checkpoint inhibitor increases the ratio of Th1 polarized T cells to Th2 polarized T cells in the subject by about 5 fold, 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold or greater. In some embodiments, administration of a combination of a BTK inhibitor (e.g. ibrutinib) and an immune checkpoint inhibitor increase the number of cytotoxic CD8+ T cells in the subject.

In some embodiments, administration of a combination of a BTK inhibitor (e.g. ibrutinib) and an immune checkpoint inhibitor decreases the expression of one or more biomarkers in a subject. In some embodiments, the biomarker is a Th2 related marker in the subject. In some embodiments, administration of a combination of a BTK inhibitor (e.g. ibrutinib) and an immune checkpoint inhibitor decreases the expression of one or more Th2 related markers selected from among CCR3, CCR4, CCR7, CCR8, CD4, CD30, CD81, CD184, CD278, c-maf, CRTH2, Gata-3, GM-CSF, IFNγR, IgD, IL-1R, IL-4, IL-5, IL-6, IL-9, IL-10, IL-13, IL-15, ST2L/T1 and Tim-1. In some embodiments, administration of a combination of a BTK inhibitor (e.g. ibrutinib) and an immune checkpoint inhibitor decreases IL-4, IL-5, IL-6, IL-10, IL-13, or IL-15 expression in the subject. In some embodiments, administration of a combination of a BTK inhibitor (e.g. ibrutinib) and an immune checkpoint inhibitor decreases IL-4 expression in the subject. In some embodiments, administration of a combination of a BTK inhibitor (e.g. ibrutinib) and an immune checkpoint inhibitor decreases IL-5 expression in the subject. In some embodiments, administration of a combination of a BTK inhibitor (e.g. ibrutinib) and an immune checkpoint inhibitor decreases IL-6 expression in the subject. In some embodiments, administration of a combination of a BTK inhibitor (e.g. ibrutinib) and an immune checkpoint inhibitor decreases IL-10 expression in the subject. In some embodiments, administration of a combination of a BTK inhibitor (e.g. ibrutinib) and an immune checkpoint inhibitor decreases IL-13 expression in the subject. In some embodiments, administration of a combination of a BTK inhibitor (e.g. ibrutinib) and an immune checkpoint inhibitor decreases IL-15 expression in the subject.

In some embodiments, administration of a combination of a BTK inhibitor (e.g. ibrutinib) and an immune checkpoint inhibitor increases the expression of one or more biomarkers in a subject. In some embodiments, the biomarker is a Th1 related marker in the subject. In some embodiments, administration of a combination of a BTK inhibitor (e.g. ibrutinib) and an immune checkpoint inhibitor increases the expression of one or more Th1 related markers selected from among CCR1, CD4, CD26, CD94, CD119, CD183, CD195, CD212, GM-CSF, Granzyme B, IFN-α, IFN-γ, IL-2, IL-12, IL-15, IL-18R, IL-23, IL-27, IL-27R, Lymphotoxin, perforin, t-bet, Tim-3, TNF-α, TRANCE, and sCD40L. In some embodiments, administration of a combination of a BTK inhibitor (e.g. ibrutinib) and an immune checkpoint inhibitor increases IFN-γ, GM-CSF, IL-2, IL-12(p70) expression in the subject. In some embodiments, administration of a combination of a BTK inhibitor (e.g. ibrutinib) and an immune checkpoint inhibitor increases IFN-γ expression in the subject. In some embodiments, administration of a combination of a BTK inhibitor (e.g. ibrutinib) and an immune checkpoint inhibitor increases GM-CSF expression in the subject. In some embodiments, administration of a combination of a BTK inhibitor (e.g. ibrutinib) and an immune checkpoint inhibitor increases IL-2 expression in the subject. In some embodiments, administration of a combination of a BTK inhibitor (e.g. ibrutinib) and an immune checkpoint inhibitor increases IL-12(p70) expression in the subject.

### Diagnostic Methods

Methods for determining the expression or presence of biomarkers described supra are well known in the art. Circulating levels of biomarkers in a blood sample obtained from a candidate subject are measured, for example, by ELISA, radioimmunoassay (RIA), electrochemiluminescence (ECL), Western blot, multiplexing technologies, or other similar methods. Cell surface expression of biomarkers are measured, for example, by flow cytometry, immunohistochemistry, Western Blot, immunoprecipitation, magnetic bead selection, and quantification of cells expressing either of these cell surface markers. Biomarker RNA expression levels could be measured by RT-PCR, Qt-PCR, microarray, Northern blot, or other similar technologies.

As disclosed herein, determining the expression or presence of the biomarker of interest at the protein and/or nucleotide level is accomplished using any detection method known to those of skill in the art. By “detecting expression” or “detecting the level of is intended determining the expression level or presence of a biomarker protein or gene in the biological sample. Thus, “detecting expression” encompasses instances where a biomarker is determined not to be expressed, not to be detectably expressed, expressed at a low level, expressed at a normal level, or overexpressed.

In certain aspects of the method provided herein, the one or more subpopulation of lymphocytes are isolated, detected or measured. In certain embodiments, the one or more subpopulations of lymphocytes are isolated, detected or measured using immunophenotyping techniques. In other embodiments, the one or more subpopulations of lymphocytes are isolated, detected or measured using fluorescence activated cell sorting (FACS) techniques.

In certain aspects, the determining step requires determining the expression or presence of a biomarker. In certain aspects, the methods described herein, the determining step requires determining the expression or presence of a combination of biomarkers.

In certain aspects, the expression or presence of these various biomarkers and any clinically useful prognostic markers in a biological sample are detected at the protein or nucleic acid level, using, for example, immunohistochemistry techniques or nucleic acid-based techniques such as in situ hybridization and RT-PCR. In one embodiments, the expression or presence of one or more biomarkers is carried out by a means for nucleic acid amplification, a means for nucleic acid sequencing, a means utilizing a nucleic acid microarray (DNA and RNA), or a means for in situ hybridization using specifically labeled probes.

In other embodiments, the determining the expression or presence of one or more biomarkers is carried out through gel electrophoresis. In one embodiment, the determination is carried out through transfer to a membrane and hybridization with a specific probe.

In other embodiments, the determining the expression or presence of one or more biomarkers carried out by a diagnostic imaging technique.

In still other embodiments, the determining the expression or presence of one or more biomarkers carried out by a detectable solid substrate. In one embodiment, the detectable solid substrate is paramagnetic nanoparticles functionalized with antibodies.

In another aspect, provided herein are methods for detecting or measuring residual lymphoma following a course of treatment in order to guide continuing or discontinuing treatment or changing from one therapeutic regimen to another comprising determining the expression or presence of one or more biomarkers from one or more subpopulation of lymphocytes in a subject wherein the course of treatment is treatment with a Btk inhibitor (e.g., ibrutinib) and an immune checkpoint inhibitor.

Methods for detecting expression of the biomarkers described herein, within the test and control biological samples comprise any methods that determine the quantity or the presence of these markers either at the nucleic acid or protein level. Such methods are well known in the art and include but are not limited to western blots, northern blots, ELISA, immunoprecipitation, immunofluorescence, flow cytometry, immunohistochemistry, nucleic acid hybridization techniques, nucleic acid reverse transcription methods, and nucleic acid amplification methods. In particular embodiments, expression of a biomarker is detected on a protein level using, for example, antibodies that are directed against specific biomarker proteins. These antibodies are used in various methods such as Western blot, ELISA, multiplexing technologies, immunoprecipitation, or immunohistochemistry techniques. In some embodiments, detection of biomarkers is accomplished by ELISA. In some embodiments, detection of biomarkers is accomplished by electrochemiluminescence (ECL).

Any means for specifically identifying and quantifying a biomarker (for example, biomarker, a biomarker of cell survival or proliferation, a biomarker of apoptosis, a biomarker of a Btk-mediated signaling pathway) in the biological sample of a candidate subject is contemplated. Thus, in some embodiments, expression level of a biomarker protein of interest in a biological sample is detected by means of a binding protein capable of interacting specifically with that biomarker protein or a biologically active variant thereof. In some embodiments, labeled antibodies, binding portions thereof, or other binding partners are used. The word “label” when used herein refers to a detectable compound or composition that is conjugated directly or indirectly to the antibody so as to generate a “labeled” antibody. In some embodiments, the label is detectable by itself (e.g., radioisotope labels or fluorescent labels) or, in the case of an enzymatic label, catalyzes chemical alteration of a substrate compound or composition that is detectable.

The antibodies for detection of a biomarker protein are either monoclonal or polyclonal in origin, or are synthetically or recombinantly produced. The amount of complexed protein, for example, the amount of biomarker protein associated with the binding protein, for example, an antibody that specifically binds to the biomarker protein, is determined using standard protein detection methodologies known to those of skill in the art. A detailed review of immunological assay design, theory and protocols are found in numerous texts in the art (see, for example, Ausubel et al., eds. (1995) Current Protocols in Molecular Biology) (Greene Publishing and Wiley-Interscience, NY)); Coligan et al., eds. (1994) Current Protocols in Immunology (John Wiley & Sons, Inc., New York, N.Y.).

The choice of marker used to label the antibodies will vary depending upon the application. However, the choice of the marker is readily determinable to one skilled in the art. These labeled antibodies are used in immunoassays as well as in histological applications to detect the presence of any biomarker or protein of interest. The labeled antibodies are either polyclonal or monoclonal. Further, the antibodies for use in detecting a protein of interest are labeled with a radioactive atom, an enzyme, a chromophoric or fluorescent moiety, or a colorimetric tag as described elsewhere herein. The choice of tagging label also will depend on the detection limitations desired. Enzyme assays (ELISAs) typically allow detection of a colored product formed by interaction of the enzyme-tagged complex with an enzyme substrate. Radionuclides that serve as detectable labels include, for example, 1-131, 1-123, 1-125, Y-90, Re-188, Re-186, At −211, Cu-67, Bi-212, and Pd-109. Examples of enzymes that serve as detectable labels include, but are not limited to, horseradish peroxidase, alkaline phosphatase, beta-galactosidase, and glucose-6-phosphate dehydrogenase. Chromophoric moieties include, but are not limited to, fluorescein and rhodamine. The antibodies are conjugated to these labels by methods known in the art. For example, enzymes and chromophoric molecules are conjugated to the antibodies by means of coupling agents, such as dialdehydes, carbodiimides, dimaleimides, and the like. Alternatively, conjugation occurs through a ligand-receptor pair. Examples of suitable ligand-receptor pairs are biotin-avidin or biotin-streptavidin, and antibody-antigen.

In certain embodiments, expression or presence of one or more biomarkers or other proteins of interest within a biological sample, for example, a sample of bodily fluid, is determined by radioimmunoassays or enzyme-linked immunoassays (ELISAs), competitive binding enzyme-linked immunoassays, dot blot (see, for example, Promega Protocols and Applications Guide, Promega Corporation (1991), Western blot (see, for example, Sambrook et al. (1989) Molecular Cloning, A Laboratory Manual, Vol. 3, Chapter 18 (Cold Spring Harbor Laboratory Press, Plainview, N.Y.), chromatography such as high performance liquid chromatography (HPLC), or other assays known in the art. Thus, the detection assays involve steps such as, but not limited to, immunoblotting, immunodiffusion, immunoelectrophoresis, or immunoprecipitation.

In certain other embodiments, the methods of the invention are useful for identifying and treating cancer, including those listed above, that are refractory to (i.e., resistant to, or have become resistant to) first-line oncotherapeutic treatments.

In some embodiments, the expression or presence of one or more of the biomarkers described herein are also determined at the nucleic acid level. Nucleic acid-based techniques for assessing expression are well known in the art and include, for example, determining the level of biomarker mRNA in a biological sample. Many expression detection methods use isolated RNA. Any RNA isolation technique that does not select against the isolation of mRNA is utilized for the purification of RNA (see, e.g., Ausubel et al., ed. (1987-1999) Current Protocols in Molecular Biology (John Wiley & Sons, New York). Additionally, large numbers of tissue samples are readily processed using techniques well known to those of skill in the art, such as, for example, the single-step RNA isolation process disclosed in U.S. Pat. No. 4,843,155.

Thus, in some embodiments, the detection of a biomarker or other protein of interest is assayed at the nucleic acid level using nucleic acid probes. The term “nucleic acid probe” refers to any molecule that is capable of selectively binding to a specifically intended target nucleic acid molecule, for example, a nucleotide transcript. Probes are synthesized by one of skill in the art, or derived from appropriate biological preparations. Probes are specifically designed to be labeled, for example, with a radioactive label, a fluorescent label, an enzyme, a chemiluminescent tag, a colorimetric tag, or other labels or tags that are discussed above or that are known in the art. Examples of molecules that are utilized as probes include, but are not limited to, RNA and DNA.

For example, isolated mRNA are used in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that hybridize to the mRNA encoded by the gene being detected. The nucleic acid probe comprises of, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a biomarker, biomarker described herein above. Hybridization of an mRNA with the probe indicates that the biomarker or other target protein of interest is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in a gene chip array. A skilled artisan readily adapts known mRNA detection methods for use in detecting the level of mRNA encoding the biomarkers or other proteins of interest.

An alternative method for determining the level of an mRNA of interest in a sample involves the process of nucleic acid amplification, e.g., by RT-PCR (see, for example, U.S. Pat. No. 4,683,202), ligase chain reaction (Barany (1991) Proc. Natl. Acad. Sci. USA 88:189 193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, biomarker expression is assessed by quantitative fluorogenic RT-PCR (i.e., the TaqMan( ) System).

Expression levels of an RNA of interest are monitored using a membrane blot (such as used in hybridization analysis such as Northern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of expression also comprises using nucleic acid probes in solution.

In one embodiment of the invention, microarrays are used to determine expression or presence of one or more biomarkers. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning Hybridization intensities for each probe on the array are determined and converted to a quantitative value representing relative gene expression levels. See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

Techniques for the synthesis of these arrays using mechanical synthesis methods are described in, e.g., U.S. Pat. No. 5,384,261, incorporated herein by reference in its entirety. In some embodiments, an array is fabricated on a surface of virtually any shape or even a multiplicity of surfaces. In some embodiments, an array is a planar array surface. In some embodiments, arrays include peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety for all purposes. In some embodiments, arrays are packaged in such a manner as to allow for diagnostics or other manipulation of an all-inclusive device.

### Samples

In some embodiments, a sample for use in a method described herein is from any tissue or fluid from a patient. Samples include, but are not limited, to whole blood, dissociated bone marrow, bone marrow aspirate, pleural fluid, peritoneal fluid, central spinal fluid, abdominal fluid, pancreatic fluid, cerebrospinal fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract. In some embodiments, a sample is a blood serum sample. In some embodiments, a sample is from a fluid or tissue that is part of, or associated with, the lymphatic system or circulatory system. In some embodiments, a sample is a blood sample that is a venous, arterial, peripheral, tissue, cord blood sample. In some embodiments, a sample is a blood cell sample containing one or more peripheral blood mononuclear cells (PBMCs). In some embodiments, the sample contains one or more circulating tumor cells (CTCs). In some embodiments, a sample contains one or more disseminated tumor cells (DTC, e.g., in a bone marrow aspirate sample).

In some embodiments, samples are obtained from a patient by any suitable means of obtaining the sample using well-known and routine clinical methods. Procedures for obtaining fluid samples from an individual are well known. For example, procedures for drawing and processing whole blood and lymph are well-known and can be employed to obtain a sample for use in the methods provided. Typically, for collection of a blood sample, an anti-coagulation agent (e.g., EDTA, or citrate and heparin or CPD (citrate, phosphate, dextrose) or comparable substances) is added to the sample to prevent coagulation of the blood. In some examples, the blood sample is collected in a collection tube that contains an amount of EDTA to prevent coagulation of the blood sample.

Further, procedures for collecting various body samples are well known in the art. For example, procedures for collecting a breast tissue sample are well known, and can be obtained by for example, fine needle aspiration biopsy, core needle biopsy, or excisional biopsy. Fixative and staining solutions can be applied to the cells or tissues for preserving the specimen and for facilitating examination.

In some instances, a sample is a cell sample, such as a cell of the hematologic malignant cell line, or the solid tumor cell line. In some instances, a hematologic malignant cell line include cells obtained from, for example, chronic lymphocytic leukemia (CLL), small lymphocytic lymphoma (SLL), high risk CLL, non-CLL/SLL lymphoma, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some instances, a solid tumor cell line include cells obtained from, for example, bladder cancer, breast cancer, colon cancer, gastroenterological cancer, kidney cancer, lung cancer, ovarian cancer, pancreatic cancer, prostate cancer, proximal or distal bile duct cancer, or melanoma.

In some instances, a sample is a hematologic malignant cel or population of hematologic malignanct cells. In some instances, the cell lines include, A20, J558, BALM-1, BALM-2, BALM-3, EL4, Jurkat, THP1, OCI-Lyl, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, or TMD8. In some instances, the cell lines are sensitive to a treatment of a combination with a TEC inhibitor (e.g. BTK inhibitor, ITK inhibitor) and an immune checkpoint inhibitor. In some instances, the cell lines are sensitive to a treatment of a combination with a BTK inhibitor and an immune checkpoint inhibitor. In some instances, the cell lines are sensitive to a treatment of a combination with ibrutinib and an immune checkpoint inhibitor. In some instances, the cell lines are sensitive to a treatment of a combination with a TEC inhibitor (e.g. BTK inhibitor such as ibrutinib, ITK inhibitor), an immune checkpoint inhibitor, and an additional anticancer agent.

In some instances, a sample is a solid tumor cell or population of solid tumor cells. In some instances, the cell lines include, 293-T, 4T1, 721, 9L, A2780, ALC, B16, B35, BCP-1, BEAS-2B, BHK-21, BR 293, BxPC3, C3H-10T1/2, COR-L23, COS-7, CT26, DH82, DU145, DuCaP, EMT6/AR1, EMT6/AR10.0, H1299, H69, HeLa, Hepa1c1c7, High Five cells, HT-29, MCF-7, MDA-MB-231, MDA-MB-468, MG63, MDCK II, MRCS, RIN-5F, or T84. In some instances, the cell lines are sensitive to a treatment of a combination with a TEC inhibitor (e.g. BTK inhibitor, ITK inhibitor) and an immune checkpoint inhibitor. In some instances, the cell lines are sensitive to a treatment of a combination with a BTK inhibitor and an immune checkpoint inhibitor. In some instances, the cell lines are sensitive to a treatment of a combination with ibrutinib and an immune checkpoint inhibitor. In some instances, the cell lines are sensitive to a treatment of a combination with a TEC inhibitor (e.g. BTK inhibitor such as ibrutinib, ITK inhibitor), an immune checkpoint inhibitor, and an additional anticancer agent.

In some embodiments, the collection of a sample from the individual is performed at regular intervals, such as, for example, one day, two days, three days, four days, five days, six days, one week, two weeks, weeks, four weeks, one month, two months, three months, four months, five months, six months, one year, daily, weekly, bimonthly, quarterly, biyearly or yearly.

In some embodiments, the collection of a sample is performed at a predetermined time or at regular intervals relative to the treatment of a combination of a TEC inhibitor (e.g. BTK inhibitor such as ibrutinib, ITK inhibitor) and an immune checkpoint inhibitor. For example, a sample is collected from a patient at a predetermined time or at regular intervals prior to, during, or following treatment or between successive treatments with a combination of a TEC inhibitor (e.g. BTK inhibitor such as ibrutinib, ITK inhibitor) and an immune checkpoint inhibitor. In some instances, a sample is collected from a patient at a predetermined time or at regular intervals prior to, during, or following treatment or between successive treatments with a combination of a TEC inhibitor (e.g. BTK inhibitor such as ibrutinib, ITK inhibitor), an immune checkpoint inhibitor, and an additional anticancer agent. In some instances, a sample is also collected from a patient at a predetermined time or at regular intervals prior to, during, or following treatment or between successive treatments of one or more of a TEC inhibitor (e.g. BTK inhibitor such as ibrutinib, ITK inhibitor), an immune checkpoint inhibitor, and/or an additional anticancer agent.

### Therapeutic Analysis-based Systems

Also described herein, in certain aspects, are systems for assessing an individual having cancer for a therapeutic treatment based on the presence or absence, or the expression level of one or more of the biomarkers described herein. For example, described herein are systems of asessing an individual having a cancer (e.g. solid tumor, hematologic cancer, relapsed, refractory, or metastasized cancer) for the treatment that comprises (a) a digital processing device comprising an operating system configured to perform executable instructions, and an electronic memory; (b) a dataset stored in the electronic memory, wherein the dataset comprises data from a biomarker described elsewhere herein, e.g. Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or a combination thereof; and (c) a computer program including instructions executable by the digital processing device to create an application that comprises (i) a first software module configured to analyze the dataset to determine the presence or absence or the expression level of a biomarker described elsewhere herein; and (ii) a second software module to assign the indivdival as a candidate for treatment with a combination of a TEC inhibitor (e.g. BTK inhibitor such as ibrutinib, ITK inhibitor) and an immune checkpoint inhibitor based on the biomarker result.

In some aspects and in accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®; and suitable personal computer operating systems include, by way of non-limiting examples, Microsoft® Windows®, Apple® Mac OS X®, UNIX®, and UNIX-like operating systems such as GNU/Linux®. In some embodiments, additional operating systems include those provided by cloud computing such as for example, mobile smart phone operating systems (e.g. Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®); media streaming device operating systems (e.g. Apple TV®, Roku®, Boxee®, Google TV®, Google Chromecast®, Amazon Fire®, and Samsung® HomeSync®); and video game console operating systems (e.g. Sony® PS3®, Sony® PS4®, Microsoft® Xbox 360®, Microsoft Xbox One, Nintendo® Wii®, Nintendo® Wii U®, and Ouya®).

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, cloud computing based storage, and the like. In further embodiments, the storage and/or memory device is a combination of devices such as those disclosed herein.

In some embodiments, the digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect™, Leap Motion™, or the like. In still further embodiments, the input device is a combination of devices such as those disclosed herein.

In some instances, the systems and methods disclosed herein include at least one computer program, or use of the same. A computer program includes a sequence of instructions, executable in the digital processing device's CPU, written to perform a specified task. In some embodiments, computer readable instructions are implemented as program modules, such as functions, objects, Application Programming Interfaces (APIs), data structures, and the like, that perform particular tasks or implement particular abstract data types. In light of the disclosure provided herein, those of skill in the art will recognize that a computer program, in certain embodiments, is written in various versions of various languages.

In some cases, the functionality of the computer readable instructions are combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

In some embodiments, the methods and systems disclosed herein include one or more databases, or use of the same. In view of the disclosure provided herein, those of skill in the art will recognize that many databases are suitable for storage and retrieval of analytical information described elsewhere herein. In various embodiments, suitable databases include, by way of non-limiting examples, relational databases, non-relational databases, object oriented databases, object databases, entity-relationship model databases, associative databases, and XML databases. In some embodiments, a database is internet-based. In further embodiments, a database is web-based. In still further embodiments, a database is cloud computing-based. In other embodiments, a database is based on one or more local computer storage devices.

In some embodiments, the methods and systems disclosed herein are performed as a service. In some embodiments, a service provider obtains a cancer samples that a customer wishes to analyze. In some embodiments, the service provider then encodes each cancer sample to be analyzed by any of the methods described herein, performs the analysis and provides a report to the customer. In some embodiments, the customer also performs the analysis and provides the results to the service provider for decoding. In some embodiments, the service provider then provides the decoded results to the customer. In some embodiments, the customer also encodes the cancer samples, analyzes the samples and decodes the results by interacting with software installed locally (at the customer's location) or remotely (e.g. on a server reachable through a network). In some embodiments, the software generates a report and transmits the report to the costumer. Exemplary customers include clinical laboratories, hospitals, and the like. In some embodiments, a customer or party is any suitable customer or party with a need or desire to use the methods, systems, pharmaceutical combinations, compositions, and/or kits of the invention.

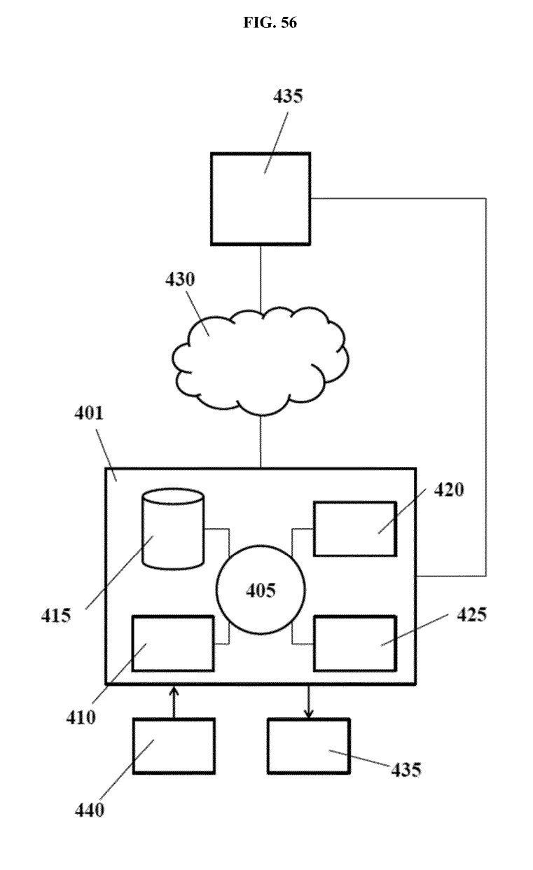

In some embodiments, the methods provided herein are processed on a server or a computer server (FIG. 56). In some embodiments, the server 401 includes a central processing unit (CPU, also “processor”) 405 which is a single core processor, a multi core processor, or plurality of processors for parallel processing. In some embodiments, a processor used as part of a control assembly is a microprocessor. In some embodiments, the server 401 also includes memory 410 (e.g. random access memory, read-only memory, flash memory); electronic storage unit 415 (e.g. hard disk); communications interface 420 (e.g. network adaptor) for communicating with one or more other systems; and peripheral devices 425 which includes cache, other memory, data storage, and/or electronic display adaptors. The memory 410, storage unit 415, interface 420, and peripheral devices 425 are in communication with the processor 405 through a communications bus (solid lines), such as a motherboard. In some embodiments, the storage unit 415 is a data storage unit for storing data. The server 401 is operatively coupled to a computer network (“network”) 430 with the aid of the communications interface 420. In some embodiments, a processor with the aid of additional hardware is also operatively coupled to a network. In some embodiments, the network 430 is the Internet, an intranet and/or an extranet, an intranet and/or extranet that is in communication with the Internet, a telecommunication or data network. In some embodiments, the network 430 with the aid of the server 401, implements a peer-to-peer network, which enables devices coupled to the server 401 to behave as a client or a server. In some embodiments, the server is capable of transmitting and receiving computer-readable instructions (e.g., device/system operation protocols or parameters) or data (e.g., sensor measurements, raw data obtained from detecting metabolites, analysis of raw data obtained from detecting metabolites, interpretation of raw data obtained from detecting metabolites, etc.) via electronic signals transported through the network 430. Moreover, in some embodiments, a network is used, for example, to transmit or receive data across an international border.

In some embodiments, the server 401 is in communication with one or more output devices 435 such as a display or printer, and/or with one or more input devices 440 such as, for example, a keyboard, mouse, or joystick. In some embodiments, the display is a touch screen display, in which case it functions as both a display device and an input device. In some embodiments, different and/or additional input devices are present such an enunciator, a speaker, or a microphone. In some embodiments, the server uses any one of a variety of operating systems, such as for example, any one of several versions of Windows®, or of MacOS®, or of Unix®, or of Linux®.

In some embodiments, the storage unit 415 stores files or data associated with the operation of a device, systems or methods described herein.

In some embodiments, the server communicates with one or more remote computer systems through the network 430. In some embodiments, the one or more remote computer systems include, for example, personal computers, laptops, tablets, telephones, Smart phones, or personal digital assistants.

In some embodiments, a control assembly includes a single server 401. In other situations, the system includes multiple servers in communication with one another through an intranet, extranet and/or the Internet.

In some embodiments, the server 401 is adapted to store device operation parameters, protocols, methods described herein, and other information of potential relevance. In some embodiments, such information is stored on the storage unit 415 or the server 401 and such data is transmitted through a network.

### Pharmaceutical Combinations/Formulations

Disclosed herein, in certain embodiments, are pharmaceutical combinations and/or compositions that comprise (a) a Btk inhibitor and an immune checkpoint inhibitor, and (b) a pharmaceutically-acceptable excipient. In some embodiments, the Btk inhibitor is ibrutinib. In some embodiments, the combination provides a synergistic therapeutic effect compared to administration of ibrutinib or the second anticancer agent alone. In some instances, the combination provides an additive therapeutic effect compared to administration of ibrutinib or the second anticancer agent alone. In some instances, the combination provides an antagonistic effect compared to administration of ibrutinib or the second anticancer agent alone. In some instances, the combination sensitizes the cancer (e.g. solid tumors, hematologic cancers) to the BTK inhibitor. In some instances, the combination sensitizes the cancer (e.g. solid tumors, hematologic cancers) to the immune checkpoint inhibitor. In some instances, the combination sensitizes the cancer (e.g. solid tumors, hematologic cancers) to both the BTK inhibitor and the immune checkpoint inhibitor. In some instances, the combination further comprises an additional anticancer agent. In some instances, the combination of a BTK inhibitor, an immune checkpoint inhibitor, and an additional anticancer agent provides a synergistic therapeutic effect, or an additive therapeutic effect compared to administrations of the BTK inhibitor, immune checkpoint inhibitor, or the additional anticancer agent alone or in dual combinations. In some instances, the combination of a BTK inhibitor, an immune checkpoint inhibitor, and an additional anticancer agent sensitizes the cancer (e.g. solid tumors, hematologic cancers) to the additional anticancer agent, or to the combination of the BTK inhibitor, the immune checkpoint inhibitor, and the additional anticancer agent.

In some embodiments, the immune checkpoint inhibitor is an inhibitor of Programmed Death-Ligand 1 (PD-L1, also known as B7-H1, CD274), Programmed Death 1 (PD-1), CTLA-4, B7H1, B7H4, OX-40, CD137, CD40, 2B4, IDO1, IDO2, VISTA, CD27, CD28, PD-L2 (B7-DC, CD273), LAG3, CD80, CD86, PDL2, B7H3, HVEM, BTLA, KIR, GAL9, TIM3, A2aR, MARCO (macrophage receptor with collageneous structure), PS (phosphatidylserine), ICOS (inducible T cell costimulator), HAVCR2, CD276, VTCN1, CD70, CD160, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, the dose of Ibrutinib is between about 10 mg to about 1000 mg. In some embodiments, the dose of Ibrutinib is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 250 mg, about 300 mg, about 350 mg, about 400 mg, about 450 mg, about 500 mg, about 550 mg, about 600 mg, about 700 mg or about 800 mg. In some embodiments, the therapeutically-effective amount of Ibrutinib is between about 40 mg and about 140 mg. In some embodiments, the therapeutically-effective amount of Ibrutinib is between about 40 mg and about 100 mg. In some embodiments, the dose of Ibrutinib is between about 40 mg and about 70 mg. In some embodiments, the dose of Ibrutinib is about 40 mg. In some embodiments, Ibrutinib is amorphous or crystalline. In some embodiments, Ibrutinib is milled or a nano-particle. In some embodiments, the pharmaceutical composition is a combined dosage form.

Pharmaceutical combinations and/or compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H.A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

A pharmaceutical combination, as used herein, refers to a mixture of Ibrutinib, an immune checkpoint inhibitor, and/or an additional therapeutic agent with other chemical components, such as carriers, stabilizers, diluents, dispersing agents, suspending agents, thickening agents, and/or excipients.

In practicing the methods of treatment or use provided herein, therapeutically effective amounts of the compounds disclosed herein are administered having a disease, disorder, or condition to be treated. In some embodiments, the mammal is a human. The therapeutically effective amounts of the compounds may vary depending on the compounds, severity of the disease, the age and relative health of the subject, and other factors.

The term “combination” as used herein, means a product that results from the mixing or combining of Ibrutinib and an immune checkpoint inhibitor (and any additional therapeutic agents) and includes both fixed and non-fixed combinations. The term “fixed combination” means that Ibrutinib and the Immune checkpoint inhibitor are both administered in a single entity or dosage form. The term “non-fixed combination” means that Ibrutinib and the immune checkpoint inhibitor are administered as separate entities or dosage forms either simultaneously, concurrently or sequentially with no specific intervening time limits, wherein such administration provides effective levels of the two compounds in the body of the patient. The latter also applies to cocktail therapy, e.g. the administration of three or more active ingredients.

In some embodiments, the pharmaceutical combination and/or composition described herein also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some embodiments, the pharmaceutical combination and/or compositions also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, pharmaceutical combination and/or compositions including a compound described herein are manufactured in a conventional manner, such as, by way of example only, by means of conventional mixing, dissolving, granulating, dragee-making, levigating, emulsifying, encapsulating, entrapping or compression processes.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite and tocopherol. In certain embodiments, antioxidants enhance chemical stability where required.

In some embodiments, compositions provided herein also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

A “carrier” or “carrier materials” include any commonly used excipients in pharmaceutics and should be selected on the basis of compatibility with compounds disclosed herein, such as, compounds of ibrutinib, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H.A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

The term “disintegrate” includes both the dissolution and dispersion of the dosage form when contacted with gastrointestinal fluid. “Disintegration agents or disintegrants” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

“Drug absorption” or “absorption” typically refers to the process of movement of drug from site of administration of a drug across a barrier into a blood vessel or the site of action, e.g., a drug moving from the gastrointestinal tract into the portal vein or lymphatic system.

An “enteric coating” is a substance that remains substantially intact in the stomach but dissolves and releases the drug in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

“Erosion facilitators” include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Flavoring agents” and/or “sweeteners” useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

A “measurable serum concentration” or “measurable plasma concentration” describes the blood serum or blood plasma concentration, typically measured in mg, μg, or ng of therapeutic agent per mL, dL, or L of blood serum, absorbed into the bloodstream after administration. As used herein, measurable plasma concentrations are typically measured in ng/ml or μg/ml.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Steady state,” as used herein, is when the amount of drug administered is equal to the amount of drug eliminated within one dosing interval resulting in a plateau or constant plasma drug exposure.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants are included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Dosage Forms

Disclosed herein, in certain embodiments, are dosage forms which comprise a BTK inhibitor and an immune checkpoint inhibitor. In some embodiments, the Btk inhibitor is ibrutinib. In some embodiments, the dosage form is a combined dosage form. In some embodiments, the dosage form is a solid oral dosage form. In some embodiments, the dosage form is a tablet, pill, or capsule. In some embodiments, the dosage form is a controlled release dosage form, delayed release dosage form, extended release dosage form, pulsatile release dosage form, multiparticulate dosage form, or mixed immediate release and controlled release formulation. In some embodiments, the dosage form comprises a controlled release coating. In some embodiments, the dosage forms comprises a first controlled release coating which controls the release of Ibrutinib and a second controlled release coating which controls the release of the Immune checkpoint inhibitor. In some embodiments, the combination provides a synergistic or additive therapeutic effect compared to administration of ibrutinib or the second anticancer agent alone.

In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof, or any combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3.

In some embodiments, the dose of Ibrutinib is between about 10 mg to about 1000 mg. In some embodiments, the dose of Ibrutinib is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 250 mg, about 300 mg, about 350 mg, about 400 mg, about 450 mg, about 500 mg, about 550 mg, about 600 mg, about 700 mg or about 800 mg. In some embodiments, the therapeutically-effective amount of Ibrutinib is between about 40 mg and about 140 mg. In some embodiments, the therapeutically-effective amount of Ibrutinib is between about 40 mg and about 100 mg. In some embodiments, the dose of Ibrutinib is between about 40 mg and about 70 mg. In some embodiments, the dose of Ibrutinib is about 40 mg. In some embodiments, Ibrutinib is amorphous or crystalline.

The pharmaceutical combination described herein may be formulated for administration via any conventional means including, but not limited to, oral, parenteral (e.g., intravenous, subcutaneous, or intramuscular), buccal, intranasal, rectal or transdermal administration routes. As used herein, the terms “subject”, “individual” and “patient” are used interchangeably and mean an animal, preferably a mammal, including a human or non-human. None of the terms require the supervision (continuous or otherwise) of a medical professional.

The pharmaceutical combination described herein are formulated into any suitable dosage form, including but not limited to, solid oral dosage forms, controlled release formulations, fast melt formulations, effervescent formulations, tablets, powders, pills, capsules, delayed release formulations, extended release formulations, pulsatile release formulations, multiparticulate formulations, and mixed immediate release and controlled release formulations.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In some embodiments, disintegrating agents are added, such as the cross-linked croscarmellose sodium, polyvinylpyrrolidone, agar, or alginic acid or a salt thereof such as sodium alginate.

Dragee cores are provided with suitable coatings. For this purpose, in some embodiments, concentrated sugar solutions are used, which, in particular embodiments, optionally contain gum arabic, talc, polyvinylpyrrolidone, carbopol gel, polyethylene glycol, and/or titanium dioxide, lacquer solutions, and suitable organic solvents or solvent mixtures. In some embodiments, dyestuffs or pigments are added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In some embodiments, in soft capsules, the active compounds are dissolved or suspended in suitable liquids, such as fatty oils, liquid paraffin, or liquid polyethylene glycols. In addition, in some embodiments, stabilizers are added. All formulations for oral administration should be in dosages suitable for such administration.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, in some embodiments, pharmaceutical formulations described herein are administered as a single capsule or in multiple capsule dosage form. In some embodiments, the pharmaceutical formulation is administered in two, or three, or four, capsules or tablets.

In some embodiments, solid dosage forms, e.g., tablets, effervescent tablets, and capsules, are prepared by mixing particles of ibrutinib, with one or more pharmaceutical excipients to form a bulk blend composition. When referring to these bulk blend compositions as homogeneous, it is meant that the particles of ibrutinib are dispersed evenly throughout the composition so that the composition can be readily subdivided into equally effective unit dosage forms, such as tablets, pills, and capsules. In some embodiments, the individual unit dosages also include film coatings, which disintegrate upon oral ingestion or upon contact with diluent. These formulations can be manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The pharmaceutical solid dosage forms described herein can include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the formulation of ibrutinib. In another embodiment, some or all of the particles of ibrutinib, are not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

In order to release the compound of one or more of the therapeutic agents described herein, from a solid dosage form matrix as efficiently as possible, disintegrants are often used in the formulation, especially when the dosage forms are compressed with binder. Disintegrants help rupturing the dosage form matrix by swelling or capillary action when moisture is absorbed into the dosage form. Suitable disintegrants for use in the solid dosage forms described herein include, but are not limited to, natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g., Klucer®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Formulators skilled in art can determine the binder level for the formulations, but binder usage level of up to 70% in tablet formulations is common

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

The term “non water-soluble diluent” represents compounds typically used in the formulation of pharmaceuticals, such as calcium phosphate, calcium sulfate, starches, modified starches and microcrystalline cellulose, and microcellulose (e.g., having a density of about 0.45 g/cm³, e.g. Avicel, powdered cellulose), and talc.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms described herein. The amounts of such additives can be readily determined by one skilled in the art, according to the particular properties desired.

In other embodiments, one or more layers of the pharmaceutical formulation are plasticized. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

Compressed tablets are solid dosage forms prepared by compacting the bulk blend of the formulations described above. In various embodiments, compressed tablets which are designed to dissolve in the mouth will include one or more flavoring agents. In other embodiments, the compressed tablets will include a film surrounding the final compressed tablet. In some embodiments, the film coating can provide a delayed release of ibrutinib or the second agent, from the formulation. In other embodiments, the film coating aids in patient compliance (e.g., Opadry® coatings or sugar coating). Film coatings including Opadry® typically range from about 1% to about 3% of the tablet weight. In other embodiments, the compressed tablets include one or more excipients.

In some embodiments, a capsule is prepared, for example, by placing the bulk blend of the formulation of ibrutinib or the second agent, described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule can be swallowed whole or the capsule can be opened and the contents sprinkled on food prior to eating. In some embodiments, the therapeutic dose is split into multiple (e.g., two, three, or four) capsules. In some embodiments, the entire dose of the formulation is delivered in a capsule form.

In various embodiments, the particles of ibrutinib, and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In another aspect, in some embodiments, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with ibrutinib, which sufficiently isolate the compound of any of ibrutinib, from other non-compatible excipients. Materials compatible with compounds of any of ibrutinib, are those that delay the release of the compounds of any of ibrutinib, in vivo.

Exemplary microencapsulation materials useful for delaying the release of the formulations including compounds described herein, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

In still other embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In other embodiments, the microencapsulating material useful for delaying the release of the pharmaceutical compositions is from the USP or the National Formulary (NF). In yet other embodiments, the microencapsulation material is Klucel. In still other embodiments, the microencapsulation material is methocel.

In some embodiments, microencapsulated compounds of any of ibrutinib, are formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, in some embodiments, other methods such as roller compaction, extrusion/spheronization, coacervation, or nanoparticle coating are used.

In one embodiment, the particles of compounds of any of ibrutinib, are microencapsulated prior to being formulated into one of the above forms. In still another embodiment, some or most of the particles are coated prior to being further formulated by using standard coating procedures, such as those described in Remington's Pharmaceutical Sciences, 20th Edition (2000).

In other embodiments, the solid dosage formulations of the compounds of any of ibrutinib, are plasticized (coated) with one or more layers. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

In other embodiments, a powder including the formulations with a compound of any of ibrutinib, described herein, is formulated to include one or more pharmaceutical excipients and flavors. In some embodiments, such a powder is prepared, for example, by mixing the formulation and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared in accordance with the present disclosure. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the compositions described herein are added to water, the acids and the base react to liberate carbon dioxide gas, thereby causing “effervescence.” Examples of effervescent salts include, e.g., the following ingredients: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. In some embodiments, the enteric coated dosage form is a compressed or molded or extruded tablet/mold (coated or uncoated) containing granules, powder, pellets, beads or particles of the active ingredient and/or other composition components, which are themselves coated or uncoated. In some embodiments, the enteric coated oral dosage form is a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the methods and compositions described herein to achieve delivery to the lower gastrointestinal tract. In some embodiments the polymers described herein are anionic carboxylic polymers. In other embodiments, the polymers and compatible mixtures thereof, and some of their properties, include, but are not limited to:

Shellac, also called purified lac, a refined product obtained from the resinous secretion of an insect. This coating dissolves in media of pH>7;

Acrylic polymers. The performance of acrylic polymers (primarily their solubility in biological fluids) can vary based on the degree and type of substitution. Examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine;

Cellulose Derivatives. Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous based system and is a spray dried CAP psuedolatex with particles<1 μm. Other components in Aquateric can include pluronics, Tweens, and acetylated monoglycerides. Other suitable cellulose derivatives include: cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-55S, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions; Poly Vinyl Acetate Phthalate (PVAP). PVAP dissolves in pH>5, and it is much less permeable to water vapor and gastric fluids.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

In some embodiments, colorants, detackifiers, surfactants, antifoaming agents, lubricants (e.g., carnuba wax or PEG) are added to the coatings besides plasticizers to solubilize or disperse the coating material, and to improve coating performance and the coated product.

In other embodiments, the formulations described herein, which include ibrutinib, are delivered using a pulsatile dosage form. A pulsatile dosage form is capable of providing one or more immediate release pulses at predetermined time points after a controlled lag time or at specific sites. Many other types of controlled release systems known to those of ordinary skill in the art and are suitable for use with the formulations described herein. Examples of such delivery systems include, e.g., polymer-based systems, such as polylactic and polyglycolic acid, plyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2^{nd} Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983.

In some embodiments, pharmaceutical formulations are provided that include particles of ibrutinib, described herein and at least one dispersing agent or suspending agent for oral administration to a subject. In some embodiments, the formulations are a powder and/or granules for suspension, and upon admixture with water, a substantially uniform suspension is obtained.

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2^{nd} Ed., pp. 754-757 (2002). In addition, in some embodiments, the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

The aqueous suspensions and dispersions described herein can remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®; a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropyl cellulose ethers (e.g., HPC, HPC-SL, and HPC-L), hydroxypropyl methylcellulose and hydroxypropyl methylcellulose ethers (e.g. HPMC K100, HPMC K4M, HPMC K15M, and HPMC K100M), carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908®, also known as Poloxamine 908®).

Wetting agents suitable for the aqueous suspensions and dispersions described herein are known in the art and include, but are not limited to, cetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carbopol 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In one embodiment, the aqueous liquid dispersion can comprise a sweetening agent or flavoring agent in a concentration ranging from about 0.001% to about 1.0% the volume of the aqueous dispersion. In another embodiment, the aqueous liquid dispersion can comprise a sweetening agent or flavoring agent in a concentration ranging from about 0.005% to about 0.5% the volume of the aqueous dispersion. In yet another embodiment, the aqueous liquid dispersion can comprise a sweetening agent or flavoring agent in a concentration ranging from about 0.01% to about 1.0% the volume of the aqueous dispersion.

In addition to the additives listed above, the liquid formulations can also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, the pharmaceutical formulations described herein can be self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. In some embodiments, SEDDS provide improvements in the bioavailability of hydrophobic active ingredients. Methods of producing self-emulsifying dosage forms are known in the art and include, but are not limited to, for example, U.S. Pat. Nos. 5,858,401, 6,667,048, and 6,960,563, each of which is specifically incorporated by reference.

It is to be appreciated that there is overlap between the above-listed additives used in the aqueous dispersions or suspensions described herein, since a given additive is often classified differently by different practitioners in the field, or is commonly used for any of several different functions. Thus, the above-listed additives should be taken as merely exemplary, and not limiting, of the types of additives that can be included in formulations described herein. The amounts of such additives can be readily determined by one skilled in the art, according to the particular properties desired.

### Intranasal Formulations

Intranasal formulations are known in the art and are described in, for example, U.S. Pat. Nos. 4,476,116, 5,116,817 and 6,391,452, each of which is specifically incorporated by reference. Formulations that include ibrutinib, which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in Remington: The Science and Practice of Pharmacy, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. In some embodiments, minor amounts of other ingredients such as pH adjusters, emulsifiers or dispersing agents, preservatives, surfactants, gelling agents, or buffering and other stabilizing and solubilizing agents are also present. The nasal dosage form should be isotonic with nasal secretions.

In some embodiments, for administration by inhalation described herein, the pharmaceutical compositions are in a form as an aerosol, a mist or a powder. Pharmaceutical compositions described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In some embodiments, in the case of a pressurized aerosol, the dosage unit is determined by providing a valve to deliver a metered amount. In some embodiments, capsules and cartridges of, such as, by way of example only, gelatin for use in an inhaler or insufflator are formulated containing a powder mix of the compound described herein and a suitable powder base such as lactose or starch.

### Buccal Formulations

In some embodiments, buccal formulations are administered using a variety of formulations known in the art. For example, such formulations include, but are not limited to, U.S. Pat. Nos. 4,229,447, 4,596,795, 4,755,386, and 5,739,136, each of which is specifically incorporated by reference. In addition, the buccal dosage forms described herein can further include a bioerodible (hydrolysable) polymeric carrier that also serves to adhere the dosage form to the buccal mucosa. The buccal dosage form is fabricated so as to erode gradually over a predetermined time period, wherein the delivery is provided essentially throughout. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, it will be appreciated that virtually any such carrier can be used, so long as the desired drug release profile is not compromised, and the carrier is compatible with ibrutinib, and any other components that are present in the buccal dosage unit. Generally, the polymeric carrier comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which can be obtained from B.F. Goodrich, is one such polymer). In some embodiments, other components are also incorporated into the buccal dosage forms described herein include, but are not limited to, disintegrants, diluents, binders, lubricants, flavoring, colorants, preservatives, and the like. In some embodiments, for buccal or sublingual administration, the compositions are in the form of tablets, lozenges, or gels formulated in a conventional manner

### Transdermal Formulations

In some embodiments, transdermal formulations described herein are administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144, each of which is specifically incorporated by reference in its entirety.

In some embodiments, the transdermal dosage forms described herein incorporate certain pharmaceutically acceptable excipients which are conventional in the art. In some embodiments, the transdermal formulations described herein include at least three components: (1) a formulation of a compound of ibrutinib; (2) a penetration enhancer; and (3) an aqueous adjuvant. In addition, transdermal formulations can include additional components such as, but not limited to, gelling agents, creams and ointment bases, and the like. In some embodiments, the transdermal formulation can further include a woven or non-woven backing material to enhance absorption and prevent the removal of the transdermal formulation from the skin. In other embodiments, the transdermal formulations described herein can maintain a saturated or supersaturated state to promote diffusion into the skin.

In some embodiments, formulations suitable for transdermal administration of compounds described herein employ transdermal delivery devices and transdermal delivery patches and can be lipophilic emulsions or buffered, aqueous solutions, dissolved and/or dispersed in a polymer or an adhesive. In some embodiments, such patches are constructed for continuous, pulsatile, or on demand delivery of pharmaceutical agents. Still further, transdermal delivery of the compounds described herein can be accomplished by means of iontophoretic patches and the like. Additionally, transdermal patches can provide controlled delivery of ibrutinib. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

### Injectable Formulations

In some embodiments, formulations include a combination of a BTK inhibitor (e.g. ibrutinib) and an immune checkpoint inhibitor, suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, fFormulations suitable for subcutaneous injection also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. In some embodiments, it is also desirable to include isotonic agents, such as sugars, sodium chloride, and the like. Prolonged absorption of the injectable pharmaceutical form can be brought about by the use of agents delaying absorption, such as aluminum monostearate and gelatin.

In some embodiments, for intravenous injections, compounds described herein are formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. In some embodiments, for other parenteral injections, appropriate formulations include aqueous or nonaqueous solutions, preferably with physiologically compatible buffers or excipients. Such excipients are generally known in the art.

In some embodiments, parenteral injections involve bolus injection or continuous infusion. In some embodiments, formulations for injection are presented in unit dosage form, e.g., in ampoules or in multi-dose containers, with an added preservative. In some embodiments, the pharmaceutical composition described herein is in a form suitable for parenteral injection as a sterile suspensions, solutions or emulsions in oily or aqueous vehicles, and contains formulatory agents such as suspending, stabilizing and/or dispersing agents. Pharmaceutical formulations for parenteral administration include aqueous solutions of the active compounds in water-soluble form. Additionally, in some embodiments, suspensions of the active compounds are prepared as appropriate oily injection suspensions. Suitable lipophilic solvents or vehicles include fatty oils such as sesame oil, or synthetic fatty acid esters, such as ethyl oleate or triglycerides, or liposomes. In some embodiments, aqueous injection suspensions contain substances which increase the viscosity of the suspension, such as sodium carboxymethyl cellulose, sorbitol, or dextran. Optionally, in some embodiments, the suspension also contains suitable stabilizers or agents which increase the solubility of the compounds to allow for the preparation of highly concentrated solutions. Alternatively, in some embodiments, the active ingredient is in powder form for constitution with a suitable vehicle, e.g., sterile pyrogen-free water, before use.

### Other Formulations

In certain embodiments, delivery systems for pharmaceutical compounds are employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein are administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

In some embodiments, the compounds described herein are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### Dosing and Treatment Regimens

In some embodiments, the amount of a TEC inhibitor that is administered in combination with an immune checkpoint inhibitor is from 10 mg/day up to, and including, 1000 mg/day. In some embodiments, the TEC inhibitor is a BTK inhibitor or an ITK inhibitor. In some embodiments, the TEC inhibitor is a BTK inhibitor.

In some embodiments, the amount of the Btk inhibitor that is administered in combination with an immune checkpoint inhibitor is from 10 mg/day up to, and including, 1000 mg/day. In some embodiments, the amount of the Btk inhibitor that is administered is from about 40 mg/day to 70 mg/day. In some embodiments, the amount of the Btk inhibitor that is administered per day is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg. In some embodiments, the BTK inhibitor is ibrutinib.

In some embodiments, the amount of ibrutinib that is administered in combination with an immune checkpoint inhibitor is from 10 mg/day up to, and including, 1000 mg/day. In some embodiments, the amount of ibrutinib that is administered is from about 40 mg/day to 70 mg/day. In some embodiments, the amount of ibrutinib that is administered per day is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg. In some embodiments, the amount of ibrutinib that is administered is about 40 mg/day. In some embodiments, the amount of ibrutinib that is administered is about 50 mg/day. In some embodiments, the amount of ibrutinib that is administered is about 60 mg/day. In some embodiments, the amount of ibrutinib that is administered is about 70 mg/day.

In some embodiments, the AUC₀₋₂₄ of ibrutinib co-administered with an immune checkpoint inhibitor is between about 50 and about 10000 ng/mL. In some embodiments, the Cmaxof Ibrutinib co-administered with an immune checkpoint inhibitor is between about 5 ng/mL and about 1000 ng/mL.

In some embodiments, the amount of an immune checkpoint inhibitor described herein that is administered in combination with a TEC inhibitor (e.g. BTK inhibitor such as ibrutinib, ITK inhibitor) is from 0.001 mg/kg up to and including 500 mg/kg. In some embodiments, the amount of an immune checkpoint inhibitor that is administered is from about 0.01 mg/kg to about 100 mg/kg. In some embodiments, the amount of an immune checkpoint inhibitor that is administered is about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, or about 95 mg/kg.

In some embodiments, the TEC inhibitor (e.g. BTK inhibitor, ITK inhibitor) is administered once per month, twice per month, three times per month, every other week, once per week, twice per week, three times per week, four times per week, five times per week, six times per week, every other day, daily, twice a day, three times a day or more frequent, continuously over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In some embodiments, the TEC inhibitor is a BTK inhibitor.

In some embodiments, the BTK inhibitor is administered once per month, twice per month, three times per month, every other week, once per week, twice per week, three times per week, four times per week, five times per week, six times per week, every other day, daily, twice a day, three times a day or more frequent, continuously over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In some embodiments, the BTK inhibitor is ibrutinib.

In some embodiments, ibrutinib is administered once per month, twice per month, three times per month, every other week, once per week, twice per week, three times per week, four times per week, five times per week, six times per week, every other day, daily, twice a day, three times a day or more frequent, continuously over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In some embodiments, ibrutinib is administered once per day, twice per day, or three times per day. In some embodiments, ibrutinib is administered once per day.

In some embodiments, an immune checkpoint inhibitor is administered once per month, twice per month, three times per month, every other week, once per week, twice per week, three times per week, four times per week, five times per week, six times per week, every other day, daily, twice a day, three times a day or more frequent, continuously over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In some embodiments, the immune checkpoint inhibitor is administered once per day, twice per day, or three times per day. In some embodiments, the immune checkpoint inhibitor is administered once per day.

In some embodiments, a TEC inhibitor (e.g. BTK inhibitor, ITK inhibitor) and an immune checkpoint inhibitor are administered sequentially or simultaneously once per month, twice per month, three times per month, every other week, once per week, twice per week, three times per week, four times per week, five times per week, six times per week, every other day, daily, twice a day, three times a day or more frequent, continuously over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In some embodiments, the TEC inhibitor is a BTK inhibitor.

In some embodiments, the BTK inhibitor and an immune checkpoint inhibitor are administered sequentially or simultaneously once per month, twice per month, three times per month, every other week, once per week, twice per week, three times per week, four times per week, five times per week, six times per week, every other day, daily, twice a day, three times a day or more frequent, continuously over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In some embodiments, the BTK inhibitor is ibrutinib.

In some embodiments, ibrutinib and an immune checkpoint inhibitor are administered sequentially or simultaneously once per month, twice per month, three times per month, every other week, once per week, twice per week, three times per week, four times per week, five times per week, six times per week, every other day, daily, twice a day, three times a day or more frequent, continuously over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

In some instances, a TEC inhibitor (e.g. BTK inhibitor such as ibrutinib, ITK inhibitor) is administered following a scheduled regimen while an immune checkpoint inhibitor is administered intermittently. In other instances, a TEC inhibitor (e.g. BTK inhibitor such as ibrutinib, ITK inhibitor) is administered intermittently while an immune checkpoint inhibitor is administered following a scheduled regimen.

In some instances, both a TEC inhibitor and an immune checkpoint inhibitor are administered intermittently. In some instances, a TEC inhibitor and an immune checkpoint inhibitor are administered intermittently with an additional anticancer agent. In some instances, the TEC inhibitor is a BTK inhibitor or an ITK inhibitor. In some instances, both a BTK inhibitor and an immune checkpoint inhibitor are administered intermittently. In some instances, a BTK inhibitor and an immune checkpoint inhibitor are administered intermittently with an additional anticancer agent. In some cases, the BTK inhibitor is ibrutinib. In some instances, both ibrutinib and an immune checkpoint inhibitor are administered intermittently. In some instances, ibrutinib and an immune checkpoint inhibitor are administered intermittently with an additional anticancer agent.

In some embodiments, a TEC inhibitor (e.g. BTK inhibitor, ITK inhibitor) and the immune checkpoint inhibitor are co-administered (e.g., in a single dosage form) with an additional anticancer agent, once per month, twice per month, three times per month, every other week, once per week, twice per week, three times per week, four times per week, five times per week, six times per week, every other day, daily, twice a day, three times a day or more frequent, continuously over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In some embodiments, the TEC inhibitor is a BTK inhibitor.

In some embodiments, a BTK inhibitor (e.g. ibrutinib) and the immune checkpoint inhibitor are co-administered (e.g., in a single dosage form) with an additional anticancer agent, once per month, twice per month, three times per month, every other week, once per week, twice per week, three times per week, four times per week, five times per week, six times per week, every other day, daily, twice a day, three times a day or more frequent, continuously over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

In some embodiments, the pharmaceutical combination and/or compositions described herein are administered to treatment-naive cancer patients. In some embodiments, a treament-naive cancer patient is a patient who has not received a treatment related to a cancer, a patient who has not received a TEC inhibitor (e.g. BTK inhibitor such as ibrutinib, ITK inhibitor) treatment, a patient who has not received an immune checkpoint inhibitor, or a patient who has not received any combinations of a TEC inhibitor, immune checkpoint inhibitor, and/or an additional anticancer agent described elsewhere herein.

In some embodiments, the pharmaceutical combination and/or compositions described herein are administered to cancer patients who have already received one or more prior treatments. In some embodiments, the one or more prior treatments include treatments such as surgery, chemotherapy, radiation therapy, and include treatments with one or more of the anticancer agents described elsewhere herein.

In some embodiments, the pharmaceutical combination and/or compositions disclosed herein are administered to patients for prophylactic, therapeutic, or maintenance treatment. In some embodiments, the compositions disclosed herein are administered for therapeutic applications. In some embodiments, the compositions disclosed herein are administered for therapeutic applications. In some embodiments, the compositions disclosed herein are administered as a maintenance therapy, for example for a patient in remission.

In some embodiments, a TEC inhibitor (e.g. BTK inhibitor, ITK inhibitor) and the immune checkpoint inhibitor are administered as a maintenance therapy. In some instances, the TEC inhibitor is a BTK inhibitor. In some embodiments, the BTK inhibitor and the immune checkpoint inhibitor are administered as a maintenance therapy. In some instances, the BTK inhibitor is ibrutinib. In some embodiments, ibrutinib and the immune checkpoint inhibitor are administered as a maintenance therapy.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, the severity of the disease, a biomarker profile (e.g. Th1:Th2 ratio, or any of the biomarkers described herein), the identity (e.g., weight) of the subject or host in need of treatment, but can nevertheless be routinely determined in a manner known in the art according to the particular circumstances surrounding the case, including, e.g., the specific agent being administered, the route of administration, and the subject or host being treated. In general, however, doses employed for adult human treatment will typically be in the range of 0.02-5000 mg per day, or from about 1-1500 mg per day. The desired dose may conveniently be presented in a single dose or as divided doses administered simultaneously (or over a short period of time) or at appropriate intervals, for example as two, three, four or more sub-doses per day.

The pharmaceutical combination and/or composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it can be expressed as the ratio between LD50 and ED50. Compounds exhibiting high therapeutic indices are preferred. The data obtained from cell culture assays and animal studies can be used in formulating a range of dosage for use in human. The dosage of such compounds lies preferably within a range of circulating concentrations that include the ED50 with minimal toxicity. The dosage may vary within this range depending upon the dosage form employed and the route of administration utilized.

### Kits/Articles of Manufacture

For use in the therapeutic methods of use described herein, kits and articles of manufacture are also described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include Ibrutinib, optionally in a composition or in combination with an immune checkpoint inhibitor as disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical combinations and/or compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### EXAMPLES

The following ingredients, formulations, processes and procedures for practicing the methods disclosed herein correspond to that described above.

### Example 1

### Combination Therapy of Ibrutinib and Either an Anti-PL-D1 Antibody or an Anti-CTLA-4 Antibody in an Ibrutinib-Resistant Mouse Tumor Model

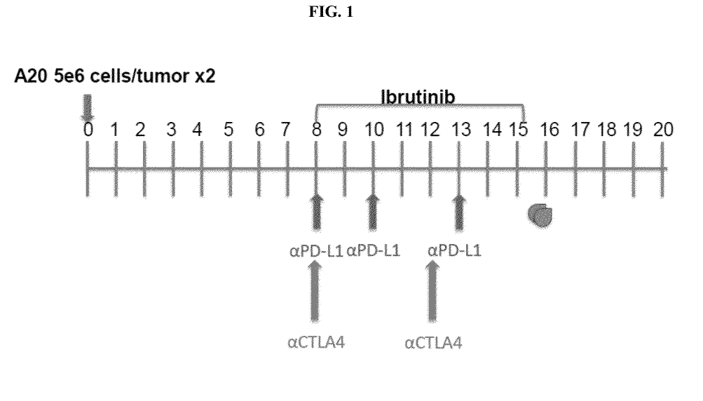

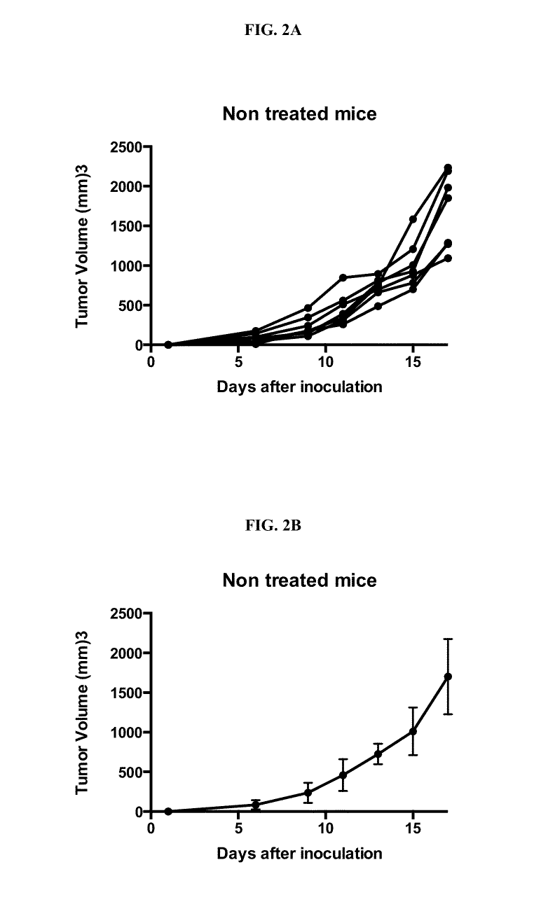

Mice were injected in both sides of their abdomens with cells from the A20 BALB/C B-cell lymphoma cell line, which are resistant to treatment with ibrutinib. Ibrutinib was injected at days 8-15 post-injection of A20 cells. Anti-PD-L1 antibody (e.g., Genentech's Anti-PDL1 antibody MPDL3280A (RG7446)) was administered at days 8, 10, and 13 post A20 injection. Anti-CTLA-4 antibody was administered at days 8 and 12 post A20 injection (FIG. 1).

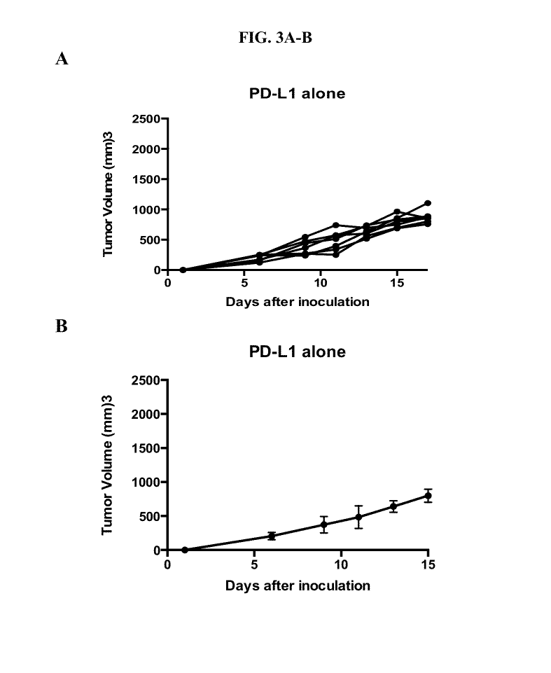

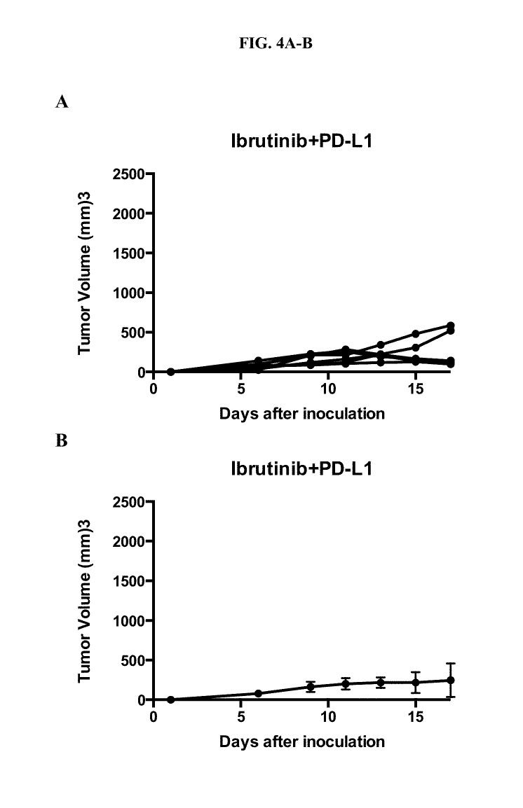

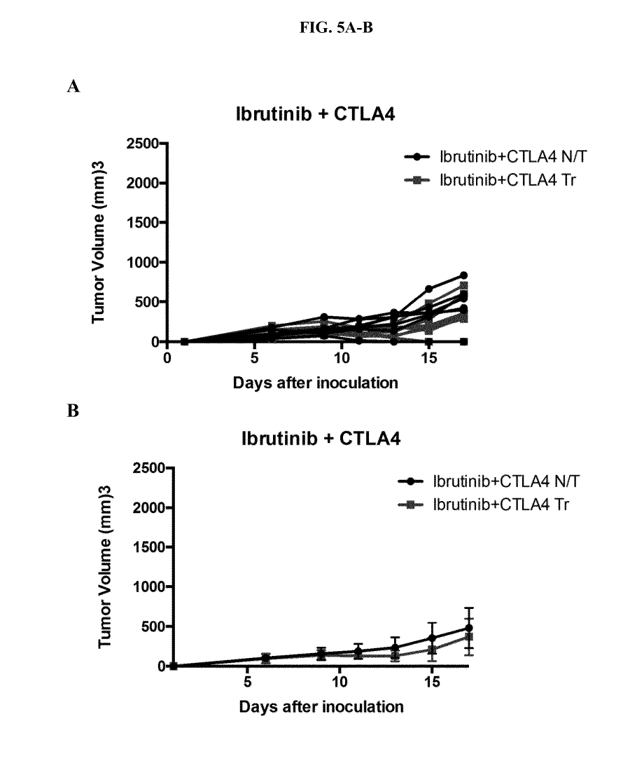

Tumor volume was measured periodically until 15 days post-injection of A20 cells. The combination of anti-PD-L1 antibody and ibrutinib was found to have a synergistic effect in reducing tumor volume as compared to anti-PD-L1 antibody alone (FIGS. 3 and 4). A similar effect was seen with the combination of ibrutinib and the anti-CTLA-4 antibody (FIG. 5).

### Example 2

### Safety and Tolerability Study of Co-Administration of Ibrutinib and an Immune Checkpoint Inhibitor

Purpose:

The purpose of this study is to establish the safety and optimal dose of orally administered Ibrutinib and an injected anti-PD-L1 antibody (e.g., Genentech's Anti-PDL1 antibody MPDL3280A (RG7446)) in patients with B-cell chronic lymphocytic leukemia/small lymphocytic lymphoma/diffuse well-differentiated lymphocytic lymphoma.

Primary Outcome Measures:

Safety and tolerability of combination of Ibrutinib and the anti-PD-L1 antibody (frequency, severity, and relatedness of adverse events).

Secondary Outcome Measures:

Pharmacokinetic/Pharmacodynamic assessments.

Tumor response—overall response rate as defined by recent guidelines on CLL and SLL (B cell lymphoma) and duration of response.

Eligibility:

18 Years and older; both genders are eligible.

Inclusion Criteria:

For treatment-naive group only: Men and women ≧65 years of age with confirmed diagnosis of CLL/SLL, who require treatment per NCl or International Working Group guidelines 11-14.

For relapsed/refractory group only: Men and women ≧18 years of age with a confirmed diagnosis of relapsed/refractory CLL/SLL unresponsive to therapy (ie, failed ≧2 previous treatments for CLL/SLL and at least 1 regimen had to have had a purine analog [eg, fludarabine] for subjects with CLL).

Body weight ≧40 kg.

ECOG performance status of ≦2.

Agreement to use contraception during the study and for 30 days after the last dose of study drug if sexually active and able to bear children.

Willing and able to participate in all required evaluations and procedures in this study protocol including swallowing capsules without difficulty.

Ability to understand the purpose and risks of the study and provide signed and dated informed consent and authorization to use protected health information (in accordance with national and local subject privacy regulations).

Exclusion Criteria:

A life-threatening illness, medical condition or organ system dysfunction which, in the investigator's opinion, could compromise the subject's safety, interfere with the absorption or metabolism of Ibrutinib PO, or put the study outcomes at undue risk.

Any immunotherapy, chemotherapy, radiotherapy, or experimental therapy within 4 weeks before first dose of study drug (corticosteroids for disease-related symptoms allowed but require 1-week washout before study drug administration).

Central nervous system (CNS) involvement by lymphoma.

Major surgery within 4 weeks before first dose of study drug.

Creatinine>1.5×institutional upper limit of normal (ULN); total bilirubin>1.5×ULN (unless due to Gilbert's disease); and aspartate aminotransferase (AST) or alanine aminotransferase (ALT)>2.5×ULN unless disease related.

Concomitant use of medicines known to cause QT prolongation or torsades de pointes.

Significant screening electrocardiogram (ECG) abnormalities including left bundle branch block, 2nd degree AV block type II, 3rd degree block, bradycardia, and QTc>470 msec.

Lactating or pregnant.

### Example 3

### Combination Therapy of Ibrutinib and Anti-PD1/PDL1 Antibody in FL Patients

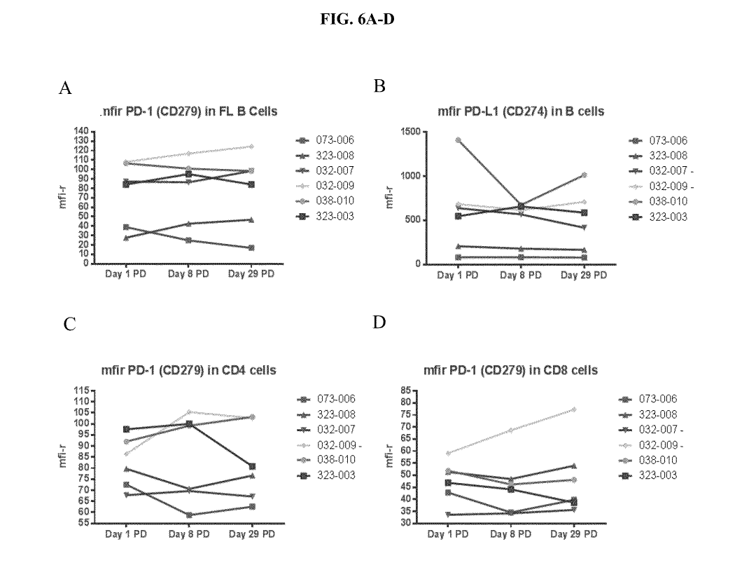

Follicular lymphoma (FL) patients were treated with a combination of ibrutinib and anti-PD1/PDL1 antibody (FIG. 6). Generally, no effect on PDL-1 expression was observed in lymphoma cells treated with ibrutinib. Some FL patients treated with ibrutinib were found to have increased PD-1 levels on their CD8+ T-cells. Generally, PD-1 levels of patients treated with ibrutinib were not decreased. The anti-PD-L1 antibody used was the antibody clone MIH1. The anti-PD-1 antibody used was the antibody clone MIH4. Accordingly, because PD-1 or PDL-1 levels in follicular lymphoma patients were not decreased, it was expected that human follicular lymphoma patients would benefit from combining anti-PD1/PDL1 with ibrutinib.

### Example 4

### Combination Therapy of Ibrutinib and Anti-PD1/PDL1 Antibody in DLBCL Tumor Models

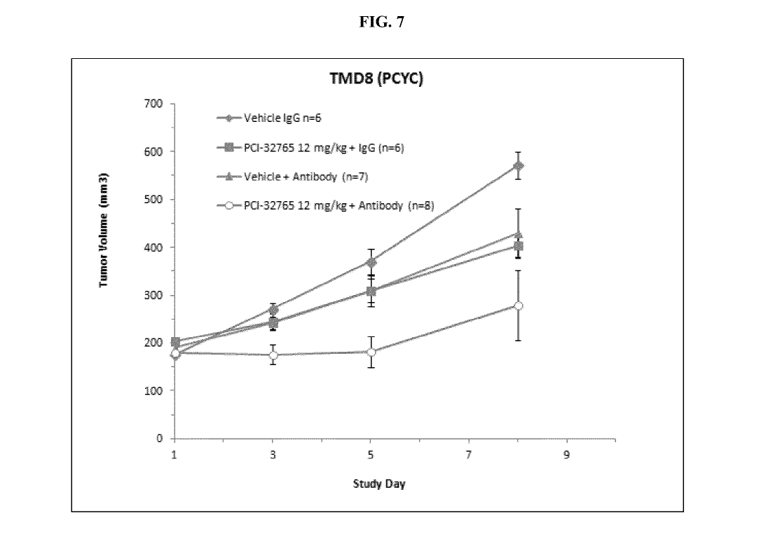

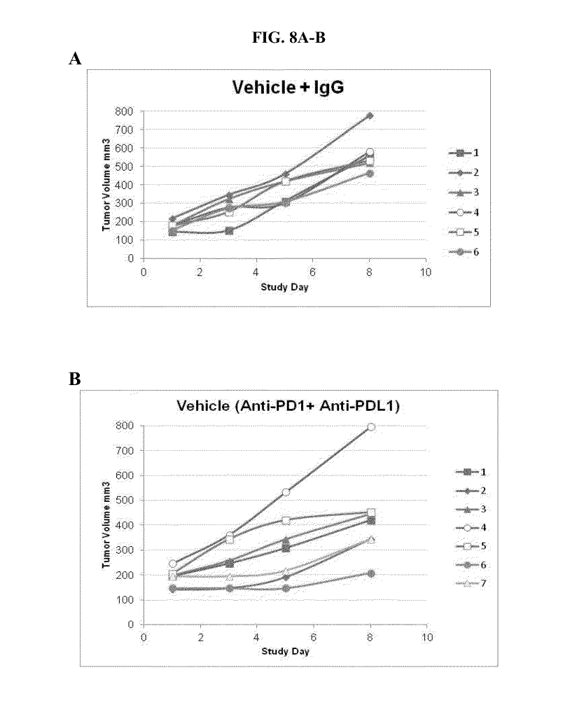

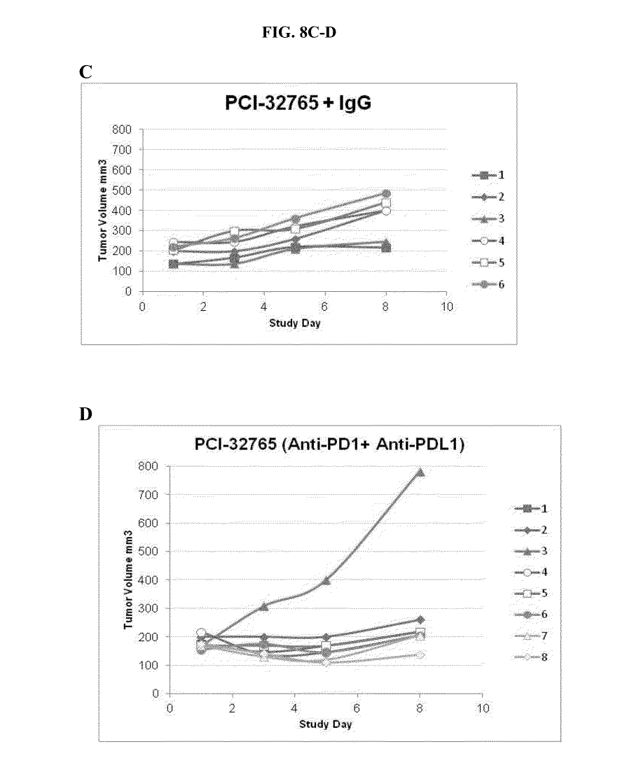

CB 17 SCID mice (6-8 weeks old) were innoculated subcutaneously with 10 million TMD8 tumor cells (ABC-DLBCL) on the flank with 100% matrigel. The tumored animals were administered with either ibrutinib at doses of 3 mg/kg or anti-PDL1/PD1 or ibrutinib and anti-PDL1 and PD1 (100 μg) intratumorly when the tumor size reached about 150 mm³. Tumor burden was determined every two days and concluded on day 8. The combination of anti-PD1/PD-L1 antibody and ibrutinib was found to have a synergistic effect in reducing tumor volume as compared to treatment with ibrutinib or anti-PD1/PD-L2 antibody alone (FIGS. 7 and 8).

### Example 5

### Combination Therapy of Ibrutinib and Anti-PD1/PDL1 Antibody in CLL Patients

RNA was isolated from chronic lymphocytic leukemia (CLL), CLL/PLL and CLL/SLL patients before treatment, during treatment with response and after relapse to ibrutinib (on average 2 years). These RNA samples were then subjected to RNAseq (Expression Analysis) and analyzed for differential expression using Bowtie for transcriptome alignment, RSEM to quantify counts to different transcripts and EBSeq to align differentially expressed genes.

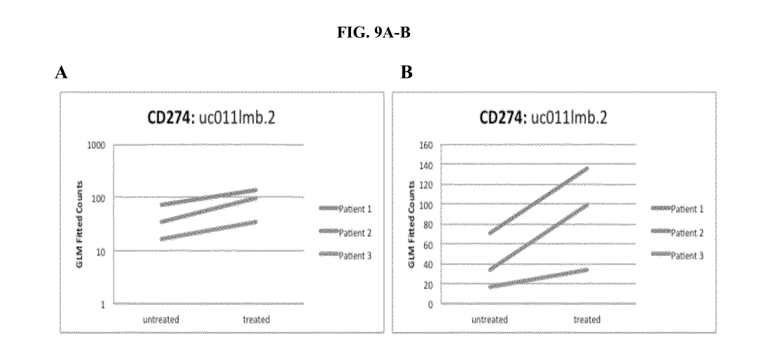

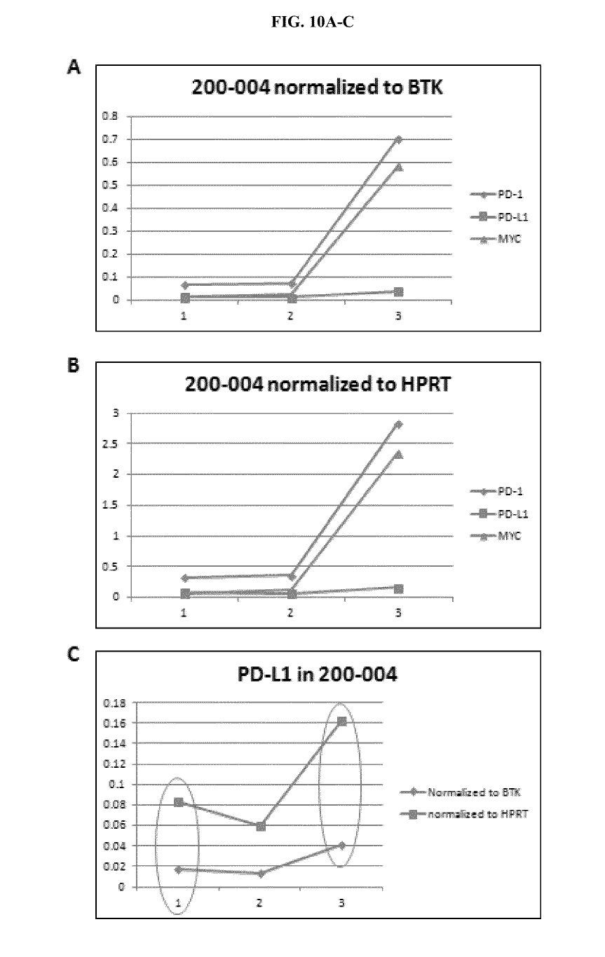

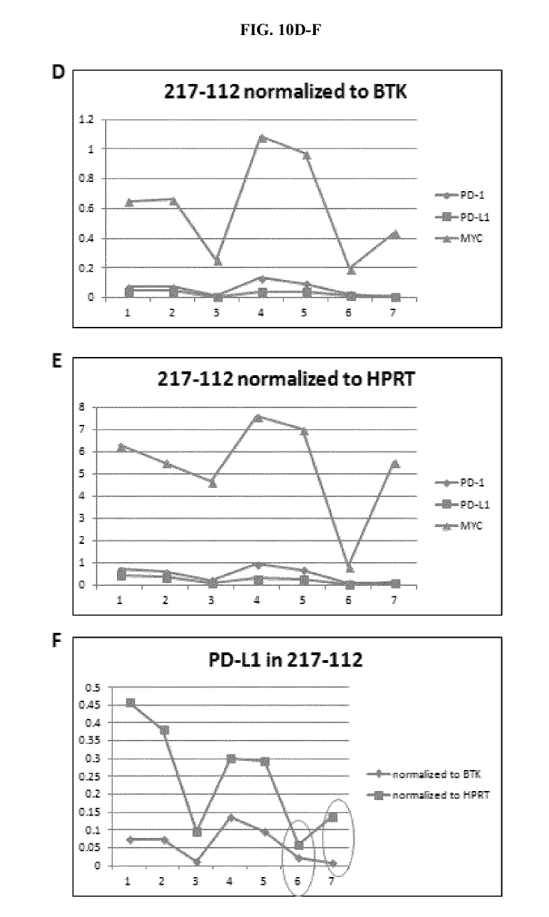

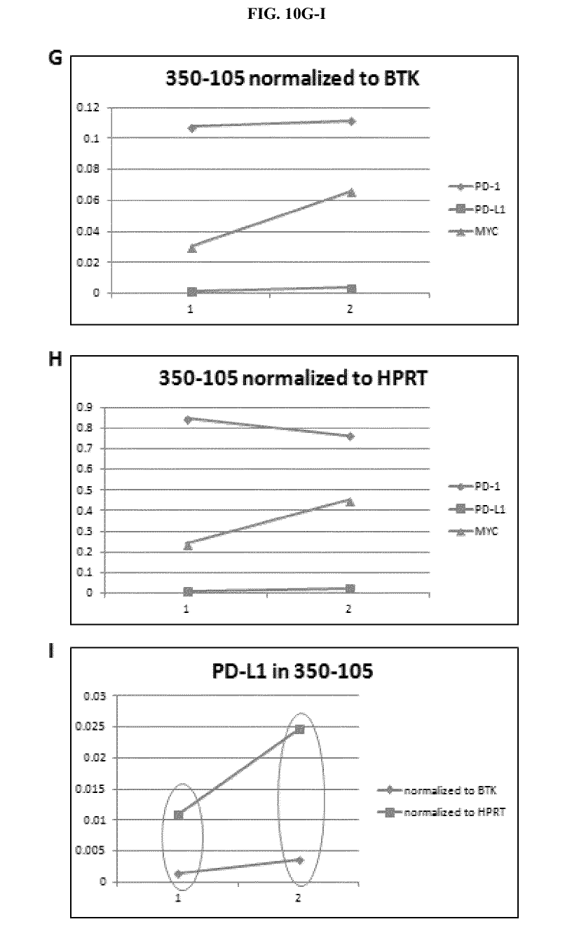

In patients resistant to ibrutinib treatment, the levels of PD1 and PD-L1 were observed to be upregulated (FIGS. 9 and 10).

### Example 6

### Combination Therapy of Ibrutinib with Anti-PD-1/PD-L1 Targeting Different Tumor Sizes in Ibrutinib-Resistant Mouse Tumor Models

### Materials

Cell line A20, a BALB/c B cell lymphoma line expressing MHC class I and class II H-2d molecules, was obtained from ATCC. A20 cells were cultured in complete Roswell Park Memorial Institute 1640 medium (cRPMI; Invitrogen) containing 10% fetal bovine serum (FBS; Thermo Scientific), 100 U/mL penicillin, 100 μg/mL streptomycin (both from Invitrogen), and 50 μM 2-ME (Sigma-Aldrich).

The mouse anti PD-L1-10F.9G2 antibody was purified from the isotype control rat hybridoma SFR8-B6 (ATCC HB-152) and was collected by Bionexus Inc. from ascites of SCID mice.

Mice were housed in the Laboratory Animal Facility of the Stanford University Medical Center (Stanford, Calif.). All experiments were approved by the Stanford Administrative Panel on Laboratory Animal Care and conducted in accordance with Stanford University Animal Facility and National Institutes of Health guidelines.

### Tumor Transplantation and Treatment

Different treatment regiments were applied at tumor sizes of either 0.7-1 cm or 0.5-0.7 cm in the largest diameter. Tumor cells were implanted into mice while in exponential growth phase (below 1.5×10⁶ cells/mL). In the first set of treatment regiment, six to eight week old female BALB/c were inoculated with 5×10⁶ A20 cells by subcutaneous (s.c.) injection to the right and left sides of their abdomen. Tumor growth was monitored with a digital caliper (Mitutoyo) every 2 to 3 days and expressed as volume (length×width×height). Mice were euthanized when s.c. tumor size reached 1.5 cm².

Therapy started when tumors reached a size of 0.7-1 cm in the largest diameter. Anti-PD-L1 (200 μg/injection; BioXcell) was given Intraperitonealy (IP) 3 times a week. Ibrutinib (6 mg/kg) was given IP daily on days 1-8.

In the second set of treatment regiment, six to eight week old female BALB/c were inoculated with 5×10⁶ A20 cells by s.c. injection and tumor growth was monitored by caliper measurement. Treatment started when tumors reached a size of 0.5-0.7 cm in the largest diameter. Ibrutinib (6 mg/kg) was given IP daily for 8 days and anti-PD-L1 (100 or 200 μg/injection) was given IP 3 times a week.

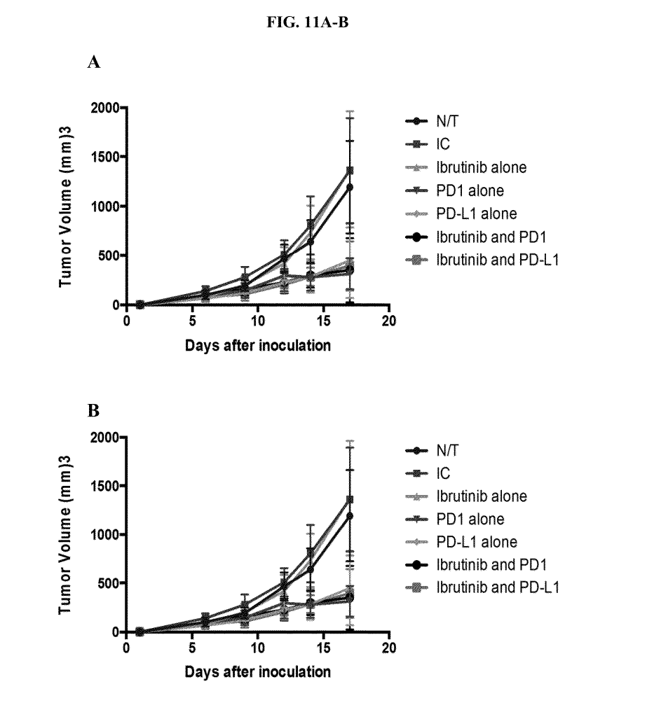

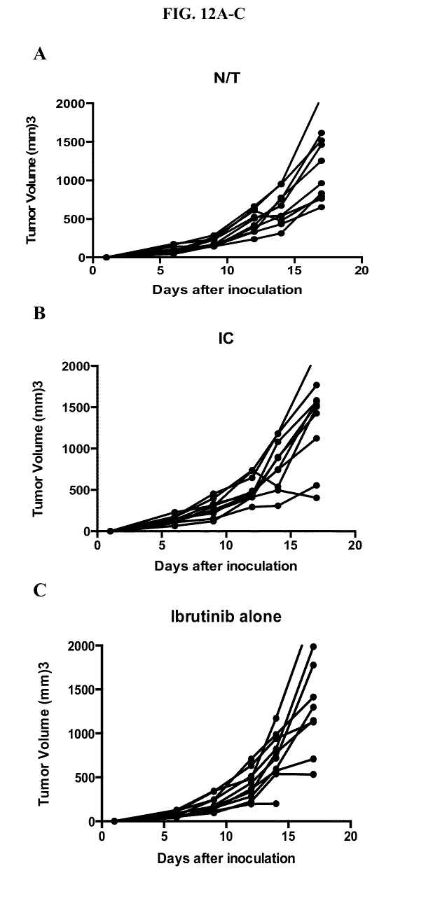

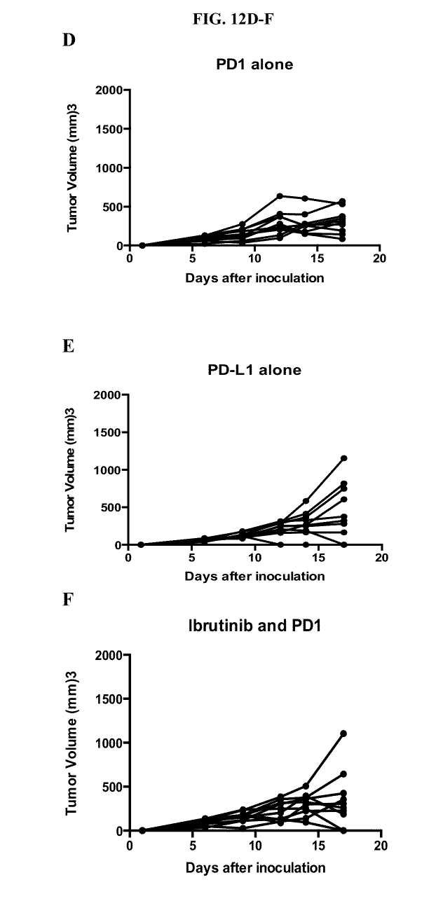

When tumor growth reached a size of 0.7-1 cm, addition of ibrutinib to anti-PD-1/PD-L1 treatment (200 μg/injection) reduced tumor size compared to anti-PD-1/PD-L1 treatment alone (FIG. 11A and FIG. 12). Similarly, survival rate of mice improved with treatment combining ibrutinib compared to anti-PD-1/PD-L1 treatment alone (FIG. 11B). The survival rate of mice treated with ibrutinib in combination with anti-PD-1 was higher than the survival rate of mice treated with ibrutinib in combination with anti-PD-L1.

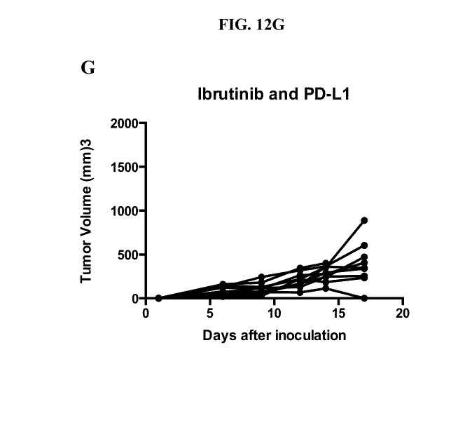

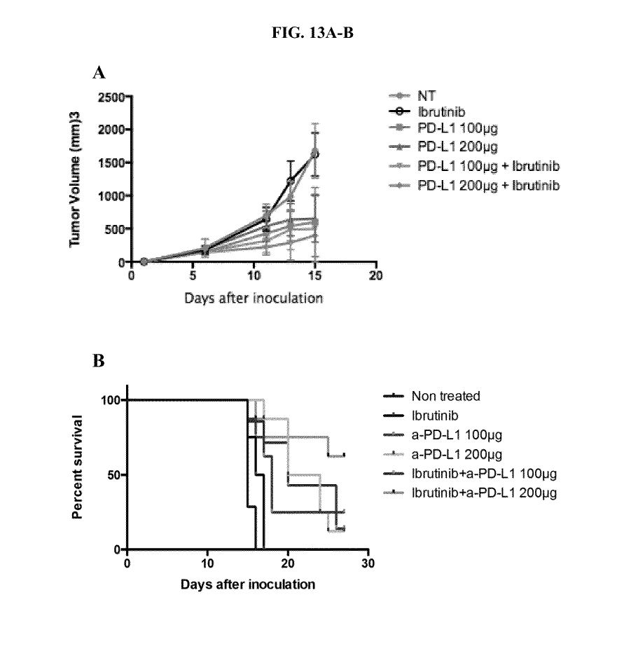

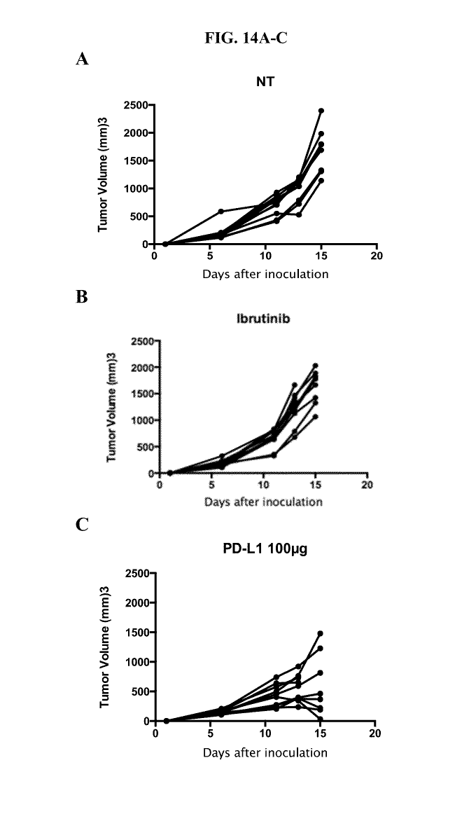

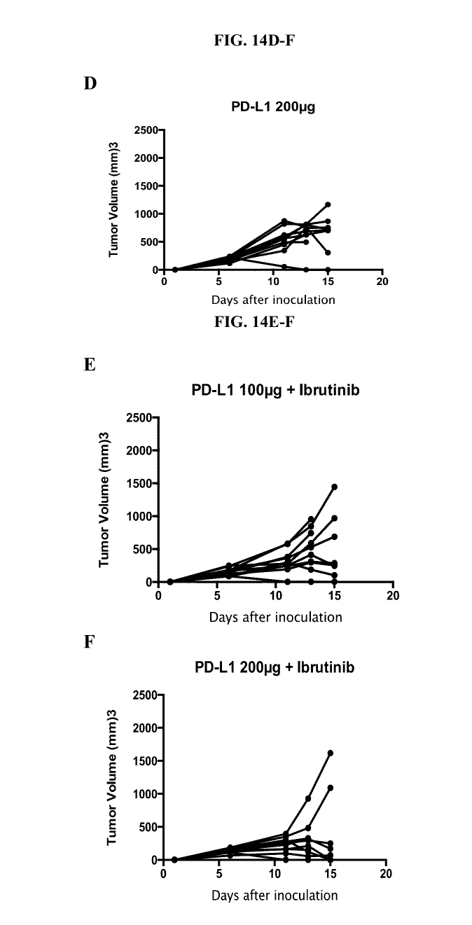

When tumor size reached 0.5-0.7 cm, addition of ibrutinib to anti-PD-L1 (α-PD-L1) treatment reduced tumor size compared to anti-PD-L1 treatment alone (FIG. 13A, FIG. 14 and FIG. 19). Survival rate of mice improved with treatment combining ibrutinib compared to anti-PD-L1 treatment alone (FIG. 13B). At an anti-PD-L1 concentration of 200 μg/injection, the survival rate of mice was greater than 50%. At an anti-PD-L1 concentration of 100 μg/injection, the survival rate of mice was below 50%.

### IFN-γ Production Assay

Single cell suspensions were made from spleens of treated mice, red cells were lysed with ammonium chloride potassium buffer (Quality Biological, Gaithersburg, Md.). Splenocytes were then cocultured with RPMI, stimulated with 0.05 μg anti-mouse CD3 mAb (BD Pharmingen), 1×10⁶ irradiated 2F3 or A20 cells for 24 hours with 0.5 μg anti-mouse CD28 mAb and in the presence of monensin (Golgistop; BD Biosciences) for the last 6 hours at 37° C. and 5% CO2. Intracellular IFN-γ and perforin expressions were assessed using BD Cytofix/Cytoperm Plus Kit per instructions.

### Flow Cytometry

Cells were surface stained in wash buffer (PBS, 1% FBS, and 0.01% sodium azide), fixed in 2% paraformaldehyde, and analyzed by flow cytometry on a FACSCalibur (BD Biosciences). Mouse Fc receptors were blocked with 1 μg FcγRIII/II-specific antibody (clone 2.4G2, rat IgG2b κ; BD Bioscience) per 1×10⁶ cells. FACS data were analyzed using Cytobank.

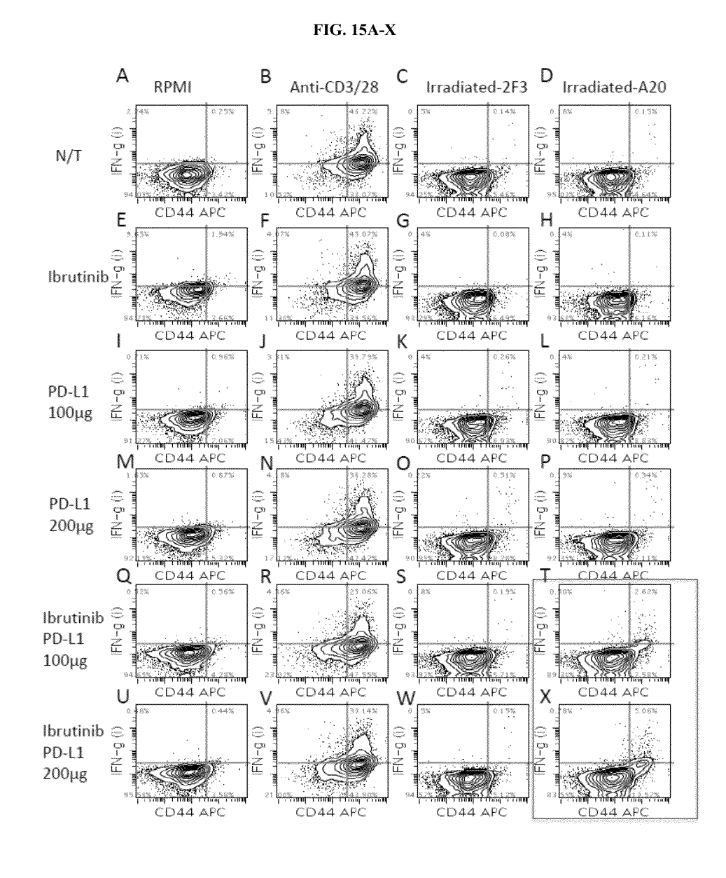

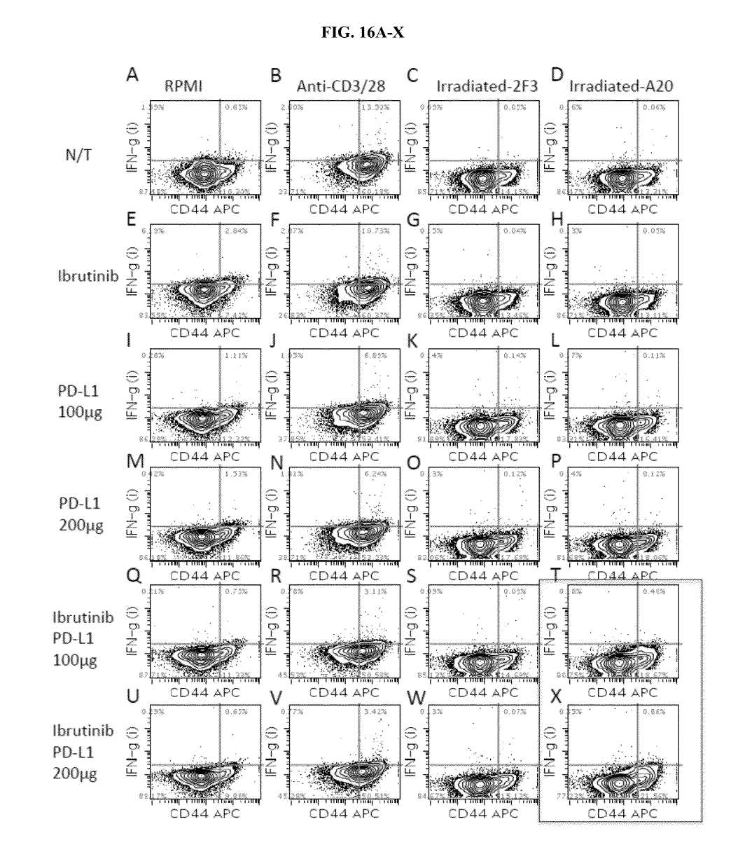

CD8+ and CD4+ T cells were subjected to treatment of either ibrutinib or anti-PD-L1 (100 μg/injection or 200 μg/injection) alone or ibrutinib in combination with anti-PD-L1 (FIG. 15 and FIG. 16). In CD8+ T cells, irradiated A20 responded to treatment of ibrutinib in combination with PD-L1 but not irradiated 2F3. Similarly in CD4+ T cells, irradiated A20 responded to treatment of ibrutinib in combination with PD-L1. 2F3 is a subclonal renal cell line and A20 is a mouse B lymphoma cell line.

### Example 7

### Combination Therapy of Ibrutinib with Anti-PD-L1 to Induce an Anti-Cancer Immune Response in a Mouse Tumor Model

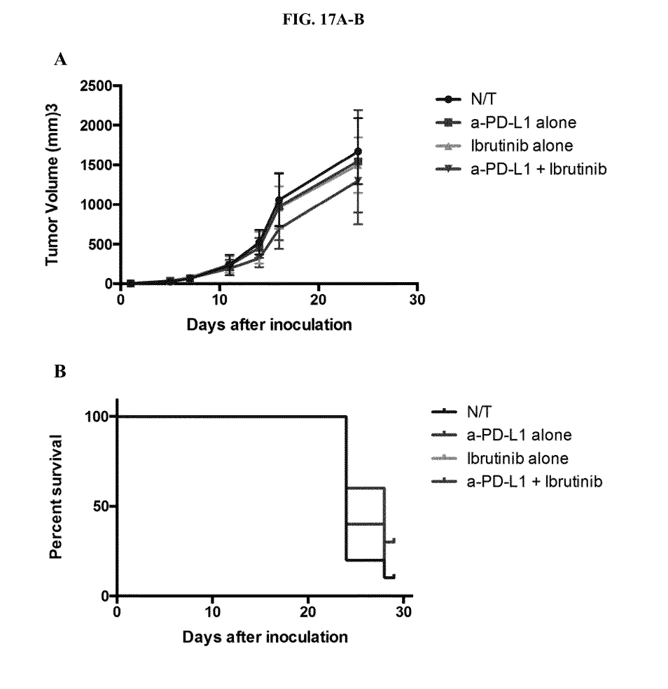

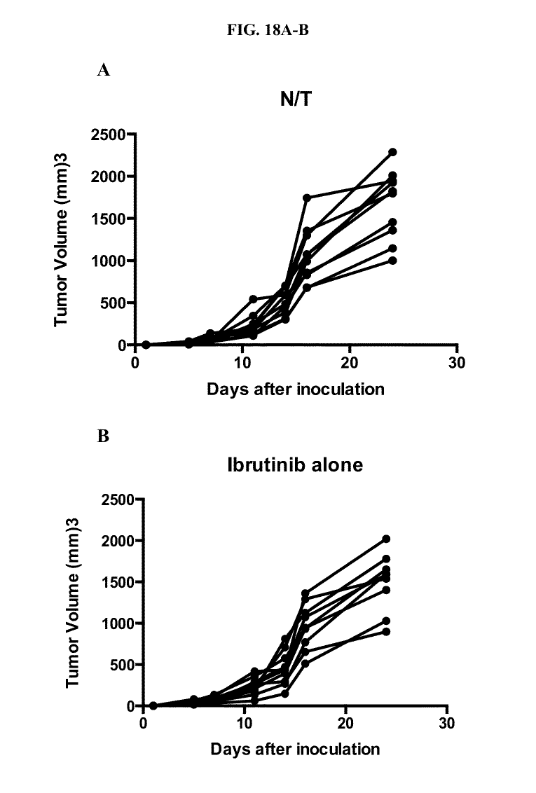

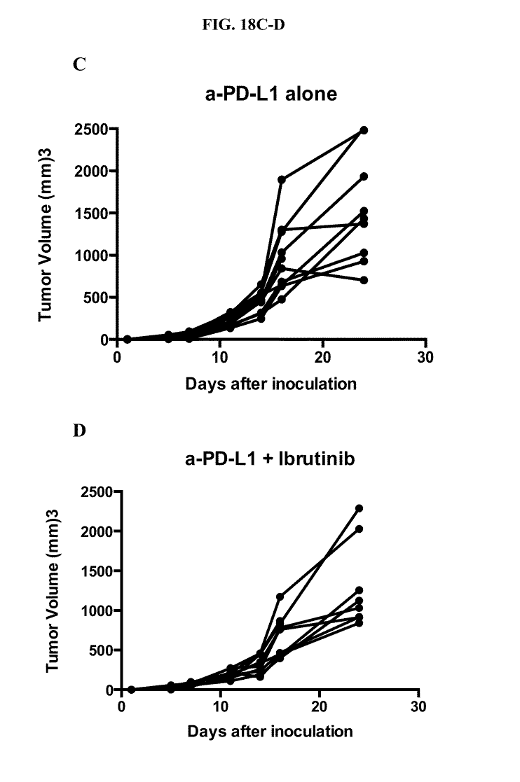

Mice were injected with cells from the 4T1 cell line, which induces an animal stage IV human breast cancer. Addition of ibrutinib to anti-PD-L1 (α-PD-L1) treatment reduced tumor size compared to anti-PD-L1 treatment alone (FIG. 17A and FIG. 18). Survival rate of mice improved with treatment combining ibrutinib compared to anti-PD-L1 treatment alone (FIG. 17B).

### Example 8

### Combination Therapy of Ibrutinib with Anti-PD-L1/Anti-PD-1 in Breast Cancer and Colon Cancer Mouse Models

### Reagents

Ibrutinib was provided by Pharmacyclics, Inc. (Sunnyvale, Calif.). Anti-mouse PD-L1, Clone 10F.9G2; and anti-mouse PD-1, clone RMP1-14, antibodies were purchased from (BioXcell West Lebanon, N.H.). The isotype control rat hybridoma, SFR8-B6 (ATCC HB-152) was produced as ascites in SCID mice by Bionexus (Oakland, Calif.).

The following monoclonal antibodies (mAbs) were used for flow cytometry: rat anti-mouse CD4-PerCP cy5.5, rat anti-mouse CD3-PerCP cy5.5, rat anti-mouse CD8a-FITC, rat anti-mouse CD44-APC, rat anti-mouse CD49b-APC, rat anti-mouse IFN-gamma-PE, rat anti-mouse perforin-PE, hamster anti-mouse CD80-PE, anti H-2 Kb-PE, and anti-Ia-PE. These antibodies and their isotype controls were purchased from either BD Biosciences or eBioscience.

### Cell Lines and Mice

The CT26 colon carcinoma line was obtained from ATCC (Manassas, Va.). The 4T1-Luc breast carcinoma cell line was a gift from the S. Strober laboratory (Stanford University) and the C. Contag laboratory (Stanford University). Tumor cells were cultured in complete medium (RPMI 1640; cellgro) containing 10% fetal bovine serum (FBS; HyClone), 100 U/mL penicillin, 100 μg/mL streptomycin, and 50 μM 2-ME (Gibco).

Six to eight week-old female BALB/c mice were purchased from JAX Laboratories. Mice were housed in the Laboratory Animal Facility of the Stanford University Medical Center (Stanford, Calif.). All experiments were approved by the Stanford Administrative Panel on Laboratory Animal Care and conducted in accordance with Stanford University Animal Facility and National Institutes of Health guidelines.

### Tumor Inoculation

4T1-luc and CT26 tumor cells (0.01×10⁶, 0.5×10⁶ respectively) were injected to the right side of the abdomen. Ibrutinib was injected by the intraperitoneal route at a dose of 6 mg/kg beginning on day 8 after tumor implantation or when tumors reached a minimal size of 5 mm in the largest diameter and continued daily for 8-14 days.

Tumor size were monitored with a digital caliper (Mitutoyo) every 2 to 3 days and expressed as volume (length×width×height). Mice were sacrificed when tumor size reached 1.5 cm² when inoculated with 2 tumors and 2 cm² when inoculated with one as per guidelines.

### Flow Cytometry

Cells were surface stained in phosphate-buffered saline (PBS), 1% FBS, and 0.01% sodium azide, fixed in 2% paraformaldehyde, and analyzed by flow cytometry on a FACSCalibur (BD Biosciences). Data were stored and analyzed using Cytobank (http://www.cytobank.org).

### Statistical Analysis

Prism software (GraphPad; La Jolla, Calif.) was used to analyze tumor growth and to determine statistical significance of differences between groups by applying an unpaired Student's t-test. P values<0.05 were considered significant.

### IFN-γ and Perforin Assay

Single cell suspensions were made from spleens of treated mice, red cells were lysed with ammonium chloride, potassium buffer (Quality Biological, Gaithersburg, Md.). Splenocytes were then co-cultured with 1×10⁶ irradiated CT26, 4T1-luc, A20 or 2F3 cells for 24 hours at 37° C. and 5% CO₂ in the presence of 0.5 μg anti-mouse CD28 mAb (BD PharMingen). Monensin (Golgistop; BD Biosciences, San Jose, Calif.) was added for the last 5 hours. Intracellular IFNγ and perforin expression was assessed using BD Cytofix/Cytoperm Plus Kit per manufacturer's instructions.

### Discussion

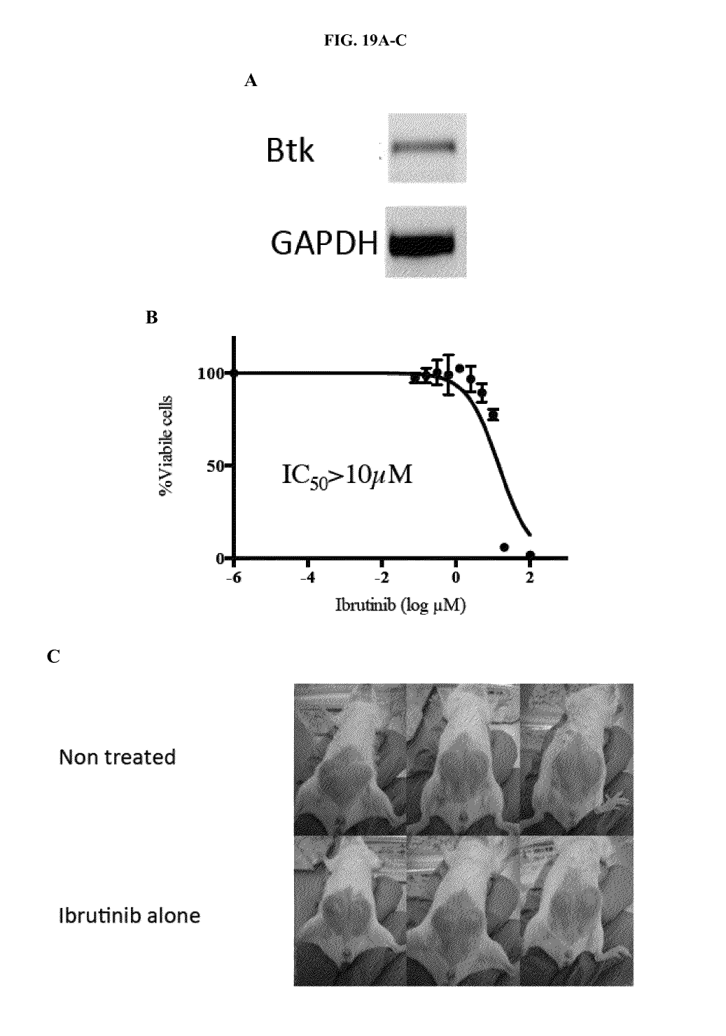

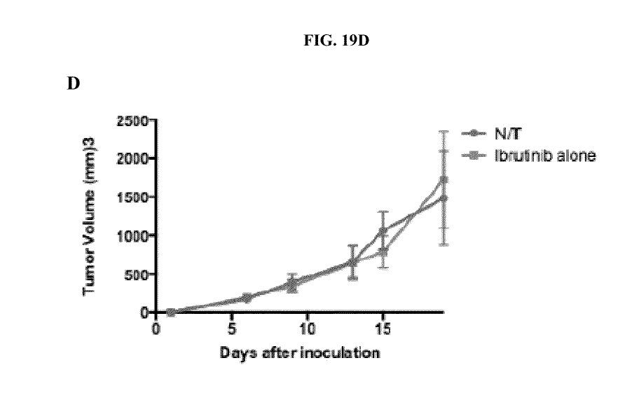

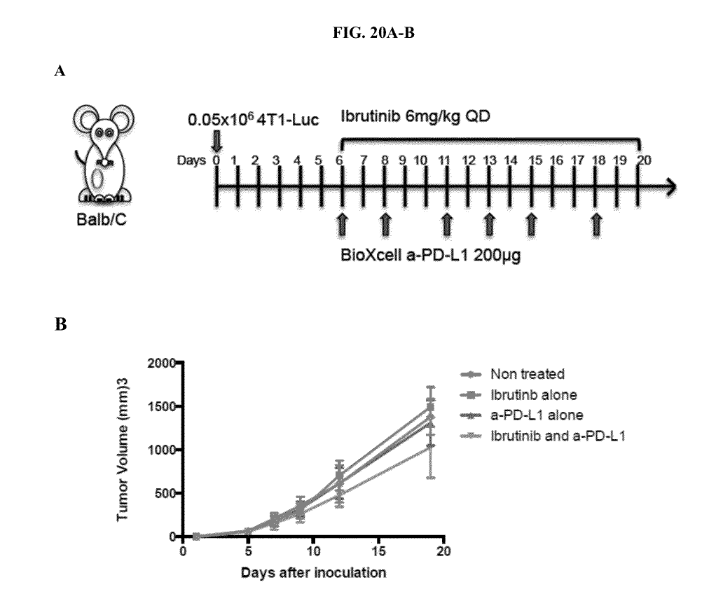

Three sets of experiments were carried out using the 4T1 breast cancer model. FIG. 20 and FIG. 21 illustrate a first set of experiments using the 4T1 breast cancer model. FIG. 20A exemplifies an ibrutinib and anti-PD-L1 antibody administration schedule in a mouse model injected with 4T1-Luc (0.05×10⁶) cells into the mammary fat pad of the mouse. Ibrutinib was administered at 6 mg/kg on days 6-20 post injection of 4T1-Luc cells. Anti-PD-L1 (200 μg) was administered on days 6, 8, 11, 13, 15 and 18 post-injection of 4T1-Luc cells. The 4T1 cell line is a model of triple negative breast cancer, and it is not sensitive to ibrutinib. After about 3-4 weeks of injection, the breast cancer metastasizes to the lung. FIG. 20B illustrates the mean tumor volume from non-treated, Ibrutinib alone, anti-PD-L1 alone, and Ibrutinib+anti-PD-L1 mice after injection with 4T1-Luc cells. FIG. 21A-21D exemplify the tumor volume from non-treated, Ibrutinib alone, anti-PD-L1 alone, and Ibrutinib+anti-PD-L1 mice after injection with 4T1-Luc cells.

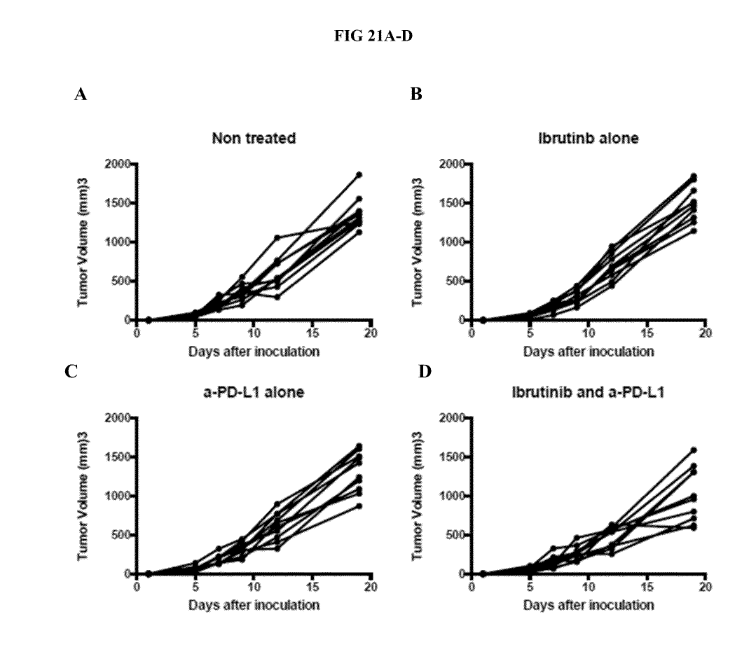

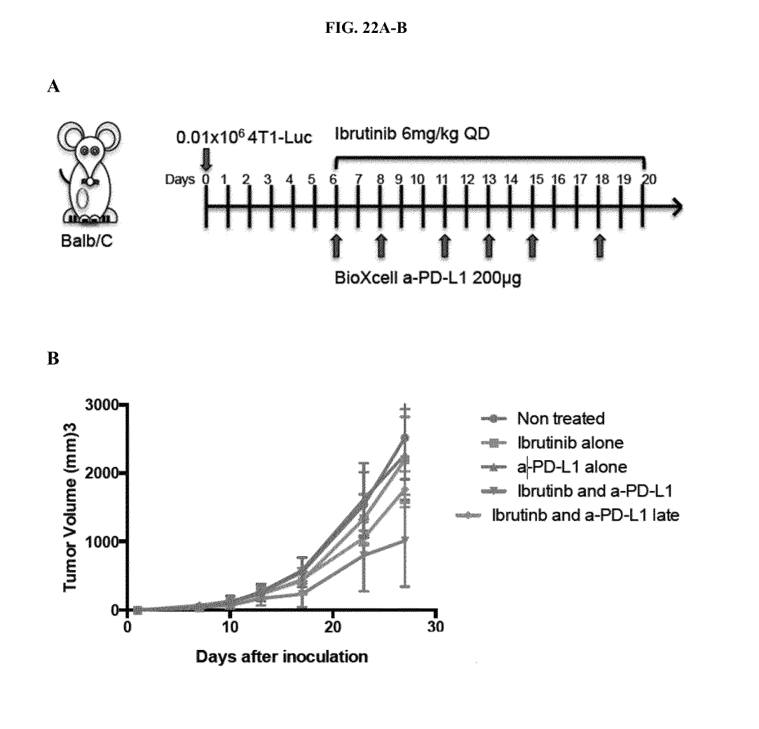

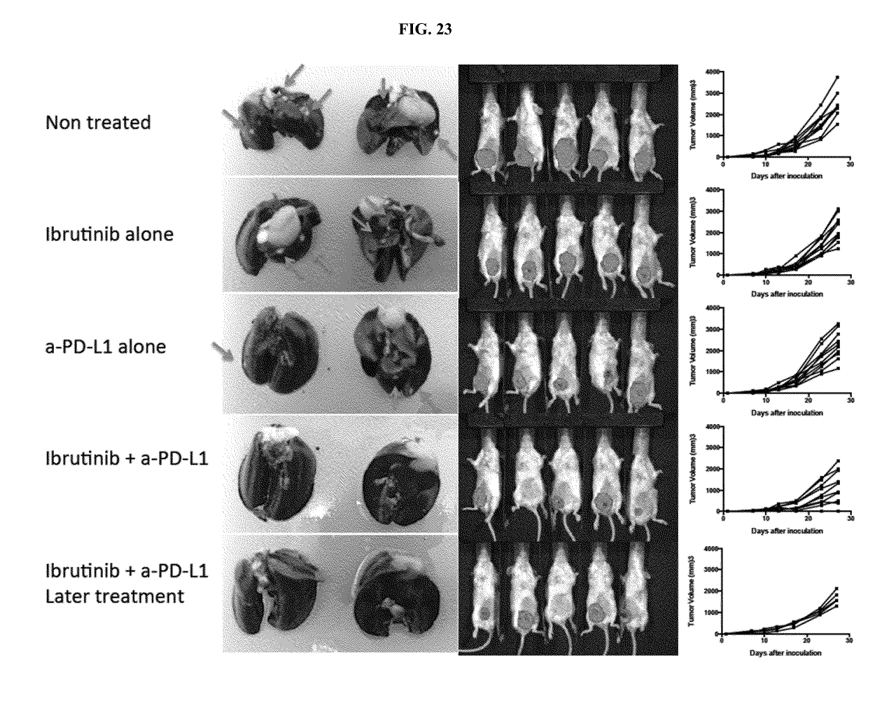

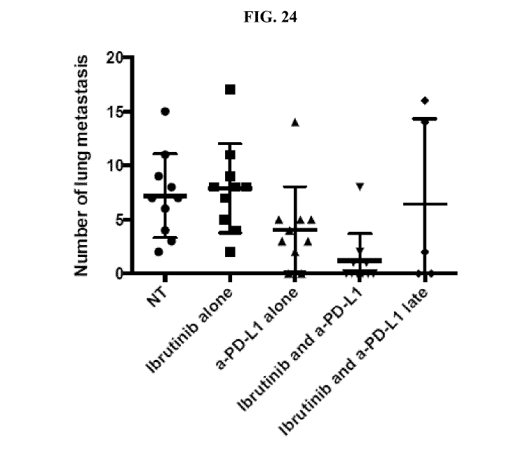

FIG. 22-FIG. 24 illustrate a second set of experiments using the 4T1 breast cancer model. FIG. 22A exemplifies an ibrutinib and anti-PD-L1 antibody administration schedule in a mouse model injected with 4T1-Luc (0.01×10⁶) cells into the mammary fat pad of the mouse. Ibrutinib was administered at 6 mg/kg on days 6-20 post injection of 4T1-Luc cells. Anti-PD-L1 (200 μg) was administered on days 6, 8, 11, 13, 15 and 18 post-injection of 4T1-Luc cells. The 4T1 cell line is a model of triple negative breast cancer, and it is not sensitive to ibrutinib. After about 3-4 weeks of injection, the breast cancer metastasizes to the lung. FIG. 22B illustrates the mean tumor volume from non-treated, Ibrutinib alone, anti-PD-L1 alone, Ibrutinib+anti-PD-L1, and ibrutinib+anti-PD-L1 (started 3 days later) mice after injection with 4T1-Luc cells. FIG. 23 exemplifies lung metastasis, bioluminescence imaging, and subcutaneous tumor growth for control (vehicle) group, ibrutinib alone group, anti-PD-L1 group, and ibrutinib+anti-PD-L1 group. The combination of ibrutinib and anti-PD-L1 effectively inhibits primary tumor growth and lung metastasis in a syngeneic 4T1 model. FIG. 24 exemplifies the number of lung metastasis in non-treated, Ibrutinib alone, anti-PD-L1 alone, Ibrutinib+anti-PD-L1, and ibrutinib+anti-PD-L1 (started 3 days later) mice after injection with 4T1-Luc cells.

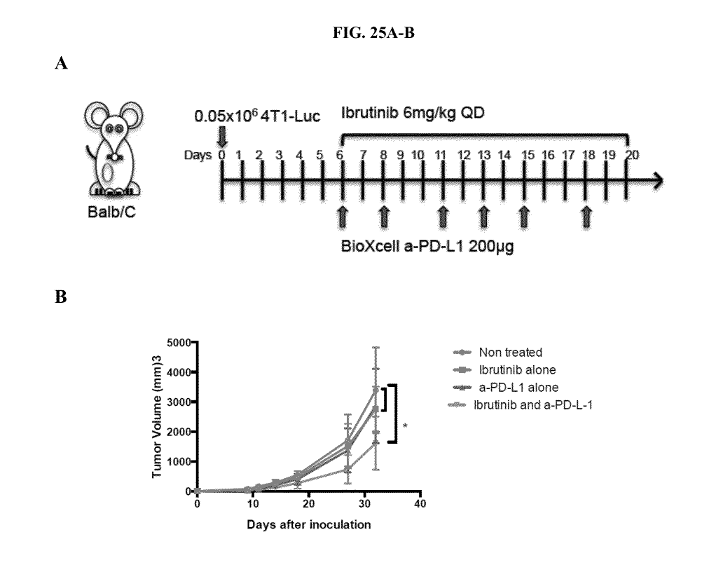

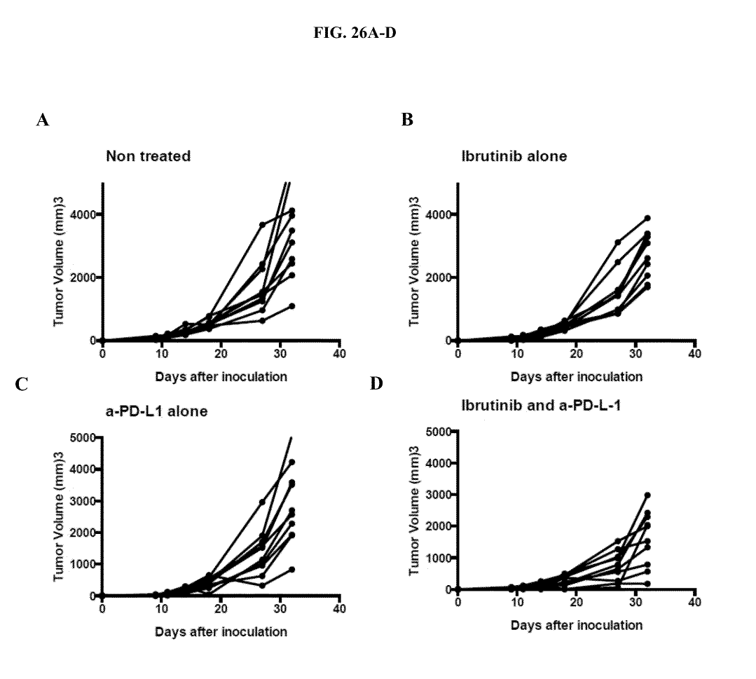

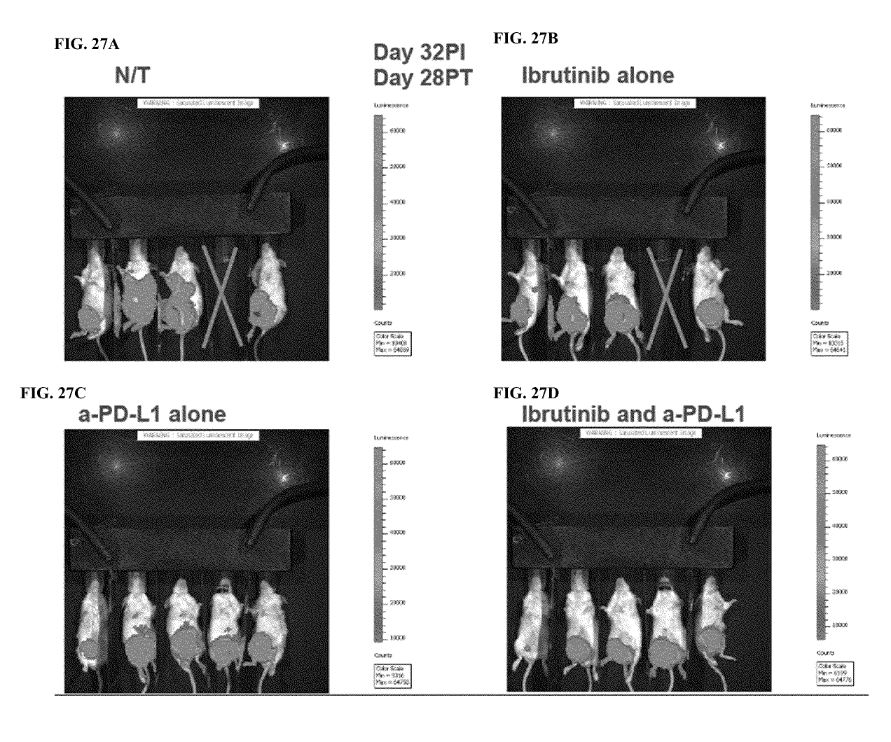

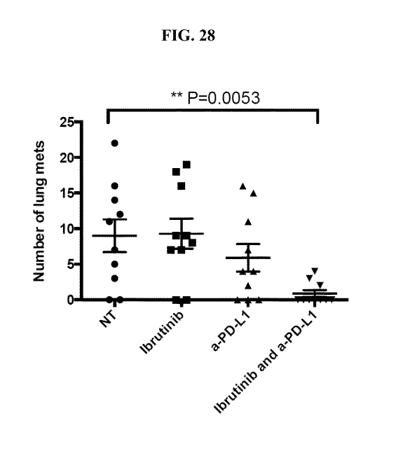

FIG. 25-FIG. 28 illustrate a third set of experiment using the 4T1 breast cancer model. FIG. 25A exemplifies an ibrutinib and anti-PD-L1 antibody administration schedule in a mouse model injected with 4T1-Luc (0.05×10⁶) cells into the mammary fat pad of the mouse. Ibrutinib was administered at 6 mg/kg on days 6-20 post injection of 4T1-Luc cells. Anti-PD-L1 (200 μg) was administered on days 6, 8, 11, 13, 15 and 18 post-injection of 4T1-Luc cells. The 4T1 cell line is a model of triple negative breast cancer, and it is not sensitive to ibrutinib. After about 3-4 weeks of injection, the breast cancer metastasizes to the lung. FIG. 25B illustrates the mean tumor volume from non-treated, Ibrutinib alone, anti-PD-L1 alone, and Ibrutinib+anti-PD-L1 mice after injection with 4T1-Luc cells. FIG. 26A-26D exemplify the tumor volume from non-treated, Ibrutinib alone, anti-PD-L1 alone, and Ibrutinib+anti-PD-L1 mice after injection with 4T1-Luc cells. FIG. 27A-27D exemplify bioluminescence imaging from non-treated, Ibrutinib alone, anti-PD-L1 alone, and Ibrutinib+anti-PD-L1 mice after injection with 4T1-Luc cells. FIG. 28 exemplifies the number of lung metastasis in non-treated, Ibrutinib alone, anti-PD-L1 alone, and Ibrutinib+anti-PD-L1 mice after injection with 4T1-Luc cells.

From these three sets of experiments, the combination of ibrutinib and anti-PD-L1 had greater effect on tumor reduction than ibrutinib and anti-PD-L1 alone. This was also observed in lung metastasis.

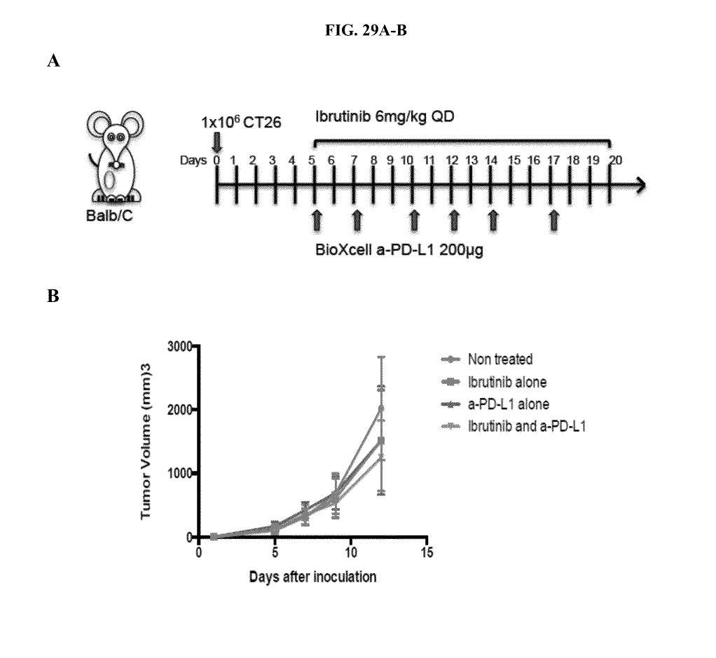

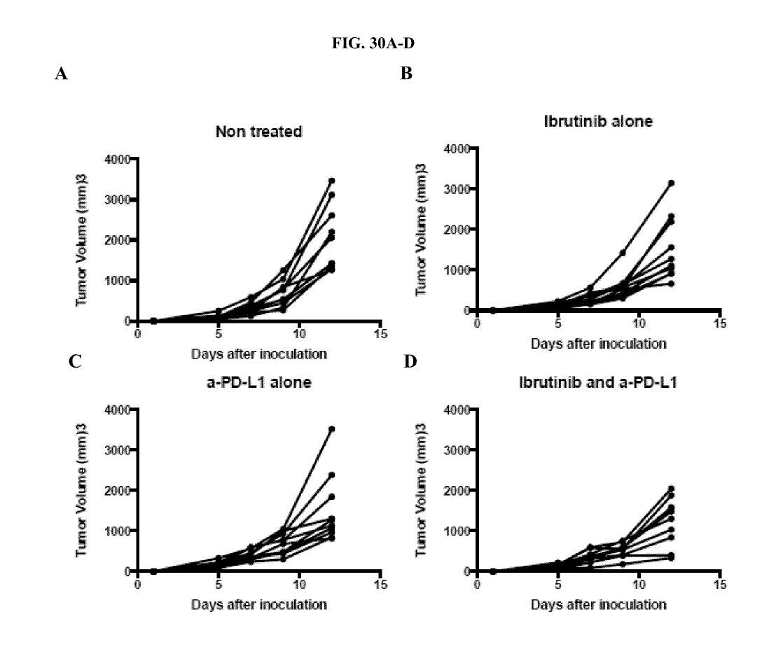

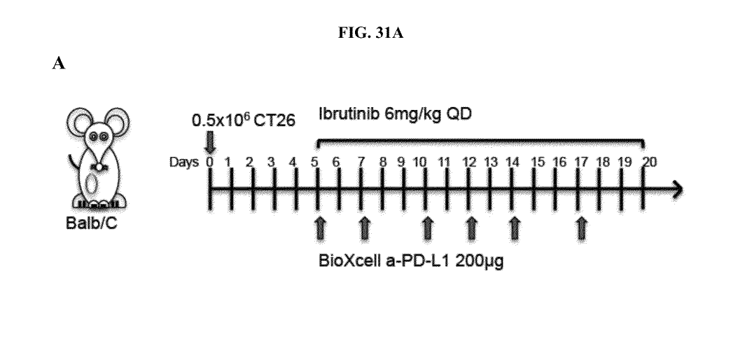

Four sets of experiments were carried out using the CT26 colon cancer model. FIG. 29 and FIG. 30 illustrate a first set of experiment using the CT26 colon cancer model. FIG. 29A exemplifies an ibrutinib and anti-PD-L1 antibody administration schedule in a mouse model injected with CT26 (1×10⁶) cells into the mammary fat pad of the mouse. Ibrutinib was administered at 6 mg/kg on days 5-20 post injection of CT26 cells. Anti-PD-L1 (200 μg) was administered on days 5, 7, 10, 12, 14, and 17 post-injection of CT26 cells. The CT26 cell line is not sensitive to ibrutinib. FIG. 29B illustrates the mean tumor volume from non-treated, Ibrutinib alone, anti-PD-L1 alone, and Ibrutinib+anti-PD-L1 mice after injection with CT26 cells. FIG. 30A-30D exemplify the tumor volume from non-treated, Ibrutinib alone, anti-PD-L1 alone, and Ibrutinib+anti-PD-L1 mice after injection with CT26 cells.

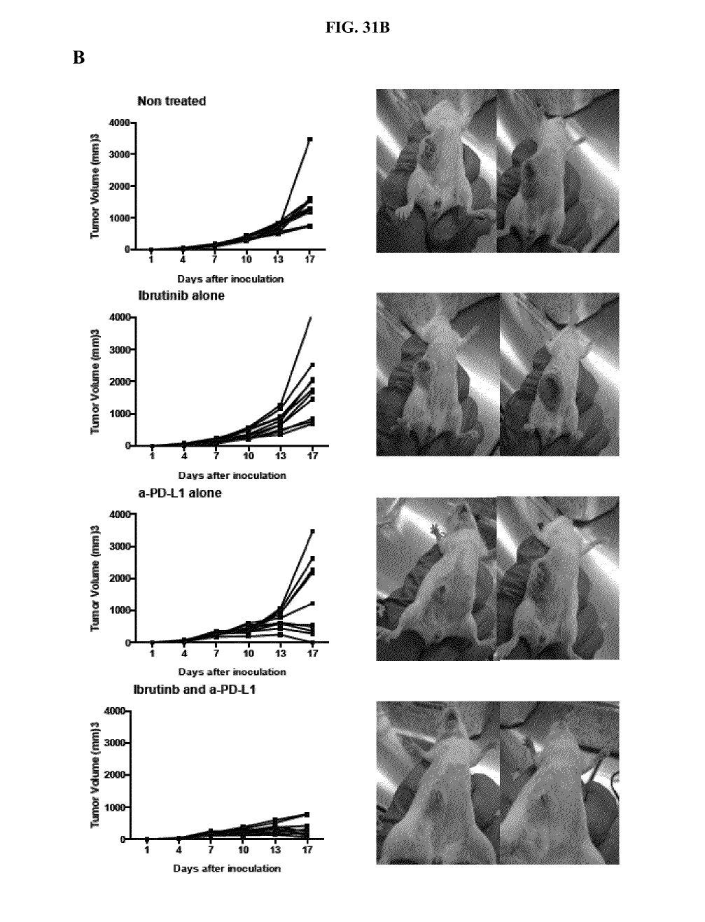

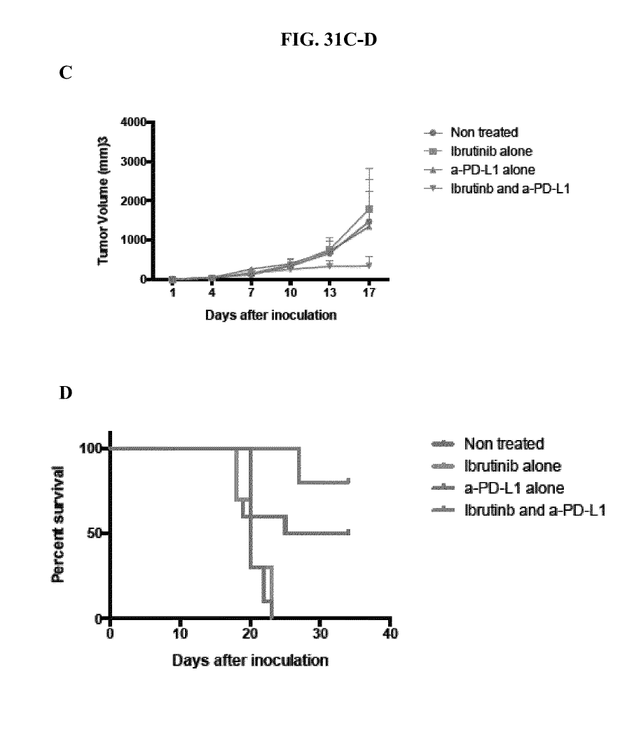

FIG. 31 illustrates a second set of experiment using the CT26 colon cancer model. FIG. 31A exemplifies an ibrutinib and anti-PD-L1 antibody administration schedule in a mouse model injected with CT26 (0.5×10⁶) cells into the mammary fat pad of the mouse. Ibrutinib was administered at 6 mg/kg on days 5-20 post injection of CT26 cells. Anti-PD-L1 (200 μg) was administered on days 5, 7, 10, 12, 14, and 17 post-injection of CT26 cells. The CT26 cell line is not sensitive to ibrutinib. FIG. 31B exemplifies the tumor volume and tumor location from non-treated, Ibrutinib alone, anti-PD-L1 alone, and Ibrutinib+anti-PD-L1 mice after injection with CT26 cells. FIG. 31C exemplifies the mean tumor volume from non-treated, Ibrutinib alone, anti-PD-L1 alone, and Ibrutinib+anti-PD-L1 mice after injection with CT26 cells. FIG. 31D exemplifies the percent surivival from non-treated, Ibrutinib alone, anti-PD-L1 alone, and Ibrutinib+anti-PD-L1 mice after injection with CT26 cells.

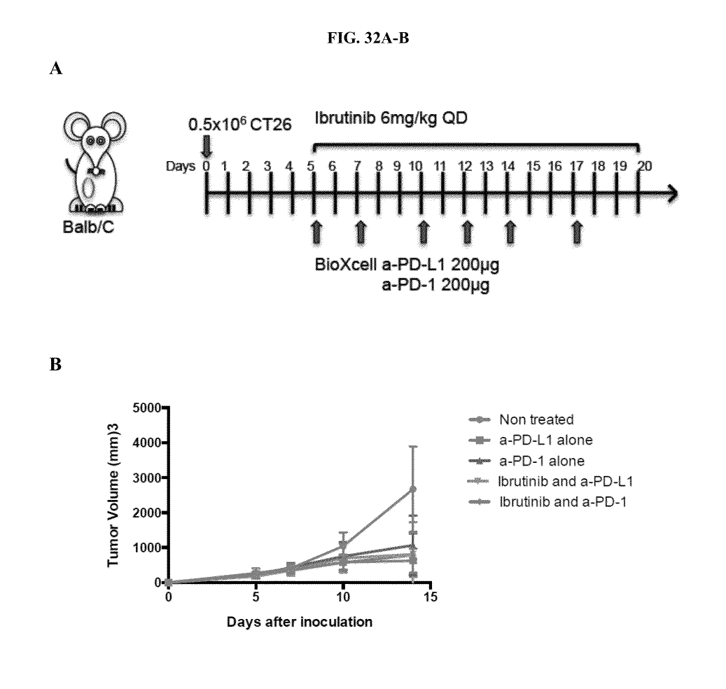

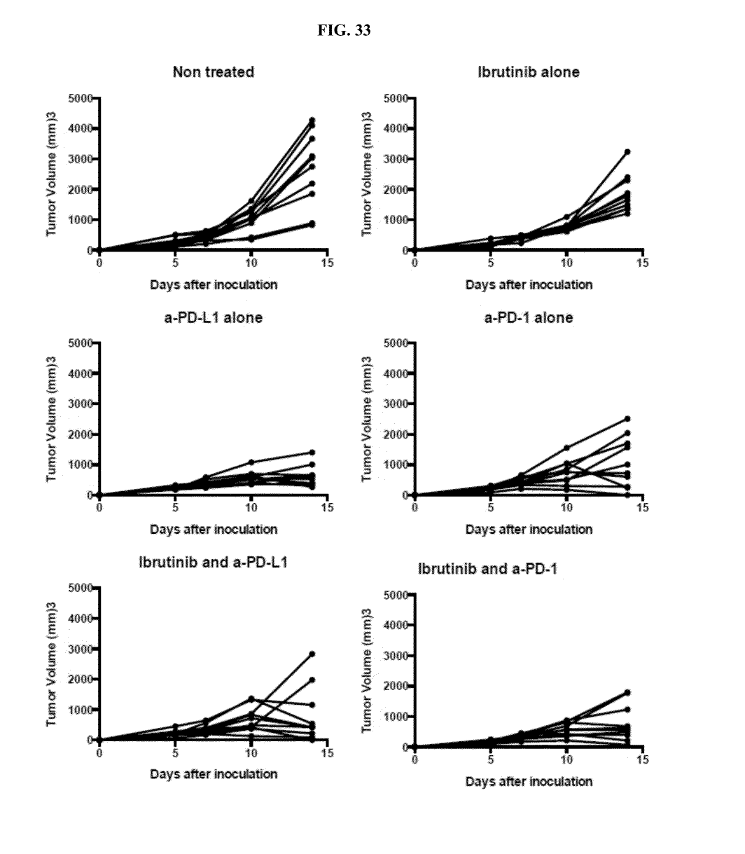

FIG. 32 and FIG. 14 exemplify a third set of experiment using the CT26 colon cancer model. FIG. 32A exemplifies an ibrutinib and anti-PD-L1 antibody administration schedule in a mouse model injected with CT26 (0.5×10⁶) cells into the mammary fat pad of the mouse. Ibrutinib was administered at 6 mg/kg on days 5-20 post injection of CT26 cells. Anti-PD-L1 (200 μg) and anti-PD-1 (200 μg) were administered on days 5, 7, 10, 12, 14, and 17 post-injection of CT26 cells. The CT26 cell line is not sensitive to ibrutinib. FIG. 32B exemplifies the mean tumor volume from non-treated, anti-PD-1 alone, anti-PD-L1 alone, Ibrutinib+anti-PD-L1, and ibrutinib+anti-PD-1 mice after injection with CT26 cells. FIG. 33 exemplifies the tumor volume from non-treated, ibrutinib alone, anti-PD-1 alone, anti-PD-L1 alone, Ibrutinib+anti-PD-L1, and ibrutinib+anti-PD-1 mice after injection with CT26 cells.

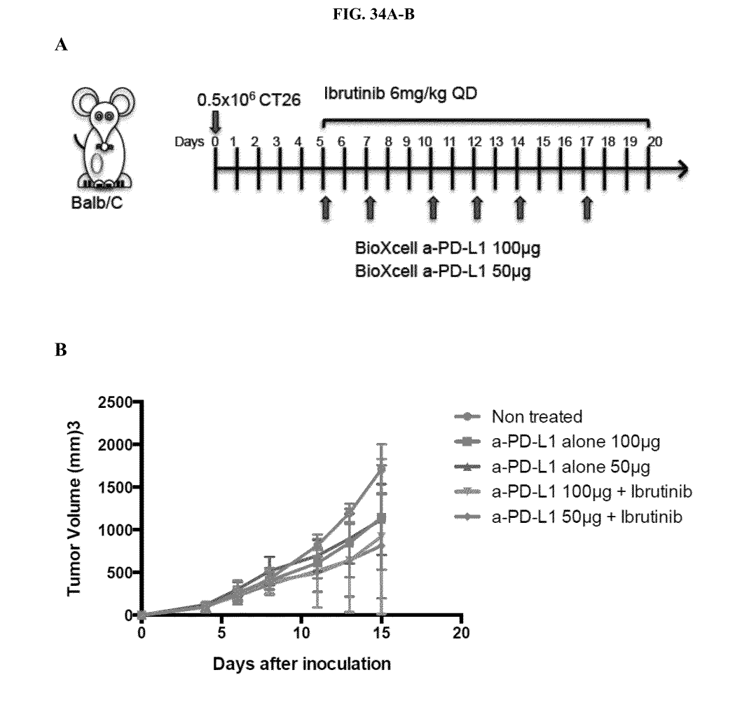

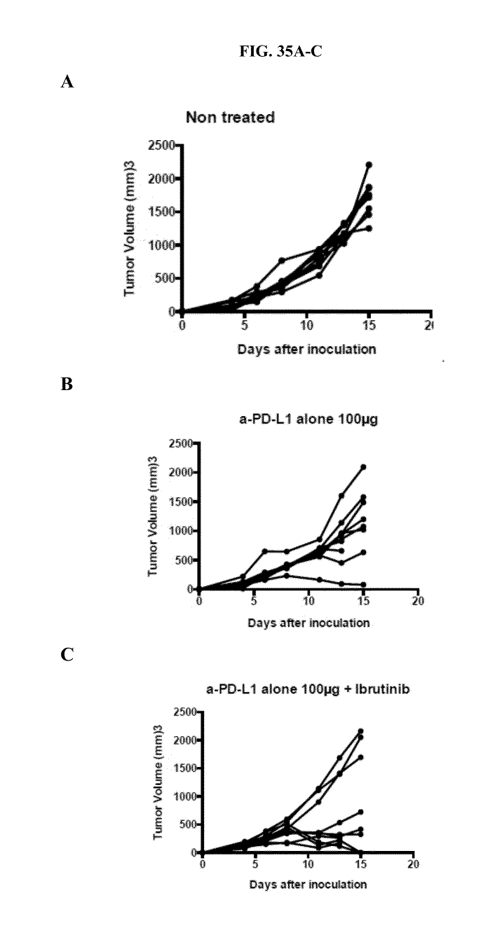

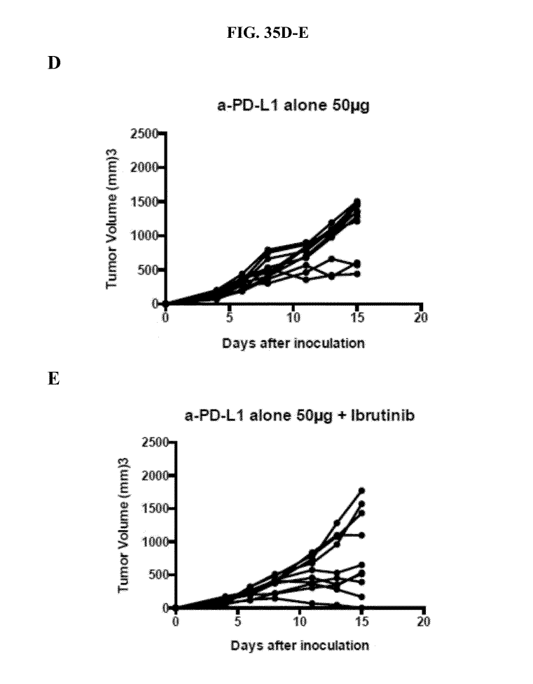

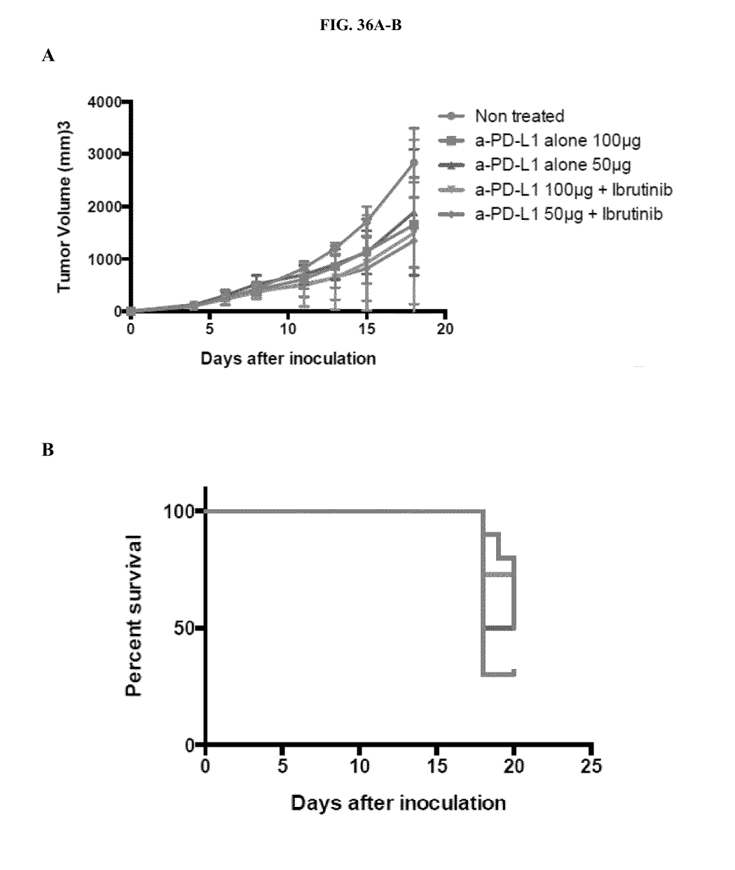

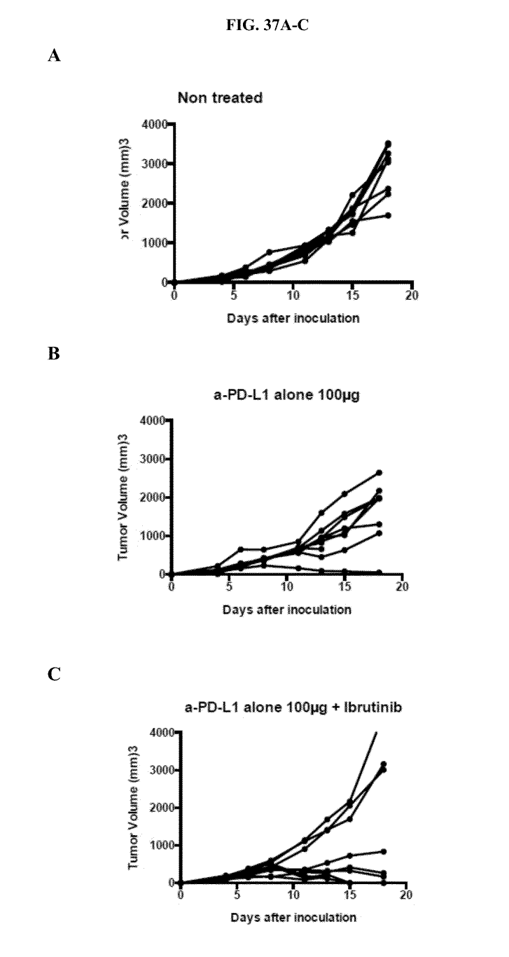

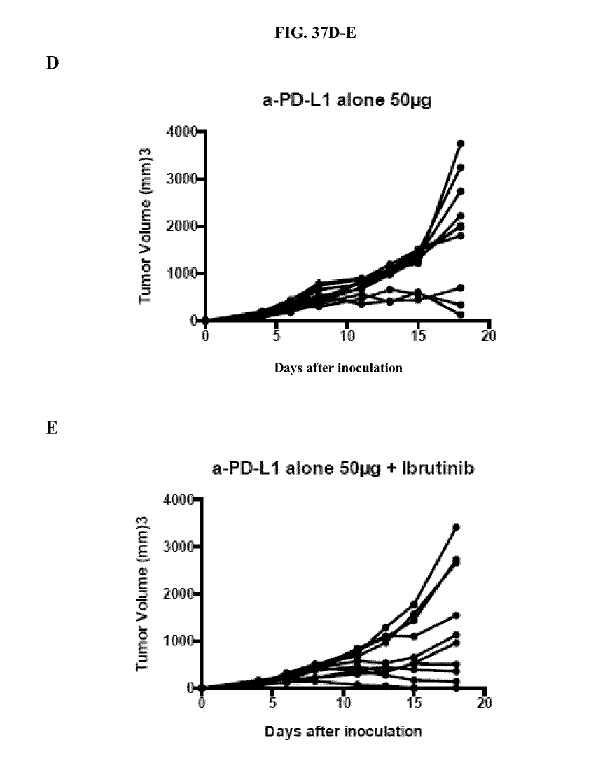

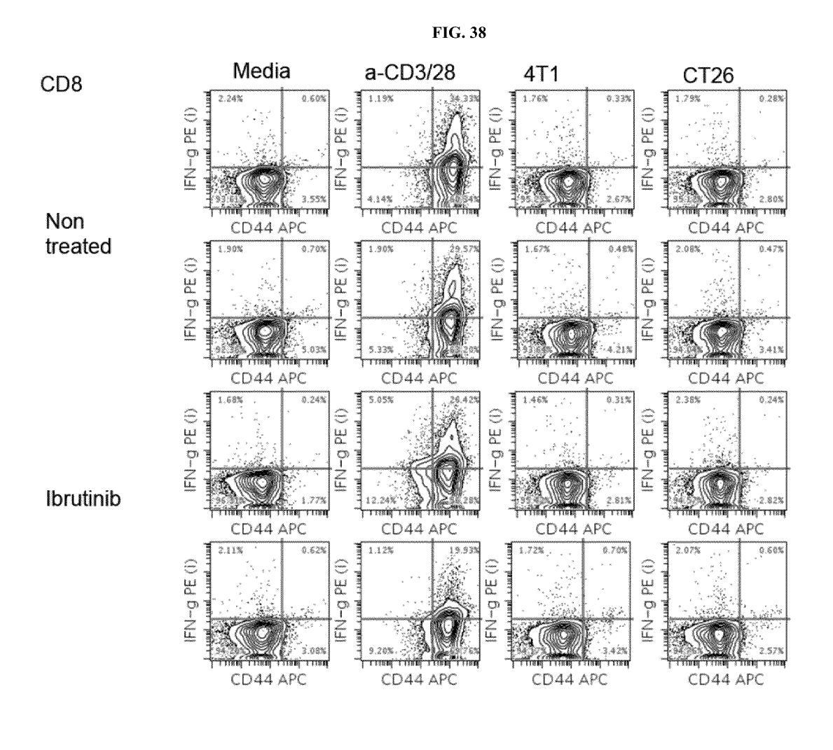

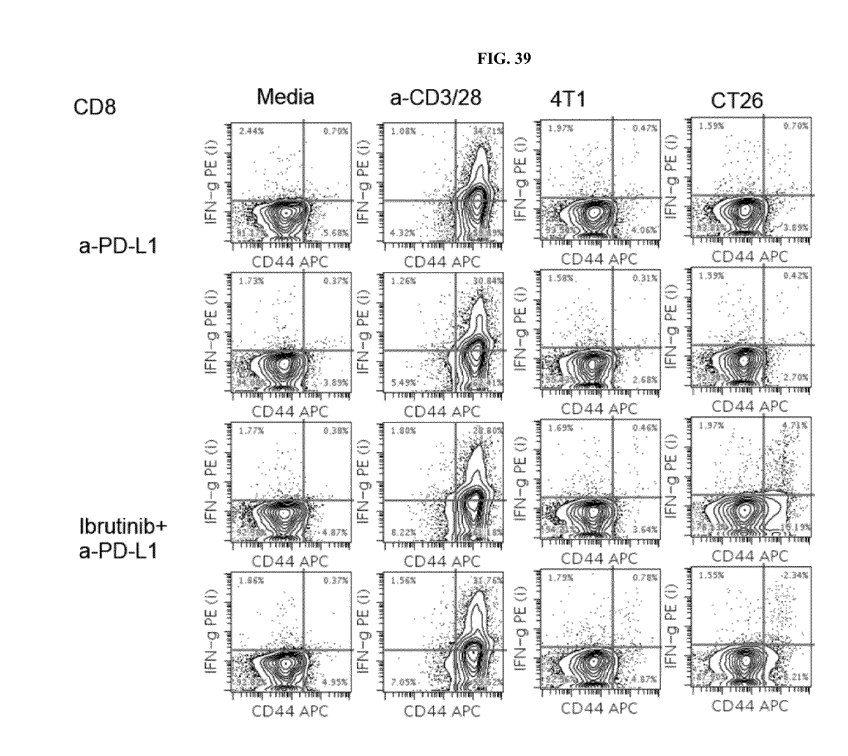

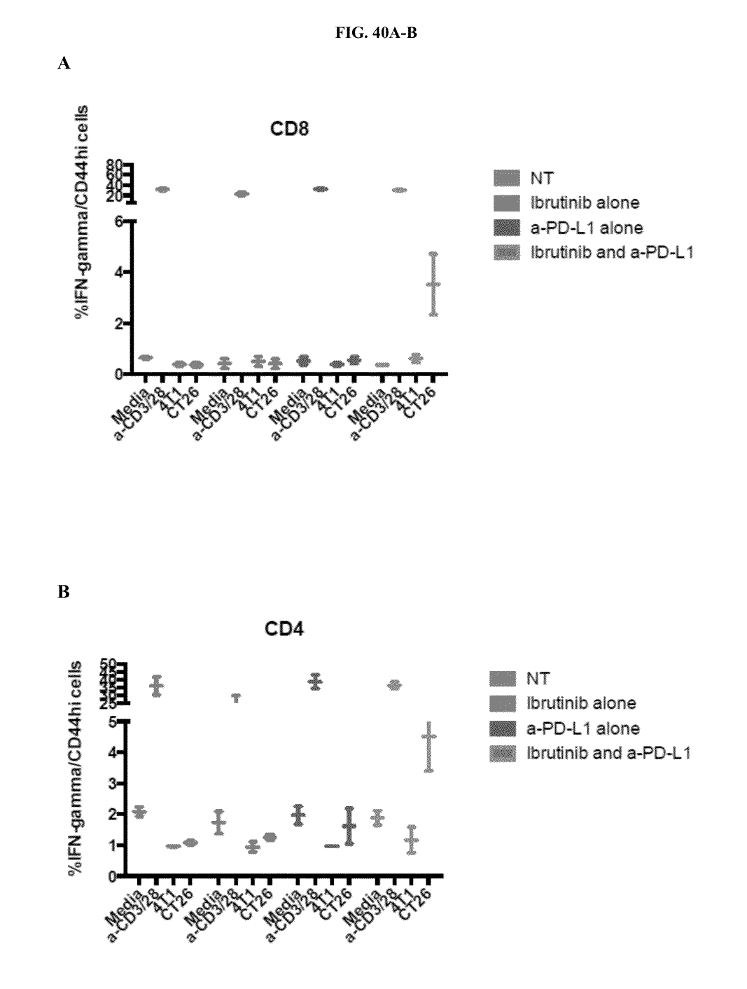

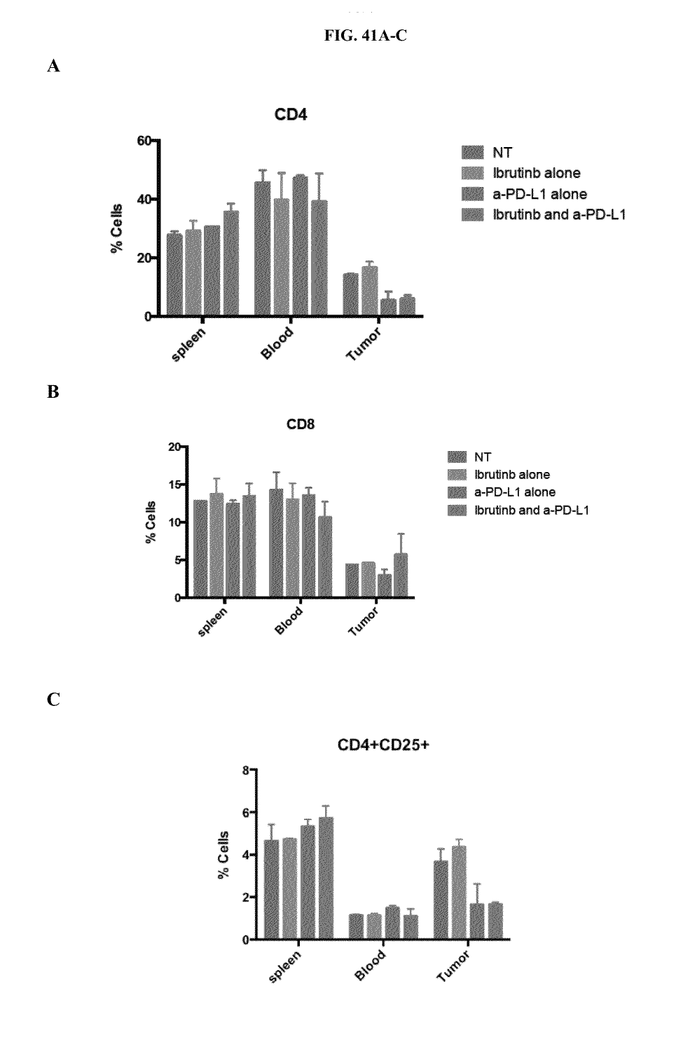

FIG. 34-FIG. 41 exemplify a fourth set of experiment using the CT26 colon cancer model. FIG. 34A exemplifies an ibrutinib and anti-PD-L1 antibody administration schedule in a mouse model injected with CT26 (0.5×10⁶) cells into the mammary fat pad of the mouse. Ibrutinib was administered at 6 mg/kg on days 5-20 post injection of CT26 cells. Anti-PD-L1 (100 μg or 50 μg) was administered on days 5, 7, 10, 12, 14, and 17 post-injection of CT26 cells. The CT26 cell line is not sensitive to ibrutinib. FIG. 34B exemplifies the mean tumor volume from non-treated, anti-PD-L1 alone at 100 μg, anti-PD-L1 alone at 50 μg, Ibrutinib+anti-PD-L1 (100 μg), and ibrutinib+anti-PD-L1 (50 μg) mice after injection with CT26 cells. FIG. 35A-35E exemplify the tumor volume from non-treated, anti-PD-L1 alone at 100 μg, anti-PD-L1 alone at 50 μg, Ibrutinib+anti-PD-L1 (100 μg), and ibrutinib+anti-PD-L1 (50 μg) mice after injection with CT26 cells. FIG. 36A exemplifies the mean tumor volume from non-treated, anti-PD-L1 alone at 100 μg, anti-PD-L1 alone at 50 μg, Ibrutinib+anti-PD-L1 (100 μg), and ibrutinib+anti-PD-L1 (50 μg) mice after injection with CT26 cells. FIG. 36B exemplifies the percent survival from non-treated, anti-PD-L1 alone at 100 μg, anti-PD-L1 alone at 50 μg, Ibrutinib+anti-PD-L1 (100 μg), and ibrutinib+anti-PD-L1 (50 μg) mice after injection with CT26 cells. FIG. 37A-37E exemplify exemplifies the tumor volume from non-treated, anti-PD-L1 alone at 100 μg, anti-PD-L1 alone at 50 μg, Ibrutinib+anti-PD-L1 (100 μg), and ibrutinib+anti-PD-L1 (50 μg) mice after injection with CT26 cells. FIG. 38 illustrates the flow cytometry analysis of CD8+ T cells with ibrutinib. Cells were either non treated or pretreated with ibrutinib and were stimulated (or unstimulated) with anti-CD3/anti-CD28. Percentages are represented in each quadrant. FIG. 39 illustrates the flow cytometry analysis of CD8+ T cells with anti-PD-L1 alone or ibrutinib+anti-PD-L1. Cells were either pretreated with anti-PD-L1 alone or with ibrutinib+anti-PD-L1 and were stimulated (or unstimulated) with anti-CD3/anti-CD28. Percentages are represented in each quadrant. FIGS. 40A and 40B illustrate IFN-γ-expressing T_{eff} cells analysis with non-treated, Ibrutinib alone, anti-PD-L1 alone, and Ibrutinib+anti-PD-L1 in CD8 and CD4 T cells. FIG. 41A-41C illustrate the percentage of antigen specific T cells from treatment with non-treated, Ibrutinib alone, anti-PD-L1 alone, and Ibrutinib+anti-PD-L1 in CD8, CD4 and CD4+/CD25+ T cells in spleen, blood, and tumor.

A set of four experiments were carried out on CT26 colon cancer model. The combination of anti-PD1 and ibrutinib inhibited CT26 tumor growth but this inhibition was not as potent as the combination of anti-PD-L1+ibrutinib. In addition, the combination therapies increased antigen specific T cells that express interferon-gamma which is important for tumor cell killing.

### Example 9

### Pilot Scale Study for Evaluating Tumor Growth in a CT26 Mouse Model

### Materials

Cell line CT26 (ATCC CRL-2638, Lot: 61559123) is a N-nitroso-N-methylurethane induced, undifferentiated colon carcinoma cell line from a BALB/c. CT26 expresses H-2d antigens. CT26 cells were cultured in complete Roswell Park Memorial Institute 1640 medium (cRPMI; Invitrogen) containing 10% fetal bovine serum (FBS; Thermo Scientific), 100 U/mL penicillin, 100 μg/mL streptomycin (both from Invitrogen). The cells were split into four T75 (75 cm²) flasks, and grown to about 80% confluence. Next, cells were further passaged into two T175 (175 cm²) flasks, and grown to about 100% confluence. Further, cells were counted and split into four T75 flasks, grown to about 80% confluence, and nineteen vials of frozen cells were prepared from the cultures. The remaining cells were split at one-fourth dilution into T175 flasks. Once the cells were ready, they were trypsinized, washed thrice with RPMI-1640, and resuspended in RPMI-1640 at three different concentrations of 1 million cells/mL, 5 million cells/mL, and 10 million cells/mL.

### Tumor Transplantation

BALB/c mice were injected in the right hind thigh (5 mice) and in the back (5 mice) with 5 million cells from the CT26 colon carcinoma cell line. Tumors were visible after 8 days and the tumor volume was measured to be about 100 mm³. No difference was detected between the size of tumors on the right hind thigh and those on the back. Tumor volumes were re-measured at day 10 and day 12. The results are listed in Table 1.

TABLE 1
Tumor volumes in pilot scale study

| Tumor Cell | | | | |
|---|---|---|---|---|
| Injection | Mouse | Tumor Volume (mm³) | | |
| Site | No. | Day 8 | Day 10 | Day 12 |
| Thigh | 1 | 49.7 | 82.1 | 133.5 |
| | 2 | 35.6 | 118.9 | 194.4 |
| | 3 | 164.1 | 298.1 | 572.4 |
| | 4 | 88.4 | 110.7 | 200 |
| | 5 | 63.9 | 78.2 | 245.2 |
| Back | 1 | 105.5 | 106 | 103.9 |
| | 2 | 77.7 | 101.6 | 214.7 |
| | 3 | 97.4 | 145.3 | 269.8 |
| | 4 | 50.2 | 59.3 | 102.8 |
| | 5 | 78.8 | 104.3 | 205.7 |

### Example 10

### Large Scale Study to Determine Optimal Inoculum of CT26 Cells for Tumor Growth

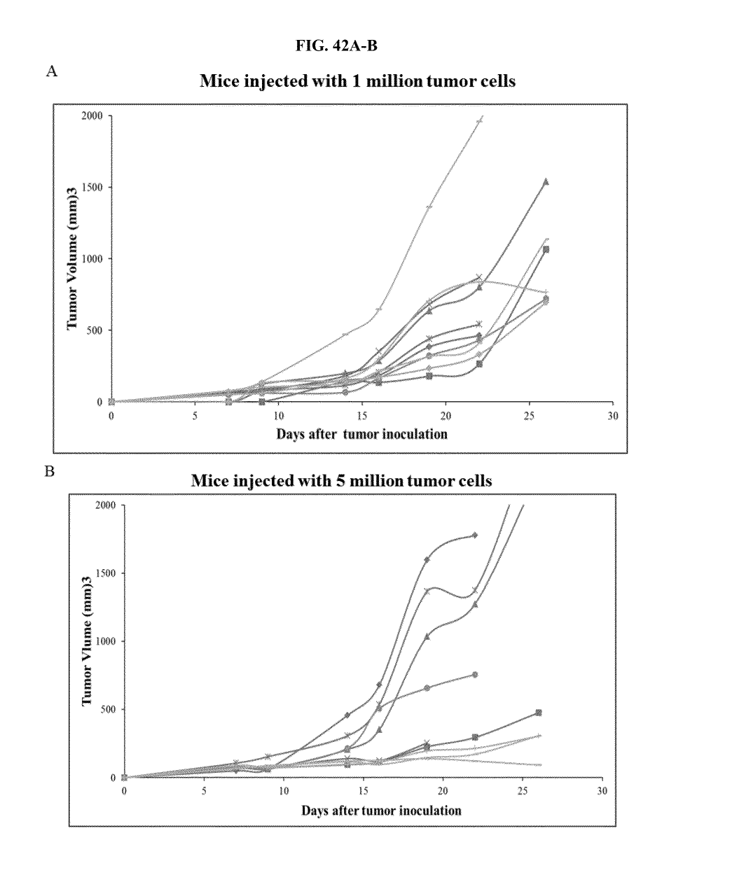

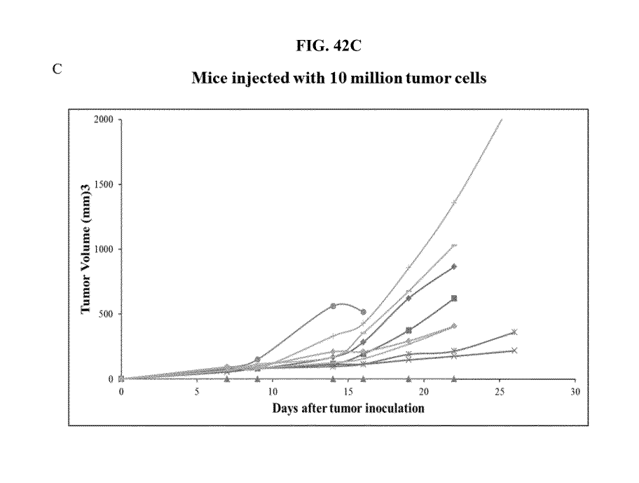

Three groups of BALB/c mice were injected in the right hind thigh with 1 million, 5 million, and 10 million cells from the CT26 colon carcinoma cell line. The CT26 cells were prepared according to the methods described in Example 9. Tumor volumes were measured 7, 9, 14, 16, 19, 22, and 26 days after injection. The results are illustrated in Tables 2-4 and FIG. 42A-C.

After seven days post injection, three out of ten mice in Group I did not show visible tumor (Table, 2 and FIG. 42A), whereas all of the nine mice in Group II (Table 3 and FIG. 42B) and nine out of ten mice in Group III (Table 4 and FIG. 42C) showed visible tumors. Although the average tumor size was similar for the Groups II and III mice, the higher dose resulted in necrosis, day 16 onwards, in several mice belonging to Group III (Table 4). Based on the above observations, 5 million CT26 cells were selected as the optimal inoculum for the subsequent experiments.

TABLE 2
Tumor volumes in mice injected with
1 million CT26 tumor cells (Group I)

| | Tumor Volume (mm)³ | | | | | | | |
|---|---|---|---|---|---|---|---|---|
| Mouse | Day | Day | Day | Day | Day | | | |
| No. | 0 | 7 | 9 | 14 | 16 | Day 19 | Day 22 | Day 26 |
| 1 | 0 | 66.6 | 89.3 | 150.4 | 185.0 | 383.4 | 462.9 | ** |
| 2 | 0 | 0.0 | 0.0 | 141.1 | 135.5 | 180.1 | 265.4 | 1064.0 |
| 3 | 0 | 66.6 | 125.8 | 199.6 | 287.8 | 636.6 | 802.0 | 1539.0 |
| 4 | 0 | 59.5 | 78.7 | 183.3 | 351.7 | 681.0 | 868.8 | ** |
| 5 | 0 | 0.0 | 75.3 | 114.7 | 207.2 | 439.1 | 541.2 | ** |
| 6 | 0 | 50.0 | 60.7 | 66.5 | 166.5 | 321.6 | 432.5 | 719.4 |
| 7 | 0 | 53.0 | 60.6 | 164.1 | 299.2 | 707.3 | 838.3 | 763.9 |
| 8 | 0 | 78.0 | 103.0 | 129.3 | 212.6 | 316.8 | 411.6 | 1136.1 |
| 9 | 0 | 78.0 | 140.6 | 470.1 | 645.7 | 1360.2 | 1956.6 | 2781.2 |
| 10 | 0 | 0.0 | 131.3 | 146.1 | 172.5 | 233.6 | 331.4 | 694.3 |
| Mean | 0 | 45.2 | 86.5 | 176.5 | 266.4 | 526.0 | 691.1 | |
| Tumor | | | | | | | | |
| Volume | | | | | | | | |
| (mm)³ | | | | | | | | |
| SEM | | 10.3 | 13.2 | 34.6 | 47.3 | 109.7 | 156.2 | |

**: Mice were sacrificed due to necrosis.

TABLE 3
Tumor volumes in mice injected with
5 million CT26 tumor cells (Group II)

| | Tumor Volume (mm)³ | | | | | | | |
|---|---|---|---|---|---|---|---|---|
| Mouse | Day | Day | Day | Day | Day | | | |
| No. | 0 | 7 | 9 | 14 | 16 | Day 19 | Day 22 | Day 26 |
| 1 | 0 | 50.0 | 61.7 | 457.1 | 679.2 | 1597.9 | 1778.6 | ** |
| 2 | 0 | 66.6 | 71.5 | 95.3 | 113.0 | 225.7 | 294.7 | 476.3 |
| 3 | 0 | 70.2 | 63.0 | 206.3 | 352.1 | 1034.3 | 1272.4 | 2239.2 |
| 4 | 0 | 64.9 | 72.6 | 139.2 | 122.9 | 254.3 | | ## |
| 5 | 0 | 106.1 | 153.2 | 306.3 | 535.8 | 1365.7 | 1374.3 | 2628.5 |
| 6 | 0 | 78.0 | 68.5 | 215.2 | 507.7 | 656.7 | 756.5 | ** |
| 7 | 0 | 89.3 | 75.3 | 98.2 | 112.2 | 196.4 | 214.7 | 304.2 |
| 8 | 0 | 66.6 | 83.4 | 116.6 | 95.0 | 147.0 | 170.9 | 307.3 |
| 9 | 0 | 67.4 | 87.7 | 121.7 | 125.5 | 139.0 | 121.7 | 92.3 |
| Mean | 0 | 73.2 | 81.9 | 195.1 | 293.7 | 624.1 | | |
| Tumor | | | | | | | | |
| Volume | | | | | | | | |
| (mm)³ | | | | | | | | |
| SEM | | 5.4 | 9.4 | 40.1 | 76.3 | 190.4 | | |

**: Mice were sacrificed due to necrosis.
##: Mice were sacrificed due to mechanical problems.

TABLE 4
Tumor volumes in mice injected with
10 million CT26 tumor cells (Group III)

| | Tumor Volume (mm)³ | | | | | | | |
|---|---|---|---|---|---|---|---|---|
| Mouse | Day | Day | Day | Day | Day | | | |
| No. | 0 | 7 | 9 | 14 | 16 | Day 19 | Day 22 | Day 26 |
| 1 | 0 | 72.6 | 80.6 | 168.8 | 285.1 | 622.8 | 865.1 | ** |
| 2 | 0 | 80.7 | 83.2 | 118.1 | 193.6 | 376.0 | 621.0 | ** |
| 3 | 0 | 0.0 | 0.0 | 0.0 | 0.0 | 0.0 | 0.0 | |
| 4 | 0 | 55.0 | 80.6 | 111.5 | 114.9 | 147.2 | 175.5 | 217.8 |
| 5 | 0 | 67.1 | 82.3 | 96.9 | 115.3 | 189.7 | 215.0 | 360.5 |
| 6 | 0 | 88.3 | 150.8 | 563.3 | 517.7 | | | ** |
| 7 | 0 | 89.3 | 77.4 | 330.5 | 429.1 | 855.8 | 1356.3 | 2178.0 |
| 8 | 0 | 86.4 | 86.4 | 125.8 | 157.7 | 269.5 | 403.8 | ** |
| 9 | 0 | 67.3 | 114.2 | 170.7 | 355.0 | 679.0 | 1031.3 | ** |
| 10 | 0 | 93.6 | 97.9 | 209.7 | 211.7 | 292.9 | 407.7 | ** |
| Mean | 0 | 77.8 | 94.8 | 210.6 | 264.5 | | | |
| Tumor | | | | | | | | |
| Volume | | | | | | | | |
| (mm)³ | | | | | | | | |
| SEM | | 4.3 | 8.0 | 50.1 | 47.7 | | | |

**: Mice were sacrificed due to necrosis.

### Example 11

### Combination Therapy of Ibrutinib with an Anti PDL-1 Antibody or an Anti-CTLA-4 Antibody in a Mouse Model

### Materials

Rat IgG2b/k anti-mouse PD-L1 (clone 10F.9G2, Bio X Cell, cat #BE0101, Lot: 5360/0814, 6.15 mg/mL) was obtained from BioXcell, and diluted to 2 mg/mL in sterile Dulbecco's Phosphate Buffered Saline (DPBS). Mouse IgG2b anti-mouse CTLA-4 (clone 9D9, Bio X Cell, cat #BE 0164, Lot: 5159/0614, 6.43 mg/mL) was obtained from BioXcell, and diluted to 1 mg/mL in sterile DPBS. Rat IgG2b/k antibody (clone LTF-2, Bio X Cell, cat #BE0090, Lot: 5101-4/0714, 8.34 mg/mL) was obtained from BioXcell, and diluted to 2 mg/mL in sterile DPBS. Mouse IgG2b (clone MPC-11, Bio X Cell, cat #BE0086, Lot: 4700-2/0414, 8.71 mg/mL) was obtained from BioXcell, and diluted to 2 mg/mL in sterile DPBS. LTF-2 and MPC-11 clones were mixed at 1:1 ratio to produce the IgG controls.

Ibrutinib (PCI-32765) was prepared at a concentration of 4.8 mg/mL in 0.5% methyl cellulose and 0.1% sodium lauryl sulfate (SLS).

### Tumor Transplantation and Treatment

A total of 140 mice were injected with 5 million CT26 tumor cells, prepared according to the methods described in Example 9, in the right hind thigh. The tumored mice were divided into 11 groups and the animals were administered with either ibrutinib (PCI-32765) alone, daily at a dose of 24 mg/kg, using 100 μl of RPMI media as vehicle, or ibrutinib, at the same daily dose, together with either anti-PD-L1 antibody (200 μg), anti-CTLA-4 antibody (100 μg), or IgGs (200 μg), in various combinations, as listed in Table 5. In certain groups, a combination of both anti-PD-L1 antibody and anti-CTLA-4 antibody were administered, each at a dose of 100 μg. Ibrutinib was given to the mice via oral lavage and the antibodies were injected intraperitoneally (i.p.). The treatment timelines were designed such that two different dosage schedules were followed for animals receiving the combination therapy. The animals belonging to Schedule 1 groups (Groups 4, 6, and 8) were administered with ibrutinib in combination with the antibodies from the first day of treatment, whereas those belonging to the Schedule 2 groups (Groups 5, 7, 9, and 11) were administered with antibodies only for the first four days and ibrutinib dosing was started on the fifth day.

TABLE 5
Treatment Groups

| Group | |
|---|---|
| No. | Treatment |
| 1 | Vehicle + IgG |
| 2 | Vehicle + anti-PD-L1 antibody |
| 3 | Vehicle + anti-CTLA-4 antibody |
| 4 | Ibrutinib + IgG (Schedule 1) |
| 5 | Ibrutinib + IgG (Schedule 2) |
| 6 | Ibrutinib + anti-PD-L1 (Schedule 1) |
| 7 | Ibrutinib + anti-PD-L1 (Schedule 2) |
| 8 | Ibrutinib + anti-CTLA-4 (Schedule 1) |
| 9 | Ibrutinib + anti-CTLA-4 (Schedule 2) |
| 10 | Ibrutinib + anti-PD-L1 + aCTLA4 |
| 11 | Ibrutinib + anti-PD-L1 + anti-CTLA-4 (Schedule 2) |

### Results

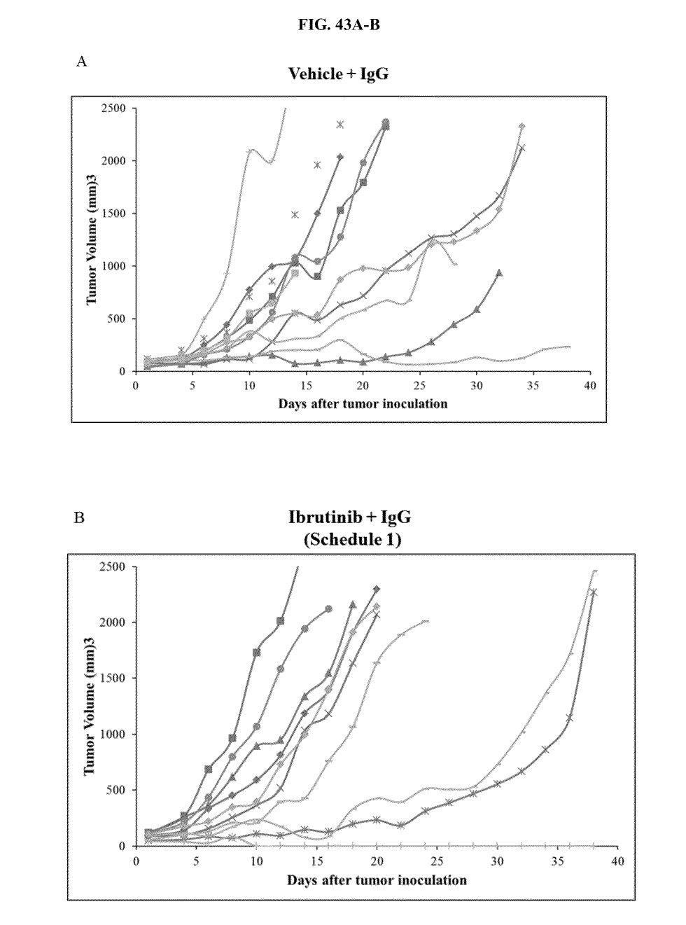

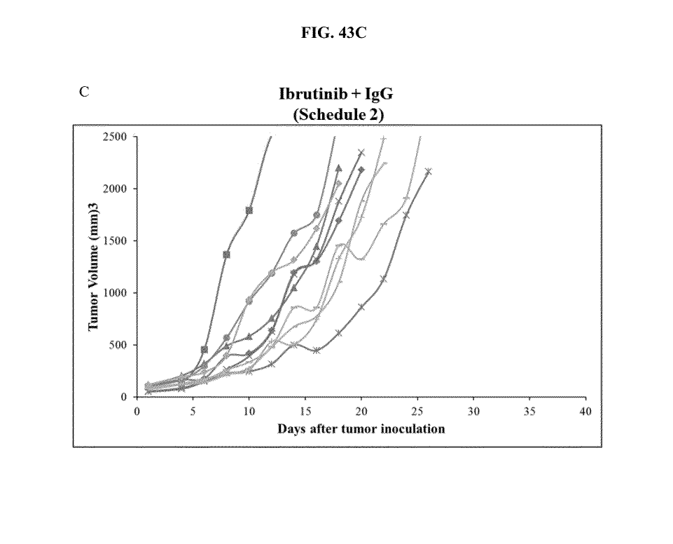

Tumor volumes were measured periodically for 40 days. Treatment with IgG alone resulted in slow growing tumors (FIG. 43A). The combination of ibrutinib and IgG controls reduced the tumor burden, when ibrutinib and IgG were administered concurrently (Schedule 1) as exemplified in FIG. 43B, but the effect was reversed with delayed dosing of ibrutinib (Schedule 2), which also resulted in slow growing tumors, as exemplified in FIG. 43C.

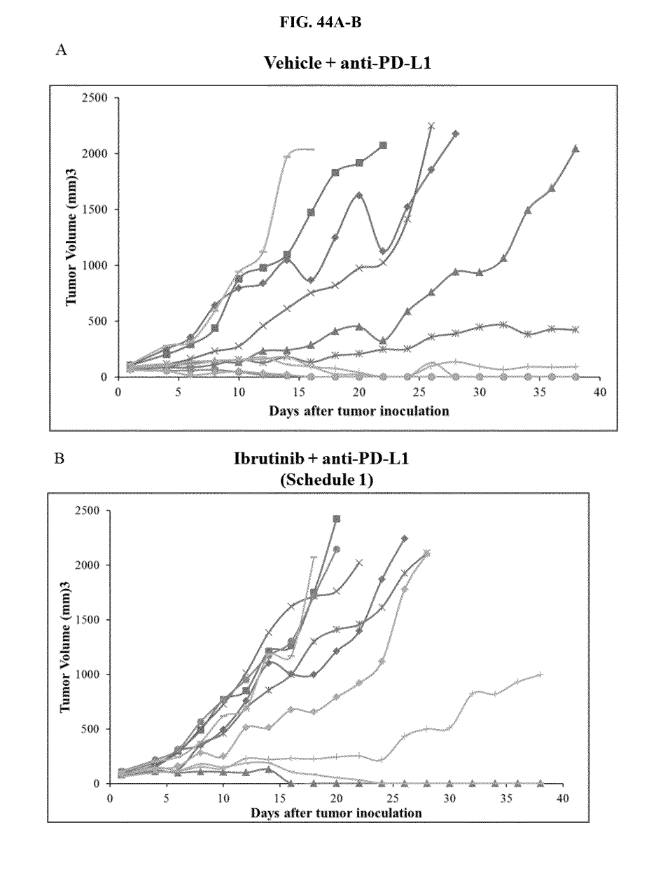

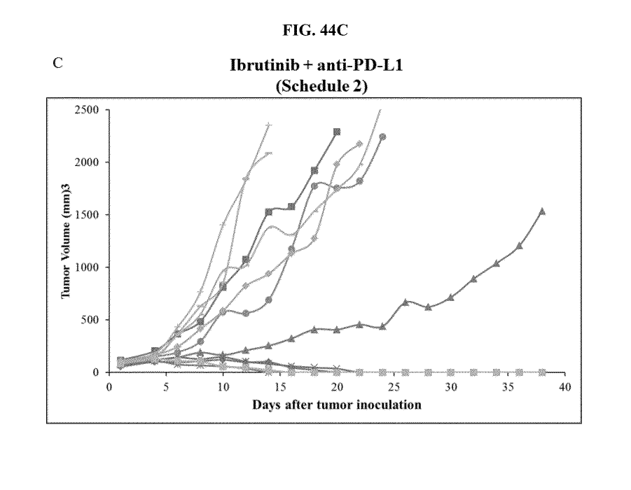

Treatment with anti-PD-L1 alone resulted in slow progression of tumors (FIG. 44A). Out of the eleven mice treated with anti-PD-L1 alone (FIG. 44A), three mice showed complete regression. The concurrent administration of ibrutinib and anti-PD-L1, following Schedule 1, antagonized the effect of anti-PD-L1, as exemplified in FIG. 44B. The antagonistic effect was much less pronounced, as shown in FIG. 44C, when Schedule 2 was followed, where ibrutinib dose was delayed and started only after 4 days of treatment with anti-PDL-1 antibody alone.

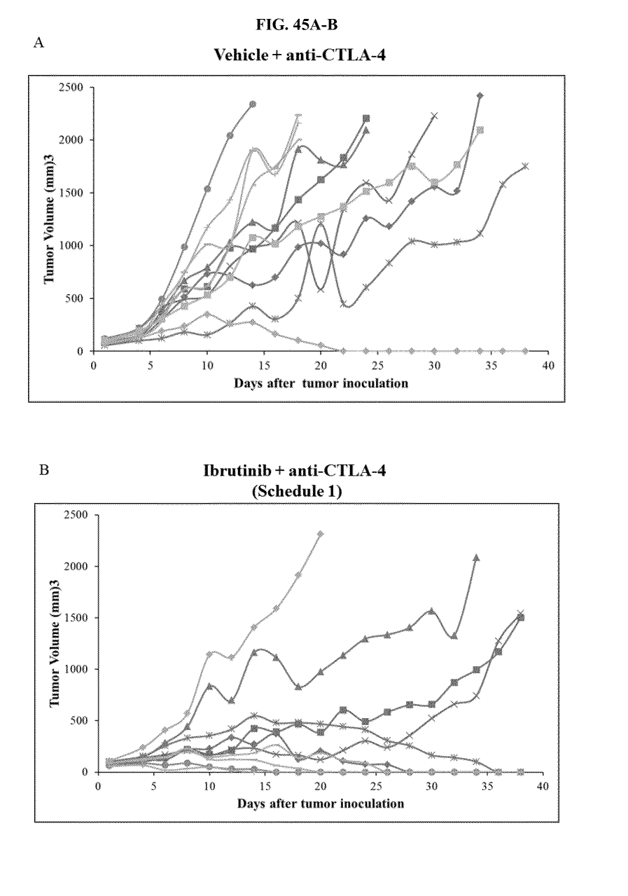

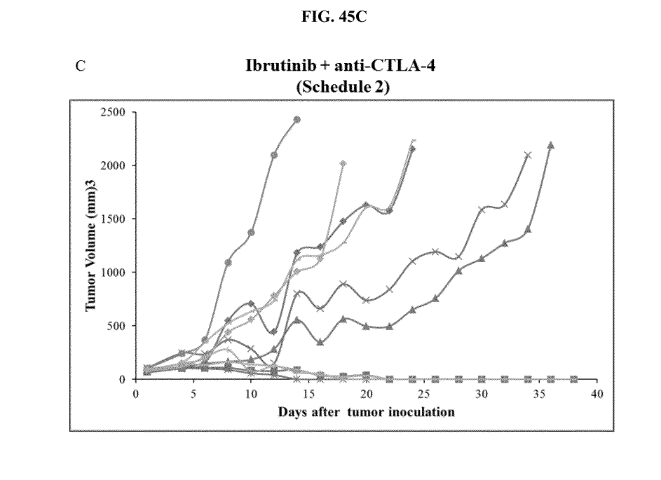

Treatment with anti-CTLA-4 antibody alone caused faster growth of tumors (FIG. 45A), but the effect was reversed by concurrent treatment with ibrutinib (FIG. 45B). Thus, combination therapy with concurrent administration of ibrutinib and anti-CTL-4 antibody, following Schedule 1, produced an apparent synergistic or additive effect. This effect was not observed when ibrutinib dosing was delayed, following Schedule 2, as exemplified in FIG. 45C.

Ten mice were treated with 100 μg each of anti-PD-L1 and anti-CTLA-4 antibodies (FIG. 46A). Out of the ten treated mice, three mice showed complete regression and two mice had tumor volume less than 200 mm³. Similarly, ten mice were treated with 100 ug each of anti-PD-L1 and anti-CTLA-4 antibodies in combination with ibrutinib (FIG. 46B). Out of the ten treated mice, three mice showed complete regression and two mice had tumor volumes less than 200 mm³.

### Example 12

### Combination Therapy of Ibrutinib and Anti-CTLA-4 Antibody in a Mouse Model

### Tumor Transplantation and Treatment

BALB/c mice were implanted subcutaneously with 5 million CT26 tumor cells of day 0. Treatment with either the IgG control or anti CTLA-4 antibody alone or in combination with ibrutinib was started on day 7, when the tumor volumes reached about 85 mm³. Ibrutinib, at a dose of 24 mg/kg, was administered daily via oral gavage. The IgG control or anti-CTLA-4 antibody (αCTLA-4) was injected, intraperitoneally, every other day for the entire duration of treatment. Mice with tumors whose size reached 2000 mm³ or above were euthanized.

### Results

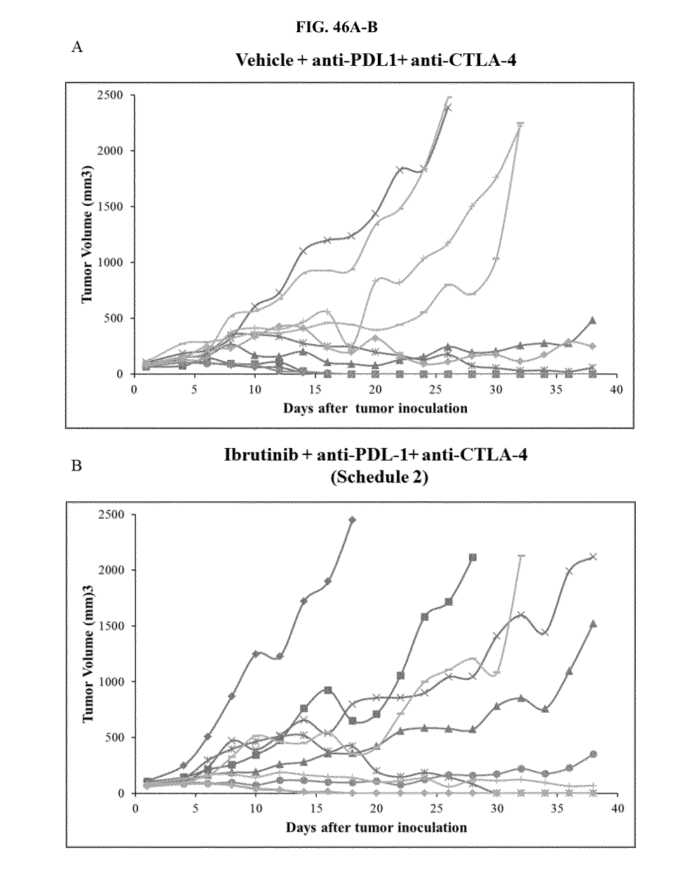

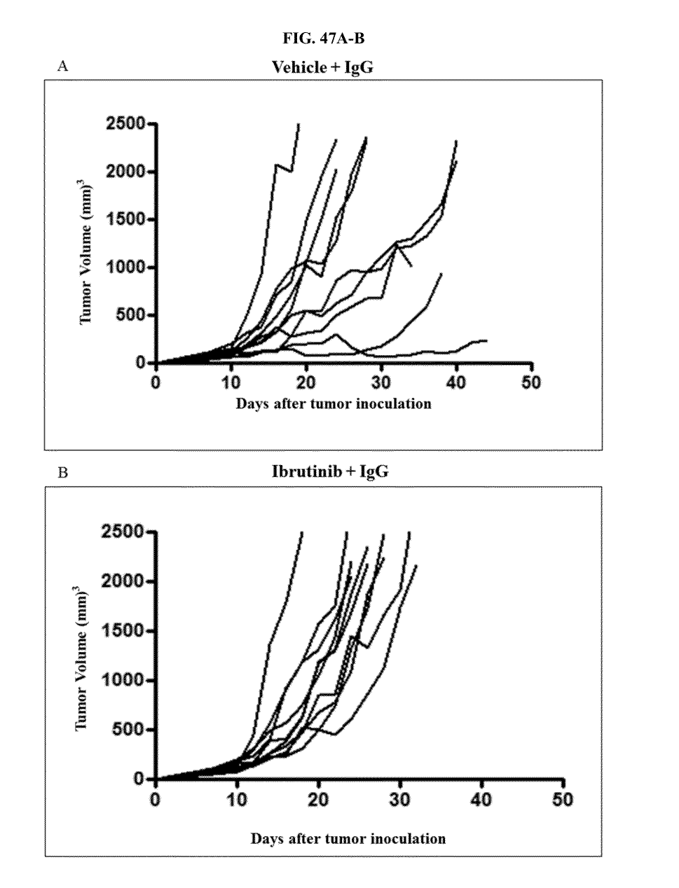

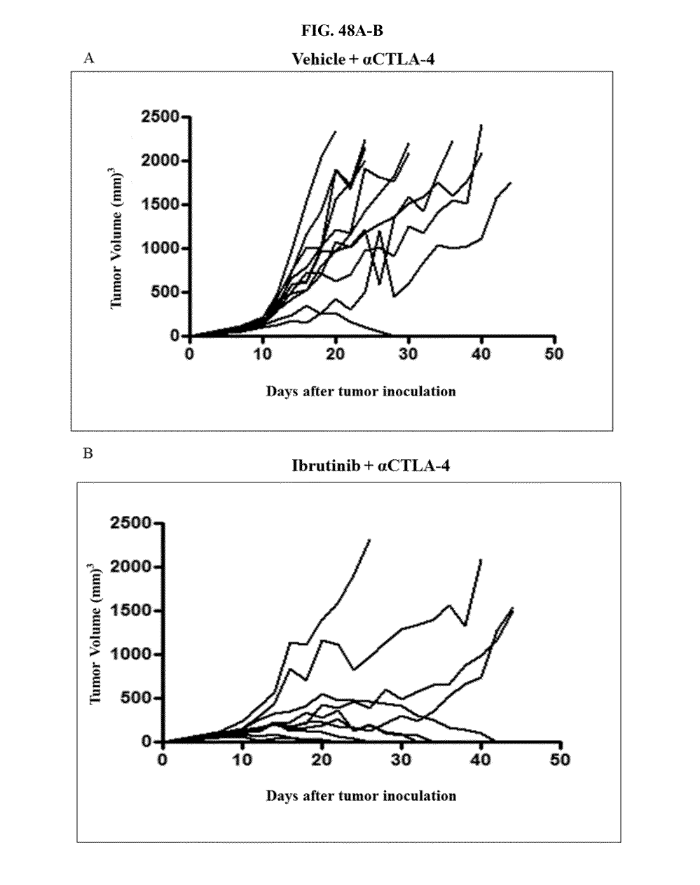

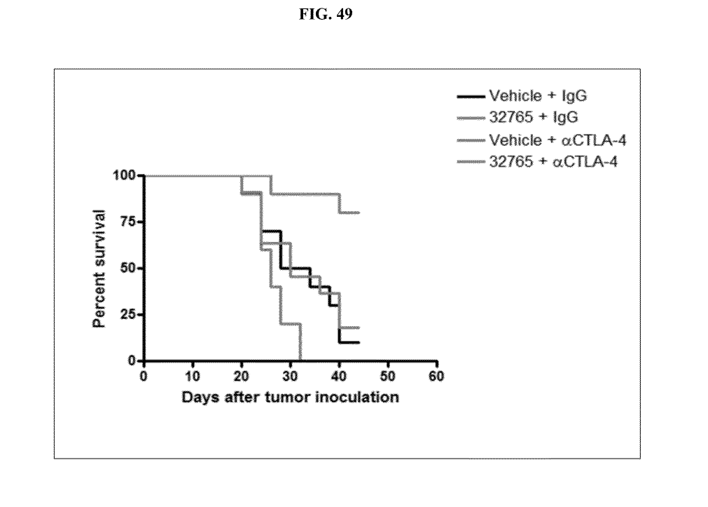

Tumor growth was not suppressed in mice treated with IgG alone or in combination with Ibrutinib (FIGS. 47A,B). Mice treated with αCTLA-4 antibody alone also had rapidly growing tumors (FIG. 48A), but combination therapy with αCTLA-4 and ibrutinib slowed down the growth of tumors (FIG. 48B). The mice were monitored for an extended period of time for tumor free survival. Six out of the ten mice, treated with a combination of αCTLA-4 and ibrutinib, remained tumor free after day 44, whereas only one out of the eleven mice treated with αCTLA-4 antibody alone remained tumor free after the same period of time, as exemplified in Table 6. None of the mice treated with IgG, alone or in combination with ibrutinib, were tumor free after day 44, also exemplified in Table 6. Survival rate of mice improved with treatment combining ibrutinib (PCI-32765) compared to αCTLA-4 treatment alone (FIG. 49).

TABLE 6
Tumor free mice after day 44

| Treatment | Tumor Free Mice/Total No. of Mice |
|---|---|
| Vehicle + IgG | 0/10 |
| Ibrutinib + IgG | 0/10 |
| Vehicle + αCTLA-4 | 1/11 |
| Ibrutinib + αCTLA-4 | 6/11 |

### Example 13

### Combination Therapy of Ibrutinib and Anti-CTLA-4 Antibody in an Ibrutinib-Resistant A20 Mouse Tumor Model

### Materials

Cell line A20 (ATCC TIB-208), a BALB/c B cell lymphoma line expressing MHC class I and class II H-2d molecules, was obtained from ATCC. A20 cells were cultured in complete Roswell Park Memorial Institute 1640 medium (cRPMI; Invitrogen) containing 10% fetal bovine serum (FBS; Thermo Scientific), 100 U/mL penicillin, 100 μg/mL streptomycin (both from Invitrogen), and 50 μM β-ME.

Ibrutinib (PCI-32765) was prepared at 4.8 mg/mL in 0.5% methylcellulose and 0.1% SLS.

### Tumor Transplantation and Treatment

Mice were implanted, in the back, with 5 million cells from the A20 BALB/C B-cell lymphoma cell line, which are resistant to treatment with ibrutinib. Ibrutinib was administered via oral gavage, daily at 24 mg/kg on days 5 to 17 post-injection of A20 cells. Anti-CTLA-4 antibody (e.g., Mouse IgG2b/k anti-mouse CTLA-4 (clone 9D9, Bio X Cell, cat #BE 0164, Lot: 5159/0414) was obtained from BioXcell, and diluted in sterile DPBS. The anti-CTLA-4 antibody was administered, alone or in combination with ibrutinib at the above mentioned dose, on days 5, 7, 9, 11, 13, and 15 post A20 injection.

### Results

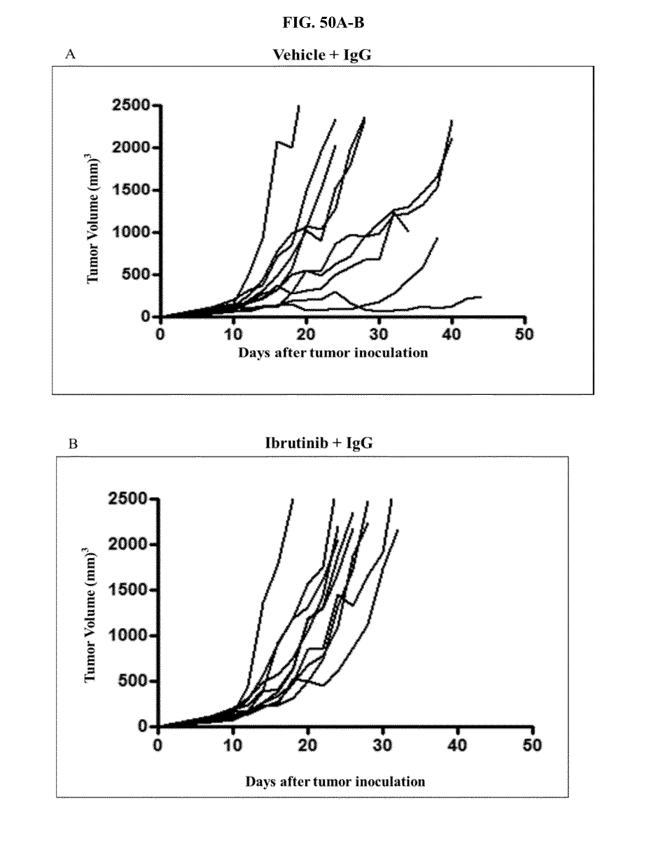

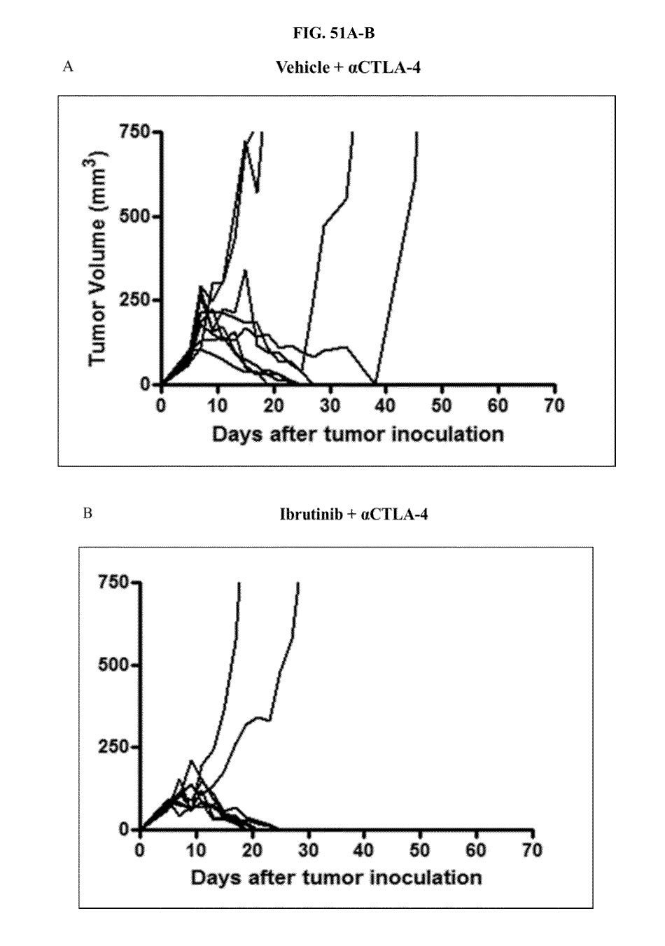

Tumor volumes were measured periodically until day 64 post injection. Tumor growth was not suppressed in mice treated with IgG alone or in combination with Ibrutinib, as exemplified in FIGS. 50A, B. Combination therapy with ibrutinib and anti-CTLA-4 antibody, in mouse injected with A20 tumor cells, produced an apparent synergistic effect and slowed down the tumor progression. The results are exemplified in FIGS. 51A, B. Seven out of the nine mice, treated with a combination of αCTLA-4 and ibrutinib, remained tumor free after day 20, and five out of the nine mice treated with αCTLA-4 antibody alone remained tumor free after the same period of time, as exemplified in Table 7. None of the mice treated with IgG, alone or in combination with ibrutinib, were tumor free after day 20, also exemplified in Table 7.

TABLE 7
Tumor free mice after day 20-A20 DLBCL model

| Treatment | Tumor Free Mice/Total No. of Mice |
|---|---|
| Vehicle + IgG | 0/9 |
| Ibrutinib + IgG | 0/9 |
| Vehicle + αCTLA-4 | 5/9 |
| Ibrutinib + αCTLA-4 | 7/9 |

### Example 14

### Tumor Specific T Cells Response in Mice Treated with a Combination of Ibrutinib and Anti-CTLA-4 Antibody

### Materials and Methods

Golden Syrian hamster IgG2/11 anti-mouse CD 28 (clone 37.51, BD 553294, NA/LE) was obtained from BD Pharmingen and prepared at a concentration of 1 mg/mL in RPMI containing 5% FBS. The Ar Hamster IgG1/k anti-mouse CD3e (clone 145-2C11, BD 553057, NA/LE) was obtained from BD Pharmingen and prepared at a concentration of 1 mg/mL in RPMI containing 5% FBS. The Fc Blocking Rat IgG2/11 anti-mouse CD 16/32 (clone 2.42G2, BD 553294, NA/LE) was obtained from BD Pharmingen and prepared at a concentration of 0.5 mg/mL.

Cell culture grade Mitomycin C (Sigme M4287) was combined with NaCl, at a ratio of 1:24, in RPMI containing 10% FBS. LIVE/DEAD Fixable Aqua Stain (Invitrogen L34957) was equilibrated to room temperature, 50 μL DMSO was added to each vial and 1 μL of the mixture was added per 1 mL of PBS. Golgistop (BD 554724, containing monensis) was obtained from BD Biosciences, and the 1× concentration was 4 μL per 6 mL of culture.

All antibodies for flow staining were anti-mouse monoclonal antibodies at 0.2 mg/mL, and 0.25 μL volume was used per sample. The AF488 conjugate was Rat IgG2b/k anti-CD4 (clone GK1.5, eBio 53-0041-82). The PE conjugates were Rat IgG2b/k anti-CD11b (clone M1/70, BD 557397), Rat (LEW) IgG2a/k anti-CD19 (clone 1D3, BD 557399), Rat (F344) IgG2a/k anti-mouse CD138 (clone 281-2, BD 561070), Rat (Lou) IgG2a/k anti-CD4 (clone H129.19, BD 553653). The PerCP-Cy5.5-conjugates were Ar Hamster IgG1/k anti-CD3e (clone 145-2C11,BD 551163), Rat (DA) IgG2a/k anti-CD4 (clone RM4-5, BD 550954). The PE-Cy7-conjugateswere Rat IgG2b/k anti-CD44 (clone IM7, BD 560569), Rat (DA) IgG2a/k anti-CD4 (clone RM4-5, BD 552775). The eFluor 660-conjugates were Rat IgG2a/k anti-ms/hu Ki-67 (clone SolA15, eBioscience 50-5698-80), Rat IgG2b/k anti-CD4 (cloneGK1.5, eBio 17-0041-81/83). The APC-Cy7 conjugates were Rat (Lou) IgG2a/k anti-CD8a (Lyt-2)(clone 53-6.7, BD 557654), Rat (LEW) IgG2b/k anti-CD4 (L3T4)(clone GK1.5, BD 552051, which blocks H129.19, RM4-5, not RM4-4). The BV421 conjugate was Rat IgG1/k anti-IFN-γ (clone XMG1.2, BD 563376). The V450 conjugate was Rat (DA) IgG2a/k anti-CD4 (clone RM4-5, BD 560468).

Single cell suspensions were made from spleens of selected mice which survived tumor free for a long term, after treatment with a combination of anti-CTLA-4 and ibrutinib, and incubated on ice overnight. The splenocyte suspension was centrifuged at 1000 rpm for 5 minutes and the red cells were lysed with ammonium chloride potassium lysis buffer (ACK lysis buffer; Life Technologies). Additionally, tumor cell suspensions (containing 15 million each of J558, A20 or 2PK3 cells) were prepared in RPMI media containing 10% FBS and pre-treated with 50 μg/mL mitomycin C for 20 minutes at 37° C. Splenocytes were then cocultured with RPMI, by plating 500,000 cells in each well of a flat bottom 96 well plate. The media was removed by centrifugation and the cells were incubated in 200 μL of either RPMI media supplemented with 5% FBS (media), media with anti-CD28 antibody, media with anti-CD28 antibody and tumor cells pretreated with mitomycin C, or media with anti-CD28 antibody and 1 μg of anti-CD3 antibody, for 48 hours. A 100 μL volume was removed from each well after 48 hours, replaced with the same volume of fresh media, and the incubation was continued for another 11 hours, after which 50 μL of fresh media containing Golgistop was added. The final incubation step, in the presence of Golgistop was for 5 hours. The cells were centrifuged to separate and remove the media, resuspended in 200 μL of PBS (or 2% FBS/PBS) and transferred to 96-well U-bottomed plates.

### Flow Cytometry

Cells stimulated following the procedure described above were incubated with 50 μL of the mouse Fc blocker antibody in fixation wash buffer (FW), washed, resuspended and surface stained with a 50 μL surface staining antibody cocktail comprising anti-CD4-AF488, anti-CD19, anti-CD11b, anti-CD138-PE, anti-CD3-PerCP Cy 5.5, anti-CD44-PE Cy7, and anti-CD8a-APC. The cells was washed and centrifuged, resuspended with 50 μl of PBS containing Aqua, and incubated on ice for 20 minutes. The cells were washed twice with FW, fixed in 1% paraformaldehyde/PBS, and stored at −4° C. The cells were subsequently thawed, washed with Permeabilization buffer/wash (PW)(ebio 00-8333-56), resuspended in wash buffer containing 1% rat serum, and incubated on ice for 10 minutes. A 50 μL volume of PW containing anti-IFN-g-BV421 and anti-Ki67 was added to the cells, incubated on ice for 30 minutes, washed twice with PW, once with PBS, and fixed with 200 μL of 1% paraformaldehyde. Compensation and Fluorescence minus one (FMO) cells were derived from naive spleens, blocked with mouse Fc blocker antibody and stained with a cocktail comprising anti-CD4, anti-CD19, anti-CD11b, anti-CD138-PE and anti-CD4-PE. The compensation cells were heat killed by incubating at 70° C. for 7 minutes for Aqua staining. The FMO samples contained 20 million splenocytes with 500,000 cells derived from either the A20 or J558 cell lines. The FMO cells were also stained with Aqua. The control and treated cells were analyzed by flow cytometry on a FACSCalibur (BD Biosciences). FACS data were analyzed using Cytobank.

### Results

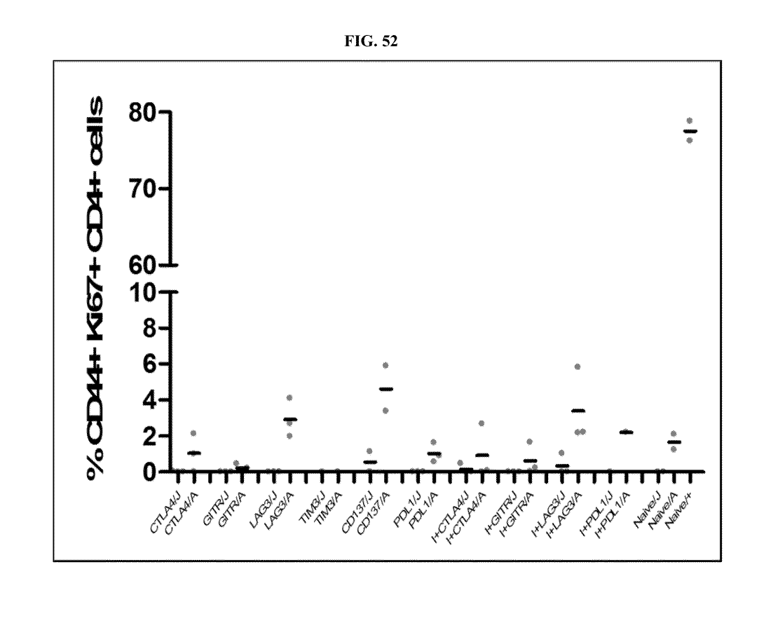

No significant secretion of IFN-γ was detected for the samples, even from cells stimulated with anti-CD3 and anti-CD28 antibodies. The readout for Ki67 was also observed upon specific tumor stimulation. Similarly, secretion was observed upon specific tumor stimulation in CD4+ cells, but was not observed in CD8 cells+. FIG. 52 exemplifies the level of immune checkpoint proteins, in CD44+ and Ki67+ cells.

### Example 15

### Tumor Growth Study in a J558 Mouse Model Using a Combination Therapy of Ibrutinib and Anti-PD-L1 Antibody

### Materials

Cell line J558 (ATCC TIB-6, Lot: 3638824), a plasmacytoma cell line from BALB/c, mouse expressing H-2d and PC.1 antigens, was obtained from ATCC. The cells were initially grown in DMEM supplemented with 10% FBS without 2-ME, but later the growth media was switched to RPMI-1640 with 10% FBS.

The rat IgG2b/k anti-mouse PD-L1 (clone 10F.9G2, Bio X Cell, Bio X Cell, cat #BE0101, Lot: 5089-4/0114, 5.2 mg/mL) was obtained from BioXcell, and diluted to 2 mg/mL in sterile Phosphate Buffered Saline (PBS). The rat IgG2b/k antibody (clone LTF-2, Bio X Cell, Bio X Cell, cat #BE0090, Lot: 4689-2/1013, 4.68 mg/mL) was obtained from BioXcell, and diluted to 2 mg/mL in sterile PBS.

Ibrutinib (PCI-32765) was prepared at 4.8 mg/mL in 0.5% methylcellulose and 0.1% SLS.

### Tumor Transplantation and Treatment

Mice were inoculated in the hind flank with 5 million J558 cells. The injection volume was 100 μL. Ibrutinib was administered via oral gavage, daily at 24 mg/kg on days 12 to 20 post-injection of J558 cells. Anti-PD-L1 antibody or the isotype controlled IgG, at 200 μg, were injected intraperitoneally on days 12, 14, 16, 18, and 20 post-injection. Most tumor volumes were less than about 100 mm³ when treatment was started.

### Results

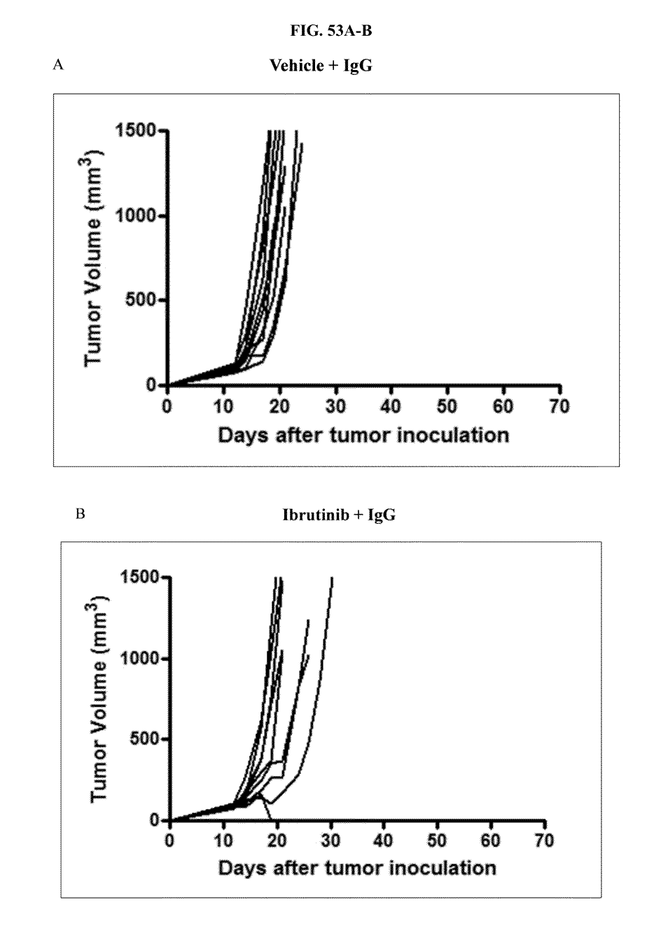

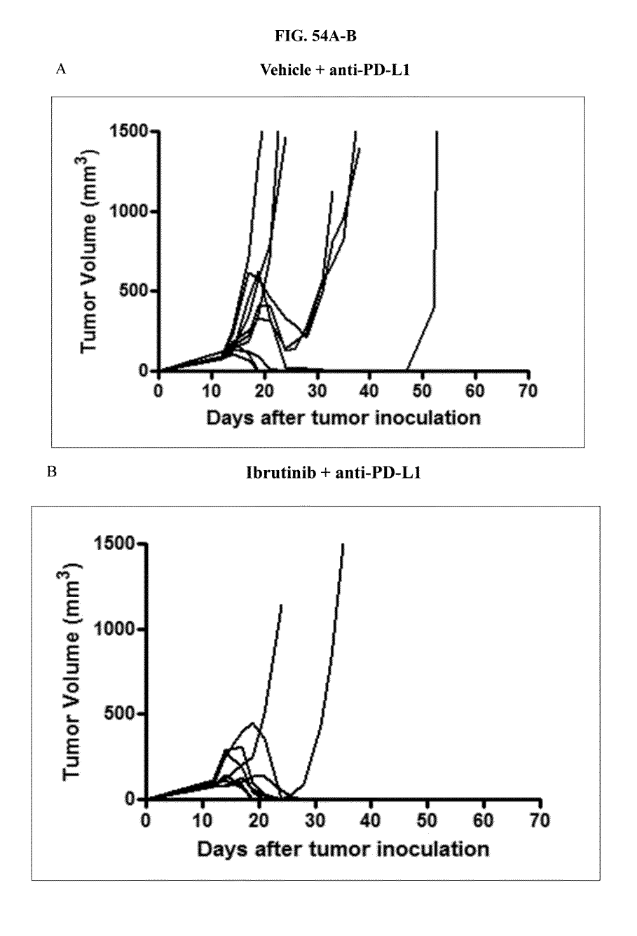

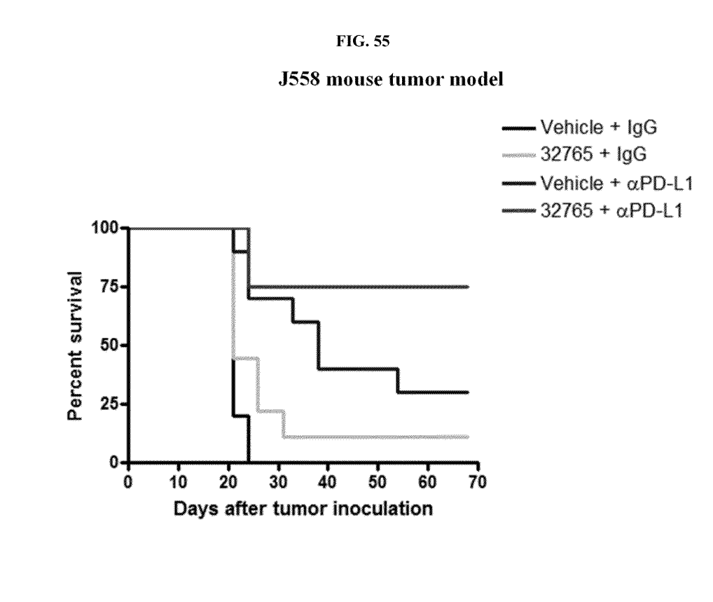

Tumor growth was not suppressed in mice treated with IgG alone or in combination with Ibrutinib (FIGS. 53A,B). Treatment with anti-PD-L1 alone had efficacy leading to tumor regression in four out of the nine mice belonging to this treatment group (FIG. 54A). In three cases regressions were not complete and tumor growth accelerated after treatment was stopped. In one mouse a late relapse was seen from completely regressed tumor. Ibrutinib, when administered in combination with the anti-PD-L1 antibody, resulted in an apparent synergistic effects, leading to regression of tumors in all but one mouse belonging to this treatment group (FIG. 54B). All the animals showed complete response except one which had a late relapse. Out of the ten mice in the ibrutinib and anti-PD-L1 antibody treatment group, two mice died and the remaining mice experienced body weight drop that was not observed in other groups. Six out of the eight mice, treated with a combination of anti-PD-L1 and ibrutinib, remained tumor free after day 20, and three out of the ten mice treated with anti-PD-L1 antibody alone remained tumor free after the same period of time, as exemplified in Table 8. Only one out of the ten mice treated with a combination of ibrutinib and IgG and none of the mice treated with IgG alone, were tumor free after day 20, also exemplified in Table 8.

TABLE 8
Tumor free mice after day 20-J558 model

| Treatment | Tumor Free Mice/Total No. of Mice |
|---|---|
| Vehicle + IgG | 0/10 |
| Ibrutinib + IgG | 1/9 |
| Vehicle + αPD-L1 | 3/10 |
| Ibrutinib + αPD-L1 | 6/8 |

### Example 16

### Tumor Specific T Cells Response in Mice Treated with a Combination of Ibrutinib and Anti-PD-L1 Antibody

### Materials and Methods

The Golden Syrian hamster IgG2/11 anti-mouse CD28 (clone 37.51, BD 553294) was prepared at 1 mg/mL. The Ar Hamster IgG1/k anti-mouse CD3e (clone 145-2C11, BD 553057, NA/LE) was prepared at 1 mg/mL. The mouse IgG2b/k anti-Ar and Syr hamster IgG1 (clone G94-56, BD 554005, NA/LE) was prepared at 1 mg/mL.

Cell culture grade Mitomycin C (Sigme M4287) was combined with NaCl, at a ratio of 1:24, in RPMI containing 10% FBS. LIVE/DEAD Fixable Aqua Stain (Invitrogen L34957) was equilibrated to room temperature, 50 μL DMSO was added to each vial and 1 μL of the mixture was added per 1 mL of PBS. Golgistop (BD 554724, containing monensis) was obtained from BD Biosciences, and the 1× concentration was 4 μL per 6 mL of culture.

All flow staining antibodies were anti-mouse monoclonal antibodies at 0.2 mg/mL and 1 uL was used per sample. The FITC-conjugate, was Rat IgG2b/k anti-CD4 (clone RM4-4, BD 553055). The Alexa Fluor 488 conjugate was Ar Hamster IgG anti-mouse CD3e (clone 145-2C11, eBio 53-0031-82). The PE-conjugates were Rat IgG2a/k anti-NKp46 (clone 29A1.4, eBio 12-3351-82) and Rat IgG2a/k anti-CD4 (clone H129.19, BD 553653). The PerCP-Cy5.5-conjugates were Rat IgG2b/k anti-CD44 (clone IM7, eBio 45-0441-82) and Rat IgG2a/k anti-CD4 (clone RM4-5, BD 553954). The PE-Cy7-conjugates were Rat IgG1/k anti-IFNγ (clone XMG1.2, eBio 25-7311-82) and Rat IgG2a/k anti-CD4 (clone RM4-5, BD 552775). The APC conjugate was Rat IgG2b/k anti-CD4 (clone GK1.5, eBio 17-0041-81). The APC-Cy7 conjugates were Rat IgG2a/k anti-CD8a (clone 53-6.7, BD 557654) and Rat IgG2b/k anti-CD4 (clone GK1.5,BD 552051). The e450 conjugate was Ar Hamster IgG anti-CD69 (clone H1.2F3, eBio 48-0691-82). The BV421 conjugate was Rat IgG2a/k anti-CD4 (clone RM4-5, BD 560468).

Spleens were collected from selected treated mice and suspended in RPMI containing 10% FBS. Spleens were reduced to single cell suspension on 70 μm strainers. Splenocytes were washed with 10 mL RPMI-1640 and resuspended in 5 mL RPMI-1640. A 2 mL volume of Lympholyte M was added below the suspension. The splenocytes were centrifuged at 2000 rpm for 20 minutes. The cells were cultured in plain media. To treat target cells with mytomycin C, 100 million J558 or A20 cells were resuspended in 1.425 mL of RPMI containing 10% FBS with 2-ME. Mitomycin C was added at 50 μg/mL and incubated for 20 minutes. The cells were washed twice with media. A set of three samples, each containing 500,000 splenocytes, were stimulated with either media and 0.5 μg anti-CD28 antibody, media with 0.5 μg anti-CD28 antibody and 500,000 J558 cells, or media with 0.5 μg anti-CD28 antibody and 500,000 A20 cells. A positive control was also set up where 500,000 splenocytes were incubated with media and 0.5 μg anti-CD28, 0.25 μg anti-CD3, and 1 μg anti-hamster. The cells were incubated for 24 hours, the last 5 hours being in the presence of GolgiStop.

### Flow Cytometry

The cells, after stimulation as described above, were harvested, centrifuged and blocked with 0.5 μg/sample Fc blocker in 2% FBS/PBS (FW) for 10 minutes over ice. A cocktail of fluorochrome antibodies comprising antiCD3-FITC, anti-CD4-APC, anti-CD8-APC-Cy7, anti-CD69-eF450, anti-NKp46-PE, anti-CD44-PerCP-Cy5.5 was added at 50 μL per sample. Aqua was also added after a short delay. The cells were permeabilized with the Fixation/Permeabilization (F/P) solution (eBio 00-5523-00), stored overnight in the dark at 4° C. The cells were washed with Permeabilization buffer/wash (PW)(ebio 00-8333-56), resuspended in 100 μL PW containing 2% Rat Serum for 15 minutes at room temperature. A 50 μL volume of PW with anti-IFNg-PE-Cy7 was added to the cells and incubated at room temperature in dark for 30 minutes. The cells were washed first with PW and then with FW, and fixed with 200 μL of 2% PFA. Compensation tubes and Fluorescence minus one (FMOs) were made from a pool of splenocytes in each set. Compensations were stained with CD4 fluorochrome antibodies and heat killed splenocytes were stained with Aqua. Samples were acquired on Cantoll several days later. The cells were analyzed by flow cytometry on a FACSCalibur (BD Biosciences). FACS data were analyzed using Cytobank.

### Results

CD44+ splenic T cells from cured mice were observed to have increased IFN-γ production after overnight incubation with J558 cells when compared with media alone, and were observed to have similar increased level when compared to overnight incubation with A20 cells. It was observed that CD8+ cells had more robust IFN-γ response than CD4+ cells.

The examples and embodiments described herein are illustrative and various modifications or changes suggested to persons skilled in the art are to be included within this disclosure. As will be appreciated by those skilled in the art, the specific components listed in the above examples may be replaced with other functionally equivalent components, e.g., diluents, binders, lubricants, fillers, and the like.

What is claimed is:

1. A method for treating a cancer comprising administering to a subject in need thereof a combination comprising a BTK inhibitor and an immune checkpoint inhibitor.

2. The method of claim 1, wherein the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof.

3. The method of claim 2, wherein the immune checkpoint inhibitor is an inhibitor of PD-L1, PD-1, CTLA4, LAG3, or TIM3.

4. The method of claim 1, wherein the cancer is a hematologic cancer, a sarcoma, or a carcinoma.

5. The method of claim 4, wherein the cancer is selected from diffuse large B-cell lymphoma (DLBCL), chronic lymphocytic leukemia (CLL), small lymphocytic lymphoma (SLL), B cell prolymphocytic leukemia (B-PLL), non-CLL/SLL lymphoma, mantle cell lymphoma, multiple myeloma, Waldenström's macroglobulinemia, bladder cancer, breast cancer, colon cancer, gastroenterological cancer, kidney cancer, lung cancer, ovarian cancer, pancreatic cancer, prostate cancer, proximal or distal bile duct cancer, melanoma, or a combination thereof.

6. The method of claim 1, wherein the cancer is a relapsed or refractory cancer, or a metastasized cancer.

7. The method of claim 1, wherein the BTK inhibitor is ibrutinib.

8. The method of claim 1, wherein the method further comprises administering an additional anticancer agent.

9. A pharmaceutical combination comprising:
a) a BTK inhibitor; and
b) an immune checkpoint inhibitor; and
c) a pharmaceutically-acceptable excipient.

10. The pharmaceutical combination of claim 9, wherein the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof.

11. The pharmaceutical combination of claim 9, wherein the BTK inhibitor is ibrutinib.

12. The pharmaceutical combination of claim 9, further comprising an additional anticancer agent.

13. A method of treating an ibrutinib-resistant cancer comprising administering to a subject in need thereof a combination comprising ibrutinib and an immune checkpoint inhibitor.

14. The method of claim 13, wherein the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof.

15. The method of claim 13, wherein the ibrutinib-resistant cancer is a hematologic cancer, a sarcoma, or a carcinoma.

16. The method of claim 15, wherein the ibrutinib-resistant cancer is selected from diffuse large B-cell lymphoma (DLBCL), chronic lymphocytic leukemia (CLL), small lymphocytic lymphoma (SLL), B cell prolymphocytic leukemia (B-PLL), non-CLL/SLL lymphoma, mantle cell lymphoma, Waldenström's macroglobulinemia, bladder cancer, breast cancer, colon cancer, gastroenterological cancer, kidney cancer, lung cancer, ovarian cancer, pancreatic cancer, prostate cancer, proximal or distal bile duct cancer, melanoma, or a combination thereof.

17. The method of claim 13, wherein the ibrutinib-resistant cancer is a relapsed or refractory cancer, or a metastasized cancer.

18. A method for treating a cancer comprising administering to a subject in need thereof a combination comprising a BTK inhibitor and an inhibitor of PD-L1, PD-1, or CTLA-4.

19. A pharmaceutical combination comprising:
a) a BTK inhibitor; and
b) an inhibitor of PD-L1, PD-1, or CTLA-4; and
c) a pharmaceutically-acceptable excipient.

20. A method for treating an ibrutinib-resistant cancer comprising administering to a subject in need thereof a combination comprising ibrutinib and an inhibitor of PD-L1, PD-1, or CTLA-4.
